(19) United States
(12) Patent Application Publication
(10) Pub. No.: US 2025/0320234 A1
(43) Pub. Date: Oct. 16, 2025

(54) LONP1 INHIBITOR COMPOUNDS, USES AND METHODS

(71) Applicant: Pretzel Therapeutics, Inc., Waltham, MA (US)

(72) Inventor: Jeremy Green, Waltham, MA (US)

(73) Assignee: Pretzel Therapeutics, Inc., Waltham, MA (US)

(21) Appl. No.: 18/716,909

(22) PCT Filed: Dec. 6, 2022
(86) PCT No.: PCT/US2022/052003

§ 371 Date: Jun. 5, 2024

Related U.S. Application Data
(60) Provisional application No. 63/286,441, filed on Dec. 6, 2021.
(60) Provisional application No. 63/290,841, filed on Dec. 17, 2021.
(60) Provisional application No. 63/319,596, filed on Mar. 14, 2022.
(60) Provisional application No. 63/358,741, filed on Jul. 6, 2022.

(51) Int. Cl. C07F 5/02; A61K 31/69; A61P 35/00

(52) U.S. Cl. CPC C07F 5/027; A61K 31/69; A61P 35/00

(57) ABSTRACT

Disclosed are compounds according to Formula (I) which inhibit LONP1, and pharmaceutical compositions comprising compounds of the disclosure. Compounds and pharmaceutical compositions of the disclosure may be useful for the treatment of diseases and disorders associated with LONP1, including oncologic diseases and disorders, such as cancer, and diseases and disorders related to mitochondrial dysfunction, such as neurodegenerative disorders, metabolic disorders, and diseases associated with the aging process. The disclosure also relates to methods of using such compounds and compositions for the treatment of such diseases and disorders.

### FIELD OF THE INVENTION

The present invention relates to novel LONP1 inhibitors, their pharmaceutically acceptable salts, and pharmaceutical compositions thereof. The present invention also relates to methods of using such compounds and compositions, including to inhibit LONP1 and to treat oncologic diseases and disorders, such as cancer, and various diseases and disorders related to mitochondrial dysfunction, such as neurodegenerative disorders, metabolic disorders, and diseases associated with the aging process.

### BACKGROUND OF THE INVENTION

The mitochondrial Lon serine protease, LONP1, is an enzyme that is a member of the AAA+ superfamily of proteases (i.e., ATP-dependent proteases (ATPases) associated with diverse cellular activities). Widely conserved across eukaryotic species, human LONP1 is a 959-amino acid protein that consists of three domains: the N-terminal domain involved in substrate binding, the AAA+ (ATPase) domain, and the C-terminal domain (named the P-domain) involved in proteolytic activity. The ATPase and protease domains are the most well-conserved across species, while the N-terminal domain is the most variable.

LONP1 performs at least four different functions: proteolysis of damaged and oxidized proteins of the mitochondrial matrix; chaperone activity, namely the correct folding of proteins imported into the mitochondria; regulation of mitochondrial protein levels, including mitochondrial transcription factor A (TFAM); and binding to mitochondrial DNA (“mtDNA”) and RNA. As for the proteolytic activity of LONP1, like all the other proteases in the AAA+ family, it binds its substrate, unfolds it using the ATPase domain, and then digests it from the N or C-terminus. Its chaperone activity, mediated by the ATP-binding domain and the N-terminal domain, is crucial for mitochondrial homeostasis, as it is involved in the assembly of mitochondrial membrane complexes.

LONP1 has multiple, natural substrates, one of which is the mtDNA binding and packaging protein TFAM. TFAM has a crucial role in transcription initiation and mtDNA replication. Inhibition of LONP1 reportedly leads to increased levels of the TFAM protein, which in turn may lead to higher levels of mtDNA.

TFAM and mtDNA have a mutual dependence for stability, whereby TFAM binds mtDNA and protects it from degradation, but when not bound to mtDNA, TFAM is rapidly degraded. LONP1 has been shown to regulate mtDNA copy number in Drosophila melanogaster by cleaving TFAM.

In human cells with severe mtDNA deficits, depletion of LONP1 can increase levels of TFAM and upregulate mtDNA content.

Another natural substrate of LONP1 is POLγA, the catalytic subunit of DNA polymerase γ (POLγ). POLγ is the main protein responsible for mitochondrial DNA (mtDNA) replication. The accessory POLγB subunit acts to stabilize POLγA and to prevent LONP1-dependent degradation. Disease causing mutations such as A467T weaken interactions between POLγA and POLγB, which in turn makes POLγA susceptible to degradation by LONP1.

LONP1 is also required during embryogenesis. A homozygous deletion of the LONP1 gene in a mouse causes embryonic lethality. In line with this observation, mutations that change LONP1 activity during embryogenesis can cause a congenital syndrome known as CODAS, characterized by Cerebral, Ocular, Dental, Auricular and Skeletal anomalies. Further supporting a role during embryogenesis, defective mitochondrial protease LONP1 has also been linked to a classical, congenital mitochondrial disease. The mutant (Tyr565His) protein displayed higher ATPase activity, but reduced protease activity. See Peter, B. et. al., “Defective Mitochondrial Protease LonP1 Can Cause Classical Mitochondrial Disease,” Hum. Mol. Genet., 27, 10, 1743-1750 (2018).

Additionally, LONP1 has a central role in the regulation of mitochondrial function, impacting bioenergetics in various cells and often causing disease (see Gibellini L. et. al., “LonP1 Differently Modulates Mitochondrial Function and Bioenergetics of Primary Versus Metastatic Colon Cancer Cells,” Front. Oncol. 8, 254 (2018). LONP1 upregulation is a characteristic shared by various types of cancer cells. Higher expression of LONP1 is correlated with tumor progression and aggressiveness. For instance, LONP1 overproduction is functionally linked to colorectal cancer cells by inducing the epithelial mesenchymal transition, an early step in the formation of metastases (see id). Furthermore, LONP1 is a regulator of mitochondrial proteostasis, which is required for maintaining the respiratory chain and degrading misfolded, oxidatively damaged or unassembled proteins. As such, inhibition of LONP1 is believed to be a mechanism by which various oncogenic diseases, such as cancers may be treated.

Similarly, multiple myeloma is an exceedingly prevalent and incurable cancer in the elderly (see Maneix, L. et al., “The Mitochondrial Protease LonP1 Promotes Proteasome Inhibitor Resistance in Multiple Myeloma,” Cancers 13, 843, 14-19 (2021)). Proteasome inhibitors are a common treatment for myeloma, but for unknown reasons, over time, a resistance to treatment develops. Compounds that inhibit LONP1 may provide a means to more thoroughly understand the molecular mechanisms that lead to such drug resistance in the treatment of multiple myeloma (see id).

While aspects of LONP1 biochemistry are known, its full physiological role in mitochondrial gene expression and homeostasis, as well as its underlying impact in the etiology of various disease states, remains unclear. LONP1 inhibitors will provide insight into, for example, the relationship between LONP1, mtDNA copy number, and human diseases. Pharmacological inhibition of LONP1 is one means by which to gain a further understanding of the role of this protease in cell physiology and the development of disease. LONP1 inhibitors have been reported, for example, in Kingsley, L. J. et al., J. Med. Chem. 64, 8, 4857-4869 (2021). In view of the numerous and varied roles of LONP1, there is a need for additional, potent, and specific inhibitors of LONP1.

### SUMMARY OF THE INVENTION

Provided are compounds, pharmaceutically acceptable salts of the compounds, pharmaceutical compositions comprising the compounds or their salts, methods of using the compounds, salts of the compounds, or pharmaceutical compositions of the compounds or their salts, and therapeutic uses of the compounds, or pharmaceutical compositions of the compounds or their salts, for treating diseases related to oncologic diseases and disorders, such as cancer, and/or various diseases and disorders related to mitochondrial dysfunction, such as neurodegenerative disorders, metabolic disorders, and diseases associated with the aging process. The compounds and their pharmaceutically acceptable salts are particularly useful as inhibitors of LONP1.

In one aspect there is provided a compound of structural Formula I:

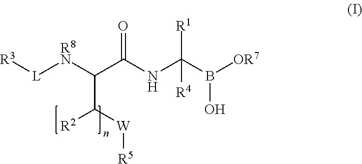

or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C1-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹¹, CONR¹¹R¹², NR¹¹R¹², SR¹¹, SO2NR¹¹R¹², C1-C4 alkyl, C1-C4 haloalkyl, C1-C4 alkoxy, phenyl, or a 5- or 6-membered heteroaryl;
n is 1 or 2;
each occurrence of R² is independently selected from H or C1-C4 alkyl;
L is C(O), C(O)O, C(O)NR⁶, S(O)₂, or a bond;
R³ is C1-C4 alkyl optionally substituted with one or more substituents each independently selected from the group consisting of deuterium, halogen, cyano, hydroxyl, C1-C4 alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C1-C4 alkoxyl, or C1-C4 alkyl that is optionally substituted with one to three substituents selected from deuterium, halogen, cyano, hydroxyl, or C1-C4 alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹¹, CONR¹¹R¹², NR¹¹R¹², SR¹¹, SO2NR¹¹R¹², C₁-C₄ alkoxyl, or C₁-C₄ alkyl that is optionally substituted with one to three substituents selected from deuterium, halogen, cyano, hydroxyl, or C₁-C4 alkoxyl;
W is selected from the group consisting of O, S, S(O), SO₂ and S(O)(NH);
R⁴ is selected from hydrogen, deuterium or C₁-C₂ alkyl;
R⁵ is hydrogen, deuterium, C₁-C₄ alkyl or C₁-C₄ alkoxyl wherein each alkyl or alkoxyl is optionally substituted with one or more substituents each independently selected from the group consisting of deuterium, halogen, cyano, hydroxyl, C₁-C₄ alkoxyl, C₁-C₅ alkyl-alkoxyl or phenyl; or
R⁵ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is NR⁹R¹⁰;
R⁶ is hydrogen, deuterium or C₁-C₂ alkyl optionally substituted with one or more of deuterium, halogen, hydroxyl and phenyl, wherein phenyl is optionally substituted with one or more substituent selected from halogen, hydroxyl and C₁-C₂ alkyl;
R⁷ is hydrogen, or R⁷ and R¹, together with the boron atom to which OR⁷ is attached form a 5-membered heteroalkyl ring;
R⁸ is selected from hydrogen, deuterium, or C₁-C₂ alkyl optionally substituted with one or more substituents each independently selected from the group consisting of halogen, hydroxyl, cyano, methoxyl and phenyl; or
R⁸, L and R³ together with the N to which R⁸ and L are attached form a saturated or unsaturated heterocycloalkyl group optionally having one or more additional heteroatoms selected from N, O and S, wherein the heterocycloalkyl is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl, and is optionally fused to an aryl or heteroaryl group which is optionally substituted with one or more substituents selected from halogen, cyano, hydroxyl, C₁-C₄ alkoxyl, or C₁-C₄ alkyl, wherein C₁-C₄ alkyl is optionally substituted with one to three substituents selected from halogen, cyano and C₁-C₄ alkoxyl, or is optionally fused to a cycloalkyl or heterocycloalkyl group which is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl;
R⁹ and R¹⁰ are each independently selected from hydrogen or C₁-C₆ alkyl that is optionally substituted with one to three substituents selected from halogen, cyano, or C₁-C₄ alkoxyl; and
R¹¹ and R¹² are each independently selected from hydrogen, deuterium, C1-C4 alkyl; C1-C4 haloalkyl, C1-C5 alkyl-alkoxyl, C3-C7 cycloalkyl, or R¹¹ and R¹² together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

Embodiments of the present disclosure include compounds of the disclosure (that is, compounds of Formula I) or their pharmaceutically acceptable salts wherein one or more hydrogen atom is substituted with a deuterium atom.

Another aspect of the disclosure is directed to pharmaceutical compositions comprising a compound of the disclosure (that is, compounds of Formula I) or a pharmaceutically acceptable salt thereof and a pharmaceutically acceptable excipient.

Other aspects of the disclosure are directed to methods of treating a disease or disorder, such as a disease or disorder characterized by mitochondrial dysfunction, such methods comprising administering to a subject in need thereof a therapeutically effective amount of a compound of the disclosure, a pharmaceutically acceptable salt thereof, or a composition comprising such as compound.

In various aspects and embodiments of the methods and therapeutic uses disclosed herein, the disease is selected from Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome. In some aspects and embodiments the disease or disorder is selected from Alzheimer's disease, Parkinson's disease, obesity, diabetes, non-alcoholic steatohepatitis (NASH), and related metabolic syndromes such as non-alcoholic fatty liver disease (NAFLD).

Other aspects of the disclosure are directed to compounds or (pharmaceutical) compositions comprising compounds of the disclosure for use in methods for treating a disease or disorder, such as a disease or disorder characterized by mitochondrial dysfunction. These therapeutic uses may comprise administering to a subject in need thereof a therapeutically effective amount of a compound of the disclosure, a pharmaceutically acceptable salt thereof, or a composition comprising such a compound. Suitable diseases or disorders are those described above and herein below.

In some embodiments, the disease to be treated with a compound or composition of the disclosure is associated with mtDNA mutations or deletions, for example: m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G and m.1555A>G (see https://mitomap.org/MITOMAP).

Additional aspects and embodiments of the disclosure relate to methods of treating cancers and compounds or compositions for use in such methods: for example, those identified in Wong, K. S. et al. “Recent Advances in Targeting Human Mitochondrial AAA+ Proteases to Develop Novel Cancer Therapeutics,” Advances in Experimental Medicine and Biology, 1158, 119-142 (2019), wherein the use or method comprising using a compound or composition of the disclosure or its pharmaceutically acceptable salt.

Further aspects and embodiments of the disclosure relate to methods of treating cancer, neurodegenerative disorders, metabolic disorders, and diseases associated with the aging process; and compounds and compositions of the disclosure for use in such methods.

Within the scope of this disclosure it is expressly intended that the various aspects, embodiments, examples and alternatives set out in the preceding paragraphs, in the claims and/or in the following description, and in particular the individual features thereof, may be taken independently or in any combination. That is, all embodiments and/or features of any aspect or embodiment can be combined in any way and/or combination, unless such features are incompatible. More particularly, it is specifically intended that any embodiment of any aspect may form an embodiment of any other aspect, and all such combinations are encompassed within the scope of the disclosure. The applicant reserves the right to change any originally filed claim or file any new claim accordingly, including the right to amend any originally filed claim to depend from and/or incorporate any feature of any other claim although not originally claimed in that manner.

### DETAILED DESCRIPTION OF THE INVENTION

Described herein are compounds and compositions (e.g. organic molecules, research tools, pharmaceutical formulations and therapeutics): uses for the compounds and compositions of the disclosure (in vitro and in vivo); as well as corresponding methods, whether diagnostic, therapeutic or for research applications. The chemical synthesis and biological testing of the compounds of the disclosure are also described. Beneficially, the compounds, compositions, uses and methods have utility in research towards and/or the treatment of diseases or disorders in animals, such as humans. Diseases or disorders which may benefit from LONP1 modulation include mitochondrial diseases, cancer and/or oncologic disease.

However, the compounds of the disclosure may also or alternatively be useful as lead molecules for the selection, screening and development of further derivatives that may have one or more improved beneficial drug property, as desired.

The disclosure also encompasses salts, solvates and functional derivatives of the compounds described herein. These compounds may be useful in the treatment of diseases or disorders characterized by mitochondrial disfunction; particularly those which may benefit from LONP1 inhibition.

Inhibitors of LONP1 are useful in compositions and methods suitable for treating many disorders, such as disorders characterized by mitochondrial dysfunction, including cancer. In some embodiments, the disease is selected from the group consisting of adrenal gland cancer, anal cancer, adenocarcinoma, angiosarcoma, bile duct cancer, bladder cancer, blastic plasmacytoid dendritic cell neoplasm, bone cancer, brain cancer, breast cancer, bronchogenic carcinoma, central nervous system (CNS) cancer, cervical cancer, cholangiocarcinoma, chondrosarcoma, colon cancer, choriocarcinoma, colorectal cancer, cancer of connective tissue, esophageal cancer, embryonal carcinoma, fibrosarcoma, gall bladder cancer, gastric cancer, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas), melanoma, Merkel cell carcinoma, mesothelioma, multiple myeloma, muscular cancer, myxosarcoma, neuroblastomas, non-small cell lung cancer, ocular cancer, oral/digestive tract cancer, osteogenic sarcoma, ovarian cancer, papillary carcinoma, pancreatic cancer, polycythemia vera, prostate cancer, rhabdomyosarcoma, renal cancer, retinal cancer, skin cancer, small cell lung carcinoma, stomach cancer, testicular cancer, throat cancer, thyroid cancer, uterine cancer, vaginal cancer, and vulvar cancer. In some embodiments, the disease is selected from the group consisting of Alzheimer's disease and Parkinson's disease. In some embodiments, the disease is selected from the group consisting of obesity, diabetes, non-alcoholic steatohepatitis (NASH), and related metabolic syndromes such as non-alcoholic fatty liver disease (NAFLD). In some embodiments, the disease is related to aging or a mitochondrial disorder.

Provided herein are compounds of Formula I, pharmaceutically acceptable salts thereof, and pharmaceutical compositions comprising such compounds or their salts that are useful in treating a condition or disease characterized by mitochondrial dysfunction.

### Definitions

Unless defined otherwise, all technical and scientific terms used herein have the same meaning as commonly understood by one of ordinary skill in the art (e.g. in organic, physical or theoretical chemistry; biochemistry and molecular biology).

Unless otherwise indicated, the practice of the present invention employs conventional techniques in chemistry and chemical methods, biochemistry, molecular biology, pharmaceutical formulation, and delivery and treatment regimens for patients, which are within the capabilities of a person of ordinary skill in the art. Such techniques are also described in the literature cited herein. All documents cited in this disclosure are herein incorporated by reference in their entirety.

Prior to setting forth the detailed description of the inventions, a number of definitions are provided that will assist in the understanding of the disclosure.

In accordance with this disclosure, the terms ‘molecule’ or ‘molecules’ are used interchangeably with the terms ‘compound’ or ‘compounds’, and sometimes the term ‘chemical structure’. The term ‘drug’ is typically used in the context of a pharmaceutical, pharmaceutical composition, medicament or the like, which has a known or predicted physiological or in vitro activity of medical significance; but such characteristics and qualities are not excluded in a molecule or compound of the disclosure. The term ‘drug’ is therefore used interchangeably with the alternative terms and phrases ‘therapeutic (agent)’, ‘pharmaceutical (agent)’, and ‘active (agent)’. Therapeutics according to the disclosure also encompass compositions and pharmaceutical formulations comprising the compounds of the disclosure.

Prodrugs and solvates of the compounds of the disclosure are also encompassed within the scope of the disclosure. The term ‘prodrug’ means a compound (e.g. a drug precursor) that is transformed in vivo to yield a compound of the disclosure or a pharmaceutically acceptable salt, solvate or ester of the compound. The transformation may occur by various mechanisms (e.g. by metabolic or chemical processes), such as by hydrolysis of a hydrolysable bond, e.g. in blood (see Higuchi & Stella (1987), “Pro-drugs as Novel Delivery Systems”, vol. 14 of the A.C.S. Symposium Series; (1987), “Bioreversible Carriers in Drug Design”, Roche, ed., American Pharmaceutical Association and Pergamon Press). The compositions and medicaments of the disclosure therefore may comprise prodrugs of the compounds of the disclosure. In some aspects and embodiments the compounds of the disclosure may be themselves prodrugs which may be metabolised in vivo to give the therapeutically effective compound.

The scope of this disclosure also includes various deuterated forms of the compounds of any of Formula I (inc. corresponding subgeneric formulas defined herein), respectively, or a pharmaceutically acceptable salt and/or a corresponding tautomer form thereof (including subgeneric formulas, as defined above). Each available hydrogen atom attached to a carbon atom may be independently replaced with a deuterium atom. A person of ordinary skill in the art will know how to synthesize deuterated forms of the compounds of Formula I disclosed herein (including subgeneric formulas, as defined above) or a pharmaceutically acceptable salt and/or a corresponding tautomer form thereof (including subgeneric formulas, as defined above) of the present disclosure. For example, deuterated materials, such as alkyl groups may be prepared by conventional techniques (see for example: methyl-d₃-amine available from Aldrich Chemical Co., Milwaukee, WI, Cat. No. 489, 689-2).

The disclosure also includes isotopically-labelled compounds which are identical to those recited in Formula I disclosed herein (inc. corresponding subgeneric formulas defined herein), respectively, or a pharmaceutically acceptable salt and/or a corresponding tautomer form thereof (including subgeneric formulas, as defined above), but for the fact that one or more atoms are replaced by an atom having an atomic mass or mass number different from the atomic mass or mass number most commonly found in nature. Examples of isotopes that can be incorporated into compounds of the disclosure include isotopes of hydrogen, carbon, nitrogen, oxygen, fluorine, iodine and chlorine such as 2 H, 3 H, 11 C, 14 C, 18 F, 123 I or 125 I. Compounds of the present disclosure and pharmaceutically acceptable salts of said compounds that contain the aforementioned isotopes and/or other isotopes of other atoms are within the scope of the present disclosure. Isotopically labelled compounds of the present disclosure, for example those into which radioactive isotopes such as 3 H or 14 C have been incorporated, are useful in drug and/or substrate tissue distribution assays. Tritiated, i.e. 3 H, and carbon-14, i.e. 14 C, isotopes are particularly preferred for their ease of preparation and detectability. 11 C and 18 F isotopes are particularly useful in PET (positron emission tomography).

In the context of the present disclosure, the terms ‘individual’, ‘subject’, or ‘patient’ are used interchangeably to indicate an animal that may be suffering from a medical (pathological) condition and may be responsive to a compound/molecule, pharmaceutical drug, medical treatment or therapeutic treatment regimen of the disclosure. The animal is suitably a mammal, such as a human, cow, sheep, pig, dog, cat, bat, mouse or rat. In particular, the subject may be a human.

The term ‘alkyl’ refers to a monovalent, optionally substituted, saturated aliphatic hydrocarbon radical. Any number of carbon atoms may be present, but typically the number of carbon atoms in the alkyl group may be from 1 to about 20, from 1 to about 12, from 1 to about 6 or from 1 to about 4. Usefully, the number of carbon atoms is indicated, for example, a C1-C12 alkyl (or C₁-C₁₂ alkyl) refers to any alkyl group containing 1 to 12 carbon atoms in the chain. An alkyl group may be a straight chain (i.e. linear), branched chain, or cyclic. ‘Lower alkyl’ refers to an alkyl of 1 to 6 carbon atoms in the chain, and may have from 1 to 4 carbon atoms, or 1 to 2 carbon atoms. Thus, representative examples of lower alkyl radicals include methyl, ethyl, n-propyl, n-butyl, n-pentyl, n-hexyl, isopropyl, isobutyl, isopentyl, amyl (C₅H₁₁), sec-butyl, tert-butyl, sec-amyl, tert-pentyl, 2-ethylbutyl, 2,3-dimethylbutyl, and the like. ‘Higher alkyl’ refers to alkyls of 7 carbons and above, including n-heptyl, n-octyl, n-nonyl, n-decyl, n-dodecyl, n-tetradecyl, n-hexadecyl, n-octadecyl, n-eicosyl, and the like, along with branched variations thereof. A linear carbon chain of say 4 to 6 carbons would refer to the chain length not including any carbons residing on a branch, whereas in a branched chain it would refer to the total number. Optional substituents for alkyl and other groups are described below.

The term ‘alkoxy’ or ‘alkoxyl’ as used herein refers to a monovalent radical of the formula RO—, where R is any alkyl, alkenyl or alkynyl as defined herein. Alkoxy groups may be optionally substituted by any of the optional substituents described herein. ‘Lower alkoxy’ has the formula RO—, where the R group is a lower alkyl, alkenyl or alkynyl. Representative alkoxy radicals include methoxyl, ethoxyl, n-propoxy, n-butoxyl, n-pentyloxyl, n-hexyloxyl, isopropoxyl, isobutoxyl, isopentyloxyl, amyloxyl, sec-butoxyl, tert-butoxyl, tert-pentyloxyl, and the like. Preferred alkoxyl groups are methoxyl and ethoxyl.

The term ‘cycloalkyl’ as used herein refers to a cyclized alkyl ring having the indicated number of carbon atoms in a specified range. Thus, for example, “C₃-C₆ cycloalkyl” encompasses each of cyclopropyl, cyclobutyl, cyclopentyl, and cyclohexyl.

The term ‘aryl’ as used herein refers to a substituted or unsubstituted aromatic carbocyclic radical containing from 5 to about 15 carbon atoms (“C₆-C₁₅ aryl”); and preferably 6 to 12 carbon atoms (“C₆-C₁₂ aryl”). An aryl group may have only one individual carbon ring, or may comprise one or more fused rings in which at least one ring is aromatic in nature. A ‘phenyl’ is a radical formed by removal of a hydrogen atom from a benzene ring, and may be substituted or unsubstituted. A ‘phenoxy’ group, therefore, is a radical of the formula RO—, wherein R is a phenyl radical. ‘Benzyl’ is a radical of the formula R—CH₂—, wherein R is phenyl, and ‘benzyloxyl’ is a radical of the formula RO—, wherein R is benzyl. The point of attachment to the base molecule on such fused aryl ring systems may be a C atom of the aromatic portion or a C or a N atom of the non-aromatic portion of the ring system. Non-limiting examples of aryl radicals include, phenyl, naphthyl, anthracenyl, benzyl, biphenyl, furanyl, pyridinyl, indanyl, anthraquinolyl, tetrahydronaphthyl, a benzoic acid radical, a furan-2-carboxylic acid radical, and the like.

The term ‘cycloaryl’ herein refers to a polycyclic group wherein an aryl group is fused to a 5- or 6-membered aliphatic ring. For example, C₆-C₁₂ cycloaryl means a C₆-C₁₂ aryl fused to a 5- or 6-membered aliphatic ring.

The term ‘heteroaryl’ as used herein refers to (i) a 5- or 6-membered ring having the characteristics of aromaticity containing at least one heteroatom selected from N, O and S, wherein each N is optionally in the form of an oxide, and (ii) a 9- or 10-membered bicyclic fused ring system, wherein the fused ring system of (ii) contains at least one heteroatom independently selected from N, O and S, wherein each ring in the fused ring system contains zero, one or more than one heteroatom, at least one ring is aromatic, each N is optionally in the form of an oxide, and each S in a ring which is not aromatic is optionally S(O) or S(O)₂. Typically, heteroaryl groups contain 5 to 14 ring atoms (‘5-14 membered heteroaryl’), and preferably 5 to 12 ring atoms (‘5-12 membered heteroaryl’). Heteroaryl rings are attached to the base molecule via a ring atom of the heteroaromatic ring, such that aromaticity is maintained. Suitable 5- and 6-membered heteroaromatic rings include, for example, pyridyl, 3-fluroropyridyl, 4-fluoropyridyl, 3-methoxypyridyl, 4-methoxypyridyl, pyrrolyl, pyrazinyl, pyrimidinyl, pyridazinyl, triazinyl, thienyl, furanyl, imidazolyl, pyrazolyl, triazolyl (i.e., 1, 2, 3-triazolyl or 1, 2, 4-triazolyl), tetrazolyl, oxazolyl, isooxazolyl, oxadiazolyl (i.e., the 1, 2, 3-, 1, 2, 4-, 1, 2, 5-(furazanyl), or 1, 3, 4-isomer), oxatriazolyl, thiazolyl, isothiazolyl, and thiadiazolyl. Suitable 9- and 10-membered heterobicyclic, fused ring systems include, for example, benzofuranyl, indolyl, indazolyl, naphthyridinyl, isobenzofuranyl, benzisoxazolyl, benzoxazolyl, benzothiazolyl, chromenyl, quinolinyl, isoquinolinyl, benzopiperidinyl, benzofuranyl, imidazo[1, 2-a]pyridinyl, benzotriazolyl, indazolyl, indolinyl, and isoindolinyl.

The term ‘heteroaryloxy’ or ‘heteroaryloxyl’ as used herein refers to an —O— heteroaryl group.

The terms ‘heterocycle’ or ‘heterocyclic’ group or ‘heterocyclyl’ as used herein refer to a monovalent radical of from about 4- to about 15-ring atoms, and preferably 3-, 4-, 5-, 6-, 7-, 8-, 9- or 10-ring members. Generally, the heterocyclic group contains one, two or three heteroatoms, selected independently from nitrogen, oxygen and sulphur. A preferred heteroatom is N. A heterocyclic group may have only one individual ring or may comprise one or more fused rings in which at least one ring contains a heteroatom. It may be fully saturated or partially saturated and may be substituted or unsubstituted as in the case or aryl and heteroaryl groups. Representative examples of unsaturated 5-membered heterocycles with only one heteroatom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolindinyl, 2- or 3-tetrahydrofuranyl, and 2- or 3-tetrahydrothiophenyl. Representative unsaturated 5-membered heterocyclic radicals having two heteroatoms include imidazolyl, oxazolyl, thiazolyl, pyrazolyl, and the like. The corresponding fully saturated and partially saturated radicals are also included. Representative examples of unsaturated 6-membered heterocycles with only one heteroatom include 2-, 3-, or 4-pyridinyl, 2H-pyranyl, and 4H-pryanyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two heteroatoms include 3- or 4-pyridazinyl, 2-, 4-, or 5-pyrimidinyl, 2-pyrazinyl, morpholino, and the like. The corresponding fully saturated and partially saturated radicals are also included, e.g. 2-piperazine. The heterocyclic radical is bonded through an available carbon atom or heteroatom in the heterocyclic ring directly to the entity or through a linker such as an alkylene such as methylene or ethylene.

The term ‘substituted’ means that one or more hydrogen atoms (attached to a carbon or heteroatom) is replaced with a selection from the indicated group of substituents, provided that the designated atom's normal valency under the existing circumstances is not exceeded. The group may be optionally substituted with particular substituents at positions that do not significantly interfere with the preparation of compounds falling within the scope of this disclosure and on the understanding that the substitution(s) does not significantly adversely affect the biological activity or structural stability of the compound. Combinations of substituents are permissible only if such combinations result in stable compounds. By ‘stable compound’ or ‘stable structure’, it is meant a compound that is sufficiently robust to survive isolation to a useful degree of purity from a reaction mixture and/or formulation into an efficacious therapeutic agent. The term ‘optionally substituted’ or ‘optional substituents’ as used herein means that the groups in question are either unsubstituted or substituted with one or more of the substituents specified. When the groups in question are substituted with more than one substituent, the substituents may be the same or different. Furthermore, the terms ‘independently’, ‘independently are’, and ‘independently selected from’ mean that the substituents in question may be the same or different.

The term ‘deuterium’ as used herein refers to an isotope of hydrogen that has one proton and one neutron in its nucleus and that has twice the mass of ordinary hydrogen. Deuterium herein is represented by the symbol ‘D’. The term ‘deuterated’ by itself or used to modify a compound or group as used herein refers to the presence of at least one deuterium atom attached to carbon. For example, the term ‘deuterated compound’ refers to a compound which contains one or more carbon-bound deuterium(s). In a deuterated compound of the present disclosure, when a particular position is designated as having deuterium, it is understood that the abundance of deuterium at that position is substantially greater than the natural abundance of deuterium, which is about 0.015%. The term ‘undeuterated’ or ‘non-deuterated’ as used herein refers to the ratio of deuterium atoms of which is not more than the natural isotopic deuterium content, which is about 0.015%; in other words, all hydrogens are present at their natural isotopic percentages. Unless otherwise stated, when a position is designated specifically as ‘H’ or ‘hydrogen’, the position is understood to have hydrogen at its natural abundance isotopic composition.

The term ‘isotopic enrichment factor’ as used herein refers to the ratio between the isotope abundance and the natural abundance of a specified isotope.

The term ‘isotopologue’ as used herein refers to a species in which the chemical structure differs from a specific compound of the disclosure only in the isotopic composition thereof.

The term ‘substantially free of other stereoisomers’ as used herein means less than 10% of other stereoisomers, preferably less than 5% of other stereoisomers, more preferably less than 2% of other stereoisomers and most preferably less than 1% of other stereoisomers are present.

The term ‘pharmaceutically acceptable salt’ as used herein refers to a salt that is not biologically or otherwise undesirable (e.g., not toxic or otherwise harmful). A salt of a compound of the disclosure is formed between an acid and a basic group of the compound, or a base and an acidic group of the compound. For example, when the compounds of the disclosure contain at least one basic group (i.e., groups that can be protonated), the disclosure includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the disclosure contain one or more acidic groups (e.g., a carboxylic acid), the disclosure includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be, found in Stahl, P. H. et al. Pharmaceutical Salts: Properties, Selection, and Use, 2nd Revised Edition, Wiley, 2011.

The terms ‘treatment’, ‘treating’ and ‘treat’ as used herein, include their generally accepted meanings, i.e., the management and care of a patient for the purpose of preventing, reducing the risk in incurring or developing a given condition or disease, prohibiting, restraining, alleviating, ameliorating, slowing, stopping, delaying, or reversing the progression or severity, and holding in check existing characteristics of a disease, disorder, or pathological condition, including the alleviation or relief of symptoms or complications, or the cure or elimination of the disease, disorder, or condition.

The term ‘therapeutically effective amount’ as used herein refers to that amount of compound of the disclosure that will elicit the biological or medical response of a tissue, system, animal, or human that is being sought by a researcher, veterinarian, medical doctor or other. As will be recognized by a person of ordinary skill in the art, a therapeutically effective amount of the compounds of the disclosure will vary and will depend on the disease treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

Unless defined otherwise, ‘room temperature’ is intended to mean a temperature of from about 18 to 28° C., typically between about 18 and 25° C., and more typically between about 18 and 22° C. As used herein, the phrase ‘room temperature’ may be shortened to ‘rt’ or ‘RT’.

### Compounds:

Disclosed herein is a compound having the structural Formula I:

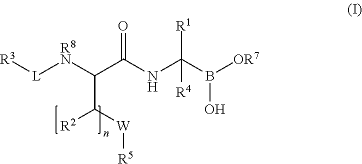

or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C1-C4 oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹¹, CONR¹¹R¹², NR¹¹R¹², SR¹¹, SO2NR¹¹R¹², C1-C4 alkyl, C1-C4 haloalkyl, C1-C4 alkoxy, phenyl, or a 5- or 6-membered heteroaryl;
n is 1 or 2;
each occurrence of R² is independently selected from H or C1-C4 alkyl;
L is C(O), C(O)O, C(O)NR⁶, S(O)₂, or a bond;
R³ is C1-C4 alkyl optionally substituted with one or more substituents each independently selected from the group consisting of deuterium, halogen, cyano, hydroxyl, C1-C4 alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹¹, CONR¹¹R¹², NR¹¹R¹², SR¹¹, SO2NR¹¹R¹², C₁-C₄ alkoxyl, or C₁-C₄ alkyl that is optionally substituted with one to three substituents selected from deuterium, halogen, cyano, hydroxyl, or C₁-C₄ alkoxyl;
W is selected from the group consisting of O, S, S(O), SO₂ and S(O)(NH);
R⁴ is selected from hydrogen, deuterium or C₁-C₂ alkyl;
R⁵ is hydrogen, deuterium, C₁-C₄ alkyl or C₁-C₄ alkoxyl wherein each alkyl or alkoxyl is optionally substituted with one or more substituents each independently selected from the group consisting of deuterium, halogen, cyano, hydroxyl, C₁-C₄ alkoxyl, C₁-C₅ alkyl-alkoxyl or phenyl; or
R⁵ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is NR⁹R¹⁰;
R⁶ is hydrogen, deuterium or C₁-C₂ alkyl optionally substituted with one or more of deuterium, halogen, hydroxyl and phenyl, wherein phenyl is optionally substituted with one or more substituent selected from halogen, hydroxyl and C₁-C₂ alkyl;
R⁷ is hydrogen, or R⁷ and R¹, together with the boron atom to which OR⁷ is attached form a 5-membered heteroalkyl ring;
R⁸ is selected from hydrogen, deuterium, or C₁-C₂ alkyl optionally substituted with one or more substituents each independently selected from the group consisting of halogen, hydroxyl, cyano, methoxyl and phenyl; or
R⁸, L and R³ together with the N to which R⁸ and L are attached form a saturated or unsaturated heterocycloalkyl group optionally having one or more additional heteroatoms selected from N, O and S, wherein the heterocycloalkyl is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl, and is optionally fused to an aryl or heteroaryl group which is optionally substituted with one or more substituents selected from halogen, cyano, hydroxyl, C₁-C₄ alkoxyl, or C₁-C₄ alkyl, wherein C₁-C₄ alkyl is optionally substituted with one to three substituents selected from halogen, cyano and C₁-C₄ alkoxyl, or is optionally fused to a cycloalkyl or heterocycloalkyl group which is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl;
R⁹ and R¹⁰ are each independently selected from hydrogen or C₁-C₆ alkyl that is optionally substituted with one to three substituents selected from halogen, cyano, or C₁-C₄ alkoxyl; and
R¹¹ and R¹² are each independently selected from hydrogen, deuterium, C1-C4 alkyl; C1-C4 haloalkyl, C1-C5 alkyl-alkoxyl, C3-C7 cycloalkyl, or R¹¹ and R¹² together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

In certain embodiments, R¹ is methyl, ethyl, n-propyl, i-propyl, n-butyl or tert-butyl, or methoxymethyl each optionally substituted with a phenyl ring. In certain embodiments, R¹ is methyl. In certain embodiments, R¹ is methyl substituted with a phenyl ring. In certain embodiments, R¹ is ethyl. In certain embodiments, R¹ is ethyl substituted with a phenyl ring. In certain embodiments, R¹ is n-propyl. In certain embodiments, R¹ is n-propyl substituted with a phenyl ring. In certain embodiments, R¹ is tert-butyl. In certain embodiments, R¹ is tert-butyl substituted with a phenyl ring. In certain embodiments, R¹ is n-butyl. In certain embodiments, R¹ is n-butyl substituted with a phenyl ring. In certain embodiments, R¹ is methoxymethyl. In certain embodiments, R¹ is methoxymethyl optionally substituted with a phenyl ring. In certain embodiments, R¹ is phenoxymethyl. In certain embodiments, R¹ is phenoxyethyl. In certain embodiments, R¹ is 2-phenyl-2-oxo-ethyl. In embodiments, R¹ is CO2H. In embodiments, R¹ is CO2R¹¹. In embodiments, R¹ is CONR¹¹R¹². In embodiments, R¹ is NR¹¹R¹². In embodiments, R¹ is SR¹¹. In embodiments, R¹ is SO2NR¹¹R¹².

In certain embodiments, n is 1 or 2. In certain embodiments, n is 1. In certain embodiments, n is 2.

In certain embodiments, W is 0. In certain embodiments, W is S. In certain embodiments, W is SO₂. In certain embodiments, W is S(O). In certain embodiments, W is S(O)(NH).

In certain embodiments, R³ is C₁-C₄ alkyl optionally substituted with one or more substituents each independently selected from the group consisting of halogen, cyano, hydroxyl, C₁-C₄ alkoxyl or phenyl.

In certain embodiments, R³ is cycloalkyl or heterocycloalkyl having one or more heteroatoms selected from N, O and S, wherein the cycloalkyl or heterocycloalkyl is optionally substituted with one or more substituents selected from halogen, cyano, hydroxyl, C₁-C₄ alkoxyl, or C₁-C₄ alkyl that is optionally substituted with one to three substituents selected from halogen, cyano, hydroxyl, or C₁-C₄ alkoxyl.

In certain embodiments, R³ is aryl or heteroaryl having one or more heteroatoms selected from N, O and S, wherein aryl or heteroaryl is optionally substituted with one or more substituents selected from halogen, cyano, hydroxyl, C₁-C₄ alkoxyl, or C₁-C₄ alkyl that is optionally substituted with one to three substituents selected from halogen, cyano, hydroxyl, or C₁-C₄ alkoxyl.

In certain embodiments, R³ is C₁-C₄ alkyl optionally substituted with one or more substituents each independently selected from the groups consisting of halogen, cyano, or C₁-C₄ alkoxyl. In certain embodiments, R³ is cycloalkyl which is optionally substituted with one or more halogen, cyano, C₁-C₄ alkoxyl, or C₁-C₄ alkyl that is optionally substituted with one to three halogen, cyano, or C₁-C₄ alkoxyl. In certain embodiments, R³ is heterocyclyl which is optionally substituted with one or more halogen, cyano, C₁-C₄ alkoxyl, or C₁-C₄ alkyl that is optionally substituted with one to three halogen, cyano, or C₁-C₄ alkoxyl. In certain embodiments, R³ is aryl which is optionally substituted with one or more halogen, cyano, C₁-C₄ alkoxyl, or C₁-C₄ alkyl that is optionally substituted with one to three halogen, cyano, or C₁-C₄ alkoxyl. In certain embodiments, R³ is cycloaryl which is optionally substituted with one or more halogen, cyano, C₁-C₄ alkoxyl, or C₁-C₄ alkyl that is optionally substituted with one to three halogen, cyano, or C₁-C₄ alkoxyl. In certain embodiments, R³ is heteroaryl which is optionally substituted with one or more halogen, cyano, C₁-C₄ alkoxyl, or C₁-C₄ alkyl that is optionally substituted with one to three halogen, cyano, or Cr C₄ alkoxyl. In certain embodiments, R³ is methyl, tert-butyl, trifluoromethyl, or phenyl. In certain embodiments, R³ is methyl, tert-butyl, phenyl optionally substituted with one or more halogen, cyano, C₁-C₄ alkoxyl, or C₁-C₄ alkyl that is optionally substituted with one to three halogen, cyano, or C₁-C₄ alkoxyl; pyridinyl optionally substituted with one or more halogen, cyano, C₁-C₄ alkoxyl, or C₁-C₄ alkyl that is optionally substituted with one to three halogen, cyano, or C₁-C₄ alkoxyl; piperidinyl optionally substituted with one or more halogen, cyano, C₁-C₄ alkoxyl, or C₁-C₄ alkyl that is optionally substituted with one to three halogen, cyano, or C₁-C₄ alkoxyl; pyrrolidinyl optionally substituted with one or more halogen, cyano, C₁-C₄ alkoxyl, or C₁-C₄ alkyl that is optionally substituted with one to three halogen, cyano, or C₁-C₄ alkoxyl; imidazolyl optionally substituted with one or more halogen, cyano, C₁-C₄ alkoxyl, or C₁-C₄ alkyl that is optionally substituted with one to three halogen, cyano, or C₁-C₄ alkoxyl; pyrazolyl optionally substituted one or more halogen, cyano, C₁-C₄ alkoxyl, or C₁-C₄ alkyl that is optionally substituted with one to three halogen, cyano, or C₁-C₄ alkoxyl; thiazolyl optionally substituted with one or more halogen, cyano, C₁-C₄ alkoxyl, or C₁-C₄ alkyl that is optionally substituted with one to three halogen, cyano, or C₁-C₄ alkoxyl; pyrazinyl optionally substituted with one or more halogen, cyano, C₁-C₄ alkoxyl, or C₁-C₄ alkyl that is optionally substituted with one to three halogen, cyano, or C₁-C₄ alkoxyl; oxazolyl optionally substituted with one or more halogen, cyano, C₁-C₄ alkoxyl, or C₁-C₄ alkyl that is optionally substituted with one to three halogen, cyano, or C₁-C₄ alkoxyl; morpholinyl optionally substituted with one or more halogen, cyano, C₁-C₄ alkoxyl, or C₁-C₄ alkyl that is optionally substituted with one to three halogen, cyano, or C₁-C₄ alkoxyl; or tetrahydropyranyl optionally substituted with one or more halogen, cyano, C₁-C₄ alkoxyl, or C₁-C₄ alkyl that is optionally substituted with one to three halogen, cyano, or C₁-C₄ alkoxyl.

In certain embodiments, R³ is methyl. In certain embodiments, R³ is tert-butyl. In certain embodiments, R³ is trifluoromethyl. In certain embodiments, R³ is phenyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is pyridinyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is piperidinyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is pyrrolidinyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is imidazolyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is pyrazolyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is thiazolyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is pyrazinyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is oxazolyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is morpholinyl optionally substituted with one or more halogen, cyano, C₁-C₄ alkoxyl; or C₁-C₄ alkyl that is optionally substituted with one to three halogen, cyano, or C₁-C₄ alkoxyl. In certain embodiments, R³ is tetrahydropyranyl optionally substituted with one or more halogen, cyano, C₁-C₄ alkoxyl; or C₁-C₄ alkyl that is optionally substituted with one to three halogen, cyano, or C₁-C₄ alkoxyl.

In certain embodiments, R³ is phenyl, optionally substituted with one to three of C₁-C₂ alkyl and C₁-C₂ alkoxyl. In certain embodiments, R³ is pyridinyl, optionally substituted with one to three of C₁-C₂ alkyl and C₁-C₂ alkoxyl. In certain embodiments, R³ is pyrazinyl, optionally substituted with one to three of C₁-C₂ alkyl and C₁-C₂ alkoxyl. In certain embodiments, R³ is morpholinyl, optionally substituted with one to three of C₁-C₂ alkyl and C₁-C₂ alkoxyl. In certain embodiments, R³ is phenyl, optionally substituted with one to three of halogen, particularly, substituted with one or two of F or Cl, and more particularly one or two Cl.

In embodiments, R³ is CO2H. In embodiments, R³ is CO2R¹¹. In embodiments, R³ is CONR¹¹R¹². In embodiments, R³ is NR¹¹R¹². In embodiments, R³ is SR¹¹. In embodiments, R³ is SO2NR¹¹R¹².

In embodiments, R¹ and R¹² are each independently selected from hydrogen, deuterium, C1-C4 alkyl; C1-C4 haloalkyl or C1-C5 alkyl-alkoxyl. In embodiments, R¹¹ and R¹² are each independently selected from hydrogen, deuterium, C1-C4 alkyl; C1-C4 haloalkyl, C1-C5 alkyl-alkoxyl or C3-C7 cycloalkyl. In embodiments, R¹ and R¹² are each independently selected from hydrogen, deuterium, C1-C2 alkyl; C1-C2 haloalkyl, C1-C2 alkyl-alkoxyl or C3-C5 cycloalkyl. In embodiments, R¹ and R¹² together with the N to which they are attached form 3 to 7 membered heterocyclic ring optionally having one or more additional heteroatoms selected from N, O and S. In any such embodiments, the C3-C7 cycloalkyl, C3-C5 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl. In embodiments, the substituents may be selected from one, two or three of deuterium, F, Cl, hydroxyl, oxo, CN, C1-C2 alkyl, C1-C2 haloalkyl or C1-C2 alkoxyl.

In certain embodiments, R⁴ is selected from hydrogen or C1-C2 alkyl. In certain embodiments, R⁴ is hydrogen. In certain embodiments, R⁴ is methyl.

In certain embodiments, each occurrence of R² is independently selected from the group consisting of hydrogen, or C₁-C₄ alkyl. In certain embodiments, R² is hydrogen. In certain embodiments, R² is methyl. In certain embodiments, a first occurrence of R² is hydrogen and a second occurrence of R² is methyl.

In certain embodiments, R⁵ is C₁-C₄ alkyl or C₁-C₄ alkoxyl optionally substituted with one or more substituents each independently selected from the group consisting of halogen, cyano, hydroxyl, C₁-C₄ alkoxyl, C₁-C₅ alkyl-alkoxyl or phenyl.

In certain embodiments, R⁵ is cycloalkyl or heterocycloalkyl having one or more heteroatoms selected from N, O and S, wherein the cycloalkyl or heterocycloalkyl is optionally substituted with one or more substituents selected from halogen, cyano, hydroxyl, C₁-C₄ alkoxyl, or C₁-C₄ alkyl that is optionally substituted with one to three substituents selected from halogen, cyano, hydroxyl, or C₁-C₄ alkoxyl and C₁-C₅ alkyl-alkoxyl.

In certain embodiments, R⁵ is aryl or heteroaryl having one or more heteroatoms selected from N, O and S, wherein aryl, or heteroaryl is optionally substituted with one or more substituents selected from halogen, cyano, hydroxyl, C₁-C₄ alkoxyl, or C₁-C₄ alkyl that is optionally substituted with one to three substituents selected from halogen, cyano, hydroxyl, or C₁-C₄ alkoxyl and C₁-C₅ alkyl-alkoxyl.

In certain embodiments, R⁵ is NR⁹R¹⁰.

In certain embodiments, R⁵ is selected from the group consisting of methyl, phenyl, methoxy, cyclopropyl, pyridinyl, benzyl, or NMe₂. In certain embodiments, R⁵ is methyl. In certain embodiments, R⁵ is phenyl. In certain embodiments, R⁵ is methoxyl. In certain embodiments, R⁵ is cyclopropyl. In certain embodiments, R⁵ is pyridinyl. In certain embodiments, R⁵ is benzyl. In certain embodiments, R⁵ is methyl substituted with one, two or three deuterium atoms.

In certain embodiments, R⁵ is NMe₂.

In certain embodiments, R⁹ and R¹⁰ are each independently selected from hydrogen or C₁-C₆ alkyl that is optionally substituted with one to three substituents selected from halogen, cyano, or C₁-C₄ alkoxy. In certain embodiments R⁹ and R¹⁰ are each methyl.

In certain embodiments, L is a bond, C(O), C(O)O, C(O)NR⁶, or SO₂. In certain embodiments, L is a bond. In certain embodiments, L is C(O). In certain embodiments, L is C(O)O. In certain embodiments, L is C(O)NR⁶. In certain embodiments, L is SO₂.

In certain embodiments, R⁶ is selected from hydrogen, deuterium, methyl or ethyl, wherein methyl or ethyl is optionally substituted with one or more substituents each independently selected from the group consisting of halogen, hydroxyl, cyano, methoxyl or phenyl.

In certain embodiments, R⁷ is hydrogen, or R⁷ and R¹, together with the boron atom to which R⁷ is attached form a 5-membered heteroalkyl ring. In certain embodiments, R⁷ is hydrogen.

In certain embodiments, R⁸ is selected from hydrogen, deuterium, methyl or ethyl, wherein methyl or ethyl is optionally substituted with one or more substituents each independently selected from the group consisting of halogen, hydroxyl, cyano, methoxyl or phenyl.

In certain embodiments, R⁸ is selected from hydrogen or C₁-C₂ alkyl optionally substituted with one or more substituents each independently selected from the group consisting of halogen, hydroxyl, cyano, methoxyl or phenyl. In certain embodiments, R⁸ is C₁-C₂ alkyl optionally substituted with one or more substituents each independently selected from the group consisting of halogen, hydroxyl, cyano, methoxyl or phenyl. In certain embodiments, R⁸ is hydrogen.

In certain embodiments, R⁸, L and R³ together with the N to which R⁸ and L are attached form a heterocycloalkyl group optionally having one or more additional heteroatoms selected from N, O and S, wherein the heterocycloalkyl is optionally substituted with one or more substituents selected from halogen, cyano, hydroxyl, C₁-C₄ alkoxyl, oxo, or C₁-C₄ alkyl that is optionally substituted with one to three substituents selected from halogen, cyano and C₁-C₄ alkoxyl, and is optionally fused to an aryl or heteroaryl group which is optionally substituted with one or more substituents selected from halogen, cyano, hydroxyl, C₁-C₄ alkoxyl, or C₁-C₄ alkyl that is optionally substituted with one to three substituents selected from halogen, cyano and C₁-C₄ alkoxyl, or is optionally fused to a cycloalkyl or heterocycloalkyl group which is optionally substituted with one or more substituents selected from halogen, cyano, hydroxyl, C₁-C₄ alkoxyl, oxo, or C₁-C₄ alkyl that is optionally substituted with one to three substituents selected from halogen, cyano and C₁-C₄ alkoxyl.

In certain embodiments, R⁸, L and R³ together with the N to which R⁸ and L are attached form a heterocycloalkyl group, wherein the heterocycloalkyl is substituted with oxo and is optionally fused to an aryl.

In certain embodiments, halogen is selected from fluoro and chloro. In certain embodiments, halogen is fluoro. In certain embodiments, halogen is chloro.

In another embodiment, the present disclosure is directed to a compound, or pharmaceutically acceptable salt thereof represented b an one of the following structures:

TABLE 1
Chemical structures of compounds according to the disclosure. Any of these
compounds may also exist in the isomeric forms, particularly the oxaborolane isomer
derivative, and such isomers are explicitly intended to be encompassed within the scope of
this disclosure.
Structure 1: 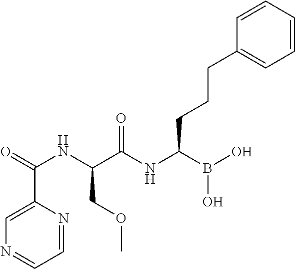 [SMILES: COC[C@@H](NC(=O)c1cnccn1)C(=O)N[C@@H](CCCc1ccccc1)B(O)O]
Structure 2: 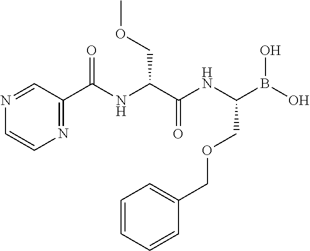 [SMILES: COC[C@@H](NC(=O)c1cnccn1)C(=O)N[C@@H](COCc1ccccc1)B(O)O]
Structure 3: 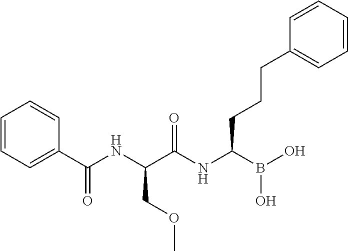 [SMILES: COC[C@@H](NC(=O)c1ccccc1)C(=O)N[C@@H](CCCc1ccccc1)B(O)O]
Structure 4: 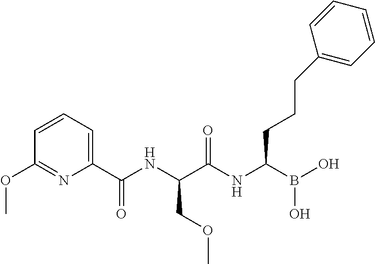 [SMILES: COC[C@@H](NC(=O)c1cccc(OC)n1)C(=O)N[C@@H](CCCc1ccccc1)B(O)O]
Structure 5: 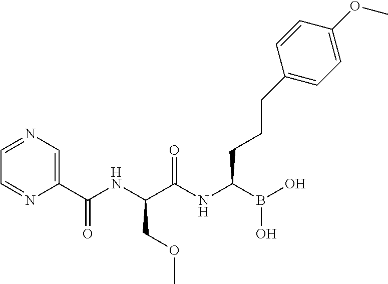 [SMILES: COC[C@@H](NC(=O)c1cnccn1)C(=O)N[C@@H](CCCc1ccc(OC)cc1)B(O)O]
Structure 6: 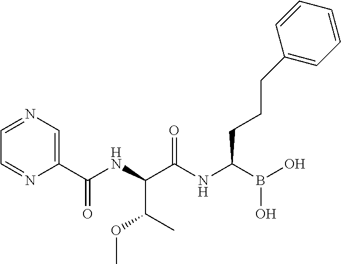 [SMILES: CO[C@@H](C)[C@@H](NC(=O)c1cnccn1)C(=O)N[C@@H](CCCc1ccccc1)B(O)O]
Structure 7: 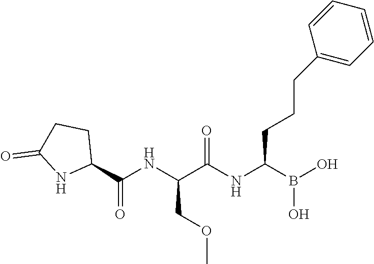 [SMILES: COC[C@@H](NC(=O)[C@@H]1CCC(=O)N1)C(=O)N[C@@H](CCCc1ccccc1)B(O)O]
Structure 8: 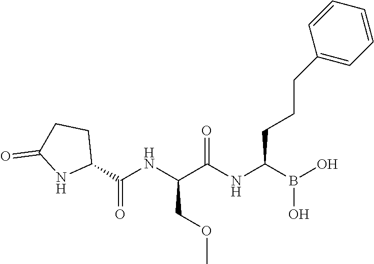 [SMILES: COC[C@@H](NC(=O)[C@H]1CCC(=O)N1)C(=O)N[C@@H](CCCc1ccccc1)B(O)O]
Structure 9: 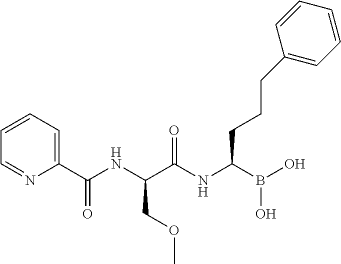 [SMILES: COC[C@@H](NC(=O)c1ccccn1)C(=O)N[C@@H](CCCc1ccccc1)B(O)O]
Structure 10: 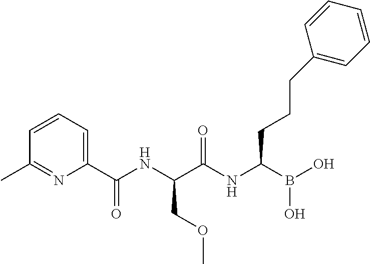 [SMILES: COC[C@@H](NC(=O)c1cccc(C)n1)C(=O)N[C@@H](CCCc1ccccc1)B(O)O]
Structure 11: 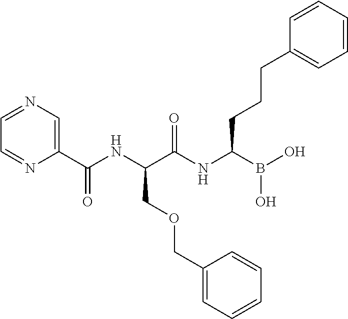 [SMILES: O=C(N[C@H](COCc1ccccc1)C(=O)N[C@@H](CCCc1ccccc1)B(O)O)c1cnccn1]
Structure 12: 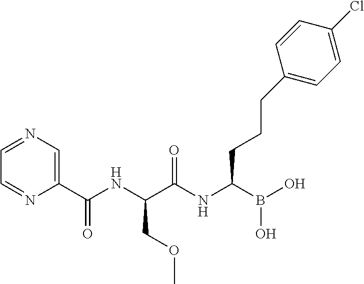 [SMILES: COC[C@@H](NC(=O)c1cnccn1)C(=O)N[C@@H](CCCc1ccc(Cl)cc1)B(O)O]
Structure 13: 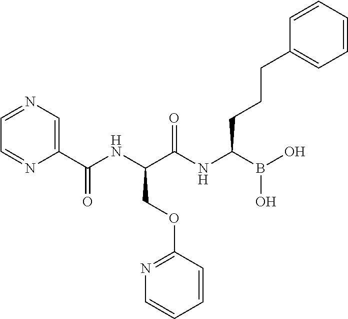 [SMILES: O=C(N[C@H](COc1ccccn1)C(=O)N[C@@H](CCCc1ccccc1)B(O)O)c1cnccn1]
Structure 14: 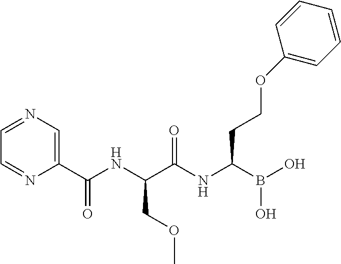 [SMILES: COC[C@@H](NC(=O)c1cnccn1)C(=O)N[C@@H](CCOc1ccccc1)B(O)O]
Structure 15: 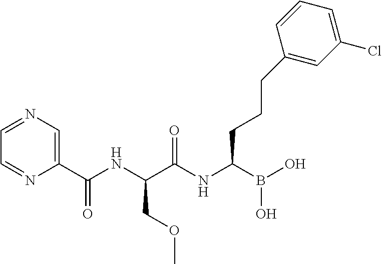 [SMILES: COC[C@@H](NC(=O)c1cnccn1)C(=O)N[C@@H](CCCc1cccc(Cl)c1)B(O)O]
Structure 16: 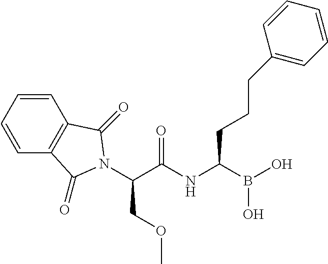 [SMILES: COC[C@H](C(=O)N[C@@H](CCCc1ccccc1)B(O)O)N1C(=O)c2ccccc2C1=O]
Structure 17: 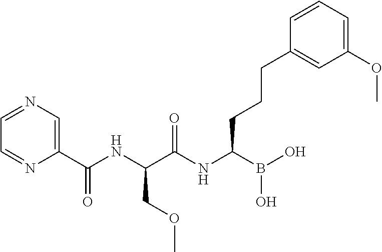 [SMILES: COC[C@@H](NC(=O)c1cnccn1)C(=O)N[C@@H](CCCc1cccc(OC)c1)B(O)O]
Structure 18: 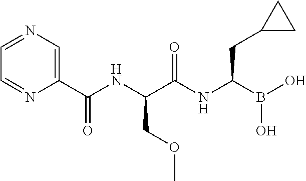 [SMILES: COC[C@@H](NC(=O)c1cnccn1)C(=O)N[C@@H](CC1CC1)B(O)O]
Structure 19: 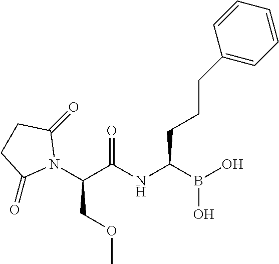 [SMILES: COC[C@H](C(=O)N[C@@H](CCCc1ccccc1)B(O)O)N1C(=O)CCC1=O]
Structure 20: 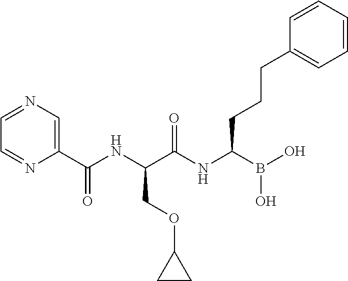 [SMILES: O=C(N[C@H](COC1CC1)C(=O)N[C@@H](CCCc1ccccc1)B(O)O)c1cnccn1]
Structure 21: 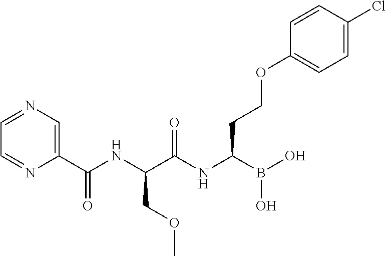 [SMILES: COC[C@@H](NC(=O)c1cnccn1)C(=O)N[C@@H](CCOc1ccc(Cl)cc1)B(O)O]
Structure 22: 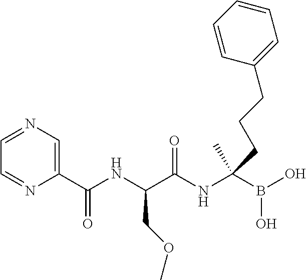 [SMILES: COC[C@@H](NC(=O)c1cnccn1)C(=O)N[C@@](C)(CCCc1ccccc1)B(O)O]
Structure 23: 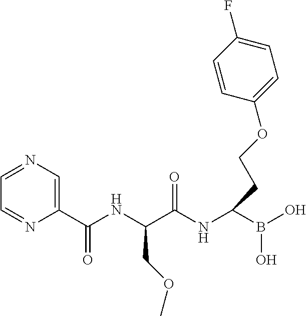 [SMILES: COC[C@@H](NC(=O)c1cnccn1)C(=O)N[C@@H](CCOc1ccc(F)cc1)B(O)O]
Structure 24: 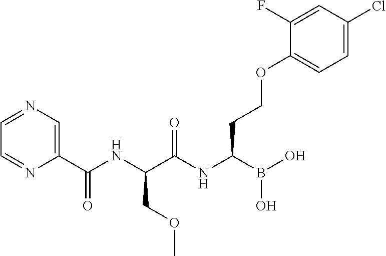 [SMILES: COC[C@@H](NC(=O)c1cnccn1)C(=O)N[C@@H](CCOc1ccc(Cl)cc1F)B(O)O]
Structure 25: 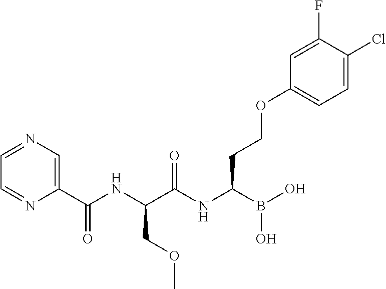 [SMILES: COC[C@@H](NC(=O)c1cnccn1)C(=O)N[C@@H](CCOc1ccc(Cl)c(F)c1)B(O)O]
Structure 26: 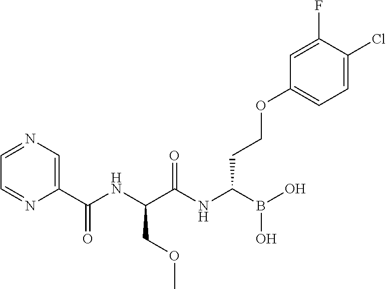 [SMILES: COC[C@@H](NC(=O)c1cnccn1)C(=O)N[C@H](CCOc1ccc(Cl)c(F)c1)B(O)O]
Structure 27: 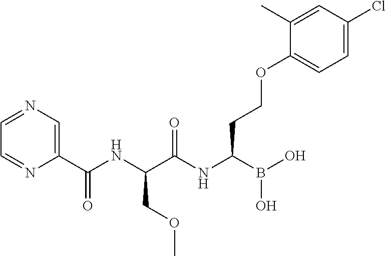 [SMILES: COC[C@@H](NC(=O)c1cnccn1)C(=O)N[C@@H](CCOc1ccc(Cl)cc1C)B(O)O]
Structure 28: 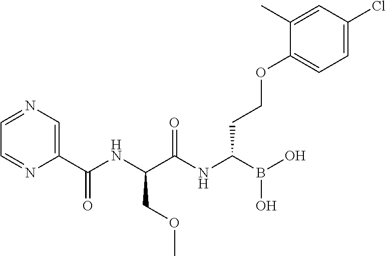 [SMILES: COC[C@@H](NC(=O)c1cnccn1)C(=O)N[C@H](CCOc1ccc(Cl)cc1C)B(O)O]
Structure 29: 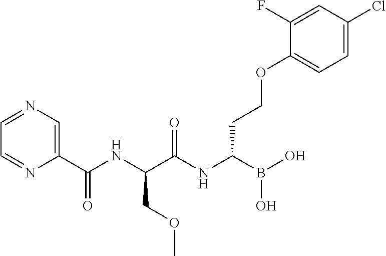 [SMILES: COC[C@@H](NC(=O)c1cnccn1)C(=O)N[C@H](CCOc1ccc(Cl)cc1F)B(O)O]
Structure 30: 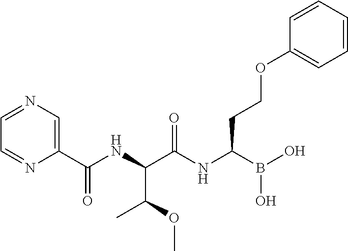 [SMILES: CO[C@@H](C)[C@@H](NC(=O)c1cnccn1)C(=O)N[C@@H](CCOc1ccccc1)B(O)O]
Structure 31: 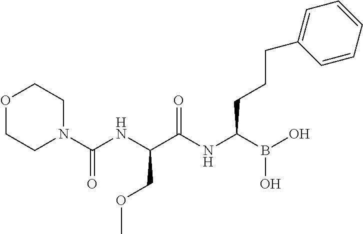 [SMILES: COC[C@@H](NC(=O)N1CCOCC1)C(=O)N[C@@H](CCCc1ccccc1)B(O)O]
Structure 32: 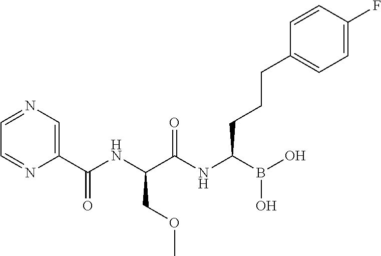 [SMILES: COC[C@@H](NC(=O)c1cnccn1)C(=O)N[C@@H](CCCc1ccc(F)cc1)B(O)O]
Structure 33: 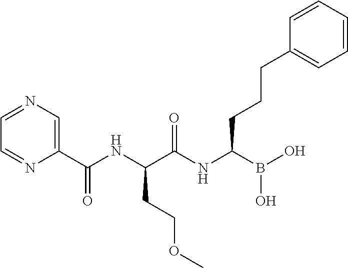 [SMILES: COCC[C@@H](NC(=O)c1cnccn1)C(=O)N[C@@H](CCCc1ccccc1)B(O)O]
Structure 34: 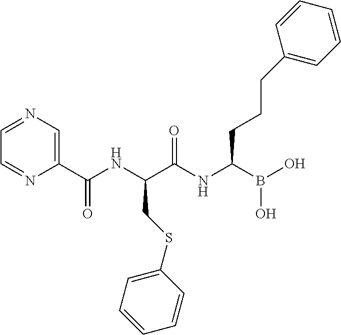 [SMILES: O=C(N[C@H](CSc1ccccc1)C(=O)N[C@@H](CCCc1ccccc1)B(O)O)c1cnccn1]
Structure 35: 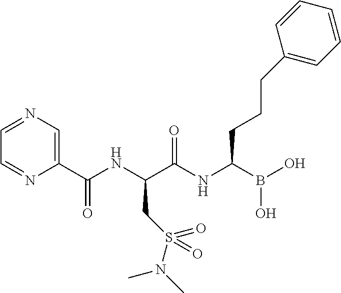 [SMILES: CN(C)S(=O)(=O)C[C@@H](NC(=O)c1cnccn1)C(=O)N[C@@H](CCCc1ccccc1)B(O)O]
Structure 36: 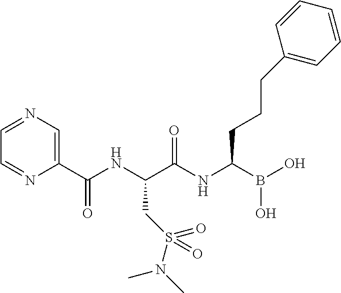 [SMILES: CN(C)S(=O)(=O)C[C@H](NC(=O)c1cnccn1)C(=O)N[C@@H](CCCc1ccccc1)B(O)O]
Structure 37: 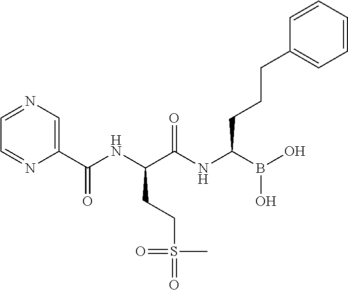 [SMILES: CS(=O)(=O)CC[C@@H](NC(=O)c1cnccn1)C(=O)N[C@@H](CCCc1ccccc1)B(O)O]
Structure 38: 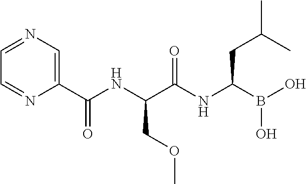 [SMILES: COC[C@@H](NC(=O)c1cnccn1)C(=O)N[C@@H](CC(C)C)B(O)O]
Structure 39: 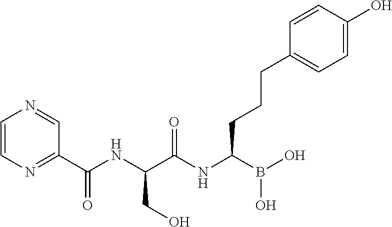 [SMILES: O=C(N[C@H](CO)C(=O)N[C@@H](CCCc1ccc(O)cc1)B(O)O)c1cnccn1]
Structure 40: 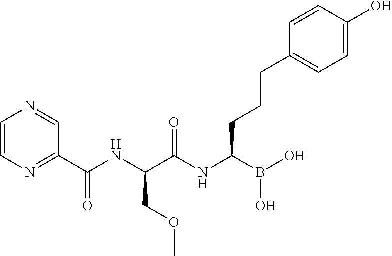 [SMILES: COC[C@@H](NC(=O)c1cnccn1)C(=O)N[C@@H](CCCc1ccc(O)cc1)B(O)O]
Structure 41: 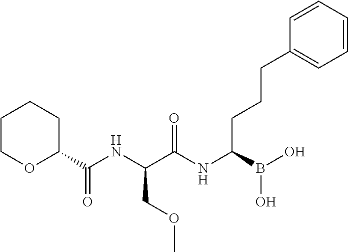 [SMILES: COC[C@@H](NC(=O)[C@H]1CCCCO1)C(=O)N[C@@H](CCCc1ccccc1)B(O)O]
Structure 42: 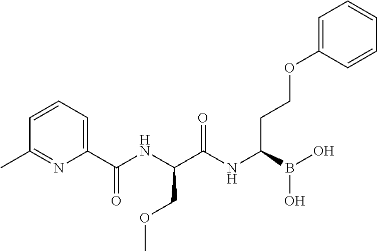 [SMILES: COC[C@@H](NC(=O)c1cccc(C)n1)C(=O)N[C@@H](CCOc1ccccc1)B(O)O]
Structure 43: 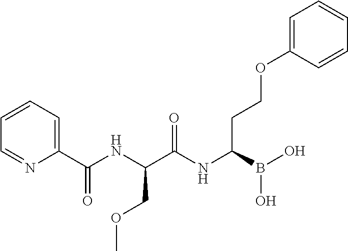 [SMILES: COC[C@@H](NC(=O)c1ccccn1)C(=O)N[C@@H](CCOc1ccccc1)B(O)O]
Structure 44: 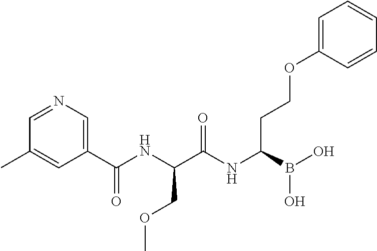 [SMILES: COC[C@@H](NC(=O)c1cncc(C)c1)C(=O)N[C@@H](CCOc1ccccc1)B(O)O]
Structure 45: 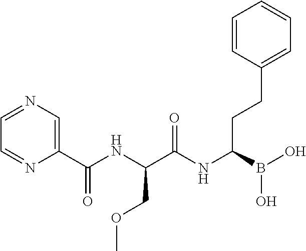 [SMILES: COC[C@@H](NC(=O)c1cnccn1)C(=O)N[C@@H](CCc1ccccc1)B(O)O]
Structure 46: 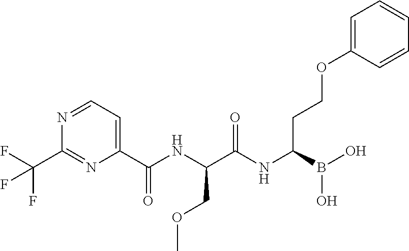 [SMILES: COC[C@@H](NC(=O)c1ccnc(C(F)(F)F)n1)C(=O)N[C@@H](CCOc1ccccc1)B(O)O]
Structure 47: 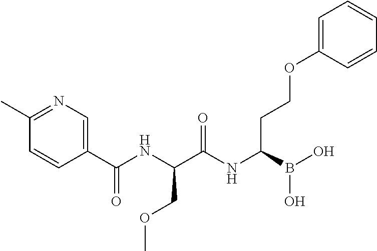 [SMILES: COC[C@@H](NC(=O)c1ccc(C)nc1)C(=O)N[C@@H](CCOc1ccccc1)B(O)O]
Structure 48: 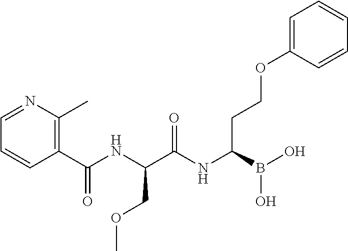 [SMILES: COC[C@@H](NC(=O)c1cccnc1C)C(=O)N[C@@H](CCOc1ccccc1)B(O)O]
Structure 49: 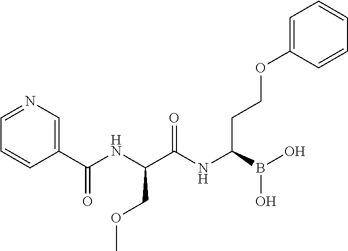 [SMILES: COC[C@@H](NC(=O)c1cccnc1)C(=O)N[C@@H](CCOc1ccccc1)B(O)O]
Structure 50: 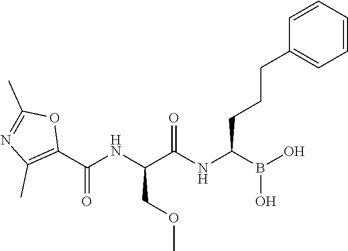 [SMILES: COC[C@@H](NC(=O)c1oc(C)nc1C)C(=O)N[C@@H](CCCc1ccccc1)B(O)O]
Structure 51: 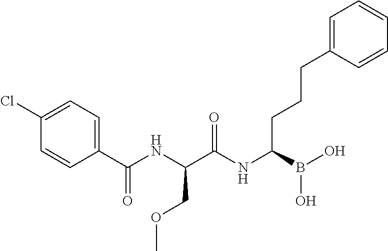 [SMILES: COC[C@@H](NC(=O)c1ccc(Cl)cc1)C(=O)N[C@@H](CCCc1ccccc1)B(O)O]
Structure 52: 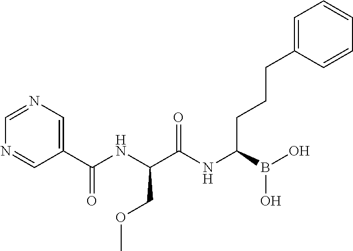 [SMILES: COC[C@@H](NC(=O)c1cncnc1)C(=O)N[C@@H](CCCc1ccccc1)B(O)O]
Structure 53: 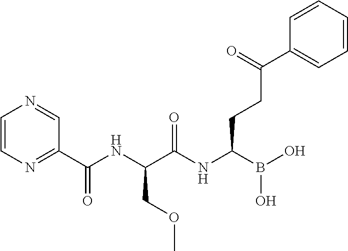 [SMILES: COC[C@@H](NC(=O)c1cnccn1)C(=O)N[C@@H](CCC(=O)c1ccccc1)B(O)O]
Structure 54: 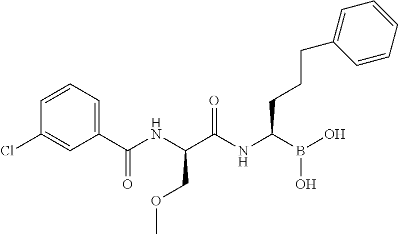 [SMILES: COC[C@@H](NC(=O)c1cccc(Cl)c1)C(=O)N[C@@H](CCCc1ccccc1)B(O)O]
Structure 55: 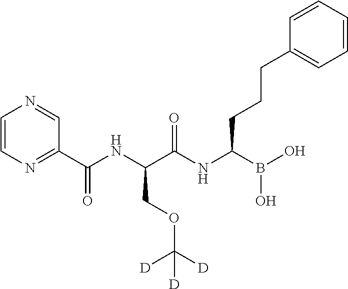 [SMILES: [2H]C([2H])([2H])OC[C@@H](NC(=O)c1cnccn1)C(=O)N[C@@H](CCCc1ccccc1)B(O)O]
Structure 56: 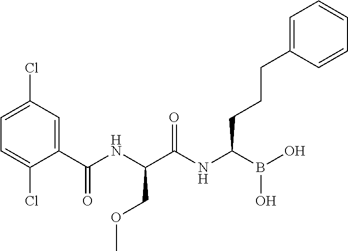 [SMILES: COC[C@@H](NC(=O)c1cc(Cl)ccc1Cl)C(=O)N[C@@H](CCCc1ccccc1)B(O)O]
Structure 57: 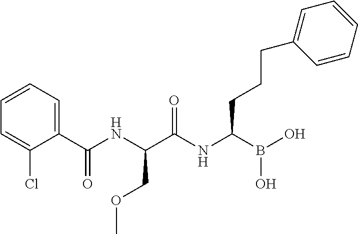 [SMILES: COC[C@@H](NC(=O)c1ccccc1Cl)C(=O)N[C@@H](CCCc1ccccc1)B(O)O]
Structure 58: 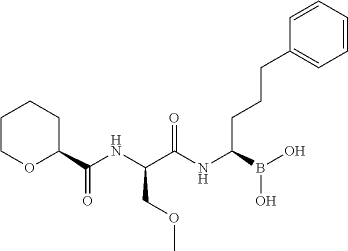 [SMILES: COC[C@@H](NC(=O)[C@@H]1CCCCO1)C(=O)N[C@@H](CCCc1ccccc1)B(O)O]
Structure 59: 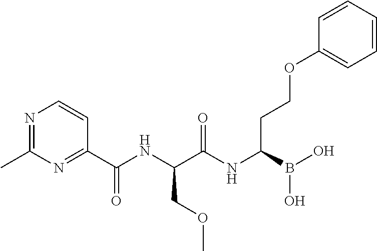 [SMILES: COC[C@@H](NC(=O)c1ccnc(C)n1)C(=O)N[C@@H](CCOc1ccccc1)B(O)O]
Structure 60:

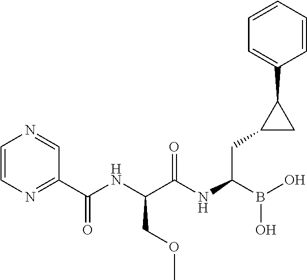 [SMILES: COC[C@@H](NC(=O)c1cnccn1)C(=O)N[C@@H](C[C@H]1C[C@@H]1c1ccccc1)B(O)O]

The compounds of the present disclosure may contain asymmetric carbon atoms (sometimes as the result of a deuterium atom) and thereby can exist as either individual stereoisomers or mixtures of enantiomers or mixtures of diastereomers. Accordingly, a compound of the present disclosure may exist as either a racemic mixture, a mixture of diastereomers, or as individual stereoisomers that are substantially free of other stereoisomers. Synthetic, separation, or purification methods to be used to obtain an enantiomer of a given compound are known in the art and are applicable for obtaining the compounds identified herein.

Unless otherwise indicated, when a disclosed compound is named or depicted by a structure without specifying the stereochemistry and has one or more chiral centers, it is understood to represent all possible stereoisomers of the compound. In other words, chiral centers that lack solid wedged or hashed wedged bonds indicate a mixture of stereoisomers.

Certain compounds of the present disclosure may be able to exist as tautomers. All tautomeric forms of these compounds, whether isolated individually or in mixtures, are within the scope of the present disclosure. For example, in instances where an —OH substituent is permitted on a heteroaromatic ring and keto-enol tautomerism is possible, it is understood that the substituent might in fact be present, in whole or in part, in the oxo (═O) form.

Compounds of the present disclosure may exist in amorphous form and/or one or more crystalline forms. As such, all amorphous and crystalline forms and mixtures thereof of the compounds of the disclosure are intended to be included within the scope of the present disclosure. In addition, some of the compounds of the present disclosure may form solvates with water (i.e., a hydrate) or common organic solvents. Such solvates and hydrates, particularly the pharmaceutically acceptable solvates and hydrates, of the compounds of this disclosure are likewise encompassed within the scope of the compounds of the disclosure and the pharmaceutically acceptable salts thereof, along with un-solvated and anhydrous forms of such compounds.

In one embodiment, deuterium isotope content at the deuterium substituted position is greater than the natural isotopic deuterium content (0.015%), more preferably greater than 50%, more preferably greater than 60%, more preferably greater than 75%, more preferably greater than 90%, more preferably greater than 95%, more preferably greater than 97%, more preferably greater than 99%. It will be understood that some variation of natural isotopic abundance may occur in any compound depending upon the source of the reagents used in the synthesis. Thus, a preparation of undeuterated compounds may inherently contain small amounts of deuterated isotopologues, such amounts being insignificant as compared to the degree of stable isotopic substitution of the deuterated compounds of the disclosure (see e.g., Gannes, L. Z. et al., Comp. Biochem. Physiol. Mol. Integr. Physiol, 119, 725 (1998)). Replacement of hydrogen with deuterium may affect the activity, toxicity, and pharmacokinetics (e.g., absorption, distribution, metabolism, and excretion (“ADME”)) of some drugs. For instance, such replacement may alter the chemical stability and biochemical reactivity of a compound through kinetic isotope effects. Because of the increased mass of deuterium relative to hydrogen, epimerization at stereogenic carbons may be slowed down when hydrogen is replaced with deuterium (see Pirali, T. et al, J. Med. Chem. 62, 5276-97 (2019)). Additionally, the presence of deuterium may affect how a molecule interacts with enzymes, thereby impacting enzyme kinetics. While in certain cases the increased mass of deuterium as compared to hydrogen can stabilize a compound and thereby improve activity, toxicity, or half-life, such impact is not predictable. In other instances, deuteration may have little to no impact on these properties, or may affect them in an undesirable manner. Whether and/or how such replacement will impact drug properties can only be determined if the drug is synthesized, evaluated, and compared to its non-deuterated counterpart (see Fukuto, J. M., et al., J. Med. Chem. 34, 2871-76 (1991)). Because some drugs have multiple sites of metabolism or more than one active sites for binding to a target, it is unpredictable as to which sites may benefit by deuterium replacement or to what extent isotope enrichment is necessary to produce a beneficial effect.

Further aspects and embodiments of the present disclosure are directed to compounds of Formula I or their pharmaceutically acceptable salts wherein one or more hydrogen is substituted with a deuterium atom.

A further aspect of the present disclosure provides a pharmaceutical composition comprising one or more compound of the first aspect of the disclosure or a pharmaceutically acceptable salt, solvate, stereoisomer or mixture of stereoisomers, tautomer, isotopic form, or pharmaceutically active metabolite thereof, or combinations thereof and one or more pharmaceutically acceptable excipient or carrier.

Further embodiments of the disclosure are methods of treating a disease characterized by mitochondrial dysfunction, such methods comprising administering to a subject in need thereof a therapeutically effective amount of a compound of the disclosure, or a pharmaceutically acceptable salt thereof. In some embodiments, the disease is selected from the group consisting of Alzheimer's disease, Parkinson's disease, obesity, diabetes, non-alcoholic steatohepatitis (NASH), and related metabolic syndromes such as non-alcoholic fatty liver disease (NAFLD), Alper's syndrome (Alpers-Huttenlocher syndrome), ataxia neuropathy syndrome (ANS), Mitochondrial DNA Depletion Syndrome (MDDS), Leigh Syndrome (Leigh Disease), Leber's Hereditary Optic Neuropathy (LHON), chronic progressive external ophthalmoplegia (CPEO), myoclonic epilepsy myopathy sensory ataxia (MEMSA), MELAS (Mitochondrial Encephalopathy, Lactic Acidosis, and Stroke-like episodes) syndrome, MERRF (myoclonus epilepsy with ragged-red fibers) syndrome, mitochondrial neurogastrointestinal encephalomyopathy (MNGIE), neuropathy, ataxia, and retinitis pigmentosa (NARP), Kearn's-Sayre Syndrome (KSS), and Pearson's Syndrome.

In some embodiments, the disease to be treated with a compounds of the disclosure or pharmaceutically acceptable salts thereof is associated with mtDNA mutations or deletions, for example m.3243A>G, m.11778G>A, m.14484T>C, m.3460G>A, m.8344A>G, m.3271T>C, m.3251A>G, m.8356T>C, m.4274T>C, m.14709T>C, m.12320A>G, m.4269A>G, m.12258C>A, m.1606G>A, m.10010T>C, m.7445A>G, and m.1555A>G (see https:mitomap.org/MITOMAP).

Additional embodiments of the disclosure relate to methods of treating cancers, such as those identified in Wong, K. S. et al. “Recent Advances in Targeting Human Mitochondrial AAA+Proteases to Develop Novel Cancer Therapeutics,” Advances in Experimental Medicine and Biology, 1158, 119-142 (2019), using a compound of the disclosure or its pharmaceutically acceptable salt.

Further aspects and embodiments are directed to one or more compounds or pharmaceutical compositions of any aspect of the disclosure for use in medicine. In particular, the compounds and pharmaceutical compositions may be for use in the treatment of a disorder or a disease characterized by a mitochondrial dysfunction as defined elsewhere herein. In embodiments, the use comprises administering to a subject in need thereof a therapeutically effective amount of a compound of the disclosure, or a pharmaceutically acceptable salt thereof, according to any aspect.

For the treatment of cancer, the compounds described herein may be administered in combination with a chemotherapeutic agent. Therapeutically effective amounts of the additional chemotherapeutic agent(s) are well known to those skilled in the art. However, it is well within the attending physician to determine the amount of other chemotherapeutic agent(s) to be delivered.

Examples of these chemotherapeutic agents include, but are not limited to, Abitrexate (Methotrexate Injection), Abraxane (Paclitaxel Injection), Actemra (Tocilizumab), Adcetris (Brentuximab Vedotin Injection), Adriamycin (Doxorubicin), Adrucil Injection (5-FU (fluorouracil)), Afinitor (Everolimus), Afinitor Disperz (Everolimus), Aldara (Imiquimod), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Avelumab, Bexxar (Tositumomab), BiCNU (Carmustine), Blenoxane (Bleomycin), Blincyto (Blinatumomab), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Cyramza (Ramucirumab), Dacogen (Decitabine), Darzalex (Daratumumab), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Durvalumab, Eligard (Leuprolide), Ellence (Ellence (epirubicin)), Eloxatin (Eloxatin (oxaliplatin)), Elspar (Asparaginase), Emcyt (Estramustine), Empliciti (Elotuzumab), Enhertu (fam-trastuzumab deruxtecan-nxki), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gazyva (Obinutuzumab), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leflunomide (SU101), Lartruvo (Olaratumab), Leukeran (Chlorambucil), Leukine (Sargramostim), Leustatin (Cladribine), Libtayo (Cemiplimab), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Portrazza (Necitumumab), Prednisone Intensol (Prednisone), Proleukin (Aldesleukin), Purinethol (Mercaptopurine), Reclast (Zoledronic acid), Revlimid (Lenalidomide), Removab (Catumaxomab), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Sarclisa (Isatuximab-irfc), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Tecentriq (Atezolizumab), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Tremelimumab, Trelstar (Triptorelin Injection), Trexall (Methotrexate), Trisenox (Arsenic trioxide), Tykerb (lapatinib), Unituxin (Dinutuximab), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (lbritumomab Tiuxetan), Zoladex (Coserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting.

Further embodiments of the disclosure are methods of treating neurodegenerative disorders, metabolic disorders, and diseases associated with the aging process.

### Dosage Forms, Medicaments and Pharmaceuticals:

The compounds, molecules or agents of the disclosure may be used to treat (e.g. cure, alleviate or prevent) one or more diseases, infections or disorders. Thus, in accordance with the disclosure, the compounds and molecules may be manufactured into medicaments or may be incorporated or formulated into pharmaceutical compositions.

The molecules, compounds and compositions of the disclosure may be administered by any convenient route, for example, methods of administration include intradermal, intramuscular, intraperitoneal, intravenous, subcutaneous, intranasal, epidural, oral, sublingual, intranasal, intravaginal, transdermal, rectally, by inhalation, or topically to the skin. Delivery systems are also known to include, for example, encapsulation in liposomes, microgels, microparticles, microcapsules, capsules, etc. Any other suitable delivery system known in the art is also envisioned in use. Administration can be systemic or local. The mode of administration may be left to the discretion of the practitioner.

The dosage administered will, of course, vary depending upon known factors, such as the pharmacodynamic properties of the particular active agent; the chosen mode and route of administration; the age, health and weight of the recipient; the nature of the disease or disorder to be treated; the extent of the symptoms; any simultaneous or concurrent treatments; the frequency of treatment; and the effect desired.

Depending on known factors, such as those noted above, the required dosage of the active agent may be administered in a single daily dose, or the total daily dosage may be administered in divided doses of e.g. two, three, or four times daily. Suitably, the therapeutic treatment regime according to the disclosure is devised for a single daily dose or for a divided daily dose of two doses.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting CDK2. Diseases or disorders which may benefit from CDK2 inhibition include, for example, proliferative diseases or disorders and cancer.

The ‘effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder. Beneficially, an effective amount of the compound or composition of the disclosure may have the effect of inhibiting LONP1. Diseases or disorders which may benefit from LONP1 inhibition include, for example, proliferative diseases or disorders and cancer.

When administered to a subject, a compound of the disclosure is suitably administered as a component of a composition that comprises a pharmaceutically acceptable carrier or vehicle. One or more additional pharmaceutical acceptable carrier (such as diluents, adjuvants, excipients or vehicles) may be combined with the compound of the disclosure in a pharmaceutical composition. Suitable pharmaceutical carriers are described in “Remington's Pharmaceutical Sciences” by E. W. Martin. Pharmaceutical formulations and compositions of the disclosure are formulated to conform to regulatory standards and according to the chosen route of administration.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are generally sterile. Water is a suitable vehicle when the compound is to be administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the disclosure can take the form of solutions, suspensions, emulsion, tablets, pills, pellets, powders, gels, capsules (for example, capsules containing liquids or powders), modified-release formulations (such as slow or sustained-release formulations), suppositories, emulsions, aerosols, sprays, suspensions, or any other form suitable for use. Other examples of suitable pharmaceutical vehicles are described in Remington's Pharmaceutical Sciences, Alfonso R. Gennaro ed., Mack Publishing Co. Easton, Pa., 19th ed., 1995, see for example pages 1447-1676.

Suitably, the therapeutic compositions or medicaments of the disclosure are formulated in accordance with routine procedures as a pharmaceutical composition adapted for oral administration (more suitably for humans). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunum, or the ileum), or the large intestine. One skilled in the art is able to prepare formulations that will not dissolve in the stomach yet will release the material in the duodenum or elsewhere in the intestine. Suitably, the release will avoid the deleterious effects of the stomach environment, either by protection of the compound (or composition) or by release of the compound (or composition) beyond the stomach environment, such as in the intestine. To ensure full gastric resistance a coating impermeable to at least pH 5.0 would be essential.

Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

While it can be beneficial to provide therapeutic compositions and/or compounds of the disclosure in a form suitable for oral administration, for example, to improve patient compliance and for ease of administration, in some embodiments, compounds or compositions of the disclosure may cause undesirable side-effects, such as intestinal inflammation which may lead to premature termination of a therapeutic treatment regime. Thus, in some embodiments, the therapeutic treatment regime is adapted to accommodate ‘treatment holidays’, e.g. one or more days of non-administration. For example, treatment regimens and therapeutic methods of the disclosure may comprise a repetitive process comprising administration of the therapeutic composition or compound for a number of consecutive days, followed by a treatment holiday of one or more consecutive days. For example, a treatment regime of the disclosure may comprise a repetitive cycle of administration of the therapeutic composition or compound for between 1 and 49 consecutive days, between 2 and 42 days, between 3 and 35 days, between 4 and 28 days, between 5 and 21 days, between 6 and 14 days, or between 7 and 10 days; followed by a treatment holiday of between 1 and 14 consecutive days, between 1 and 12 days, between 1 and 10 days, or between 1 and 7 days (e.g. 1, 2, 3, 4, 5, 6 or 7 days).

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the compound or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the disclosure is via pulmonary or nasal delivery.

Additives may be included to enhance cellular uptake of the therapeutic agent of the disclosure, such as the fatty acids oleic acid, linoleic acid and linolenic acid.

The therapeutic agents of the disclosure may also be formulated into compositions for topical application to the skin of a subject.

Where the disclosure provides more than one active compound/agent for use in combination, generally, the agents may be formulated separately or in a single dosage form, depending on the prescribed most suitable administration regime for each of the agents concerned. When the therapeutic agents are formulated separately, the pharmaceutical compositions of the disclosure may be used in a treatment regime involving simultaneous, separate or sequential administration with the other one or more therapeutic agent. The other therapeutic agent(s) may comprise a compound of the disclosure or a therapeutic agent known in the art.

Specific and general embodiments of the disclosure will now be described by way of the following non-limiting examples.

### EXAMPLES

The examples and preparations provided below further illustrate and exemplify the compounds of the present disclosure and methods of preparing such compounds. It is to be understood that the scope of the present disclosure is not limited in any way by the scope of the following examples and preparations.

The structures of the compounds are confirmed by either elemental analysis or nuclear magnetic resonance (NMR), where peaks assigned to the characteristic protons in the title compound are presented where appropriate. ¹H NMR shift (6) are given in parts per million (ppm) down field from an internal reference standard.

Preparative SFC method: Separation was performed on PIC-SOLUTION-175 instrument by using Reflect (R,R) WHELK-01 column (21.1 mm×250 mm), 5μ operating at 35° C., maintaining flow rate of 60 ml/min using 65% CO2 in super critical state and 35% methanol as mobile phase, run for 12 minutes at 100 bar (detection at 230 nm).

The following abbreviations are used in the synthetic schemes above and the examples below. If an abbreviation used herein is not defined, it has its generally accepted meaning:

| | |
|---|---|
| Abu | Aminobutanoic acid |
| ACN | Acetonitrile |
| Bn | Benzyl |
| BnBr | Benzyl bromide |
| CD₃OD | Deuterated Methanol |
| CuCl₂ | Copper chloride |
| D₂O | Deuterated water |
| DCM | Dichloromethane |
| DIPEA | Diisopropylethylamine |
| DMSO | Dimethylsulfoxide |
| DMSO-d₆ | Deuterated dimethylsulfoxide |
| Et | Ethyl |
| EtOAc | Ethyl acetate |
| Et₃N | Triethylamine |
| EtOH | Ethanol |
| Et₂O | Diethyl ether |
| FA | Formic acid |
| h | hour |
| H₂O | water |
| HBr | Hydrobromic acid |
| HCl | Hydrochloric acid |
| HPLC | High Performance Liquid Chromatography |
| I₂ | Iodine |
| IBCF | Isobutyl chloroformate |
| K₂CO₃ | Potassium carbonate |
| KOAc | Potassium acetate |
| KOtBu | Potassium tert-butoxide |
| LAF | Laminar Flow Box |
| LiOH | Lithium hydroxide |
| min | minutes |
| Me | Methyl |
| MeCN | Acetonitrile |
| MeI | Methyl iodide |
| MeMgBr | Methyl magnesium bromide |
| MeO or OMe | Methoxy |
| MeOD | Deuterated Methanol |
| MeOH | Methanol |
| MgSO₄ | Magnesium sulfate |
| MOD | Modified |
| MS | Mass spectrometry |
| N₂ | Nitrogen |
| NaH | Sodium hydride |
| NMM | N-Methylmorpholine |
| NMR | Nuclear magnetic resonance |
| NaOAc | Sodium acetate |
| NaOH | Sodium hydroxide |
| Na₂CO₃ | Sodium carbonate |
| Na₂SO₄ | Sodium sulfate |
| NaH | Sodium hydride |
| NaHCO₃ | Sodium bicarbonate |
| NH₄Cl | Ammonium chloride |
| NH₄OAc | Ammonium acetate |
| Pd₂(dba)₃ | Tris(dibenzylideneacetone)dipalladium(0) |
| POCl₃ | Phosphoryl chloride |
| Prep-HPLC | Preparative high performance liquid chromatography |
| Prep-TLC | Preparative thin layer chromatography |
| PTSA | p-Toluenesulfonic acid |
| Ph | Phenyl |
| rt | Room temperature (18 to 22° C.) |
| SiO₂ | Silica gel |
| t-BuOH | Tert-butanol |
| THF | Tetrahydrofuran |

### Preparation of the Compounds:

The starting materials and reagents used in each step in the preparation are known and can be readily prepared or purchased from commercial sources.

The compound obtained in each step can also be used for the next reaction as a reaction mixture thereof, or after obtaining a crude product thereof. Alternatively, the compound obtained in each step can be isolated and/or purified from the reaction mixture by a separation means such as concentration, crystallization, recrystallization, distillation, solvent extraction, fractionation, chromatography and the like according to a conventional method.

In each reaction step, while the reaction time varies depending on the reagents and solvents to be used, unless otherwise specified, it is generally 1 min to 48 hr, preferably 10 min to 8 hr.

In the reaction of each step, while the reaction temperature varies depending on the reagents and solvents to be used, unless otherwise specified, it is generally −78° C. to 300° C., preferably −78° C. to 150° C.

In the reaction of each step, unless otherwise specified, a reagent is used in 0.5 equivalent to 20 equivalents, preferably 0.8 equivalent to 5 equivalents, relative to the substrate. When a reagent is used as a catalyst, the reagent is used in 0.001 equivalent to 1 equivalent, preferably 0.01 equivalent to 0.2 equivalent, relative to the substrate. When the reagent is also a reaction solvent, the reagent is used in a solvent amount.

In the reaction of each step, unless otherwise specified, it is performed without solvent or by dissolving or suspending in a suitable solvent. Specific examples of the solvent include the following. Alcohols: methanol, ethanol, tert-butyl alcohol, 2-methoxyethanol and the like; ethers: diethyl ether, diphenyl ether, tetrahydrofuran, 1,2-dimethoxyethane and the like; aromatic hydrocarbons: chlorobenzene, toluene, xylene and the like; saturated hydrocarbons: cyclohexane, hexane and the like; amides: N,N-dimethylformamide, N-methylpyrrolidone and the like; halogenated hydrocarbons: dichloromethane, carbon tetrachloride and the like; nitriles: acetonitrile and the like; sulfoxides: dimethyl sulfoxide and the like; aromatic organic bases: pyridine and the like; acid anhydrides: acetic anhydride and the like; organic acids: formic acid, acetic acid, trifluoroacetic acid and the like; inorganic acids: hydrochloric acid, sulfuric acid and the like; esters: ethyl acetate and the like; ketones: acetone, methyl ethyl ketone and the like; and water. Two or more kinds of the above-mentioned solvents may be used by mixing at an appropriate ratio.

Unless otherwise specified, the reaction of each step is performed according to a known method, for example, the methods described in “Reactions and Syntheses: In the Organic Chemistry Laboratory 2nd Edition” (Lutz F. Tietze, Theophil Eicher, Ulf Diederichsen, Andreas Speicher, Nina Schüzenmeister) Wiley, 2015; “Organic Syntheses Collective Volumes 1-12” (John Wiley & Sons Inc); “Comprehensive Organic Transformations, Third Edition” (Richard C. Larock) Wiley, 2018 and the like.

In each step, protection or deprotection of a functional group is performed by a known method, for example, the methods described in “Protective Groups in Organic Synthesis, 4^{th} Ed.” (Theodora W. Greene, Peter G. M. Wuts) Wiley-Interscience, 2007; “Protecting Groups 3rd Ed.” (P. J. Kocienski) Thieme, 2004 and the like.

Deuterated LONP1 inhibitors of the present disclosure can be prepared using chemical reactions known to a person of ordinary skill in the art using deuterated starting materials or reagents. Deuterium-containing reagents are well known in the art and can be prepared using known procedures or purchased from commercial sources. The deuterated compounds obtained can be characterized by analytical techniques known to persons of ordinary skill in the art. For example, nuclear magnetic resonance (‘NMR’) can be used to determine a compound's structure while mass spectroscopy (‘MS’) can be used to determine the amount of deuterium atom in the compound by comparison to its non-deuterated form.

The Examples and preparations provided below further illustrate and exemplify the compounds of the present disclosure and methods of preparing such compounds. It is to be understood that the scope of the present disclosure is not limited in any way by the scope of the following examples and preparations.

The structures of the compounds are confirmed by either elemental analysis or NMR, where peaks assigned to the characteristic protons in the title compound are presented where appropriate. ¹H NMR shift (δH) are given in parts per million (ppm) down field from an internal reference standard.

### Example 1: Synthesis of ((R)-1-((R)-3-methoxy-2-(pyrazine-2-carboxamido)propanamido)-4-phenylbutyl)boronic acid

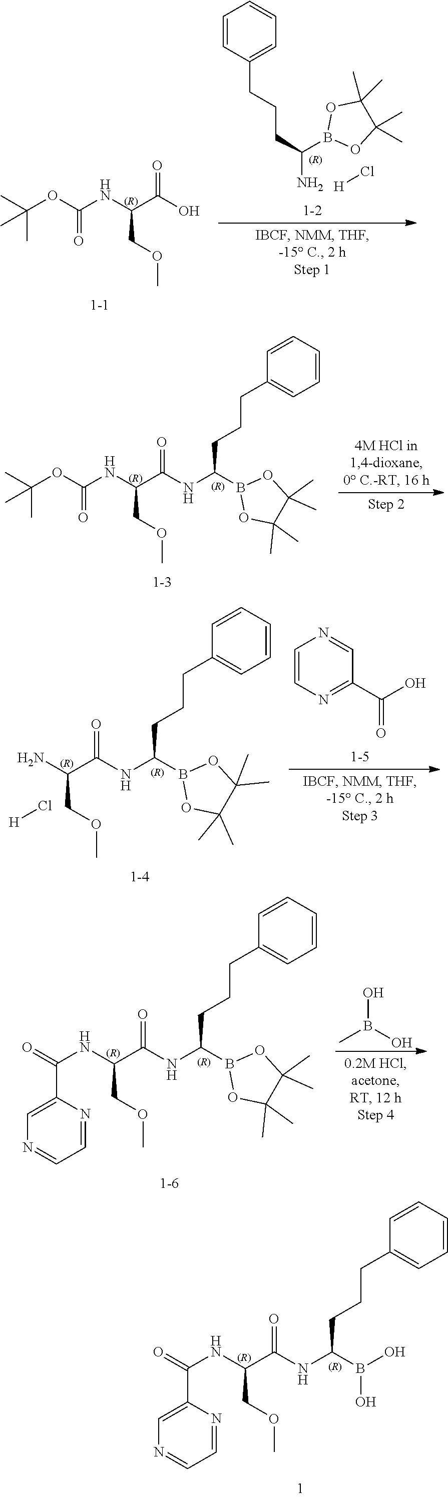

Synthesis of tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate [Step 1]: To a stirred solution of N-(tert-butoxycarbonyl)-O-methyl-D-serine (1-1, 695 mg, 3.2 mmol) in THF (8 mL) was added IBCF (0.4 mL, 3.2 mmol) and NMM (0.35 mL, 3.2 mmol) at −15° C. Reaction mixture was stirred at same temperature for 30 min. (R)-4-phenyl-1-(4,4,5,5-tetramethyl-1,3,2-dioxaborolan-2-yl)butan-1-amine (1-2, 900 mg, 2.9 mmol) in DMF (1 mL) followed by NMM (0.3 mL, 2.9 mmol) was added to the reaction mixture at −15° C. It was gradually warmed to 0° C. and stirred for 2 h. LCMS of crude reaction mass confirmed the formation of desired product. It was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure to afford tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate (1-3, 1.3 g). [M−H]⁻: 475.4.

Synthesis of (R)-2-amino-3-methoxy-N—((R)-4-phenyl-1-(4,4,5,5-tetramethyl-1,3,2-dioxaborolan-2-yl)butyl)propanamide hydrochloride [Step 2]: To a solution of tert-butyl ((R)-3-methoxy-1-oxo-1-(((R)-4-phenyl-1-(4,4,5,5-tetramethyl-1,3,2-dioxaborolan-2-yl)butyl)amino) propan-2-yl)carbamate (1-3, 1.3 g, 2.7 mmol) in 1,4-dioxane (10 mL) was added 4 M HCl in dioxane (8.0 mL, 27.3 mmol) at 0° C. It was gradually warmed to 25° C. and stirred for 16 h. TLC showed complete consumption of starting material to form new polar spot. Volatiles were removed under reduced pressure to get crude (R)-2-amino-3-methoxy-N—((R)-4-phenyl-1-(4,4,5,5-tetramethyl-1,3,2-dioxaborolan-2-yl)butyl)propanamide hydrochloride (1-4, 980 mg). The crude was directly used for next step without purification.

Synthesis of N—((R)-3-methoxy-1-oxo-1-(((R)-4-phenyl-1-(4,4,5,5-tetramethyl-1,3,2-dioxaborolan-2-yl)butyl)amino)propan-2-yl)pyrazine-2-carboxamide [Step 3]: To a stirred solution of pyrazine-2-carboxylic acid (1-5, 325 mg, 2.6 mmol) in THF (8 mL) was added IBCF (0.34 mL, 2.6 mmol) and NMM (0.3 mL, 2.6 mmol) at −15° C. Reaction mixture was stirred at same temperature for 30 min. Then (R)-2-amino-3-methoxy-N—((R)-4-phenyl-1-(4,4,5,5-tetramethyl-1,3,2-dioxaborolan-2-yl)butyl)propanamide hydrochloride (1-4, 980 mg, 2.4 mmol) in DMF (1 mL) followed by NMM (0.3 mL, 2.4 mmol) was added to the reaction mixture under same condition. It was gradually warmed to 0° C. and stirred for 2 h. LCMS of the crude reaction mass confirmed the formation of desired product. It was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure. The crude material was purified by RP PREP-HPLC purification and lyophilized to afford N—((R)-3-methoxy-1-oxo-1-(((R)-4-phenyl-1-(4,4,5,5-tetramethyl-1,3,2-dioxaborolan-2-yl)butyl)amino)propan-2-yl) pyrazine-2-carboxamide (1-6, 110 mg). [M−H]+=481.2 and RT (min)=2.83, [M-84+H]+=399.2. ¹H NMR (400 MHz, CD₃OD) δ 9.23 (brs, 1H), 8.80 (d, 1H), 8.70 (brs, 1H), 7.22-7.09 (m, 5H), 4.98 (t, 1H), 3.89-3.86 (m, 1H), 3.78-3.75 (m, 1H), 3.37 (s, 3H), 2.65-2.57 (m, 3H), 1.67-1.35 (m, 4H), 1.19-1.16 (m, 3H).

Synthesis of ((R)-1-((R)-3-methoxy-2-(pyrazine-2-carboxamido)propanamido)-4-phenyl butyl)boronic acid [Step 4]: To a stirred solution of N—((R)-3-methoxy-1-oxo-1-(((R)-4-phenyl-1-(4,4,5,5-tetramethyl-1,3,2-dioxaborolan-2-yl)butyl)amino)propan-2-yl)pyrazine-2-carboxamide (1-6, 110 mg, 0.23 mmol) and methylboronic acid (135 mg, 2.3 mmol) in acetone (4 mL) was added 0.2 M HCl (4 mL) and the reaction mixture was stirred at RT overnight. TLC and LCMS showed full conversion of starting material with formation of new polar spot. The volatiles were evaporated and the residue was purified by RP prep HPLC purification and lyophilized to afford ((R)-1-((R)-3-methoxy-2-(pyrazine-2-carboxamido)propanamido)-4-phenylbutyl)boronic acid (Compound 1, 49 mg). [M−H]: 399.3; ¹H NMR (400 MHz, CD₃OD) δ 9.23 (brs, 1H), 8.81 (d, 1H), 8.70-8.69 (m, 1H), 7.22-7.08 (m, 5H), 4.98 (t, 1H), 3.89-3.85 (m, 1H), 3.78-3.75 (m, 1H), 3.37 (s, 3H), 2.65-2.56 (m, 3H), 1.68-1.46 (m, 4H).

### Example 2: ((R)-2-(benzyloxy)-1-((R)-3-methoxy-2-(pyrazine-2-carboxamido)propanamido) ethyl)boronic acid

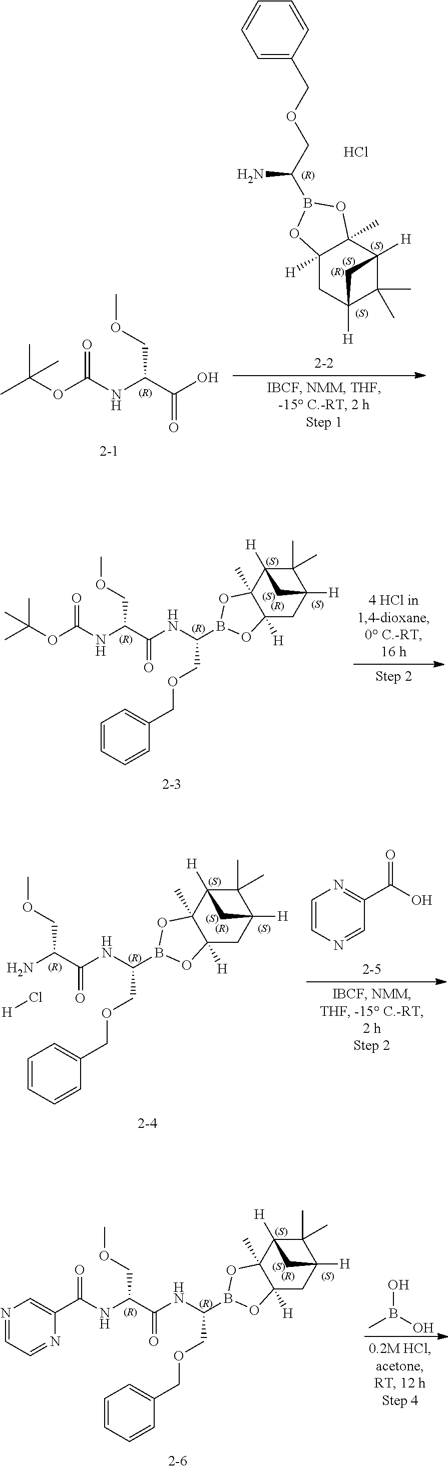

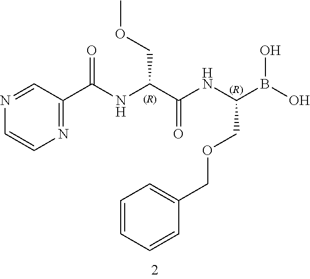

Synthesis of tert-butyl ((R)-1-(((R)-2-(benzyloxy)-1-((3aS,4S,6S,7aR)-3a,5,5-trimethyl hexahydro-4,6-methanobenzo[d][1,3,2]dioxaborol-2-yl)ethyl)amino)-3-methoxy-1-oxopropan-2-yl)carbamate [Step 1]: To a stirred solution of in N-(tert-butoxycarbonyl)-O-methyl-D-serine (2-1, 65 mg, 0.3 mmol) in THF (3 mL), was added IBCF (0.04 mL, 0.3 mmol) followed by NMM (0.04 mL, 0.3 mmol) at −15° C. and the reaction mixture was stirred at that temperature for 1h. ((R)-2-(benzyloxy)-1-((3aS,4S,6S,7aR)-3a,5,5-trinethylhexahydro-4,6-methanobenzo[d][1,3,2]dioxaborol-2-yl)ethan-1-amine hydrochloride (2-2, 100 mg, 0.3 mmol) and NMM (0.04 mL, 0.3 mmol) were added and the reaction mixture was stirred at RT for 2h. TLC and LCMS showed full consumption of starting material with formation of new spot. The reaction mixture was diluted with EtOAc and washed subsequently with 0.1 M HCl (2×30 mL), 5% K₂CO₃ (2×30 mL), water (2×30 mL) and brine. The organic phase was dried over Na₂SO₄, filtered and evaporated to yield tert-butyl ((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-3-methoxy-1-oxopropan-2-yl)carbamate (2-3, 120 mg). The crude was used in the next step without further purification. [M+H]+=531.0.

Synthesis of (R)-2-amino-N—((R)-2-(benzyloxy)-1-((3aS,4S,6S,7aR)-3a,5,5-trimethyl hexa hydro-4,6-methanobenzo[d][1,3,2]dioxaborol-2-yl)ethyl)-3-methoxypropanamide hydrochloride [Step 2]: To a stirred solution of tert-butyl ((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl) amino)-3-methoxy-1-oxopropan-2-yl)carbamate (2-3, 350 mg, 0.7 mmol) in 1,4-dioxane (4 mL), 4 M HCl in dioxane (3.3 mL, 13.2 mmol) was added in ice cold condition and the reaction mixture was stirred at RT for 2h. TLC showed full conversion of starting material with formation of new polar spot. LCMS showed the formation of desired product. The solvent was evaporated to yield (R)-2-amino-N—((R)-2-(benzyloxy)-1-((3aS,4S,6S,7aR)-3a,5,5-trimethylhexahydro-4,6-methanobenzo[d][1,3,2]dioxaborol-2-yl)ethyl)-3-methoxypropanamide hydrochloride (2-4, 300 mg). [M+H]⁺=430.9.

Synthesis of N—((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-3-methoxy-1-oxopropan-2-yl) pyrazine-2-carboxamide [Step 3]: To a stirred solution of in pyrazine-2-carboxylic acid (2-5, 100 mg, 0.8 mmol) in THF (5 mL) was added IBCF (0.11 mL, 0.8 mmol) followed by NMM (0.11 mL, 0.8 mmol) at −15° C. and the reaction mixture was stirred at that temperature for 1h. (R)-2-amino-N—((R)-2-(benzyloxy)-1-((3aS,4S,6S,7aR)-3a,5,5-trimethylhexahydro-4,6-methanobenzo [d][1,3,2]dioxaborol-2-yl)ethyl)-3-methoxypropanamide hydrochloride (2-4, 350 mg, 0.8 mmol) and NMM (0.11 mL, 0.8 mmol) were added and the reaction mixture was stirred at RT for 2h. TLC and LCMS showed full consumption of starting material with formation of new spot. The reaction mixture was diluted with EtOAc and washed subsequently with 0.1 M HCl (2×30 mL), 5% K₂CO₃ (2×30 mL), water (2×30 mL) and brine. The organic phase was dried over Na₂SO₄, filtered and evaporated to yield a brown gum. The crude was purified by prep HPLC purification and lyophilized to afford N—((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-3-methoxy-1-oxopropan-2-yl)pyrazine-2-carboxamide (2-6, 80 mg). [M+H]⁺=537.0.

Synthesis of ((R)-2-(benzyloxy)-1-((R)-3-methoxy-2-(pyrazine-2-carboxamido) propanamido)ethyl)boronic acid [Step 4]: To a stirred solution of N—((R)-1-(((R)-2-(benzyloxy)-1-((3aS,4S,6S,7aR)-3a,5,5-trimethylhexahydro-4,6-methanobenzo[d][1,3,2]dioxaborol-2-yl)ethyl)amino)-3-methoxy-1-oxopropan-2-yl)pyrazine-2-carboxamide (80 mg, 0.15 mmol) and methylboronic acid (2-6, 135 mg, 2.2 mmol) in acetone (2 mL) was added 0.2 M HCl (1.0 mL, 0.1 mmol) and the reaction mixture was stirred at RT overnight. TLC and LCMS showed full conversion of starting material with formation of new polar spot. The volatiles were evaporated and the residue was purified via prep HPLC purification and lyophilized to afford ((R)-2-(benzyloxy)-1-((R)-3-methoxy-2-(pyrazine-2-carboxamido)propanamido)ethyl)boronic acid (Compound 2, 40 mg). [M−H]+: 400.9. ¹H NMR (400 MHz, CD₃OD) δ 9.24 (s, 1H), 8.80 (d, 1H), 8.70 (d, 1H), 7.34-7.23 (m, 5H), 5.06 (t, 1H), 4.49 (d, 2H), 3.90-3.86 (m, 1H), 3.81-3.77 (m, 1H), 3.59-3.56 (m, 1H), 3.46-3.43 (m, 1H), 3.39 (brs, 3H), 2.95 (d, 1H).

### Example 3: ((R)-1-((R)-2-benzamido-3-methoxypropanamido)-4-phenylbutyl)boronic acid

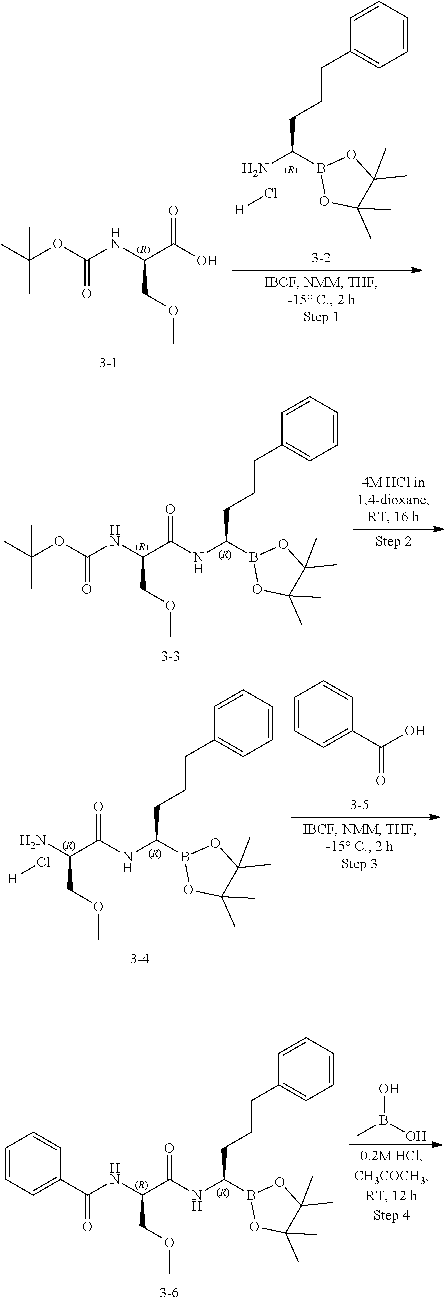

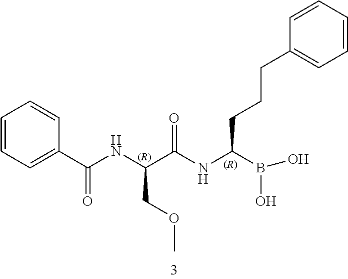

Synthesis of tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate [Step 1]: To a stirred solution of N-(tert-butoxycarbonyl)-O-methyl-D-serine (3-1, 770 mg, 3.5 mmol) in THF (15 mL) were added NMM (0.4 mL, 3.5 mmol), IBCF (0.4 mL, 3.2 mmol) at −15° C. and the reaction mixture was stirred at this temperature for 1 h. To this solution were added (R)-4-phenyl-1-(4,4,5,5-tetramethyl-1,3,2-dioxaborolan-2-yl)butan-1-amine hydrochloride (3-2, 1 g, 3.2 mmol) and NMM (0.4 mL, 3.5 mmol) and the reaction mixture was stirred at ambient temperature for 2 h. Reaction mixture was diluted with ethyl acetate and washed subsequently with 0.1 M HCl, 5% K₂CO₃, water and brine. The organic extracts was dried over Na₂SO₄, filtered and evaporated to afford tert-butyl ((R)-3-methoxy-1-oxo-1-(((R)-4-phenyl-1-(4,4,5,5-tetramethyl-1,3,2-dioxaborolan-2-yl)butyl)amino) propan-2-yl)carbamate (3-3, 1.5 g). The product was used for next step without further purification. [M−H]⁻: 474.9.

Synthesis of (R)-2-amino-3-methoxy-N—((R)-4-phenyl-1-(4,4,5,5-tetramethyl-1,3,2-dioxaborolan-2-yl)butyl)propanamide hydrochloride [Step 2]: To a stirred solution of tert-butyl ((R)-3-methoxy-1-oxo-1-(((R)-4-phenyl-1-(4,4,5,5-tetramethyl-1,3,2-dioxaborolan-2-yl) butyl)amino)propan-2-yl)carbamate (3-3, 300 mg, 0.6 mmol) in 1,4-Dioxane (2 mL), add 4 M HCl-dioxane (2 mL, 8 mmol) was added in ice cold condition and the reaction mixture was stirred at ambient temperature for 2 h. Product was triturated with n-pentane to give (R)-2-amino-3-methoxy-N—((R)-4-phenyl-1-(4,4,5,5-tetramethyl-1,3,2-dioxaborolan-2-yl)butyl) propanamide hydrochloride (3-4, 250 mg). The product was used for next step without further purification. [M−H]⁻: 375.4.

Synthesis of N—((R)-3-methoxy-1-oxo-1-(((R)-4-phenyl-1-(4,4,5,5-tetramethyl-1,3,2-dioxaborolan-2-yl)butyl)amino)propan-2-yl)benzamide [Step 3]: To a stirred solution of Benzoic Acid (3-5, 75 mg, 0.6 mmol) in THF (7 mL), NMM (0.07 mL, 0.7 mmol), IBCF (0.08 mL, 0.6 mmol) were added at −15° C. and the reaction mixture was stirred at this temperature for 30 min. To this solution were added (R)-2-amino-3-methoxy-N—((R)-4-phenyl-1-(4,4,5,5-tetramethyl-1,3,2-dioxaborolan-2-yl)butyl) propanamide hydrochloride (3-4, 250 mg, 0.6 mmol) and NMM (0.07 mL, 0.7 mmol) and the reaction mixture was stirred at ambient temperature for 2 h. The reaction mixture was diluted with ethylacetate and washed subsequently with 0.1 M HCl, 5% K₂CO₃, water and brine. The organic phase was dried over Na₂SO₄, filtered and evaporated under reduced pressure. The product was purified by RP prep HPLC purification and the eluent was lyophilized to afford N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (3-6, 50 mg). [M−H]⁻: 479.2.

Synthesis of ((R)-1-((R)-2-benzamido-3-methoxypropanamido)-4-phenylbutyl)boronic acid [Step 4]: To a stirred solution of N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (3-6, 50 mg, 0.1 mmol) in acetone (3 mL) was added methylboronic acid (60 mg, 1 mmol) followed by drop wise addition of 0.2 M HCl (3 mL). The reaction mixture was allowed to stir at ambient temperature for 16 h. All the volatiles were evaporated at room temperature and residue was dissolved in acetonitrile and deionized water and freeze-dried to obtain solid. The crude solid was purified through RP prep HPLC purification and lyophilized to afford ((R)-1-((R)-2-benzamido-3-methoxypropanamido)-4-phenylbutyl)boronic acid (Compound 3, 35 mg). [M−H]⁻=397.3. ¹H NMR (400 MHz, CD₃OD) δ_{H}7.85 (d, 2H), 7.55 (t, 1H), 7.46 (t, 2H), 7.22-7.09 (m, 5H), 4.98 (t, 1H), 3.80-3.74 (m, 2H), 3.37 (s, 3H), 2.63-2.58 (m, 3H), 1.70-1.63 (m, 2H), 1.57-1.51 (2H).

### Example 4: ((R)-1-((R)-3-methoxy-2-(6-methoxypicolinamido)propanamido)-4-phenylbutyl) boronic acid

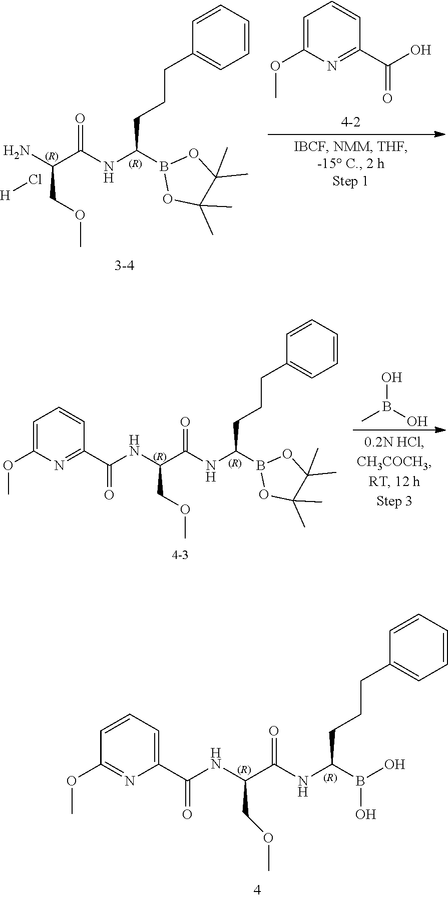

Synthesis of 6-methoxy-N—((R)-3-methoxy-1-oxo-1-(((R)-4-phenyl-1-(4,4,5,5-tetramethyl-1,3,2-dioxaborolan-2-yl)butyl)amino)propan-2-yl)picolinamide [Step 1]: To a stirred solution of 6-methoxypicolinic acid (4-2, 140 mg, 0.9 mmol) in THF (10 mL), NMM (0.1 mL, 0.8 mmol), IBCF (0.1 mL, 0.7 mmol) were added at −15° C. and the reaction mixture was stirred at this temperature for 30 min. Then (R)-2-amino-3-methoxy-N—((R)-4-phenyl-1-(4,4,5,5-tetramethyl-1,3,2-dioxaborolan-2-yl)butyl) propanamide hydrochloride (3-4, 300 mg, 0.7 mmol) and NMM (0.1 mL, 0.8 mmol) were added and the reaction mixture was stirred at ambient temperature for 2 h. Reaction mixture was diluted with ethyl acetate and washed subsequently with 0.1 M HCl, 5% K₂CO₃, water and brine. The organic extracts was dried over Na₂SO₄, filtered and evaporated under reduced pressure. The product was purified by reverse phase prep HPLC to afford 6-methoxy-N—((R)-3-methoxy-1-oxo-1-(((R)-4-phenyl-1-(4,4,5,5-tetramethyl-1,3,2-dioxaborolan-2-yl)butyl)amino) propan-2-yl)picolinamide (4-3, 50 mg). [M−H]⁻: 510.3.

Synthesis of ((R)-1-((R)-3-methoxy-2-(6-methoxypicolinamido)propanamido)-4-phenyl butyl)boronic acid [Step 2]: To a solution of 6-methoxy-N—((R)-3-methoxy-1-oxo-1-(((R)-4-phenyl-1-(4,4,5,5-tetramethyl-1,3,2-dioxaborolan-2-yl)butyl)amino) propan-2-yl)picolinamide (4-3, 50 mg, 0.1 mmol) in acetone (3 mL) was added methylboronic acid (60 mg, 1 mmol) followed by drop wise addition of 0.2 M HCl (2 mL). The reaction mixture was allowed to stirring at ambient temperature for 16 h. All the volatiles were evaporated under reduced pressure and residue was dissolved in acetonitrile and deionized water and freeze-dried to obtain solid. The product was purified through RP prep HPLC purification to afford ((R)-1-((R)-3-methoxy-2-(6-methoxypicolinamido)propanamido)-4-phenylbutyl)boronic acid (Compound 4, 35 mg). [M−H]⁻: 428.4. ¹H NMR (400 MHz, CD₃OD) δ_{H} 7.83 (t, 1H), 7.68 (d, 1H), 7.22-7.14 (m, 4H), 7.11 (d, 1H), 7.01 (d, 1H), 4.92 (s, 1H), 3.99 (s, 3H), 3.90-3.86 (m, 1H), 3.77-3.73 (m, 1H), 3.38 (s, 3H), 2.66 (d, 1H), 2.62-2.60 (m, 2H), 1.69-1.58 (m, 2H), 1.57-1.49 (m, 2H).

### Example 5: ((R)-1-((R)-3-methoxy-2-(pyrazine-2-carboxamido)propanamido)-4-(4-methoxy phenyl)butyl)boronic acid

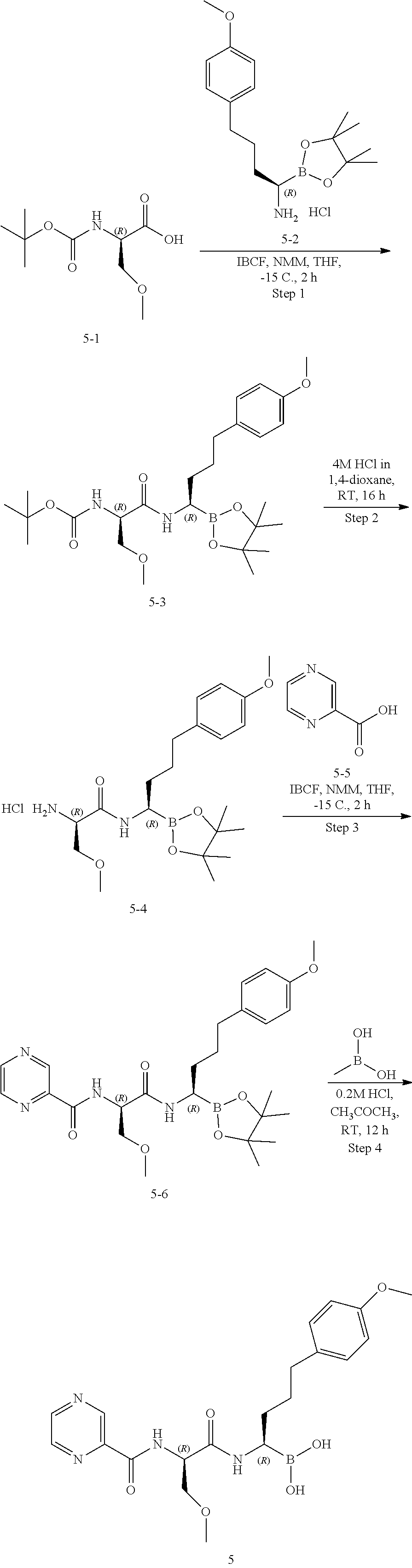

Synthesis of tert-butyl ((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)carbamate [Step 1]: To a stirred solution of N-(tert-butoxycarbonyl)-O-methyl-D-serine (5-1, 142 mg, 0.64 mmol) in THF (6 mL) was added IBCF (0.09 mL, 0.64 mmol) and NMM (0.09 mL, 0.64 mmol) at −15° C. Reaction mixture was stirred at same temperature for 30 min. Then (R)-4-(4-methoxyphenyl)-1-(4,4,5,5-tetramethyl-1,3,2-dioxaborolan-2-yl)butan-1-amine hydrochloride (5-2, 200 mg, 0.58 mmol) in DMF (0.5 mL) followed by NMM (0.8 mL, 0.58 mmol) was added to the reaction mixture at −15° C. It was gradually warmed to 0° C. and stirred for 2 h. LCMS confirmed the formation of desired product. It was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure to afford tert-butyl ((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)carbamate (5-3, 230 mg). [M−H]⁻: 505.5

Synthesis of (R)-2-amino-4-morpholino-4-oxo-N—((S)-4-phenyl-1-(4,4,5,5-tetramethyl-1,3,2-dioxaborolan-2-yl)butyl)butanamide hydrochloride [Step 2]: To a solution of tert-butyl ((R)-3-methoxy-1-(((R)-4-(4-methoxyphenyl)-1-(4,4,5,5-tetramethyl-1,3,2-dioxaborolan-2-yl)butyl) amino)-1-oxopropan-2-yl)carbamate (5-3, 200 mg, 0.4 mmol) in 1,4 Dioxane (2 mL) was added 4 M HCl in dioxane (2.0 mL, 8.0 mmol) at 0° C. It was gradually warmed to 25° C. and stirred for 16 h. TLC showed complete consumption of starting material to form new polar spot. Volatiles were removed under reduced pressure to get (R)-2-amino-3-methoxy-N—((R)-4-(4-methoxyphenyl)-1-(4,4,5,5-tetramethyl-1,3,2-dioxaborolan-2-yl)butyl)propanamide hydrochloride (5-4, 170 mg). Crude was directly used for next step without purification. [M−H]⁻: 405.3.

Synthesis of N—((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)pyrazine-2-carboxamide [Step 3]: To a stirred solution of pyrazine-2-carboxylic acid (5-5, 60 mg, 0.4 mmol) in THF (6 mL) was added IBCF (0.06 mL, 0.4 mmol) and NMM (0.06 mL, 0.4 mmol) at −15° C. The reaction mixture was stirred at same temperature for 30 min. Then (R)-2-amino-3-methoxy-N—((R)-4-(4-methoxyphenyl)-1-(4,4,5,5-tetramethyl-1,3,2-dioxaborolan-2-yl)butyl)propanamide hydrochloride (5-4, 170 mg, 0.38 mmol) in DMF (1 mL) followed by NMM (0.05 mL, 0.38 mmol) was added to the reaction mixture under same condition. It was gradually warmed to 0° C. and stirred for 2 h. LCMS of the crude reaction mass confirmed the formation of desired product. It was neutralized with saturated aqueous 0.1 M HCl solution and extracted with ethyl acetate. The combined organic layer was washed with 5% K₂CO₃ solution, water, brine, dried over Na₂SO₄, filtered and evaporated under reduced pressure to afford N—((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)pyrazine-2-carboxamide (5-6, 180 mg). [M−H]⁻: 511.4.

Synthesis of ((R)-1-((R)-3-methoxy-2-(pyrazine-2-carboxamido)propanamido)-4-(4-methoxyphenyl)butyl)boronic acid [Step 4]: To a stirred solution of N—((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)pyrazine-2-carboxamide (5-6, 170 mg, 0.3 mmol) and methylboronic acid (198 mg, 3.0 mmol) in acetone (5 mL) was added 0.2 M HCl (5 mL) and the reaction mixture was stirred at RT overnight. TLC and LCMS showed full conversion of starting material with formation of new polar spot. The volatiles were evaporated and the crude material was purified by RP prep HPLC purification and lyophilized to afford ((R)-1-((R)-3-methoxy-2-(pyrazine-2-carboxamido) propanamido)-4-(4-methoxyphenyl)butyl)boronic acid (Compound 5, 20 mg). [M−H]: 429.4. ¹H NMR (400 MHz, CD₃OD) δ 9.23 (s, 1H), 8.81 (d, 1H), 8.70 (s, 1H), 7.06 (d, 2H), 6.77 (d, 2H), 4.98 (t, 1H), 3.89-3.72 (m, 2H), 3.73 (s, 3H), 3.37 (s, 3H), 2.64 (t, 1H), 2.55-2.50 (m, 2H), 1.63-1.43 (m, 4H).

### Example 6: N—((R)-5-morpholino-1,5-dioxo-1-(((R)-4-phenyl-1-(4,4,5,5-tetramethyl-1,3,2-dioxaborolan-2-yl)butyl)amino)pentan-2-yl)pyrazine-2-carboxamide

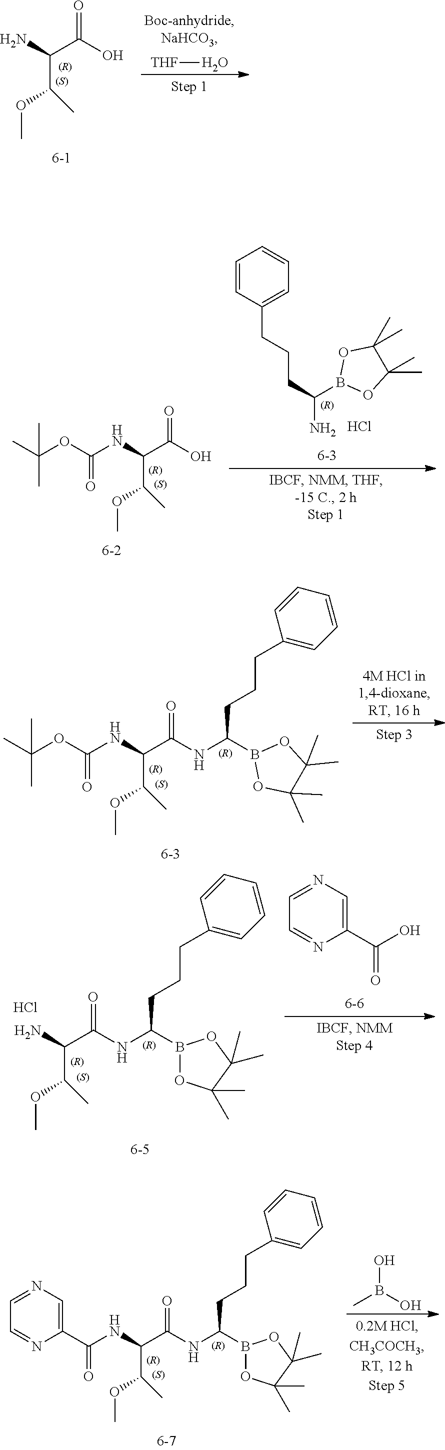

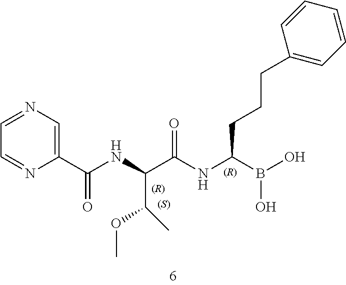

Synthesis of N-(tert-butoxycarbonyl)-O-methyl-D-threonine [Step 1]: To a stirred solution of O-methyl-D-threonine (6-1, 500 mg, 3.8 mmol) in THF (30 mL) and water (30 mL) was added NaHCO₃ (950 mg, 11 mmol) followed by Boc-anhydride (1.3 mL, 5.6 mmol) at 0° C. and then the reaction mixture was allowed to stir at RT for 18 h. The reaction was quenched by KHSO₄ solution to pH=4 and extracted with EtOAc. Organic solvents were evaporated to afford N-(tert-butoxycarbonyl)-O-methyl-D-threonine (6-2, 850 mg) as crude which was forwarded to next step directly. ¹H NMR (400 MHz, CDCl₃) δ 5.26 (d, 1H), 4.31 (d, 1H), 3.96 (d, 1H), 3.34 (s, 3H), 1.44 (s, 9H), 1.20 (d, 3H).

Synthesis of tert-butyl ((2R,3S)-3-methoxy-1-oxo-1-(((R)-4-phenyl-1-(4,4,5,5-tetramethyl-1,3,2-dioxaborolan-2-yl)butyl)amino)butan-2-yl)carbamate [Step 2]: To a stirred solution of N-(tert-butoxycarbonyl)-O-methyl-D-threonine (6-2, 180 mg, 0.8 mmol) in THF (4 mL) was added IBCF (0.1 mL, 0.6 mmol) and NMM (0.1 mL, 0.7 mmol) at −15° C. Reaction mixture was stirred at same temperature for 30 min. (R)-4-phenyl-1-(4,4,5,5-tetramethyl-1,3,2-dioxaborolan-2-yl)butan-1-amine hydrochloride (6-3, 200 mg, 0.6 mmol) in DMF (2 mL) followed by NMM (0.1 mL, 0.6 mmol) was added to reaction mixture at −15° C. It was gradually warmed to 0° C. and stirred for 2 h. LCMS of crude reaction mass confirmed the formation of desired product. It was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure to afford tert-butyl ((2R,3S)-3-methoxy-1-oxo-1-(((R)-4-phenyl-1-(4,4,5,5-tetramethyl-1,3,2-dioxaborolan-2-yl)butyl)amino)butan-2-yl)carbamate (6-4, 300 mg). Crude was used directly without further purification. [M−H]⁻: 489.

Synthesis of (2R,3S)-2-amino-3-methoxy-N—((R)-4-phenyl-1-(4,4,5,5-tetramethyl-1,3,2-dioxaborolan-2-yl)butyl)butanamide [Step 3]: To a solution of tert-butyl ((2R,3S)-3-methoxy-1-oxo-1-(((R)-4-phenyl-1-(4,4,5,5-tetramethyl-1,3,2-dioxaborolan-2-yl)butyl)amino)butan-2-yl) carbamate (6-4, 300 mg, 0.6 mmol) in 1,4-Dioxane (6 mL) was added 4 M HCl in 1,4 Dioxane (2.5 mL, 9 mmol) at 0° C. It was gradually warmed to 25° C. and stirred for 16 h. TLC showed complete consumption of starting material to form new polar spot. Volatiles were removed under reduced pressure to get (2R,3S)-2-amino-3-methoxy-N—((R)-4-phenyl-1-(4,4,5,5-tetramethyl-1,3,2-dioxaborolan-2-yl)butyl)butanamide (6-5, 200 mg). Crude was directly used for next step without purification. [M−H]⁻: 389.

Synthesis of N-((2R,3S)-3-methoxy-1-oxo-1-(((R)-4-phenyl-1-(4,4,5,5-tetramethyl-1,3,2-dioxaborolan-2-yl)butyl)amino)butan-2-yl)pyrazine-2-carboxamide [Step 4]: To a stirred solution of pyrazine-2-carboxylic acid (6-6, 76 mg, 0.6 mmol) in THF (5 mL) under argon atmosphere was added IBCF (0.1 mL, 0.5 mmol) followed by NMM (0.1 mL, 0.5 mmol) at −15° C. and stirred for 45 min. (2R,3S)-2-amino-3-methoxy-N—((R)-4-phenyl-1-(4,4,5,5-tetramethyl-1,3,2-dioxaborolan-2-yl)butyl)butanamide (6-5, 200 mg, 0.5 mmol) followed by NMM (0.1 mL, 0.5 mmol) was added to it and stirred for 2 h. The reaction was quenched with water and diluted with EtOAc. Organic layer was collected and washed successively with 0.1 M aqueous HCl, 5% aqueous K₂CO₃ solution, water and brine, dried over anhydrous Na₂SO₄ and concentrated under reduced pressure to get crude material was purified by prep HPLC purification and lyophilized to afford N-((2R,3S)-3-methoxy-1-oxo-1-(((R)-4-phenyl-1-(4,4,5,5-tetramethyl-1,3,2-dioxaborolan-2-yl)butyl)amino)butan-2-yl)pyrazine-2-carboxamide (6-7, 100 mg). [M−H]⁻: 495.3.

Synthesis of N—((R)-5-morpholino-1,5-dioxo-1-(((R)-4-phenyl-1-(4,4,5,5-tetramethyl-1,3,2-dioxaborolan-2-yl)butyl)amino)pentan-2-yl)pyrazine-2-carboxamide [Step 5]: To a stirred solution of N-((2R,3S)-3-methoxy-1-oxo-1-(((R)-4-phenyl-1-(4,4,5,5-tetramethyl-1,3,2-dioxaborolan-2-yl)butyl)amino)butan-2-yl)pyrazine-2-carboxamide (6-7, 80 mg, 0.2 mmol) and methylboronic acid (96 mg, 1.6 mmol) in acetone (4 mL) was added 0.2 M HCl (4.0 mL) and the reaction mixture was stirred at RT overnight. TLC and LCMS showed full conversion of starting material with formation of new polar spot. The volatiles were evaporated and the residue was re-dissolved in acetone and deionized water and freeze-dried to obtain crude. Crude material was purified by prep HPLC purification and lyophilized to afford ((R)-1-((2R,3S)-3-methoxy-2-(pyrazine-2-carboxamido)butanamido)-4-phenylbutyl)boronic acid (Compound 6, 40 mg). [M−H]⁻: 413. ¹H NMR (400 MHz, CD3OD) δ 9.25 (s, 1H), 8.82 (d, 1H), 8.71 (s, 1H), 7.23-7.16 (m, 4H), 7.14-7.08 (m, 1H), 4.08-4.05 (m, 1H), 3.33 (s, 3H), 2.65-2.57 (m, 4H), 1.69-1.64 (m, 2H), 1.61-1.45 (m, 2H), 1.22 (d, 3H).

### Example 7: ((R)-1-((R)-3-methoxy-2-((S)-5-oxopyrrolidine-2-carboxamido)propanamido)-4-phenylbutyl)boronic acid

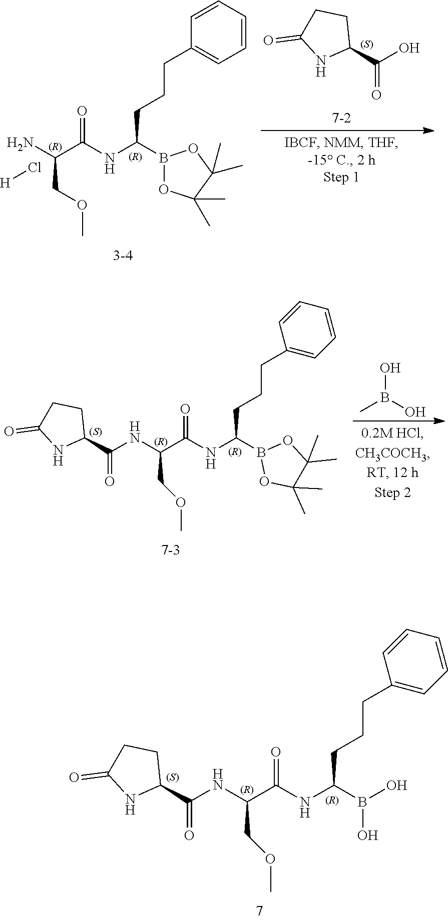

Synthesis of (S)—N—((R)-3-methoxy-1-oxo-1-(((R)-4-phenyl-1-(4,4,5,5-tetramethyl-1,3,2-dioxaborolan-2-yl)butyl)amino)propan-2-yl)-5-oxopyrrolidine-2-carboxamide [Step 1]: To a stirred solution of (S)-5-oxopyrrolidine-2-carboxylic acid (7-2, 103 mg, 0.8 mmol) in THF (5 mL) were added IBCF (0.08 mL, 0.7 mmol) and NMM (0.09 mL, 0.8 mmol) at −15° C. Reaction mixture was stirred at same temperature for 30 min. (R)-2-amino-3-methoxy-N—((R)-4-phenyl-1-(4,4,5,5-tetramethyl-1,3,2-dioxaborolan-2-yl)butyl)propanamide hydrochloride (3-4, 300 mg, 0.7 mmol) in DMF (0.2 mL) and NMM (0.08 mL, 0.7 mmol) were added to reaction mixture at same condition. The reaction mixture was gradually warmed to 0° C. and stirred for 2 h. The reaction mixture was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure. LCMS of organic phase didn't show desired mass peak but aqueous part showed mass of corresponding boronic acid. Crude material was purified by prep HPLC purification and lyophilized to afford (S)—N—((R)-3-methoxy-1-oxo-1-(((R)-4-phenyl-1-(4,4,5,5-tetramethyl-1,3,2-dioxaborolan-2-yl)butyl)amino)propan-2-yl)-5-oxopyrrolidine-2-carboxamide (7-3, 110 mg). [M−H]⁻: 486.3.

Synthesis of ((R)-1-((R)-3-methoxy-2-((S)-5-oxopyrrolidine-2-carboxamido)propanamido)-4-phenylbutyl)boronic acid [Step 2]: To a stirred solution of (S)—N—((R)-3-methoxy-1-oxo-1-(((R)-4-phenyl-1-(4,4,5,5-tetramethyl-1,3,2-dioxaborolan-2-yl)butyl)amino)propan-2-yl)-5-oxopyrrolidine-2-carboxamide (7-3, 47 mg, 0.08 mmol) and methylboronic acid (47 mg, 0.8 mmol) in acetone (5 mL) was added 0.2 M HCl (5.0 mL) and the reaction mixture was stirred at RT overnight. The volatiles were evaporated and the residue was dissolved in acetone and deionized water and freeze-dried to obtain crude. Crude material was purified by prep HPLC purification and lyophilized to afford ((R)-1-((R)-3-methoxy-2-((S)-5-oxopyrrolidine-2-carboxamido)propanamido)-4-phenylbutyl)boronic acid (Compound 7, 31 mg). [M−H]⁻: 404.2. ¹H NMR (400 MHz, CD₃OD) δ 7.24-7.10 (m, 5H), 4.75 (t, 1H), 4.27-4.23 (m, 1H), 3.73-3.70 (m, 1H), 3.64-3.61 (m, 1H), 3.33 (s, 3H), 2.64-2.59 (m, 3H), 2.42-2.28 (m, 4H), 2.05 (brs, 1H), 1.68-1.45 (m, 4H).

### Example 8: Synthesis of ((R)-1-((R)-3-methoxy-2-((R)-5-oxopyrrolidine-2-carboxamido) propanamido)-4-phenylbutyl)boronic acid

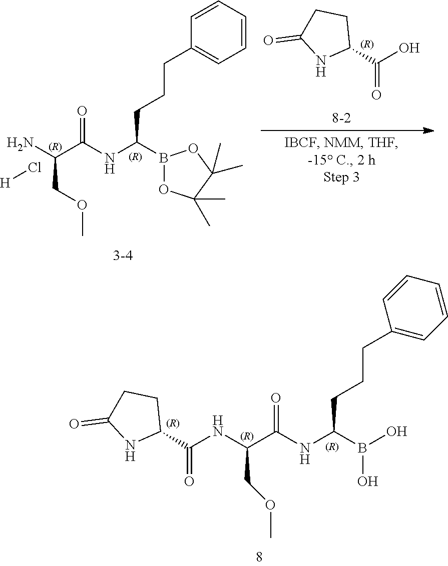

Synthesis of ((R)-1-((R)-3-methoxy-2-((R)-5-oxopyrrolidine-2-carboxamido)propanamido)-4-phenylbutyl)boronic acid [Step 3]: To a stirred solution of (R)-5-oxopyrrolidine-2-carboxylic acid (8-2, 34 mg, 0.3 mmol) in THF (5 mL) was added IBCF (0.03 mL, 0.2 mmol) and NMM (0.03 mL, 0.3 mmol) at −15° C. Reaction mixture was stirred at same temperature for 30 min. (R)-2-amino-3-methoxy-N—((R)-4-phenyl-1-(4,4,5,5-tetramethyl-1,3,2-dioxaborolan-2-yl)butyl) propanamide hydrochloride (3-4, 100 mg, 0.24 mmol) in DMF (0.5 mL) followed by NMM (0.03 mL, 0.24 mmol) was added to reaction mixture under same condition. It was gradually warmed to 0° C. and stirred for 2 h. It was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure. LCMS of aqueous phase showed corresponding boronic acid mass peak organic layer didn't contain any desired mass peak. Aqueous part was lyophilized and purified via prep HPLC purification and lyophilized to afford directly ((R)-1-((R)-3-methoxy-2-((R)-5-oxopyrrolidine-2-carboxamido)propanamido)-4-phenylbutyl)boronic acid (Compound 8, 15 mg). [M−H]⁻: 404.3. 1H NMR (400 MHz, CD₃OD) δ 7.24-7.10 (m, 5H), 4.76 (t, 1H), 4.25-4.22 (m, 1H), 3.72-3.68 (m, 1H), 3.65-3.61 (m, 1H), 3.33 (s, 3H), 2.66-2.58 (m, 3H), 2.42-2.27 (m, 4H), 2.10-2.05 (m, 1H), 1.68-1.47 (m, 4H).

### Example 9: ((R)-1-((R)-3-methoxy-2-(picolinamido)propanamido)-4-phenylbutyl)boronic acid PZL-1780

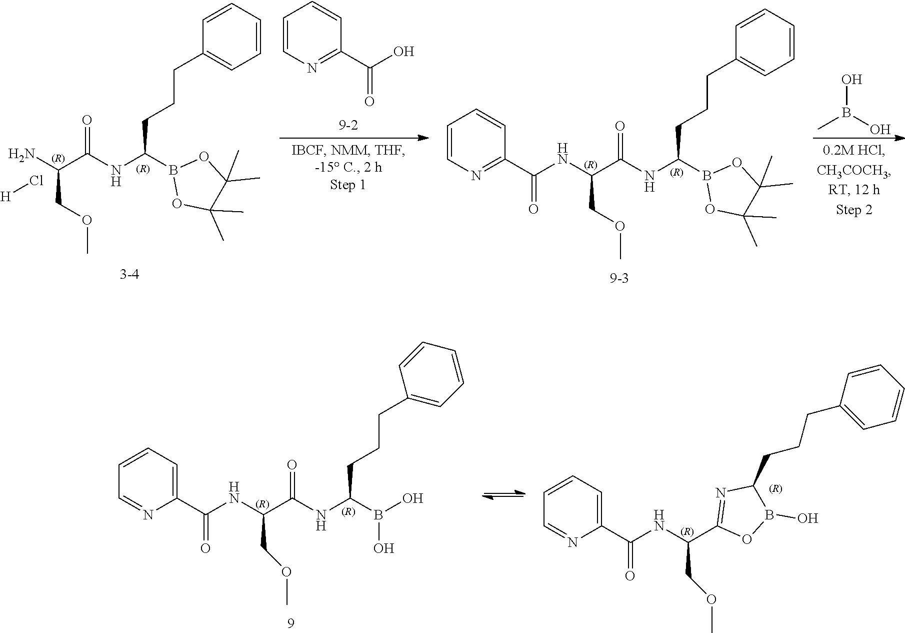

Synthesis of N—((R)-3-methoxy-1-oxo-1-(((R)-4-phenyl-1-(4,4,5,5-tetramethyl-1,3,2-dioxaborolan-2-yl)butyl)amino)propan-2-yl)picolinamide [Step 1]: To a stirred solution of picolinic acid (9-2, 120 mg, 1 mmol) in THF (10 mL), NMM (0.1 mL, 0.8 mmol), IBCF (0.1 mL, 0.8 mmol) were added at −15° C. and the reaction mixture was stirred at this temperature for 30 min. Then (R)-2-amino-3-methoxy-N—((R)-4-phenyl-1-(4,4,5,5-tetramethyl-1,3,2-dioxaborolan-2-yl) butyl)propanamide hydrochloride (3-4, 300 mg, 0.7 mmol) and NMM (0.1 mL, 0.8 mmol) were added and the reaction mixture was stirred at RT for 2 h. After completion of the reaction (monitored by TLC and LC-MS), the reaction mixture was diluted with EtOAc and washed subsequently with 0.1 M HCl, 5% K₂CO₃, water and brine. The organic phase was dried over Na₂SO₄, filtered and evaporated to yield a brown gum. The crude was purified by RP prep HPLC purification and the eluent was lyophilized to afford N—((R)-3-methoxy-1-oxo-1-(((R)-4-phenyl-1-(4,4,5,5-tetramethyl-1,3,2-dioxaborolan-2-yl)butyl)amino)propan-2-yl)picolinamide (9-3, 60 mg). [M−H]⁻: 480.4.

Synthesis of ((R)-1-((R)-3-methoxy-2-(picolinamido)propanamido)-4-phenylbutyl)boronic acid [Step 2]: To a solution of N—((R)-3-methoxy-1-oxo-1-(((R)-4-phenyl-1-(4,4,5,5-tetramethyl-1,3,2-dioxaborolan-2-yl)butyl)amino)propan-2-yl)picolinamide (9-3, 60 mg, 0.1 mmol) in acetone (3 mL) was added methylboronic acid (75 mg, 1.2 mmol), followed by drop wise addition of 0.2 M HCl (3 mL). The reaction mixture was allowed to stirring at room temperature overnight. All the volatiles were evaporated at room temperature. The crude was re-dissolved in acetonitrile and deionized water and freeze-dried to obtain solid. The crude solid was purified through RP prep HPLC purification and lyophilized to afford ((R)-1-((R)-3-methoxy-2-(picolinamido)propanamido)-4-phenylbutyl)boronic acid (Compound 9, 24 mg). [M−H]⁻: 398.4. ¹H NMR (400 MHz, DMSO-d₆+2 drops D₂O) δ 8.64 (d, 1H), 8.04 (d, 2H), 7.63 (t, 1H), 7.23 (t, 2H), 7.13 (t, 3H), 4.64 (t, 1H), 3.66-3.63 (m, 1H), 3.28 (s, 3H), 3.20 (s, 1H), 2.55-2.53 (m, 2H), 1.57-1.47 (m, 4H). The analytical data suggests that the final compound is in equilibrium with the oxaborolane derivative.

### Example 10: ((R)-1-((R)-3-methoxy-2-(6-methylpicolinamido)propanamido)-4-phenylbutyl) boronic acid

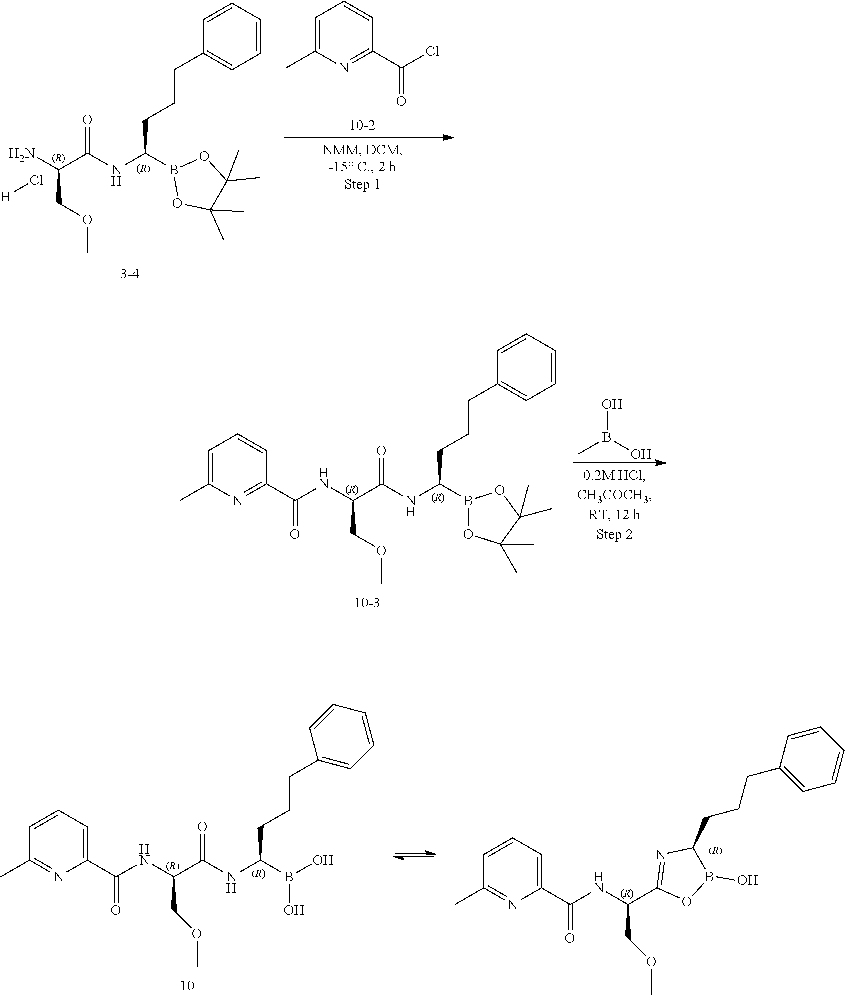

Synthesis of N—((R)-3-methoxy-1-oxo-1-(((R)-4-phenyl-1-(4,4,5,5-tetramethyl-1,3,2-dioxaborolan-2-yl)butyl)amino)propan-2-yl)-6-methylpicolinamide [Step 1]: To a stirred solution of (R)-2-amino-3-methoxy-N—((R)-4-phenyl-1-(4,4,5,5-tetramethyl-1,3,2-dioxaborolan-2-yl)butyl)propanamide (3-4, 300 mg, 0.7 mmol) in DCM (10 mL), NMM (0.1 mL, 0.7 mmol) was added at −15° C. and the reaction mixture was stirred at this temperature for 10 min. To the above solution, 6-methylpicolinoyl chloride (10-2, 110 mg, 0.7 mmol) was added and the reaction mixture was stirred at RT for 2 h. After completion of the reaction (monitored by LCMS), the reaction mixture was diluted with DCM and washed with water and brine. The organic phase was dried over Na₂SO₄, filtered and evaporated to obtain crude. The crude was purified by RP PREP-HPLC to afford N—((R)-3-methoxy-1-oxo-1-(((R)-4-phenyl-1-(4,4,5,5-tetramethyl-1,3,2-dioxaborolan-2-yl)butyl)amino)propan-2-yl)-6-methylpicolinamide (10-3, 35 mg). [M−H]⁻: 494.3.

Synthesis of ((R)-1-((R)-3-methoxy-2-(6-methylpicolinamido)propanamido)-4-phenylbutyl) boronic acid [Step 2]: To a solution of N—((R)-3-methoxy-1-oxo-1-(((R)-4-phenyl-1-(4,4,5,5-tetramethyl-1,3,2-dioxaborolan-2-yl)butyl)amino)propan-2-yl)-6-methylpicolinamide (10-3, 45 mg, 0.09 mmol) in acetone (2 mL) was added methylboronic acid (54 mg, 0.9 mmol), followed by drop wise addition of 0.2 M HCl (2 mL). The reaction mixture was allowed to stirring at room temperature overnight. All the volatiles were evaporated at room temperature. The crude was redissolved in acetonitrile and deionized water and freeze-dried to obtain ((R)-1-((R)-3-methoxy-2-(6-methylpicolinamido)propanamido)-4-phenylbutyl)boronic acid (Compound 10, 20 mg). [M−H]⁻: 412.1. ¹H NMR (400 MHz, CD₃OD) δ 8.39-8.34 (m, 0.5H), 8.08-8.00 (m, 0.5H), 7.90-7.82 (m, 2H), 7.45 (d, 1H), 7.22-7.08 (m, 4H), 6.95-6.93 (m, 1H), 3.89-3.73 (m, 3H), 3.38 (s, 3H), 2.69-2.60 (m, 5H), 2.57 (s, 1H), 1.68-1.66 (m, 2H), 1.54-1.48 (in, 2H). The analytical data suggests that the final compound is in equilibrium with the oxaborolane derivative.

### Example 11: ((R)-1-((R)-3-(benzyloxy)-2-(pyrazine-2-carboxamido)propanamido)-4-phenyl butyl)boronic acid

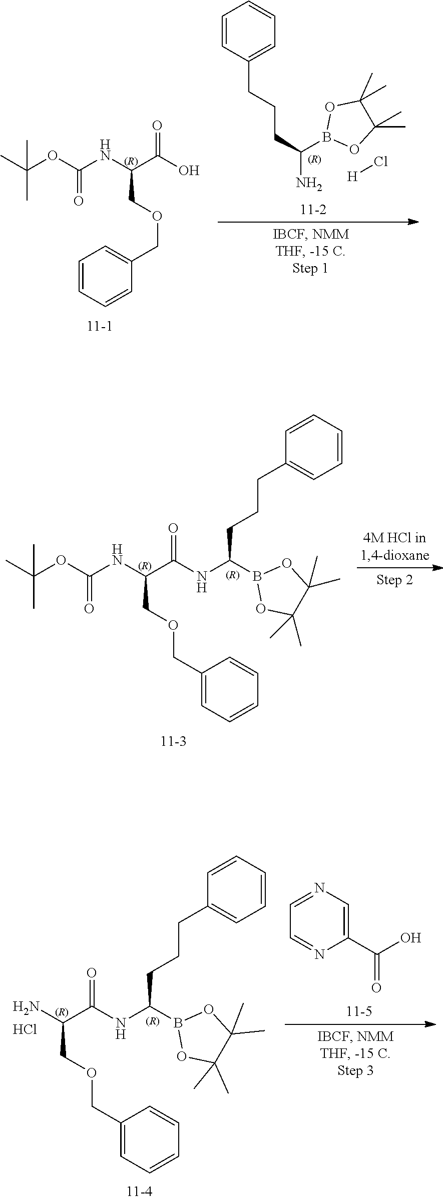

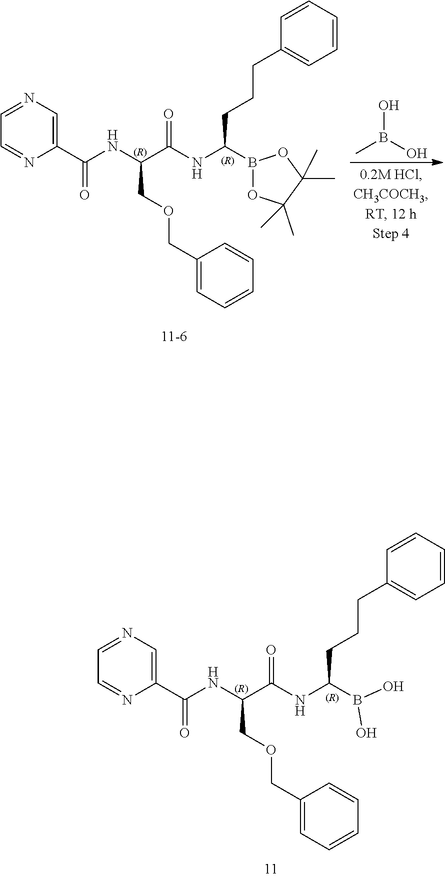

Synthesis of tert-butyl ((R)-3-(benzyloxy)-1-oxo-1-(((R)-4-phenyl-1-(4,4,5,5-tetramethyl-1,3,2-dioxaborolan-2-yl)butyl)amino)propan-2-yl)carbamate [Step 1]: To a stirred solution of O-benzyl-N-(tert-butoxycarbonyl)-D-serine (11-1, 520 mg, 1.8 mmol) in THF (8 mL) were added IBCF (0.24 mL, 1.8 mmol) and NMM (0.24 mL, 1.8 mmol) at −15° C. Reaction mixture was stirred at same temperature for 30 min. followed by the addition of (R)-4-phenyl-1-(4,4,5,5-tetramethyl-1,3,2-dioxaborolan-2-yl)butan-1-amine hydrochloride (11-2, 500 mg, 1.6 mmol) in DMF (1 mL) and NMM (0.21 mL, 1.6 mmol) to the reaction mixture at −15° C. The reaction was gradually warmed to 0° C. and stirred for 2 h. LCMS of crude reaction mass confirmed the formation of desired product. The reaction was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure to afford tert-butyl ((R)-3-(benzyloxy)-1-oxo-1-(((R)-4-phenyl-1-(4,4,5,5-tetramethyl-1,3,2-dioxaborolan-2-yl)butyl) amino)propan-2-yl)carbamate (11-3, 600 mg). [M−H]⁻: 551.5.

Synthesis of (R)-2-amino-3-(benzyloxy)-N—((R)-4-phenyl-1-(4,4,5,5-tetramethyl-1,3,2-dioxaborolan-2-yl)butyl)propanamide hydrochloride [Step 2]: To a solution of tert-butyl ((R)-3-(benzyloxy)-1-oxo-1-(((R)-4-phenyl-1-(4,4,5,5-tetramethyl-1,3,2-dioxaborolan-2-yl)butyl) amino)propan-2-yl)carbamate (11-3, 200 mg, 0.36 mmol) in 1,4 Dioxane (2 mL) was added 4 M HCl in dioxane (2.0 mL, 8.0 mmol) at 0° C. The reaction was gradually warmed to 25° C. and stirred for 16 h. TLC showed complete consumption of starting material. Volatiles were removed under reduced pressure to get (R)-2-amino-3-(benzyloxy)-N—((R)-4-phenyl-1-(4,4,5,5-tetramethyl-1,3,2-dioxaborolan-2-yl)butyl)propanamide hydrochloride (11-4, 180 mg). Crude was directly used for next step without purification. [M−H]⁻: 451.4 (Boronic ester) and [M−H]⁻: 369.3 (Boronic acid).

Synthesis of N—((R)-3-(benzyloxy)-1-oxo-1-(((R)-4-phenyl-1-(4,4,5,5-tetramethyl-1,3,2-dioxaborolan-2-yl)butyl)amino)propan-2-yl)pyrazine-2-carboxamide [Step 3]: To a stirred solution of pyrazine-2-carboxylic acid (11-5, 56 mg, 0.45 mmol) in THF (6 mL) were added IBCF (0.06 mL, 0.45 mmol) and NMM (0.06 mL, 0.45 mmol) at −15° C. The reaction mixture was stirred at same temperature for 30 min followed by the addition of (R)-2-amino-3-(benzyloxy)-N—((R)-4-phenyl-1-(4,4,5,5-tetramethyl-1,3,2-dioxaborolan-2-yl)butyl)propanamide hydrochloride (11-4, 200 mg, 0.4 mmol) in DMF (1 mL) and NMM (0.05 mL, 0.4 mmol) to the reaction mixture. The mixture was gradually warmed to 0° C. and stirred for 2 h. LCMS of the crude reaction mass confirmed the formation of desired product. The reaction mixture was neutralized with saturated aqueous 0.1 M HCl solution and extracted with ethyl acetate. Combined organic layer was washed with 5% K₂CO₃ solution, water, brine, dried over Na₂SO₄, filtered and evaporated under reduced pressure to afford N—((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)pyrazine-2-carboxamide (11-6, 180 mg). [M−H]⁻: 557.2.

Synthesis of ((R)-1-((R)-3-(benzyloxy)-2-(pyrazine-2-carboxamido)propanamido)-4-phenyl butyl)boronic acid [Step 4]: To a stirred solution of N—((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl) pyrazine-2-carboxamide (11-6, 200 mg, 0.36 mmol) and methylboronic acid (214 mg, 3.6 mmol) in acetone (5 mL) was added 0.2 M HCl (5 mL) and the reaction mixture was stirred at RT overnight. TLC and LCMS showed full conversion of starting material with formation of new polar spot. The volatiles were evaporated and the crude material was purified by RP prep HPLC purification and lyophilized to afford ((R)-1-((R)-3-(benzyloxy)-2-(pyrazine-2-carboxamido)propanamido)-4-phenylbutyl)boronic acid (Compound 11, 30 mg). [M−H]⁻: 475.3. ¹H NMR (400 MHz, CD₃OD) δ 9.22 (s, 1H), 8.80 (d, 1H), 8.70 (s, 1H), 7.35-7.25 (m, 5H), 7.21-7.09 (m, 5H), 5.01 (t, 1H), 4.56 (s, 2H), 4.00-3.97 (m, 1H), 3.88-3.84 (m, 1H), 2.66-2.54 (m, 3H), 1.67-1.47 (m, 4H).

### Example 12: ((R)-4-(4-chlorophenyl)-1-((R)-3-methoxy-2-(pyrazine-2-carboxamido) propanamido)butyl)boronic acid

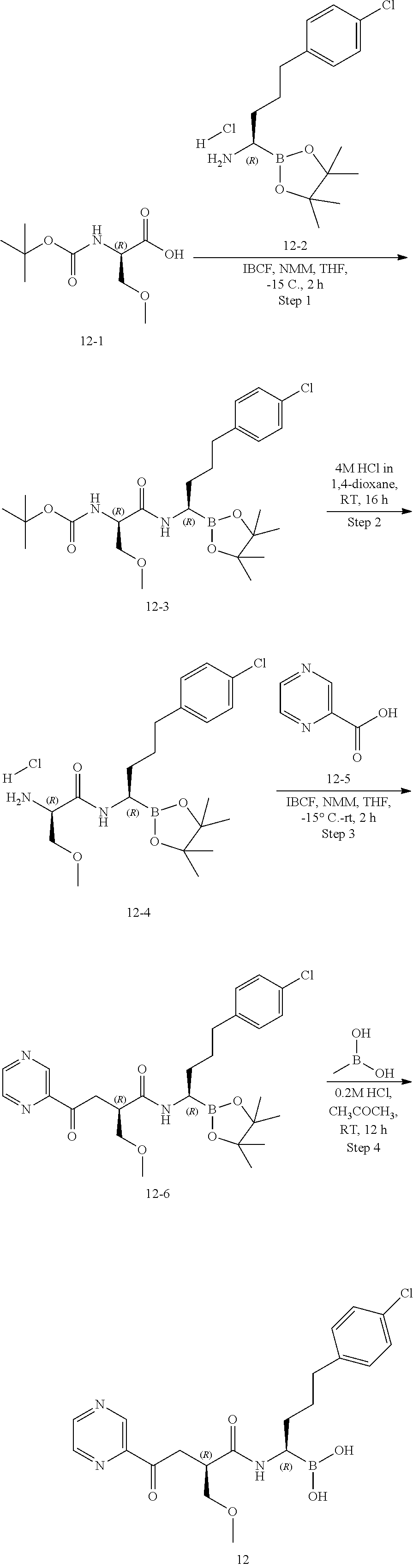

Synthesis of tert-butyl ((R)-1-(((R)-4-(4-chlorophenyl)-1-(4,4,5,5-tetramethyl-1,3,2-dioxaborolan-2-yl)butyl)amino)-3-methoxy-1-oxopropan-2-yl)carbamate [Step 1]: To a stirred solution of (N-(tert-butoxycarbonyl)-O-methyl-D-serine (12-1, 349 mg, 1.6 mmol) in THF (8 mL) was added IBCF (0.2 mL, 1.6 mmol) followed by NMM (0.2 mL, 1.6 mmol) at −15° C. and stirred for 30 min. A solution of (R)-4-(4-chlorophenyl)-1-(4,4,5,5-tetramethyl-1,3,2-dioxaborolan-2-yl)butan-1-amine hydrochloride (12-2, 500 mg, 1.44 mmol) in DMF (1 mL) was added to it followed by NMM (0.2 mL, 1.44 mmol) at −15° C. It was gradually warmed to 0° C. and stirred for 2 h. LCMS of crude reaction mass confirmed the formation of desired product. It was neutralized with saturated aqueous 0.1 M HCl solution and extracted with ethyl acetate. Combined organic layer was washed with 5% K₂CO₃ solution, water and brine, dried over Na₂SO₄ and evaporated under reduced pressure to get crude tert-butyl (R)-2-(((R)-4-phenyl-1-(4,4,5,5-tetramethyl-1,3,2-dioxaborolan-2-yl)butyl)carbamoyl)pyrrolidine-1-carboxylate (12-3, 500 mg). [M−H]⁻: 509.1.

Synthesis of (R)-2-amino-N—((R)-4-(4-chlorophenyl)-1-(4,4,5,5-tetramethyl-1,3,2-dioxaborolan-2-yl)butyl)-3-methoxypropanamide hydrochloride [Step 2]: To a solution of tert-butyl (R)-2-(((R)-4-phenyl-1-(4,4,5,5-tetramethyl-1,3,2-dioxaborolan-2-yl)butyl)carbamoyl) pyrrolidine-1-carboxylate (12-3, 500 mg, 0.97 mmol) in 1,4 Dioxane (6 mL) was added 4 M HCl in 1,4-dioxane (5 mL) at 0° C. It was gradually warmed to 25° C. and stirred for 16 h. TLC showed complete consumption of starting material to form new polar spot. Volatiles were removed under reduced pressure to get crude (R)-2-amino-N—((R)-4-(4-chlorophenyl)-1-(4,4,5,5-tetramethyl-1,3,2-dioxaborolan-2-yl)butyl)-3-methoxypropanamide hydrochloride (12-4, 400 mg). [M−H]⁻: 409.4.

Synthesis of N—((R)-1-(((R)-4-(4-chlorophenyl)-1-(4,4,5,5-tetramethyl-1,3,2-dioxaborolan-2-yl)butyl)amino)-3-methoxy-1-oxopropan-2-yl)pyrazine-2-carboxamide [Step 3]: To a stirred solution of pyrazine-2-carboxylic acid (12-5, 153 mg, 1.12 mmol) in THF (10 mL) was added IBCF (0.16 mL, 1.12 mmol) and NMM (0.16 mL, 1.12 mmol) at −15° C. and stirred for 30 min. A solution of (R)-2-amino-N—((R)-4-(4-chlorophenyl)-1-(4,4,5,5-tetramethyl-1,3,2-dioxaborolan-2-yl)butyl)-3-methoxypropanamide hydrochloride (12-4, 500 mg, 1 mmol) in DMF (1 mL) was added to it followed by NMM (0.15 mL, 1 mmol). It was gradually warmed to 0° C. and stirred for 2 h. LCMS of the crude reaction mass confirmed the formation of desired product. It was neutralized with saturated aqueous 0.1 M HCl solution and extracted with ethyl acetate (thrice). The combined organic layer was washed with 5% K₂CO₃ solution, water and brine, dried over Na₂SO₄ and evaporated under reduced pressure to get crude N—((R)-1-(((R)-4-(4-chlorophenyl)-1-(4,4,5,5-tetramethyl-1,3,2-dioxaborolan-2-yl)butyl)amino)-3-methoxy-1-oxopropan-2-yl)pyrazine-2-carboxamide (12-6, 450 mg). [M−H]: 515.4.

Synthesis of ((R)-4-(4-chlorophenyl)-1-((R)-3-methoxy-2-(pyrazine-2-carboxamido) propanamido)butyl)boronic acid [Step 4]: To a stirred solution of N—((R)-1-(((R)-4-(4-chlorophenyl)-1-(4,4,5,5-tetramethyl-1,3,2-dioxaborolan-2-yl)butyl)amino)-3-methoxy-1-oxopropan-2-yl)pyrazine-2-carboxamide (12-6, 430 mg, 0.83 mmol) and methylboronic acid (500 mg, 8.3 mmol) in acetone (5 mL) was added 0.2 M HCl (5 mL) and stirred at 25° C. for 16h. TLC and LCMS showed full conversion of starting material with formation of new polar spot. The volatiles were evaporated and the crude material was purified by prep HPLC purification and lyophilized to afford ((R)-4-(4-chlorophenyl)-1-((R)-3-methoxy-2-(pyrazine-2-carboxamido) propanamido)butyl)boronic acid (Compound 12, 60 mg). [M−H]⁻: 433.4. ¹H NMR (400 MHz, CD₃OD) δ 9.23 (s, 1H), 8.80 (d, 1H), 8.70 (t, 1H), 7.21 (d, 2H), 7.14 (d, 2H), 4.98 (t, 1H), 3.89-3.86 (m, 1H), 3.78-3.75 (m, 1H), 3.30 (s, 3H), 2.65-2.55 (m, 3H), 1.67-1.44 (m, 4H).

### Example 13: ((R)-4-phenyl-1-((R)-2-(pyrazine-2-carboxamido)-3-(pyridin-2-yloxy) propanamido)butyl)boronic acid

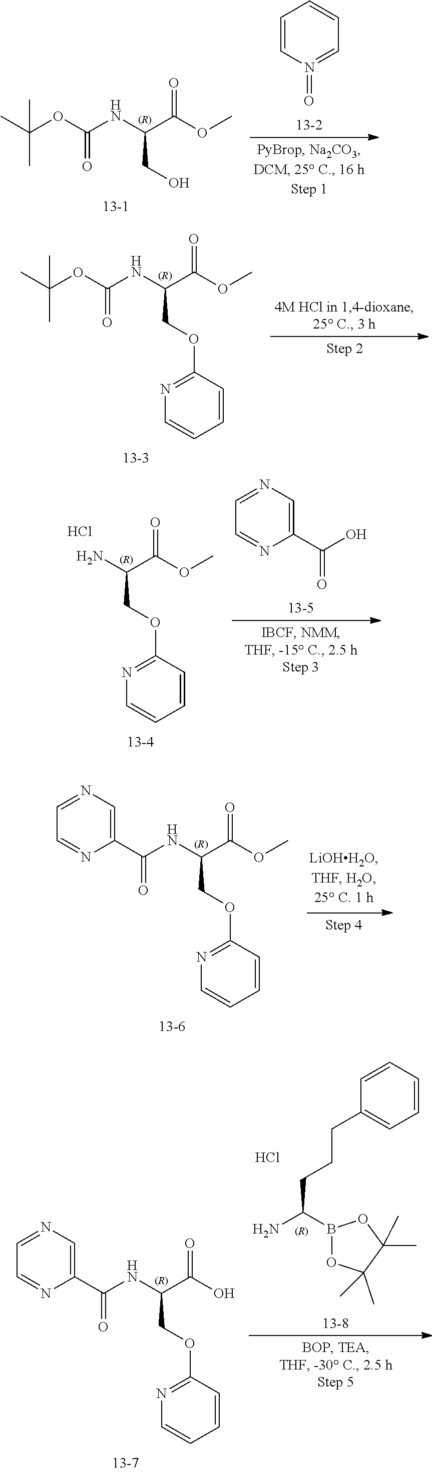

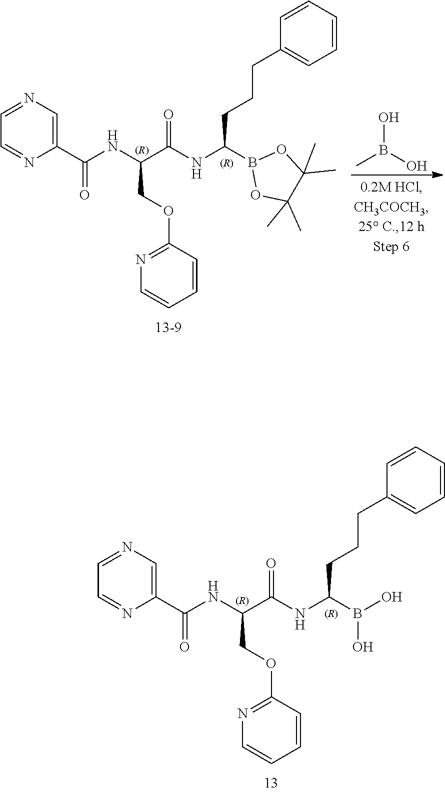

Synthesis of methyl N-(tert-butoxycarbonyl)-O-(pyridin-2-yl)-D-serinate [Step 1]: To a solution of methyl (2R)-2-(tert-butoxycarbonylamino)-3-hydroxy-propanoate (13-1, 6.9 g, 31.5 mmol) in DCM (15 mL) was added Na₂CO₃ (6.8 g, 21.0 mmol) and 4 Å molecular sieves (250 mg). Pyridine 1-oxide (13-2, 3 g, 31.5 mmol) was added to it followed by PyBrop (6.8 g, 14.7 mmol) and stirred at room temperature for 18 h. The reaction was monitored by TLC. Volatiles were removed under reduced pressure and the crude was purified by column chromatography to afford methyl N-(tert-butoxycarbonyl)-O-(pyridin-2-yl)-D-serinate (13-3, 1 g). [M+H]⁺: 297.2. ¹H NMR (400 MHz, DMSO-d₆) δ 8.14 (d, 1H), 7.70 (t, 1H), 7.41 (d, 1H), 6.99 (t, 1H), 6.79 (d, 1H), 4.55-4.36 (m, 3H), 3.64 (s, 3H), 1.37 (s, 9H).

Synthesis of methyl O-(pyridin-2-yl)-D-serinate [Step 2]: To a stirred solution of methyl N-(tert-butoxycarbonyl)-O-(pyridin-2-yl)-D-serinate (13-3, 1 g, 3.3 mmol) in 1,4-dioxane (5 mL) under inert atmosphere was added 4M HCl in dioxane (8 mL, 33.7 mmol) dropwise at ice cold condition and stirred at 25° C. for 3 h. The reaction was monitored by TLC. Volatiles were removed under reduced pressure to get crude methyl O-(pyridin-2-yl)-D-serinate (13-4, 700 mg). [M+H]+: 197.0.

Synthesis of methyl N-(pyrazine-2-carbonyl)-O-(pyridin-2-yl)-D-serinate [Step 3]: To a stirred solution of pyrazine-2-carboxylic acid (13-5, 696 mg, 5.6 mmol) in THF (5 mL) was added IBCF (0.7 mL, 5.6 mmol) followed by NMM (0.6 mL, 5.6 mmol) at −15° C. and stirred for 30 min. A solution of methyl O-(pyridin-2-yl)-D-serinate hydrochloride (13-4, 1.2 g, 5.1 mmol) in THF (4 mL) and DMF (2 mL) was added dropwise followed by NMM (0.5 mL, 5.1 mmol). It was gradually warmed to 0° C. and stirred for 2 h. The reaction was monitored by LCMS. The reaction mixture was diluted with Ethyl acetate and washed with 0.1 (N) HCl (twice), aqueous 5% K₂CO₃ solution (twice), water (twice) and brine, dried over anhydrous Na₂SO₄ and concentrated under reduced pressure. The crude was purified by column chromatography to afford methyl N-(pyrazine-2-carbonyl)-O-(pyridin-2-yl)-D-serinate (13-6, 400 mg). [M+H]+: 303.0. ¹H NMR (400 MHz, CDCl₃) δ 9.38 (s, 1H), 8.90 (d, 1H), 8.74 (s, 1H), 8.55 (s, 1H), 8.13 (d. 1H), 7.57 (t, 1H), 6.89 (t, 1H), 677 (d, 1H), 5.15-5.13 (m, 1H), 4.86-4.71 (m, 2H), 3.79 (s, 3H).

Synthesis of N-(pyrazine-2-carbonyl)-O-(pyridin-2-yl)-D-serine [step 4]: To a solution methyl N-(pyrazine-2-carbonyl)-O-(pyridin-2-yl)-D-serinate (13-6, 393 mg, 1.3 mmol) in THF (5 mL) and water (1 mL) was added LiOH·H₂O (55 mg, 1.3 mmol) at 0° C. and stirred for 1 h at 25° C. The reaction was monitored by LCMS. The resulting mixture was concentrated in vacuo, diluted with water and acidified with 0.2 M HCl (pH: 3) and lyophilized. The crude was purified via prep HPLC purification to afford N-(pyrazine-2-carbonyl)-O-(pyridin-2-yl)-D-serine (13-7, 50 mg). [M+H]+: 289.0. ¹H NMR (400 MHz, DMSO-d₆) δ 9.16 (d, 1H), 8.91 (d, 1H), 8.85 (d, 1H), 8.74-8.73 (m, 1H), 8.34 (s, 1H), 8.09-8.08 (m, 1H), 7.63-7.59 (m, 1H), 7.40 (bs, 1H), 6.90-6.87 (m, 1H), 6.69 (d, 1H), 4.74-4.71 (m, 1H), 4.54-4.51 (m, 1H), 4.24-4.23 (m, 1H).

Synthesis of N—((R)-1-oxo-1-(((R)-4-phenyl-1-(4,4,5,5-tetramethyl-1,3,2-dioxaborolan-2-yl) butyl)amino)-3-(pyridin-2-yloxy)propan-2-yl)pyrazine-2-carboxamide [step 5]: To a solution of N-(pyrazine-2-carbonyl)-O-(pyridin-2-yl)-D-serine (13-7, 41 mg, 0.14 mmol) in THF (2 mL) was added TEA (0.05 mL, 0.38 mmol) at −30° C. and stirred for 30 min. A solution of (1R)-4-phenyl-1-(4,4,5,5-tetramethyl-1,3,2-dioxaborolan-2-yl)butan-1-amine hydrochloride (13-8, 40 mg, 0.12 mmol) in THF (1 mL) was added followed by BOP (85 mg, 0.2 mmol) and stirred for 2 h. The reaction was monitored by LCMS. The reaction mixture was diluted with Ethyl acetate and washed with 5% K₂CO₃ solution and water, dried over anhydrous Na₂SO₄ and concentrated. The crude was purified by prep HPLC purification and lyophilized to afford N—((R)-1-oxo-1-(((R)-4-phenyl-1-(4,4,5,5-tetramethyl-1,3,2-dioxaborolan-2-yl)butyl)amino)-3-(pyridin-2-yloxy)propan-2-yl) pyrazine-2-carboxamide (13-9, 30 mg). [M−H]⁻: 544.4.

Synthesis of ((R)-4-phenyl-1-((R)-2-(pyrazine-2-carboxamido)-3-(pyridin-2-yloxy) propanamido)butyl)boronic acid [Step 6]: A mixture of N—((R)-1-oxo-1-(((R)-4-phenyl-1-(4,4,5,5-tetramethyl-1,3,2-dioxaborolan-2-yl)butyl)amino)-3-(pyridin-2-yloxy)propan-2-yl) pyrazine-2-carboxamide (13-9, 25 mg, 0.04 mmol) and methylboronic acid (27 mg, 0.45 mmol) were dissolved in acetone (1 mL) and 0.2 M HCl (1 mL) and stirred at 25° C. for 16 h. The reaction was monitored by LCMS. Volatiles were evaporated and the crude was purified by Prep HPLC to afford ((R)-4-phenyl-1-((R)-2-(pyrazine-2-carboxamido)-3-(pyridin-2-yloxy)propanamido)butyl) boronic acid (Compound 13, 10 mg). [M−H]⁻: 462.2, ¹H NMR (400 MHz, CD₃OD) δ 9.23 (s, 1H), 8.80 (s, 1H), 8.71 (s, 1H), 8.20-8.19 (m, 1H), 7.66 (t, 1H), 7.22-7.00 (m, 5H), 6.99 (t, 1H), 6.74 (d, 1H), 5.18 (t, 1H), 4.80 (s, 2H), 2.69-2.54 (m, 3H), 1.63-1.28 (m, 5H).

### Example 14: ((R)-1-((R)-3-methoxy-2-(pyrazine-2-carboxamido)propanamido)-3-phenoxy propyl)boronic acid

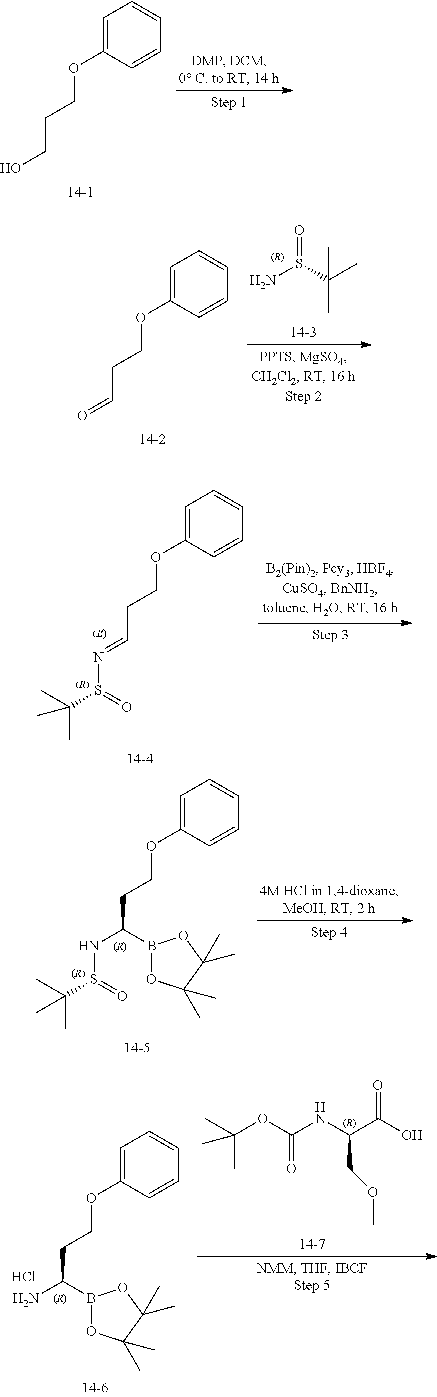

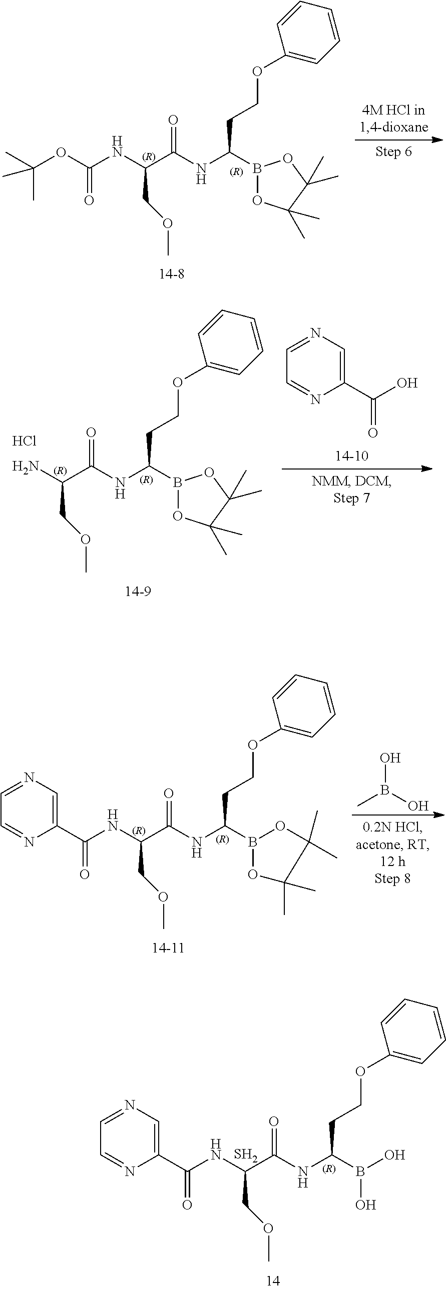

Synthesis of 3-phenoxypropanal [Step 1]: To the stirred a solution of 3-phenoxypropan-1-ol (14-1, 2 g, 13.1 mmol) in a DCM (50 mL) was added DMP (8.36 g, 19.7 mmol) at 0° C. and stirred at RT for 16 h. The reaction was monitored by TLC and LCMS. The reaction mixture was filtered through celite pad and washed with DCM (twice). Combined filtrate liquid was washed with saturated aqueous NaHCO₃ and brine, dried over Na₂SO₄ and concentrated under reduced pressure. The crude was purified by flash column chromatography to afford 3-phenoxypropanal (14-2, 1.4 g). ¹H NMR (400 MHz, CDCl₃) δ 9.89 (s, 1H), 7.30-7.21 (m, 2H), 6.97-6.94 (t, 1H), 6.90-6.81 (d, 2H), 4.32-429 (t, 2H). 2.91-2.87 (m, 2H),

Synthesis of (R,E)-2-methyl-N-(3-phenoxypropylidene)propane-2-sulfinamide [Step 2]: To a solution of (R)-2-methylpropane-2-sulfinamide (14-3, 1.1 g, 9.08 mmol) in DCM (10 mL) under nitrogen atmosphere were added PPTS (114 mg, 0.45 mmol), MgSO4 (5.46 g, 45.4 mmol) followed by a solution of 3-phenoxypropanal (14-2, 1.5 g, 9.98 mmol) in DCM (5 mL) at ice cold condition and stirred at ambient temperature for 16 h. The reaction was monitored by TLC and LCMS. Reaction mixture was filtered through celite bed and the bed was washed with EtOAc. Combined filtrate liquid was concentrated under reduced pressure. The crude was purified by combiflash chromatography to afford (R,E)-2-methyl-N-(3-phenoxypropylidene)propane-2-sulfinamide (14-4, 1.4 g). [M+H]⁺: 254.2.

Synthesis of (R)-2-methyl-N—((R)-3-phenoxy-1-(4,4,5,5-tetramethyl-1,3,2-dioxaborolan-2-yl)propyl)propane-2-sulfinamide [Step 3]: To a solution of PCy3·HBF4 (35 mg, 0.09 mmol) in Toluene (6 mL) was added a solution of CuSO₄.5H₂O (39 mg, 0.16 mmol) in 1.6 mL water followed by Benzyl amine (0.034 mL, 0.32 mmol) at room temperature under argon atmosphere and stirred vigorously for 30 min at room temperature. A solution of (R,E)-2-methyl-N-(3-phenoxypropylidene)propane-2-sulfinamide (14-4, 800 mg, 3.16 mmol) in Toluene (10 mL) was added to it at 0° C. followed by the addition of B₂(Pin)₂ (1.6 g, 6.32 mmol) and stirred at room temperature for 16 h. The reaction was monitored by LCMS. Reaction mass was filtered through a small bed of de-activated silica gel. The bed was washed with EtOAc. Combined filtrate liquid was concentrated at 30° C. under reduced pressure and the crude was purified using deactivated silica to afford (R)-2-methyl-N—((R)-3-phenoxy-1-(4,4,5,5-tetramethyl-1,3,2-dioxaborolan-2-yl)propyl)propane-2-sulfinamide (14-5, 500 mg). [M−H]⁻: 380.2. ¹H NMR (400 MHz, CD₃OD) δ 7.26-7.22 (m, 2H), 6.93-6.88 (m, 3H), 4.15-4.08 (m, 2H), 3.21-3.17 (m, 1H), 2.14-2.07 (m, 2H), 1.25 (s, 9H), 1.20 (s, 12H).

Synthesis of (R)-3-phenoxy-1-(4,4,5,5-tetramethyl-1,3,2-dioxaborolan-2-yl)propan-1-amine hydrochloride [Step 4]: To the solution of (R)-2-methyl-N—((R)-3-phenoxy-1-(4,4,5,5-tetramethyl-1,3,2-dioxaborolan-2-yl)propyl)propane-2-sulfinamide (14-5, 500 mg, 1.3 mmol) in 1,4-dioxane (5 mL) was added MeOH (0.52 mL, 13.1 mmol) and 4 M HCl in 1,4-dioxane (0.5 mL) under ice cool condition and stirred at room temperature for 2 h. The reaction was monitored by LCMS. Volatiles were removed under reduced pressure and lyophilized to get (R)-3-phenoxy-1-(4,4,5,5-tetramethyl-1,3,2-dioxaborolan-2-yl)propan-1-amine hydrochloride (14-6, 250 mg). ¹H NMR (400 MHz, CD₃OD) δ 7.29-7.25 (m, 2H), 6.95-6.91 (m, 2H), 4.15-4.06 (m, 2H), 3.03 (m, 1H), 2.26-2.15 (m, 2H), 1.32-1.18 (m, 6H).

Synthesis of tert-butyl ((R)-3-methoxy-1-oxo-1-(((R)-3-phenoxy-1-(4,4,5,5-tetramethyl-1,3,2-dioxaborolan-2-yl)propyl)amino)propan-2-yl)carbamate [Step 5]: To a stirred solution of N-(tert-butoxycarbonyl)-O-methyl-D-serine (14-7, 596 mg, 2.7 mmol) in THF (5 mL) were added IBCF (0.4 mL, 2.9 mmol) and NMM (0.4 mL, 2.97 mmol) at −15° C. and stirred for 30 min. A solution of (R)-3-phenoxy-1-(4,4,5,5-tetramethyl-1,3,2-dioxaborolan-2-yl)propan-1-amine hydrochloride (14-6, 775 mg, 2.47 mmol) in THF (2 ml) was added to it dropwise followed by NMM (0.4 mL, 2.9 mmol) and stirred at 0° C. for 2 h. The reaction was monitored by LCMS. The reaction mixture was diluted with ethyl acetate and washed with 0.1 M HCl solution (twice), 10% aqueous K₂CO₃ solution (twice), water (twice) and brine, dried over anhydrous Na₂SO₄ and evaporated under reduced pressure at 30° C. to afford tert-butyl ((R)-3-methoxy-1-oxo-1-(((R)-3-phenoxy-1-(4,4,5,5-tetramethyl-1,3,2-dioxaborolan-2-yl)propyl)amino)propan-2-yl)carbamate (14-8, 900 mg). [M−H]⁻: 477.4.

Synthesis of (R)-2-amino-3-methoxy-N—((R)-3-phenoxy-1-(4,4,5,5-tetramethyl-1,3,2-dioxaborolan-2-yl)propyl)propanamide hydrochloride [Step 6]: To the stirred solution tert-butyl ((R)-3-methoxy-1-oxo-1-(((R)-3-phenoxy-1-(4,4,5,5-tetramethyl-1,3,2-dioxaborolan-2-yl) propyl)amino)propan-2-yl)carbamate (14-8, 1.3 g, 2.7 mmol) in 1,4-dioxane (10 mL) was added 4 M HCl in 1,4-dioxane (5 mL) at ice cold condition and stirred at room temperature for 16 h. The reaction was monitored by LCMS. Volatiles were removed under reduced pressure and washed with pentane and dried to get (R)-2-amino-3-methoxy-N—((R)-3-phenoxy-1-(4,4,5,5-tetramethyl-1,3,2-dioxaborolan-2-yl)propyl)propanamide (14-9, 1 g). [M−H]⁻: 376.2.

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)pyrazine-2-carboxamide [Step 7]: To the stirred solution of pyrazine-2-carbonyl chloride (14-10, 412 mg, 2.89 mmol) in 0CM (2 mL) NMM (0.39 mL, 2.89 mmol) was added followed by (R)-2-amino-3-methoxy-N—((R)-3-phenoxy-1-(4,4,5,5-tetramethyl-1,3,2-dioxaborolan-2-yl)propyl)propanamide (14-9, 1 g, 2.4 mmol) at −15° C. and stirred at same temperature for 2 h. The reaction was monitored by LCMS. The reaction mixture was diluted with DCM and washed with water and brine, dried over Na2SO4 and evaporated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)pyrazine-2-carboxamide (14-11, 1 g). [M−H]⁻: 483.4.

Synthesis of ((R)-1-((R)-3-methoxy-2-(pyrazine-2-carboxamido)propanamido)-3-phenoxy propyl)boronic acid [Step 8]: To a stirred solution of N—((R)-3-methoxy-1-oxo-1-(((R)-3-phenoxy-1-(4,4,5,5-tetramethyl-1,3,2-dioxaborolan-2-yl)propyl)amino)propan-2-yl)pyrazine-2-carboxamide (14-4, 600 mg, 1.24 mmol) in acetone (10 ml) were added methylboronic acid (742 mg, 12.4 mmol) and 0.2 M HCl (10 mL) at ice cold condition and stirred at room temperature for 12h. Volatiles were removed under reduced pressure and purified by prep HPLC purification and lyophilized to afford ((R)-1-((R)-3-methoxy-2-(pyrazine-2-carboxamido)propanamido)-3-phenoxy propyl)boronic acid (Compound 14, 93 mg). [M−H]⁻: 401. ¹H NMR (400 MHz, DMSO-d₆+2 drops D₂O) δ: 9.19 (s, 1H), 8.90 (s, 1H), 8.76 (s, 1H), 8.67-8.66 (d, 1H), 7.26-7.22 (m, 2H), 6.90-6.84 (m, 3H), 4.69 (m, 1H), 3.93-3.89 (m, 2H), 3.70-3.61 (m, 2H), 3.29-3.26 (m, 1H), 3.15 (s, 3H), 1.96-1.87 (m, 2H).

### Example 15: Synthesis of ((R)-4-(3-chlorophenyl)-1-((R)-3-methoxy-2-(pyrazine-2-carboxamido)propanamido)butyl)boronic acid

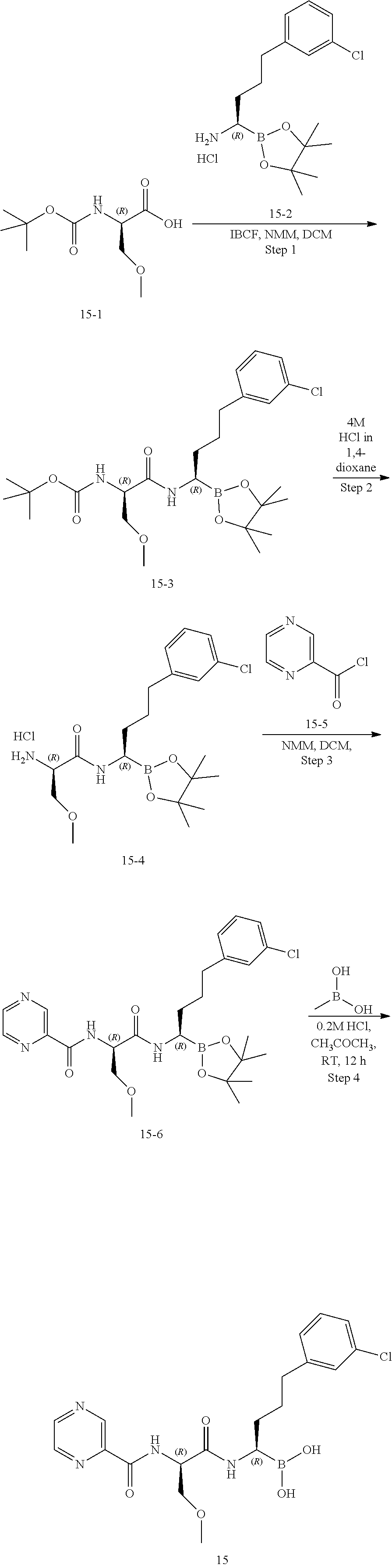

Synthesis of tert-butyl ((R)-1-(((R)-4-(3-chlorophenyl)-1-(4,4,5,5-tetramethyl-1,3,2-dioxaborolan-2-yl)butyl)amino)-3-methoxy-1-oxopropan-2-yl)carbamate [Step 1]: To the stirred solution N-(tert-butoxycarbonyl)-O-methyl-D-serine (15-1, 380 mg, 1.73 m mol) in THF (5 mL) at −10° C. were added IBCF (0.23 mL, 1.73 mmol) and NMM (0.24 mL, 1.73 mmol) drop wise. The reaction mixture was allowed to stir for 30 minutes at the same temperature and solution of (R)-4-(3-chlorophenyl)-1-(4,4,5,5-tetramethyl-1,3,2-dioxaborolan-2-yl)butan-1-amine hydrochloride (15-2, 500 mg, 1.44 mmol) in THF (10 mL) was added dropwise, followed by the addition of NMM (0.24 mL, 1.73 mmol).The reaction mixture was stirred at the same temperature for 2.5h. The reaction mixture was diluted with ethyl acetate (25 mL) and was washed with cold aq. 0.1 M HC solution (10 ml, ×2), followed by 10% aq. K₂CO₃ solution and finally with brine solution. Organic phase was dried over Na2SO₄, filtered and evaporated under reduced pressure at 30° C. to afford tert-butyl ((R)-1-(((R)-4-(3-chlorophenyl)-1-(4,4,5,5-tetramethyl-1,3,2-dioxaborolan-2-yl)butyl) amino)-3-methoxy-1-oxopropan-2-yl)carbamate (15-3, 800 mg). This crude material which was used in the next step without purification. [M−H]⁻: 509.2.

Synthesis of (R)-2-amino-N—((R)-4-(3-chlorophenyl)-1-(4,4,5,5-tetramethyl-1,3,2-dioxaborolan-2-yl)butyl)-3-methoxypropanamxide hydrochloride [Step 2]: To the stirred solution of tert-butyl ((R)-1-(((R-4-(3-chlorophenyl)-1-(4,4,5,5-tetramethyl-1,3,2-dioxaborolan-2-yl)butyl)amino)-3-methoxy-1-oxopropan-2-yl)carbamate (15-3, 800 mg, 1.57 m-mol) in 1,4-dioxane (10 mL) was added HCl (4 M in 1,4-dioxane, 4 mL) was added dropwise at ice cool condition and the reaction mixture was allowed to stir for 4 h at room temperature. The Reaction mixture was concentrated under reduced pressure 30° C. and lyophilized to afford (R)-2-amino-N—((R)-4-(3-chlorophenyl)-1-(4,4,5,5-tetramethyl-1,3,2-dioxaborolan-2-yl)butyl)-3-methoxy propanamide hydrochloride (15-4, 600 mg). [M−H]⁻: 409.4.

Synthesis of N—((R)-1-(((R)-4-(3-chlorophenyl)-1-(4,4,5,5-tetramethyl-1,3,2-dioxaborolan-2-yl)butyl)amino)-3-methoxy-1-oxopropan-2-yl)pyrazine-2-carboxamide [Step 3]: To the stirred solution of (R)-2-amino-N—((R)-4-(3-chlorophenyl)-1-(4,4,5,5-tetramethyl-1,3,2-dioxaborolan-2-yl)butyl)-3-methoxypropanamide hydrochloride (15-4, 600 mg, 1.34 mmol) in DCM (10 mL) was added NMM (0.22 mL, 1.61 mmol) at 0° C., followed by the addition of pyrazine-2-carbonyl chloride (15-5, 229 mg, 1.61 mmol). The reaction was allowed to stir for 2 h at room temperature. The reaction mixture was diluted with DCM and washed with water and brine. Organic layer was dried over Na₂SO₄, filtered and evaporated under reduced pressure at 30° C. to get N—((R)-1-(((R)-4-(3-chlorophenyl)-1-(4,4,5,5-tetramethyl-1,3,2-dioxaborolan-2-yl)butyl)amino)-3-methoxy-1-oxopropan-2-yl)pyrazine-2-carboxamide (15-6, 700 mg yield). This crude material was used in the next step without purification. [M−H]⁻: 515.4.

Synthesis of ((R)-4-(3-chlorophenyl)-1-((R)-3-methoxy-2-(pyrazine-2-carboxamido) propanamido)butyl)boronic acid [Step 4]: To the stirred solution of N—((R)-1-(((R)-4-(3-chlorophenyl)-1-(4,4,5,5-tetramethyl-1,3,2-dioxaborolan-2-yl)butyl)amino)-3-methoxy-1-oxopropan-2-yl)pyrazine-2-carboxamide (15-6, 700 mg, 1.35 mmol) and methylboronic acid (116 mg, 1.94 mmol) in acetone (5 mL) was added HCl (0.2 M in water, 1.5 mL) at ice cool condition and the reaction mixture was allowed to stir at room temperature for 16 h. The reaction mixture was evaporated under reduced pressure and lyophilized to get crude material which was purified by reverse phase PREP-HPLC. Pure fraction was lyophilized to afford ((R)-4-(3-chlorophenyl)-1-((R)-3-methoxy-2-(pyrazine-2-carboxamido)propanamido)butyl)boronic acid (Compound 15, 125 mg). [M−H]⁻: 433.4. ¹H NMR (400 MHz, CD₃OD) δ 9.23 (s, 1H), 8.81-8.80 (d, 1H), 8.70 (s, 1H), 7.22-7.18 (m, 2H), 7.13-7.08 (m, 1H), 4.99-4.97 (m, 1H), 3.89-3.86 (m, 1H), 3.79-3.75 (m, 1H), 3.37 (s, 3H), 2.66-2.57 (m, 3H), 1.66-1.48 (in, 4H).

### Example 16: ((R)-1-((R)-2-(1,3-dioxoisoindolin-2-yl)-3-methoxypropanamido)-4-phenyl butyl)boronic acid

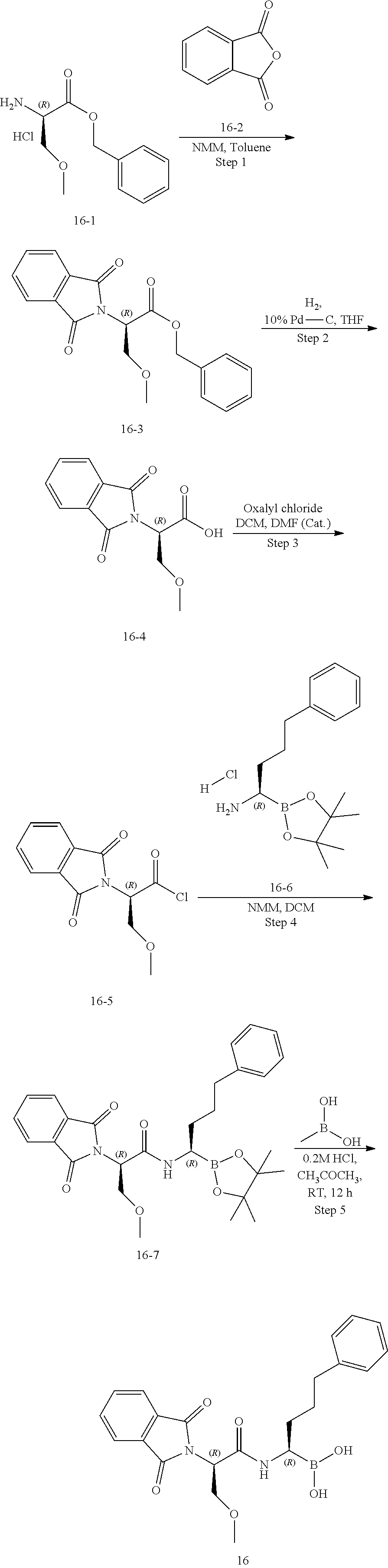

Synthesis of benzyl (R)-2-(1,3-dioxoisoindolin-2-yl)-3-methoxypropanoate [step 1]: To a solution of benzyl O-methyl-D-serinate hydrochloride (16-1, 2 g, 8 mmol) in toluene (25 mL) was added NMM (2.7 mL, 24 mmol) at 0° C. and stirred for 10 min. To the above solution was added isobenzofuran-1,3-dione (16-2, 1.2 g, 8 mmol) and continued stirring at 100° C. for 12 h. After completion solvent was removed under reduced pressure. It was diluted with cold H₂O was and extracted with EtOAc, the organic layer was washed with 0.2 M HCl and brine, dried over Na₂SO₄ and concentrated under reduced pressure to afford benzyl (R)-2-(1,3-dioxoisoindolin-2-yl)-3-methoxypropanoate (16-3, 2 g). The product was used further without purification. [M+H]⁺: 340.0.

Synthesis of (R)-2-(1,3-dioxoisoindolin-2-yl)-3-methoxypropanoic acid [step 2]: In a 2-necked round-bottom flask, purged and maintained under nitrogen was placed a solution of benzyl (R)-2-(1,3-dioxoisoindolin-2-yl)-3-methoxypropanoate (16-3, 1 g, 3 mmol) in THF (40 mL) was added 10% Pd—C (370 mg, 3.5 mmol). Reaction mixture was stirred at ambient temperature under hydrogen balloon pressure for 16 h. The reaction mixture was filtered through celite pad and the pad was further washed with THF. The combined filtrate liquid was concentrated under a vacuum. The product was purified via silica gel column chromatography using 70% EtOAc in hexane as eluent to yield (R)-2-(1,3-dioxoisoindolin-2-yl)-3-methoxypropanoic acid (16-4, 700 mg). [M+H]*: 249.8. ¹H NMR (400 MHz, DMSO-d₆) δ 13.44 (s, 1H), 7.95-7.89 (m, 4H), 5.09 (q, 1H), 4.00 (t, 1H), 3.90-3.86 (m, 1H), 3.22 (s, 3H).

Synthesis of (R)-2-(1,3-dioxoisoindolin-2-yl)-3-methoxypropanoyl chloride [step 3]: In an oven-dried round-bottom flask to a solution of (R)-2-(1,3-dioxoisoindolin-2-yl)-3-methoxypropanoic acid (16-4, 450 mg, 1.8 mmol) in anhydrous DCM (15 mL) and DMF (0.05 mL) was added oxalyl dichloride (0.3 mL, 3.6 mmol) dropwise at 0° C. The reaction mixture was allowed to stir at ambient temperature for 12 h. After completion solvent was removed under N₂ atmosphere to obtain (R)-2-(1,3-dioxoisoindolin-2-yl)-3-methoxypropanoyl chloride (16-5, 400 mg). The product was used further without purification. [M+H]⁺: 263.8.

Synthesis of (R)-2-(1,3-dioxoisoindolin-2-yl)-3-methoxy-N—((R)-4-phenyl-1-(4,4,5,5-tetra methyl-1,3,2-dioxaborolan-2-yl)butyl)propenamide [step 4]: To a stirred solution of ((R)-4-phenyl-1-(4,4,5,5-tetramethyl-1,3,2-dioxaborolan-2-yl)butan-1-amine hydrochloride (16-6, 560 mg, 1.8 mmol) in DCM (15 mL), NMM (0.25 mL, 2.2 mmol) and (R)-2-(1,3-dioxoisoindolin-2-yl)-3-methoxypropanoyl chloride (16-5, 480 mg, 1.8 mmol) were added at −15° C. and the reaction mixture was stirred at this temperature for 30 min. then slowly bring it to ambient temperature and stirred for 2 h. After completion the reaction mixture was diluted with DCM and washed subsequently with 0.1 M HCl, water and brine. The organic phase was dried over Na₂SO₄, filtered and evaporated under reduced pressure to afford a brown gummy product. Product was purified by RP prep HPLC and the eluent was lyophilized to afford (R)-2-(1,3-dioxoisoindolin-2-yl)-3-methoxy-N—((R)-4-phenyl-1-(4,4,5,5-tetramethyl-1,3,2-dioxaborolan-2-yl)butyl)propanamide (16-7, 40 mg). [M−H]⁻: 505.5.

Synthesis of ((R)-1-((R)-2-(1,3-dioxoisoindolin-2-yl)-3-methoxypropanamido)-4-phenyl butyl)boronic acid [step 5]: To a solution of (R)-2-(1,3-dioxoisoindolin-2-yl)-3-methoxy-N—((R)-4-phenyl-1-(4,4,5,5-tetramethyl-1,3,2-dioxaborolan-2-yl)butyl)propanamide (16-7, 40 mg, 0.08 mmol) in acetone (2 mL) was added methylboronic acid (50 mg, 0.8 mmol), followed by drop wise addition of 0.2 M HCl (2 mL). The reaction mixture was allowed to stirring at room temperature overnight. All the volatiles were evaporated at room temperature. The crude was diluted in acetonitrile and deionized water and freeze-dried to obtain solid. The crude solid was purified through RP PERP-HPLC purification and lyophilized to afford diasteromeric mixture (25 mg, 75%). The diasteromeric mixture was purified by PREP-HPLC Chiral (SFC) to afford ((R)-1-((R)-2-(1,3-dioxoisoindolin-2-yl)-3-methoxypropanamido)-4-phenylbutyl)boronic acid (Compound 16, 10 mg). [M−H]⁻: 423.5. ¹H NMR (400 MHz, DMSO-d₆+2 drops D₂O) δ 7.91-7.86 (m, 4H), 7.25 (t, 2H), 7.14 (t, 3H), 4.97-4.93 (m, 1H), 3.98-3.93 (m, 2H), 3.21 (s, 3H), 2.99 (d, 1H), 2.49 (s, 2H), 1.47-1.44 (m, 4H). Stereochemistry of the diastereomers was randomly assigned.

### Example 17: ((R)-1-((R)-3-methoxy-2-(pyrazine-2-carboxamido)propanamido)-4-(3-methoxyphenyl)butyl)boronic acid

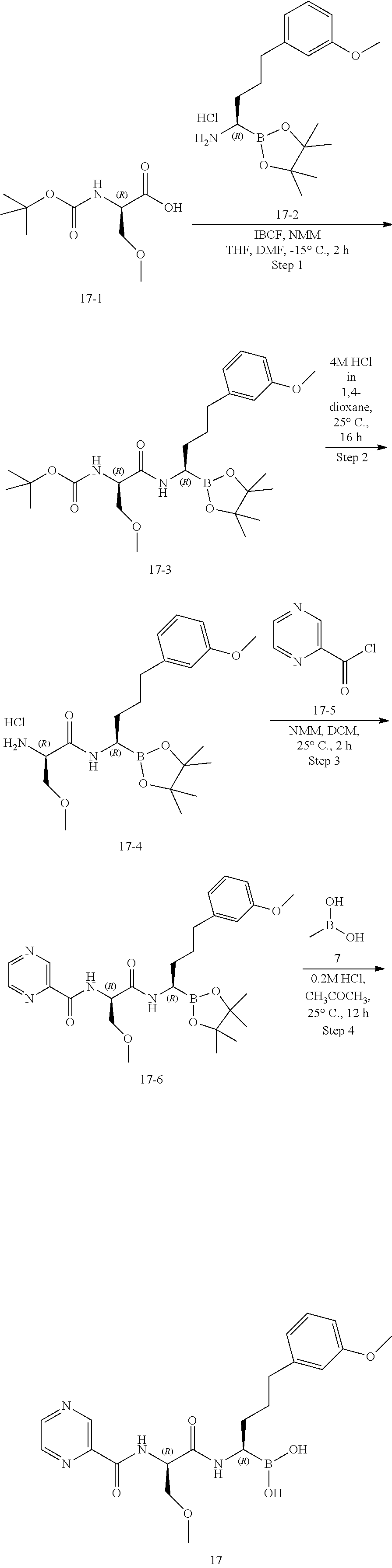

Synthesis of tert-butyl ((R)-3-methoxy-1-(((R)-4-(3-methoxyphenyl)-1-(4,4,5,5-tetramethyl-1,3,2-dioxaborolan-2-yl)butyl)amino)-1-oxopropan-2-yl)carbamate [Step 1]: To a stirred solution of N-(tert-butoxycarbonyl)-O-methyl-D-serine (17-1, 142 mg, 0.64 mmol) in THF (6 mL) was added IBCF (0.09 mL, 0.64 mmol) and NMM (0.09 mL, 0.64 mmol) at −15° C. and stirred for 30 min. A solution of (R)-4-(3-methoxyphenyl)-1-(4,4,5,5-tetramethyl-1,3,2-dioxaborolan-2-yl)butan-1-amine hydrochloride (17-2, 200 mg, 0.58 mmol) in DMF (0.5 mL) was added followed by NMM (0.8 mL, 0.58 mmol) at −15° C. It was gradually warmed to 0° C. and stirred for 2 h. LCMS of crude reaction mass confirmed the formation of desired product. It was neutralized with saturated aqueous 0.1 M HCl solution and extracted with ethyl acetate. Combined organic layer was washed with 5% K₂CO₃ solution, water and brine, dried over Na₂SO₄ and evaporated under reduced pressure to get crude tert-butyl ((R)-3-methoxy-1-(((R)-4-(3-methoxyphenyl)-1-(4,4,5,5-tetramethyl-1,3,2-dioxaborolan-2-yl)butyl)amino)-1-oxopropan-2-yl)carbamate (17-3, 260 mg). [M−H]⁻: 505.4.

Synthesis of (R)-2-amino-3-methoxy-N—((R)-4-(3-methoxyphenyl)-1-(4,4,5,5-tetramethyl-1,3,2-dioxaborolan-2-yl)butyl)propanamide hydrochloride [Step 2]: To a solution of tert-butyl ((R)-3-methoxy-1-(((R)-4-(3-methoxyphenyl)-1-(4,4,5,5-tetramethyl-1,3,2-dioxaborolan-2-yl)butyl)amino)-1-oxopropan-2-yl)carbamate (17-3, 400 mg, 0.8 mmol) in 1,4-dioxane (4 mL) was added 4 M HCl in 1,4-dioxane (2.0 mL) at 0° C. It was gradually warmed to 25° C. and stirred for 16 h. TLC showed complete consumption of starting material. Volatiles were removed under reduced pressure to get crude (R)-2-amino-3-methoxy-N—((R)-4-(3-methoxyphenyl)-1-(4,4,5,5-tetramethyl-1,3,2-dioxaborolan-2-yl)butyl)propanamide hydrochloride (17-4, 300 mg). [M−H]⁻: 405.1.

Synthesis of N—((R)-3-methoxy-1-(((R)-4-(3-methoxyphenyl)-1-(4,4,5,5-tetramethyl-1,3,2-dioxaborolan-2-yl)butyl)amino)-1-oxopropan-2-yl)pyrazine-2-carboxamide [Step 3]: To a solution of (R)-2-amino-3-methoxy-N—((R)-4-(3-methoxyphenyl)-1-(4,4,5,5-tetramethyl-1,3,2-dioxaborolan-2-yl)butyl)propanamide hydrochloride (17-4, 240 mg, 0.6 mmol) in DCM (10 mL) was added NMM (0.12 mL, 1.2 mmol) and stirred for 5 min under argon atmosphere. To this mixture pyrazine-2-carbonyl chloride (17-5, 110 mg, 0.8 mmol) was added and continued stirring at 20° C. for 2 h. The reaction monitored by TLC. Upon completion the reaction mixture was diluted with DCM and washed with water and brine. Combined organic layer was dried over Na₂SO₄ and evaporated to get crude which was purified by prep-HPLC to get N—((R)-3-methoxy-1-(((R)-4-(3-methoxyphenyl)-1-(4,4,5,5-tetramethyl-1,3,2-dioxaborolan-2-yl)butyl)amino)-1-oxopropan-2-yl)pyrazine-2-carboxamide (17-6, 40 mg). [M−H]⁻: 511.4.

Synthesis of ((R)-1-((R)-3-methoxy-2-(pyrazine-2-carboxamido)propanamido)-4-(3-methoxyphenyl)butyl)boronic acid [Step 4]: To a stirred solution of N—((R)-3-methoxy-1-(((R)-4-(3-methoxyphenyl)-1-(4,4,5,5-tetramethyl-1,3,2-dioxaborolan-2-yl)butyl)amino)-1-oxopropan-2-yl)pyrazine-2-carboxamide (17-6, 30 mg, 0.06 mmol) and methylboronic acid (35 mg, 0.6 mmol) in acetone (1 mL) was added 0.2 M HCl (1 mL) and the reaction mixture was stirred at RT overnight. TLC and LCMS showed full conversion of starting material with formation of new polar spot. The volatiles were evaporated and the crude material was purified by RP prep HPLC purification and lyophilized to afford ((R)-1-((R)-3-methoxy-2-(pyrazine-2-carboxamido)propanamido)-4-(3-methoxyphenyl)butyl)boronic acid (Compound 17, 10 mg). [M−H]⁻: 429.4. ¹H NMR (400 MHz, CD₃OD) δ 9.23 (s, 1H), 8.80 (d, 1H), 8.69 (d, 1H), 7.11 (t, 1H), 6.74-6.66 (m, 3H), 4.97 (t, 1H), 3.86-3.74 (m, 5H), 3.37 (s, 3H), 2.64 (t, 1H), 2.59-2.54 (m, 2H), 1.63-1.43 (m, 5H).

### Example 18: Synthesis of ((R)-2-cyclopropyl-1-((R)-3-methoxy-2-(pyrazine-2-carboxamido) propanamido)ethyl)boronic acid

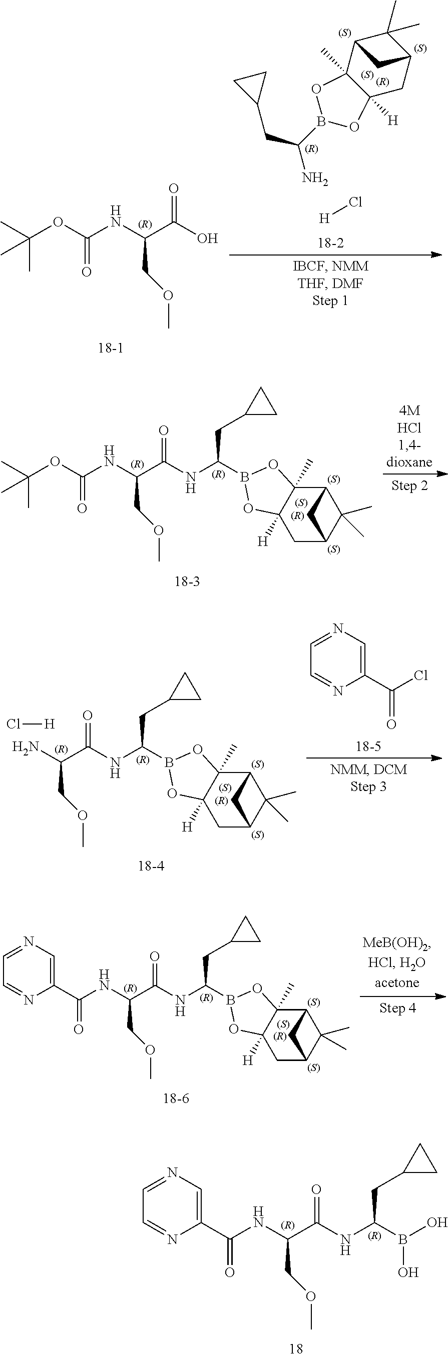

Synthesis of tert-butyl ((R)-1-(((R)-2-cyclopropyl-1-((3aS,4S,6S,7aR)-3a,5,5-trimethyl hexahydro-4,6-methanobenzo[d][1,3,2]dioxaborol-2-yl)ethyl)amino)-3-methoxy-1-oxopropan-2-yl)carbamate [Step 1]: To a stirred solution of N-(tert-butoxycarbonyl)-O-methyl-D-serine (18-1, 238 mg, 1.1 mmol) in THF (4 mL) was added NMM (0.15 mL, 1.37 mmol) followed by IBCF (0.16 mL, 1.24 mmol) at −15° C. The reaction mixture was stirred at same temperature for 30 min. A solution of (R)-2-cyclopropyl-1-((3aS,4S,6S,7aR)-3a,5,5-trimethylhexahydro-4,6-methanobenzo[d][1,3,2]dioxaborol-2-yl)ethan-1-amine hydrochloride (18-2, 325 mg, 1.1 mmol) in THF (2 mL) and DMF (1 mL) was added to the reaction mixture at −15° C. It was gradually warmed to 0° C. and stirred for 2 h. The reaction mass was neutralized with saturated aqueous 0.1 M HCl and extracted with ethyl acetate (thrice). Combined organic layer was washed with 5% aqueous K₂CO₃ solution, water and brine, dried over anhydrous Na₂SO₄, and evaporated under reduced pressure to afford tert-butyl ((R)-1-(((R)-2-cyclopropyl-1-((3aS,4S,6S,7aR)-3a,5,5-trimethyl hexahydro-4,6-methanobenzo[d][1,3,2]dioxaborol-2-yl)ethyl)amino)-3-methoxy-1-oxopropan-2-yl)carbamate (18-3, 400 mg). [M−H]⁻: 463.2.

Synthesis of (R)-2-amino-N—((R)-2-cyclopropyl-1-((3aS,4S,6S,7aR)-3a,5,5-trimethyl hexa hydro-4,6-methanobenzo[d][1,3,2]dioxaborol-2-yl)ethyl)-3-methoxypropanamide hydrochloride [Step 2]: To a stirred solution of tert-butyl ((R)-1-(((R)-2-cyclopropyl-1-((3aS,4S,6S,7aR)-3a,5,5-trimethylhexahydro-4,6-methanobenzo[d][1,3,2]dioxaborol-2-yl)ethyl) amino)-3-methoxy-1-oxopropan-2-yl)carbamate (18-3, 400 mg, 0.86 mmol) in 1,4-dioxane (5 mL) was added 4 M HCl in 1,4-dioxane (2.2 mL, 8.61 mmol) at ice cold condition. The reaction mixture was allowed to stir at 0° C. for 5 h. Volatiles were removed under reduced pressure and triturated with n-pentane to afford (R)-2-amino-N—((R)-2-cyclopropyl-1-((3aS,4S,6S,7aR)-3a,5,5-trimethylhexahydro-4,6-methanobenzo[d][1,3,2]dioxaborol-2-yl)ethyl)-3-methoxypropanamide hydrochloride (18-4, 300 mg). [M−H]⁻: 363.2.

Synthesis of N—((R)-1-(((R)-2-cyclopropyl-1-((3aS,4S,6S,7aR)-3a,5,5-trimethylhexahydro-4,6-methanobenzo[d][1,3,2]dioxaborol-2-yl)ethyl)amino)-3-methoxy-1-oxopropan-2-yl) pyrazine-2-carboxamide [Step 3]: To a stirred solution of pyrazine-2-carbonyl chloride (18-5, 128 mg, 0.89 mmol) in DCM (6 mL) was added NMM (0.12 mL, 0.89 mmol) at −15° C. followed by the addition of (R)-2-amino-N—((R)-2-cyclopropyl-1-((3aS,4S,6S,7aR)-3a,5,5-trimethylhexahydro-4,6-methanobenzo[d][1,3,2]dioxaborol-2-yl)ethyl)-3-methoxypropanamide hydrochloride (18-4, 300 mg, 0.75 mmol) and stirred for 2 h. The reaction mixture was diluted with 0CM and washed with water and brine, dried over anhydrous sodium sulfate and concentrated under reduced pressure. The product was purified by prep HPLC purification and lyophilized to afford N—((R)-1-(((R)-2-cyclopropyl-1-((3aS,4S,6S,7aR)-3a,5,5-trimethylhexahydro-4,6-methanobenzo [d][1,3,2]dioxaborol-2-yl)ethyl)amino)-3-methoxy-1-oxopropan-2-yl)pyrazine-2-carboxamide (18-6, 40 mg). [M−H]⁻: 469.3.

Synthesis of ((R)-2-cyclopropyl-1-((R)-3-methoxy-2-(pyrazine-2-carboxamido) propanamido)ethyl)boronic acid, PZL-0002234 [Step 4]: To a stirred solution of N—((R)-1-(((R)-2-cyclopropyl-1-((3aS,4S,6S,7aR)-3a,5,5-trimethylhexahydro-4,6-methanobenzo [d][1,3,2]dioxaborol-2-yl)ethyl)amino)-3-methoxy-1-oxopropan-2-yl)pyrazine-2-carboxamide (18-6, 40 mg, 0.08 mmol) in acetone (3 mL) was added methylboronic acid (48 mg, 0.80 mmol) and 0.2 M HCl (3.0 mL) at ice cold condition. The reaction mixture was stirred at ambient temperature for 3 h. Volatiles were evaporated under reduced pressure. The product was purified by prep HPLC purification and lyophilized to afford ((R)-2-cyclopropyl-1-((R)-3-methoxy-2-(pyrazine-2-carboxamido)propanamido)ethyl)boronic acid (Compound 18, 6.0 mg). ¹H NMR (400 MHz, CD₃OD) δ 9.24 (s, 1H), 8.81-8.80 (m, 1H), 8.71-8.70 (m, 1H), 5.01-4.99 (m, 1H), 3.91-3.87 (m, 1H), 3.80-3.76 (m, 1H), 3.40 (s, 3H), 2.80-2.76 (m, 1H), 1.32-1.25 (m, 2H), 0.79-0.77 (m, 1H), 0.43-0.36 (m, 2H), 0.02-0.01 (m, 2H). [M−H]⁻: 335.0.

### Example 19: ((R)-1-((R)-2-(2,5-dioxopyrrolidin-1-yl)-3-methoxypropanamido)-4-phenyl butyl)boronic acid

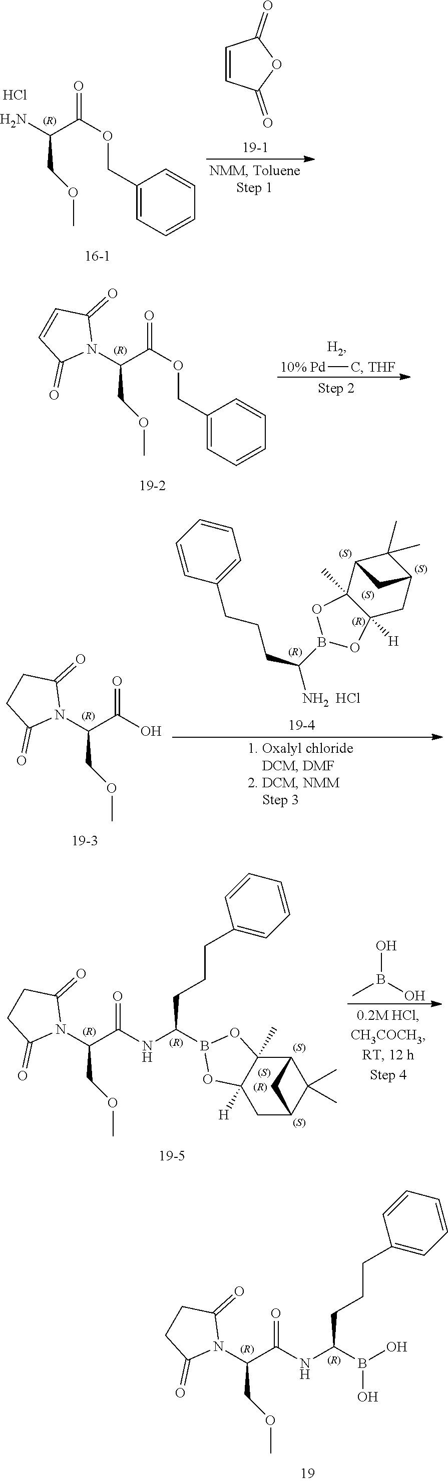

Synthesis of benzyl (R)-2-(2,5-dioxo-2,5-dihydro-1H-pyrrol-1-yl)-3-methoxypropanoate [Step 1]: To a stirred solution of benzyl O-methyl-D-serinate hydrochloride (16-1, 1.2 g, 4.88 mmol) in toluene (15 mL) was added NMM (1.6 mL, 14.7 mmol) followed by furan-2,5-dione (19-1, 575 mg, 5.86 mmol) at ambient temperature. The reaction mixture was stirred at 110° C. for 16 h. Volatiles were removed under reduced pressure. The residue was diluted with EtOAc and washed with water and brine, dried over anhydrous sodium sulphate and concentrated under reduced pressure. The product was purified by flash chromatography to afford benzyl (R)-2-(2,5-dioxo-2,5-dihydro-1H-pyrrol-1-yl)-3-methoxypropanoate (19-2, 450 mg). [M+H]⁺: 290.0.

Synthesis of (R)-2-(2,5-dioxopyrrolidin-1-yl)-3-methoxypropanoic acid [Step 2]: To a stirred solution of benzyl (R)-2-(2,5-dioxo-2,5-dihydro-1H-pyrrol-1-yl)-3-methoxypropanoate (19-2, 200 mg, 0.69 mmol) in THF (5 mL) was added 10% Pd—C (88 mg) under inert atmosphere and the reaction mixture was stirred under H₂ balloon pressure for 5 h at ambient temperature. The reaction mixture was filtered through a celite bed and the filtrate was evaporated under reduced pressure to afford (R)-2-(2,5-dioxopyrrolidin-1-yl)-3-methoxypropanoic acid (19-3, 190 mg). ¹H NMR (400 MHz, DMSO-d₆) δ 4.87-4.83 (m, 1H), 3.91-3.86 (m, 1H), 3.80-3.76 (m, 1H), 3.20 (s, 3H), 2.73 (s, 4H).

Synthesis of (R)-2-(2,5-dioxopyrrolidin-1-yl)-3-methoxy-N—((R)-4-phenyl-1-((3aS,4S,6S,7aR)-3a,5,5-trimethylhexahydro-4,6-methanobenzo[d][1,3,2]dioxaborol-2-yl)butyl)propanamide [Step 3]: To an ice cooled mixture of (R)-2-(2,5-dioxopyrrolidin-1-yl)-3-methoxypropanoic acid (19-3, 190 mg, 0.94 mmol) in DCM (5 mL) was added oxalyl dichloride (0.16 mL, 1.89 mmol) followed by catalytic amount of DMF. The reaction mixture was stirred at ambient temperature for 2 h. Volatiles were evaporated under reduced pressure to afford (R)-2-(2,5-dioxopyrrolidin-1-yl)-3-methoxypropanoyl chloride.

To a stirred solution of (1R)-4-phenyl-1-[(1S,2S,6R,8S)-2,9,9-trimethyl-3,5-dioxa-4-boratricyclo[6.1.1.02,6]decan-4-yl]butan-1-amine hydrochloride (19-4, 220 mg, 0.61 mmol) in DCM (3 mL) was added NMM (0.20 mL, 1.81 mmol) followed by a solution of (R)-2-(2,5-dioxopyrrolidin-1-yl)-3-methoxypropanoyl chloride in DCM (3 mL) at ice cool condition. The reaction mixture was stirred for 2 h at ambient temperature. The reaction mixture was diluted with DCM, washed with water and brine, dried over anhydrous sodium sulphate and concentrated under reduced pressure. The product was purified by PREP-HPLC purification and lyophilized to afford (R)-2-(2,5-dioxopyrrolidin-1-yl)-3-methoxy-N—((R)-4-phenyl-1-((3aS,4S,6S,7aR)-3a,5,5-trimethylhexahydro-4,6-methanobenzo[d][1,3,2]dioxaborol-2-yl)butyl)propanamide (19-5, 90 mg). [M−H]⁻: 469.3.

Synthesis of ((R)-1-((R)-2-(2,5-dioxopyrrolidin-1-yl)-3-methoxypropanamido)-4-phenyl butyl)boronic acid [Step 4]: To a stirred solution of (R)-2-(2,5-dioxopyrrolidin-1-yl)-3-methoxy-N—((R)-4-phenyl-1-((3aS,4S,6S,7aR)-3a,5,5-trimethylhexahydro-4,6-methanobenzo [d][1,3,2]dioxaborol-2-yl)butyl)propanamide (19-5, 95 mg, 0.21 mmol) in acetone (4 mL) was added 0.2 M HCl (4.0 mL) and methylboronic acid (124 mg, 2.07 mmol) at ice cool condition and the reaction mixture was stirred at ambient temperature for 16 h. The volatiles were evaporated under reduced pressure and the product was purified by prep HPLC purification and lyophilized to afford ((R)-1-((R)-2-(2,5-dioxopyrrolidin-1-yl)-3-methoxypropanamido)-4-phenylbutyl)boronic acid (Compound 19, 35 mg). ¹H NMR (400 MHz, CD₃OD) δ 7.24-7.20 (m, 2H), 7.17-7.15 (m, 2H), 7.13-7.09 (m, 1H), 5.14-5.09 (m, 1H), 4.01-3.96 (m, 2H), 3.33-3.32 (m, 3H), 2.73 (s, 4H), 2.68-2.64 (m, 1H), 2.59-2.55 (m, 2H), 1.67-1.61 (m, 2H), 1.60-1.55 (m, 1H), 1.53-1.47 (m, 1H). [M−H]⁻: 375.5.

### Example 20: ((R)-1-((R)-3-cyclopropoxy-2-(pyrazine-2-carboxamido)propanamido)-4-phenylbutyl)boronic acid

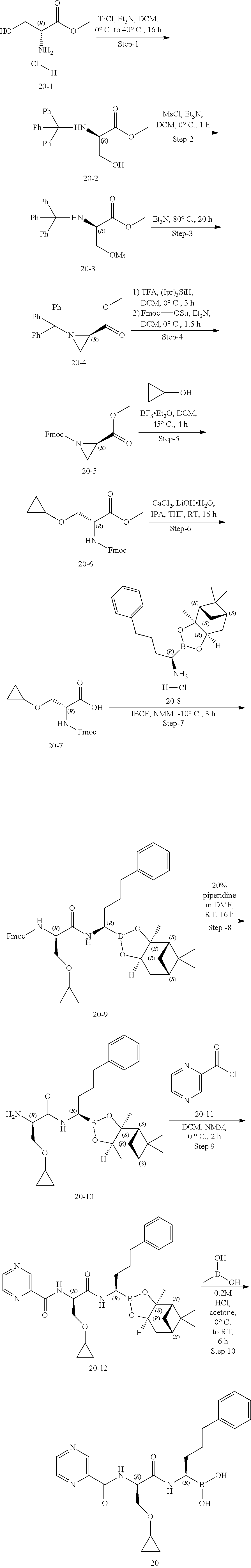

Synthesis of methyl trityl-D-serinate [Step 1]: To a stirred solution of methyl D-serinate hydrochloride (20-1, 5.0 g, 32.1 mmol) and Et₃N (11 mL, 80.3 mmol) in dichloromethane (25 mL) was added solution of trityl chloride (8.96 g, 32.1 mmol) in dichloromethane (25 mL) in portion at 0° C. The reaction mixture was finally allowed to stir at 40° C. for 16 h. After completion of reaction, the reaction mixture was diluted with ethylacetate and washed with aqueous NaHSO₄ solution and finally with brine solution. Organic layer was dried over anhydrous Na₂SO₄, filtered and concentrated under reduced pressure. Product was purified by combi flash column chromatography (0-50% EtOAc in Hexane) to afford methyl trityl-D-serinate (20-2, 8.2 g). ¹H NMR (400 MHz, CDCl₃) δ_{H} 7.48-7.46 (d, 6H), 7.28-7.16 (m, 9H), 3.70-3.68 (m, 1H), 3.57-3.53 (m, 2H), 3.29 (s, 3H), 2.97 (bs, 1H), 2.28 (bs, 1H).

Synthesis of methyl O-(methylsulfonyl)-N-trityl-D-serinate, 3, [Step 2]: To the stirred solution of methyl methyl trityl-D-serinate (20-2, 4.0 g, 11.1 mmol) in dichloromethane (50 mL) were added mesyl chloride (0.94 mL, 12.2 mmol) followed by the dropwise addition of Et₃N (2.3 mL, 16.6 mmol) at 0° C. The resulting solution was allowed to stir at 0° C. for 1h. Reaction mixture was then successively washed with 10% aqueous NaHSO₄ solution followed by brine solution. Organic layer was dried over anhydrous Na₂SO₄, filtered and concentrated to afford methyl 0-(methylsulfonyl)-N-trityl-D-serinate (20-3, 4.8 g). ¹H NMR (400 MHz, CDCl₃) δ_{H} 7.48-7.46 (m, 6H), 7.28-7.18 (m, 9H), 4.43-4.39 (m, 1H), 4.25-4.21 (m, 1H), 3.88-3.87 (m, 1H), 3.63 (m, 1H), 3.26 (s, 3H), 2.98 (s, 3H).

Synthesis of methyl (R)-1-tritylaziridine-2-carboxylate, 4, [Step 3]: To the stirred solution of methyl O-(methylsulfonyl)-N-trityl-D-serinate (20-3, 4.8 g, 10.9 mmol) in DME (40 mL) was added Et₃N (3.0 mL, 21.8 mmol) at ambient temperature and the reaction mixture was stirred at 80° C. for 30 h. The reaction mixture was diluted with ethylacetate (100 mL) and washed with water and brine solution. The organic layer was dried over anhydrous Na₂SO₄, filtered and evaporated under reduced pressure. product was purified by combi flash column chromatography (0-10% EtOAc in Hexane) to afford methyl (R)-1-tritylaziridine-2-carboxylate (20-4, 3.2 g). 1H NMR (400 MHz, CDCl₃) δ 7.50-7.48 (m, 5H), 7.29-7.19 (m, 10H), 3.75 (s, 3H), 2.25 (s, 1H), 1.89-1.87 (m, 1H), 1.41-1.40 (m, 1H).

Synthesis of 1-((9H-fluoren-9-yl)methyl) 2-methyl (R)-aziridine-1,2-dicarboxylate, 5, [Step 4]: To the stirred solution of methyl (R)-1-tritylaziridine-2-carboxylate (20-4, 2.0 g, 5.82 mmol) and tri-isopropylsilane (1.3 mL, 6.41 mmol) in dichloromethane (20 mL), was added solution of TFA (0.70 mL, 9.20 mmol) in dichloromethane (2 mL) dropwise at 0° C. The reaction mixture stirred for 1.5 h at same temperature. To this reaction mixture was added extra tri-isoproppylsilane (0.2 ml, 0.98 mmol) and TFA (0.25 ml, 3.3 mmol) at 0° C. and stirred for 1.5h. Upon completion of the reaction, the solvent was removed under reduced pressure. The resulting residue was dissolved in dichloromethane (15 mL) and added Fmoc-OSu (3.9 g, 11.6 mmol) followed by dropwise addition of Et₃N (2.4 mL, 17.5 mmol) in dichloromethane (5 mL) at 0° C. Resulting reaction mixture was stirred at 0° C. for additional 1.5h. Reaction mixture was washed with aqueous NH₄Cl solution, followed by aqueous NaHSO₄ solution and finally with brine solution. Organic phase was dried over anhydrous Na₂SO₄, filtered and concentrated under reduced pressure. Resulting residue was immediately purified by column chromatography using neutral alumina (0-25% EtOAc in Hexane) to afford 1-((9H-fluoren-9-yl)methyl) 2-methyl (R)-aziridine-1,2-dicarboxylate (20-5, 1.5 g). [M+H]⁺: 324.3.

Synthesis of methyl N-(((9H-fluoren-9-yl)methoxy)carbonyl)-O-cyclopropyl-D-serinate [Step 5]: To the stirred solution of 1-((9H-fluoren-9-yl)methyl) 2-methyl (R)-aziridine-1,2-dicarboxylate (20-5, 1.5 g, 4.64 mmol) and cyclopropanol (0.50 mL, 7.93 mmol) in dichloromethane (10 mL) was added BF₃·Et₂O (0.99 mL, 8.03 mmol) at −45° C. and stirred at same temperature for 4 h. Reaction mixture was diluted with dichloromethane and washed with water and brine solution. Organic layer was dried over Na₂SO₄, filtered and concentrated under reduced pressure. Product which was purified by combi flash column chromatography (0-20% EtOAc in hexane) to afford methyl N-(((9H-fluoren-9-yl)methoxy)carbonyl)-O-cyclopropyl-D-serinate (20-6, 650 mg). [M+H]⁺: 382.1.

Synthesis of N-(((9H-fluoren-9-yl)methoxy)carbonyl)-O-cyclopropyl-D-serine [Step 6]: To the stirred solution of methyl N-(((9H-fluoren-9-yl)methoxy)carbonyl)-O-cyclopropyl-D-serinate (20-6, 300 mg, 0.79 mmol) in a solvent mixture of THF (2 mL), IPA (8 mL) and water (2 mL) was added LiOH·H₂O (79 mg, 1.89 mmol) and CaCl₂ (1.3 g, 11.8 mmol). The reaction mixture was stirred at ambient temperature for 16 h. Reaction mixture was concentrated under reduced pressure. Residue was taken in water and washed with 20% Et₂O in hexane (two times). Aqueous part was acidified with NaHSO₄ and extracted with ethylacetate (two times). Combined organic layer was dried over Na₂SO₄, filtered and concentrated under educed pressure to afford solid which was triturated with 5% Et₂O in hexane to afford N-(((9H-fluoren-9-yl)methoxy)carbonyl)-O-cyclopropyl-D-serine (20-7, 170 mg). [M+H]⁺: 368.2.

Synthesis of (9H-fluoren-9-yl)methyl ((R)-3-cyclopropoxy-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)carbamate [Step 7]: To the stirred solution of N-(((9H-fluoren-9-yl)methoxy)carbonyl)-O-cyclopropyl-D-serine (20-7, 440 mg, 1.20 mmol) in THF (10 mL), IBCF (0.19 mL, 1.44 mmol) followed by NMM (0.20 mL, 1.44 mmol) were added at −10° C. Then the reaction mixture was allowed to stir for 30 min at same temperature. To this solution was added (R)-4-phenyl-1-((3aS,4S,6S,7aR)-3a,5,5-trimethylhexahydro-4,6-methanobenzo[d][1,3,2]dioxaborol-2-yl)butan-1-amine hydrochloride (20-8, 436 mg, 1.20 mmol) dissolving in THF (10 mL) dropwise followed by the addition of NMM (1.20 eq, 0.20 mL, 1.44 mmol) and stirred at the same temperature for 2.5 h. Reaction mixture was diluted with ethylacetate (50 mL) and was washed with cold aqueous 0.1 M HCl solution (two times), followed by 10% aqueous K₂CO₃ solution and finally with brine solution. Organic layer was dried over Na₂SO₄, filtered and evaporated under reduced pressure at 30° C. to afford 9H-fluoren-9-yl)methyl ((R)-3-cyclopropoxy-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)carbamate (20-9, 1.0 g) which was used in the next step without purification. [M+H]⁺: 677.3.

Synthesis of (R)-2-amino-3-cyclopropoxy-N—((R)-4-phenyl-1-((3aS,4S,6S,7aR)-3a,5,5-trimethylhexahydro-4,6-methanobenzo[d][1,3,2]dioxaborol-2-yl)butyl)propanamide [Step 8]: To the 9H-fluoren-9-yl)methyl ((R)-3-cyclopropoxy-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl) amino)propan-2-yl)carbamate (20-9, 1.0 g, 1.48 mmol) was added piperidine (20% w\v solution in DMF, 20 mL) at ambient temperature and stirred at ambient temperature for 16 h. The reaction mixture was diluted with cold water and extracted with ethyl acetate (two times). Combined organic extracts were washed with brine, dried over Na₂SO₄, filtered and concentrated under reduced pressure at 30° C. to afford (R)-2-amino-3-cyclopropoxy-N—((R)-4-phenyl-1-((3aS,4S,6S,7aR)-3a,5,5-trimethylhexahydro-4,6-methanobenzo[d][1,3,2]dioxaborol-2-yl)butyl) propanamide (20-10, 400 mg) which was used in the next step without purification. [M−H]⁻: 455.3.

Synthesis of N—((R)-3-cyclopropoxy-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide [Step 9]: To a stirred solution of (R)-2-amino-3-cyclopropoxy-N—((R)-4-phenyl-1-((3aS,4S,6S,7aR)-3a,5,5-trimethylhexahydro-4,6-methanobenzo[d][1,3,2]dioxaborol-2-yl)butyl)propanamide (20-10, 400 mg, 0.880 mmol) in dichloromethane (15 mL) was added NMM (0.15 mL, 1.32 mmol) at 0° C. and stirred for 10 min. To this resulting solution was added pyrazine-2-carbonyl chloride (20-11, 152 mg, 1.06 mmol) and stirred at ambient temperature for 2 h. The reaction mixture was diluted with dichloromethane and washed with aqueous NaHCO₃ solution, followed by brine solution. The organic layer was dried over anhydrous Na₂SO₄, filtered and concentrated under reduced pressure. product was purified by reverse phase PREP-HPLC to afford N—((R)-3-cyclopropoxy-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl) amino)propan-2-yl)pyrazine-2-carboxamide (20-12, 25 mg). [M−H]⁻: 559.4.

Synthesis of ((R)-1-((R)-3-cyclopropoxy-2-(pyrazine-2-carboxamido)propanamido)-4-phenylbutyl)boronic acid [Step 10]: To a stirred solution of N—((R)-3-cyclopropoxy-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (20-12, 25 mg, 0.045 mmol) in acetone (2 mL), were added Methylboronic acid (27 mg, 0.45 mmol) followed by HCl (0.2 M in water, 2.0 mL) at 0° C. and the reaction mixture was stirred at ambient temperature for 6 h. After completion of the reaction, reaction mixture was concentrated under reduced pressure at 30° C. and lyophilized. Resulting solid was purified by reverse phase PREP-HPLC to afford ((R)-1-((R)-3-cyclopropoxy-2-(pyrazine-2-carboxamido)propanamido)-4-phenylbutyl)boronic acid (Compound 20, 12 mg). ¹H NMR (400 MHz, CD₃OD) δ_{H} 9.24 (s, 1H), 8.81 (s, 1H), 8.70 (s, 1H), 7.22-7.08 (m, 5H), 4.96 (m, 1H), 4.00-3.97 (m, 1H), 3.90-3.86 (m, 1H), 3.38 (m, 1H), 2.64-2.56 (m, 3H), 1.64-1.45 (m, 4H), 0.53-0.47 (m, 4H). [M−H]⁻: 425.5.

### Example 21: ((R)-3-(4-chlorophenoxy)-1-((R)-3-methoxy-2-(pyrazine-2-carboxamido) propanamido)propyl)boronic acid

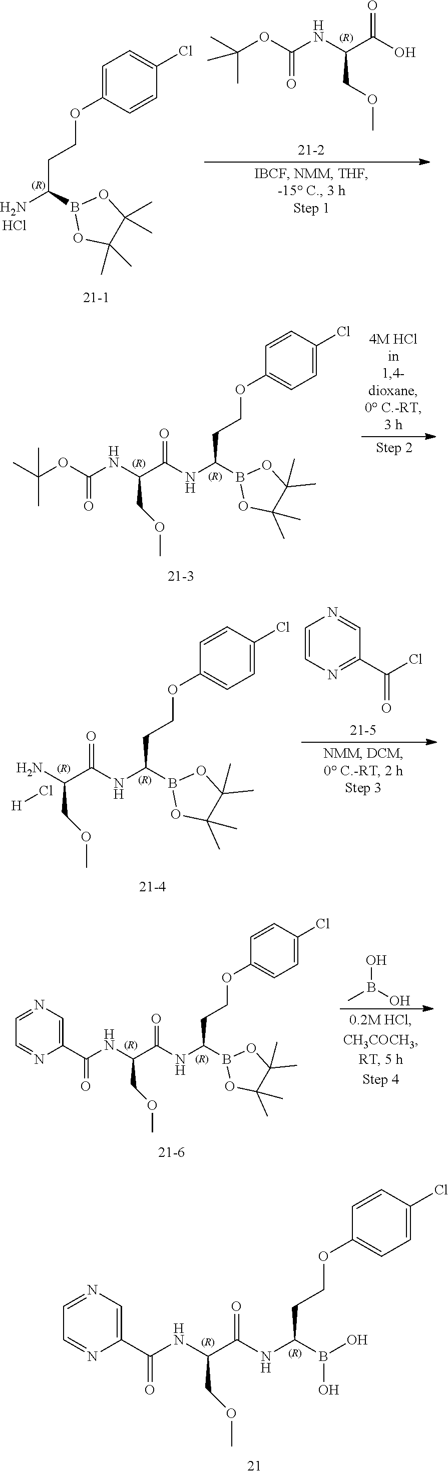

Synthesis of tert-butyl ((R)-1-(((R)-3-(4-chlorophenoxy)-1-(4,4,5,5-tetramethyl-1,3,2-dioxaborolan-2-yl)propyl)amino)-3-methoxy-1-oxopropan-2-yl)carbamate [Step 1]: To a stirred solution of N-(tert-butoxycarbonyl)-O-methyl-D-serine (21-2, 227 mg, 1.03 mmol) in THF (4 mL) was added IBCF (0.13 mL, 0.95 mmol) followed by NMM (0.14 mL, 1.03 mmol) at −15° C. and stirred for 30 min. A solution of (R)-3-(4-chlorophenoxy)-1-(4,4,5,5-tetramethyl-1,3,2-dioxaborolan-2-yl)propan-1-amine hydrochloride (21-1, 0.86 mmol) in THF (4 mL) was added dropwise followed by NMM (0.14 mL, 1.03 mmol) to the reaction mixture at −15° C. and then allowed to warm to 0° C. and stirred for 2 h. The reaction mixture was diluted with ethyl acetate and washed with 0.1 M HCl (twice), 5% aqueous K₂CO₃ solution (twice), water (twice) and brine, dried over anhydrous Na₂SO₄ and concentrated under reduced pressure at 30° C. to give tert-butyl ((R)-1-(((R)-3-(4-chlorophenoxy)-1-(4,4,5,5-tetramethyl-1,3,2-dioxaborolan-2-yl)propyl) amino)-3-methoxy-1-oxopropan-2-yl)carbamate (21-3, 460 mg). This product was forwarded to the next step without further purification. [M−H]⁻: 511.3.

Synthesis of (R)-2-amino-N—((R)-3-(4-chlorophenoxy)-1-(4,4,5,5-tetramethyl-1,3,2-dioxaborolan-2-yl)propyl)-3-methoxypropanamide hydrochloride [Step 2]: To a stirred solution of tert-butyl ((R)-1-(((R)-3-(4-chlorophenoxy)-1-(4,4,5,5-tetramethyl-1,3,2-dioxaborolan-2-yl)propyl)amino)-3-methoxy-1-oxopropan-2-yl)carbamate (21-3, 460 mg, 0.89 mmol) in 1,4-dioxane (5 mL), was added 4 M HCl in 1,4-dioxane (2.5 mL, 9.87 mmol) dropwise at 0° C. The reaction mixture was allowed to warm to ambient temperature and stirred for 3 h. Volatiles were evaporated under reduced pressure and lyophilized to afford (R)-2-amino-N—((R)-3-(4-chlorophenoxy)-1-(4,4,5,5-tetramethyl-1,3,2-dioxaborolan-2-yl)propyl)-3-methoxypropanamide hydrochloride (21-4, 300 mg). [M−H]⁻: 411.4.

Synthesis of N—((R)-1-(((R)-3-(4-chlorophenoxy)-1-(4,4,5,5-tetramethyl-1,3,2-dioxaborolan-2-yI)propyl)amino)-3-methoxy-1-oxopropan-2-yl)pyrazine-2-carboxamide [Step 3]: To a stirred solution of (R)-2-amino-N—((R)-3-(4-chlorophenoxy)-1-(4,4,5,5-tetramethyl-1,3,2-dioxaborolan-2-yl)propyl)-3-methoxypropanamide hydrochloride (21-4, 350 mg, 0.78 mmol) in dichloromethane (5 mL) was added NMM (0.43 mL, 3.90 mmol) at 0° C. followed by the addition of pyrazine-2-carbonyl chloride (21-5, 111 mg, 0.78 mmol) and stirred for 15 min at 0° C. The reaction mixture was allowed to warm to ambient temperature and stirred for 2 h. The reaction mixture was diluted with dichloromethane and washed with ice-cold water, brine, dried over anhydrous Na₂SO₄, filtered and concentrated under reduced pressure to afford N—((R)-1-(((R)-3-(4-chlorophenoxy)-1-(4,4,5,5-tetramethyl-1,3,2-dioxaborolan-2-yl)propyl)amino)-3-methoxy-1-oxopropan-2-yl)pyrazine-2-carboxamide (21-6, 380 mg). [M−H]⁻: 517.4.

Synthesis of ((R)-3-(4-chlorophenoxy)-1-((R)-3-methoxy-2-(pyrazine-2-carboxamido) propanamido)propyl)boronic acid [Step 4]: To a stirred solution of N—((R)-1-(((R)-3-(4-chlorophenoxy)-1-(4,4,5,5-tetramethyl-1,3,2-dioxaborolan-2-yl)propyl)amino)-3-methoxy-1-oxopropan-2-yl)pyrazine-2-carboxamide (21-6, 380 mg, 0.73 mmol) and methylboronic acid (438 mg, 7.32 mmol) in acetone (4 mL) was added 0.2 M HCl (3.7 mL) and stirred at ambient temperature for 5 h. All volatiles were evaporated under reduced pressure and purified by PREP-HPLC and lyophilized to afford ((R)-3-(4-chlorophenoxy)-1-((R)-3-methoxy-2-(pyrazine-2-carboxamido)propanamido)propyl)boronic acid (Compound 21, 16 mg). [M−H]: 435.2, ¹H NMR (400 MHz, CD₃OD) δ_{H}: 9.24 (d, 1H), 8.81 (d, 1H), 8.71-8.70 (m, 1H), 7.20-7.18 (m, 2H), 6.88-6.85 (m, 2H), 5.00 (t, 1H), 4.01 (t, 2H), 3.92-3.89 (m, 1H), 3.80-3.77 (m, 1H), 3.39 (s, 3H), 2.89 (t, 1H), 1.98-1.89 (m, 2H).

### Example 22: ((S)-2-((R)-3-methoxy-2-(pyrazine-2-carboxamido)propanamido)-5-phenyl pentan-2-yl)boronic acid

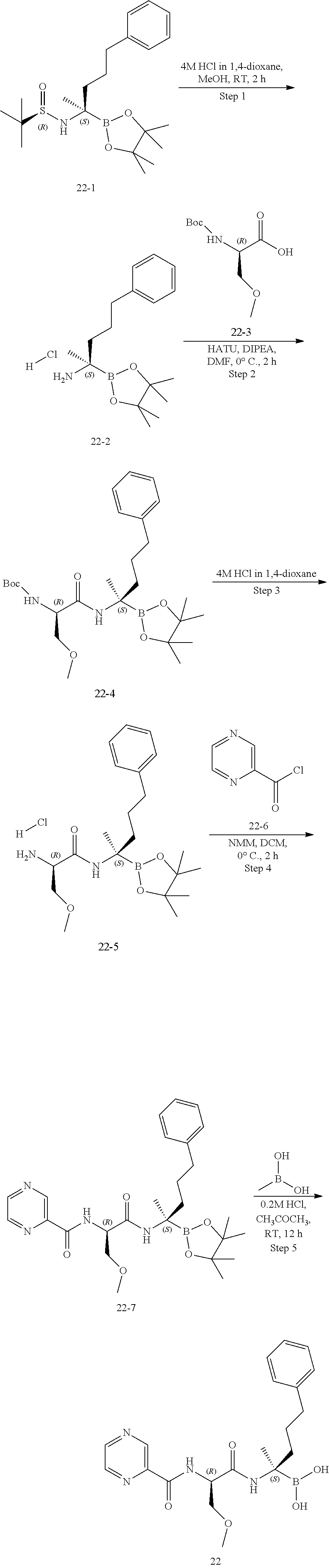

Synthesis of (S)-5-phenyl-2-(4,4,5,5-tetramethyl-1,3,2-dioxaborolan-2-yl)pentan-2-amine hydrochloride [Step 1]: To a stirred solution of (R)-2-methyl-N—((S)-5-phenyl-2-(4,4,5,5-tetramethyl-1,3,2-dioxaborolan-2-yl)pentan-2-yl)propane-2-sulfinamide (22-1, 100 mg, 0.25 mmol) in 1,4-dioxane (2 mL) were added methanol (0.1 mL, 2.5 mmol) followed by 4 M HCl in 1,4-dioxane (0.06 mL, 0.25 mmol) in ice cold condition and stirred at ambient temperature for 2 h. Volatiles were removed under reduced pressure to give (S)-5-phenyl-2-(4,4,5,5-tetramethyl-1,3,2-dioxaborolan-2-yl)pentan-2-amine hydrochloride (22-2, 100 mg). [M+H]⁺: 290.1 and 207.8 (mass peak of corresponding boronic acid).

Synthesis of tert-butyl ((R)-3-methoxy-1-oxo-1-(((S)-5-phenyl-2-(4,4,5,5-tetramethyl-1,3,2-dioxaborolan-2-yl)pentan-2-yl)amino)propan-2-yl)carbamate [Step 2]: To a stirred solution of N-(tert-butoxycarbonyl)-O-methyl-D-serine (22-3, 73 mg, 0.3 mmol) in DMF (2 mL) was added HATU (158 mg, 0.4 mmol) followed by DIPEA (0.1 mL, 0.5 mmol) at 0° C. and stirred for 30 min. (S)-5-phenyl-2-(4,4,5,5-tetramethyl-1,3,2-dioxaborolan-2-yl)pentan-2-amine hydrochloride (22-2, 90 mg, 0.27 mmol) was added and stirred at 0° C. for 2 h. Reaction mixture was quenched with 5% aqueous K₂CO₃ solution and extracted with EtOAc (thrice). Combined organic layer was washed with water (thrice) and brine, dried over anhydrous Na₂SO₄ and evaporated under reduced pressure to give tert-butyl ((R)-3-methoxy-1-oxo-1-(((S)-5-phenyl-2-(4,4,5,5-tetramethyl-1,3,2-dioxaborolan-2-yl)pentan-2-yl)amino)propan-2-yl)carbamate (22-4, 150 mg). [M−H]⁻: 489.5.

Synthesis of (R)-2-amino-3-methoxy-N—((S)-5-phenyl-2-(4,4,5,5-tetramethyl-1,3,2-dioxaborolan-2-yl)pentan-2-yl)propanamide hydrochloride [Step 3]: To a solution of tert-butyl ((R)-3-methoxy-1-oxo-1-(((S)-5-phenyl-2-(4,4,5,5-tetramethyl-1,3,2-dioxaborolan-2-yl) pentan-2-yl)amino)propan-2-yl)carbamate (22-4, 150 mg, 0.3 mmol) in 1,4-dioxane (3 mL) was added 4 M HCl in 1,4-dioxane (1 mL) at 0° C. and stirred at ambient temperature 16 h. Volatiles were removed under reduced pressure to give (R)-2-amino-3-methoxy-N—((S)-5-phenyl-2-(4,4,5,5-tetramethyl-1,3,2-dioxaborolan-2-yl)pentan-2-yl)propanamide hydrochloride (22-5, 135 mg). [M−H]⁻: 389.4.

Synthesis of N—((R)-3-methoxy-1-oxo-1-(((S)-5-phenyl-2-(4,4,5,5-tetramethyl-1,3,2-dioxaborolan-2-yl)pentan-2-yl)amino)propan-2-yl)pyrazine-2-carboxamide [Step 4]: To a stirred solution of (R)-2-amino-3-methoxy-N—((S)-5-phenyl-2-(4,4,5,5-tetramethyl-1,3,2-dioxaborolan-2-yl)pentan-2-yl)propanamide hydrochloride (22-5, 200 mg, 0.5 mmol) in DCM (10 mL) was added NMM (0.06 mL, 0.9 mmol) at 0-5° C. Pyrazine-2-carbonyl chloride (22-6, 100 mg, 0.7 mmol) was added to the reaction mixture and stirred at ambient temperature for 1.5 h. Reaction mixture was diluted with water and extracted with DCM. Combined organic layer was washed with water and brine, dried over anhydrous Na₂SO₄ and evaporated under reduced pressure. The product was purified by prep HPLC purification and lyophilized to afford N—((R)-3-methoxy-1-oxo-1-(((S)-5-phenyl-2-(4,4,5,5-tetramethyl-1,3,2-dioxaborolan-2-yl)pentan-2-yl) amino)propan-2-yl)pyrazine-2-carboxamide (22-7, 200 mg). [M−H]⁻: 495.4.

Synthesis of ((S)-2-((R)-3-methoxy-2-(pyrazine-2-carboxamido)propanamido)-5-phenyl pentan-2-yl)boronic acid [Step 5]: To a stirred solution of N—((R)-3-methoxy-1-oxo-1-(((S)-5-phenyl-2-(4,4,5,5-tetramethyl-1,3,2-dioxaborolan-2-yl)pentan-2-yl)amino)propan-2-yl)pyrazine-2-carboxamide (22-7, 150 mg, 0.3 mmol) and methylboronic acid (180 mg, 3.0 mmol) in acetone (2.0 mL) was added 0.2 M HCl (2.0 mL) and stirred at ambient temperature for 16 h. Volatiles were evaporated under reduced pressure and purified by prep HPLC purification and lyophilized to afford ((S)-2-((R)-3-methoxy-2-(pyrazine-2-carboxamido)propanamido)-5-phenylpentan-2-yl) boronic acid (Compound 22, 7 mg). [M−H]⁻: 413.0. ¹H NMR (400 MHz, DMSO-d₆+2 drops of D₂O at 80° C.) δ_{H}: 9.73 (s, 1H), 8.37 (s, 1H), 8.66 (s, 1H), 7.19-7.06 (m, 5H), 4.73 (t, 1H), 3.71-3.60 (m, 2H), 3.36 (s, 3H), 2.41-2.32 (m, 2H), 1.53-1.40 (m, 4H), 1.08 (s, 3H).

### Example 23: ((R)-3-(4-fluorophenoxy)-1-((R)-3-methoxy-2-(pyrazine-2-carboxamido) propanamido)propyl)boronic acid

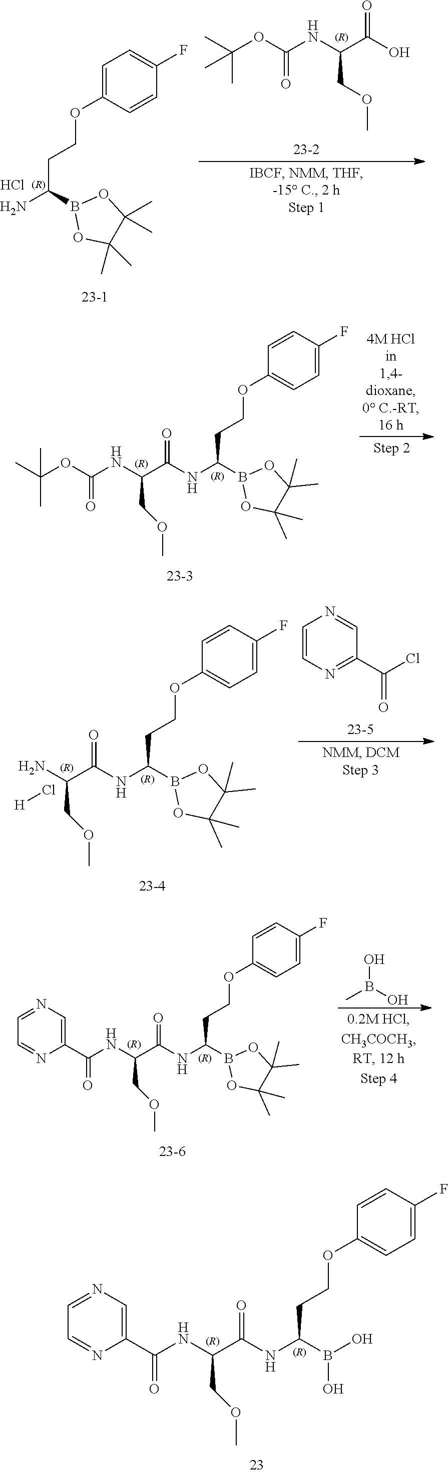

Synthesis of tert-butyl ((R)-1-(((R)-3-(4-fluorophenoxy)-1-(4,4,5,5-tetramethyl-1,3,2-dioxaborolan-2-yl)propyl)amino)-3-methoxy-1-oxopropan-2-yl)carbamate [Step 1]: To a stirred solution of N-(tert-butoxycarbonyl)-O-methyl-D-serine (23-2, 793 mg, 3.6 mmol) in tetrahydrofuran (8 mL) was added isobutyl chloroformate (IBCF, 0.35 mL, 2.7 mmol) and N-methylmorpholine (NMM, 0.36 mL, 3.3 mmol) at −15° C. and stirred at same temperature for 30 min. A solution of (R)-3-(4-fluorophenoxy)-1-(4,4,5,5-tetramethyl-1,3,2-dioxaborolan-2-yl)propan-1-amine hydrochloride (23-1, 1 g, 3.3 mmol) in tetrahydrofuran (7 mL) followed by N-methylmorpholine (0.33 mL, 3 mmol) were added to the reaction mixture at −15° C. and gradually warmed to 0° C. and stirred for 2 h. The reaction mixture was neutralized with 0.1 M HCl and extracted with ethyl acetate (twice). The combined organic extract was washed with 5% aqueous K₂CO₃, water and brine, dried over anhydrous Na₂SO₄ and concentrated under reduced pressure to afford tert-butyl ((R)-1-(((R)-3-(4-fluorophenoxy)-1-(4,4,5,5-tetramethyl-1,3,2-dioxaborolan-2-yl)propyl)amino)-3-methoxy-1-oxopropan-2-yl)carbamate (23-3, 1.5 g). [M−H]⁻: 495.1.

Synthesis of (R)-2-amino-N—((R)-3-(4-fluorophenoxy)-1-(4,4,5,5-tetramethyl-1,3,2-dioxaborolan-2-yl)propyl)-3-methoxypropanamide hydrochloride [Step 2]: To a stirred solution of tert-butyl ((R)-1-(((R)-3-(4-fluorophenoxy)-1-(4,4,5,5-tetramethyl-1,3,2-dioxaborolan-2-yl)propyl)amino)-3-methoxy-1-oxopropan-2-yl)carbamate (23-3, 1.5 g, 3 mmol) in 1,4-dioxane (15 mL) was added 4 M HCl in 1,4-dioxane (7.6 mL, 30 mmol) at ice cold condition and stirred at 0° C. for 2 h. The reaction mixture was evaporated under reduced pressure and triturated with n-pentane and dried to afford (R)-2-amino-N—((R)-3-(4-fluorophenoxy)-1-(4,4,5,5-tetramethyl-1,3,2-dioxaborolan-2-yl)propyl)-3-methoxypropanamide hydrochloride (23-4, 1.4 g). [M−H]⁻: 395.4, [M−83]⁻: 313.4. Corresponding boronic acid mass peak was observed in LCMS.

Synthesis of N—((R)-1-(((R)-3-(4-fluorophenoxy)-1-(4,4,5,5-tetramethyl-1,3,2-dioxaborolan-2-yl)propyl)amino)-3-methoxy-1-oxopropan-2-yl)pyrazine-2-carboxamide [Step 3]: To a stirred solution of (R)-2-amino-N—((R)-3-(4-fluorophenoxy)-1-(4,4,5,5-tetramethyl-1,3,2-dioxaborolan-2-yl)propyl)-3-methoxypropanamide hydrochloride (23-4, 600 mg, 1.4 mmol) in dichloromethane (6 mL) was added N-methylmorpholine (0.3 mL, 4.2 mmol) at −15° C. followed by the addition of pyrazine-2-carbonyl chloride (22-5, 296 mg, 2.1 mmol) and stirred for 2 h. The reaction mixture was diluted with dichloromethane and washed with water. The aqueous extract was further washed with dichloromethane. The combined organic extract was washed with brine, dried over anhydrous sodium sulfate and concentrated under reduced pressure. The crude product was purified by prep HPLC purification and lyophilized to afford N—((R)-1-(((R)-3-(4-fluorophenoxy)-1-(4,4,5,5-tetramethyl-1,3,2-dioxaborolan-2-yl)propyl)amino)-3-methoxy-1-oxopropan-2-yl)pyrazine-2-carboxamide (23-6, 40 mg). [M−H]⁻: 501.4, [M-83]⁻: 419.2. Corresponding boronic acid mass peak was observed.

Synthesis of ((R)-3-(4-fluorophenoxy)-1-((R)-3-methoxy-2-(pyrazine-2-carboxamido) propanamido)propyl)boronic acid [Step 4]: To a stirred solution of N—((R)-1-(((R)-3-(4-fluorophenoxy)-1-(4,4,5,5-tetramethyl-1,3,2-dioxaborolan-2-yl)propyl)amino)-3-methoxy-1-oxopropan-2-yl)pyrazine-2-carboxamide (6, 40 mg, 0.08 mmol) in acetone (8 mL) was added 0.2 M HCl (8 mL) and methylboronic acid (48 mg, 0.80 mmol)) at ice cold condition and stirred at ambient temperature for 16 h. Volatiles were evaporated under reduced pressure and the crude product was purified by prep HPLC purification and lyophilized to afford ((R)-3-(4-fluorophenoxy)-1-((R)-3-methoxy-2-(pyrazine-2-carboxamido)propanamido)propyl)boronic acid (Compound 23, 28 mg). [M−H]⁻: 419.4. ¹H NMR (400 MHz, DMSO-d₆+2 drop D₂O) d_{H}: 9.15 (s, 1H), 8.85 (s, 1H), 8.71 (s, 1H), 7.04-6.99 (m, 2H), 6.87-6.84 (m, 2H), 4.68-4.65 (m, 1H), 3.93-3.90 (m, 2H), 3.71-3.67 (m, 1H), 3.64-3.60 (m, 1H), 3.29-3.26 (m, 1H), 3.24 (s, 3H), 2.01-1.97 (m, 1H), 1.95-1.86 (m, 1H).

### Example 24: ((R)-3-(4-chloro-2-fluorophenoxy)-1-((R)-3-methoxy-2-(pyrazine-2-carboxamido)propanamido)propyl)boronic acid

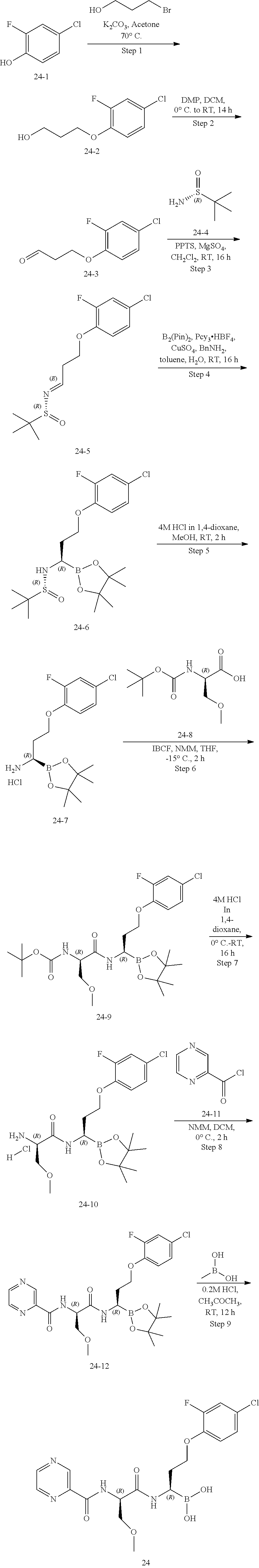

Synthesis of 3-(4-chloro-2-fluorophenoxy)propan-1-ol [Step 1]: To a stirred solution of 4-chloro-2-fluoro-phenol (24-1, 4.0 g, 27.3 mmol) in acetone (40 mL) was added 3-bromopropan-1-ol (4.2 g, 30.0 mmol) and K₂CO₃ (4.5 g, 32.8 mmol) at 25° C. The resulting mixture was heated at 60° C. and stirred for 20 h. The reaction mixture was filtered through celite bed and washed with acetone thrice. The filtrate was concentrated under reduced pressure and the crude product was purified by flash column chromatography to afford 3-(4-chloro-2-fluorophenoxy)propan-1-ol (24-2, 5.5 g). ¹H NMR (400 MHz, CDCl₃) δ_{H}; 7.09-7.05 (m, 1H), 7.03-7.00 (m, 1H), 6.88 (t, 1H), 4.14 (t, 2H), 3.85 (t, 1H), 2.07-1.92 (m, 2H).

Synthesis of 3-(4-chloro-2-fluoro-phenoxy)propanal [Step 2]: To a stirred solution of 3-(4-chloro-2-fluoro-phenoxy)propan-1-ol (24-2, 4.0 g, 19.5 mmol) in dichloromethane (100 mL) was added Dess-Martin periodinane (12.4 g, 29.3 mmol) at 0° C. The reaction mixture was allowed to warm to ambient temperature and stirred for 16 h. The reaction mixture was filtered through celite pad and thoroughly washed with dichloromethane. The combined filtrate was washed with aq. NaHCO₃. The organic layer was collected and washed with water and brine, dried over anhydrous Na₂SO₄, filtered and concentrated under reduced pressure and the crude product was purified by flash column chromatography to afford 3-(4-chloro-2-fluoro-phenoxy)propanal (24-3, 1.7 g). ¹H NMR (400 MHz, CDCl₃) δ_{H}; 9.86 (s, 1H), 7.10-6.96 (m, 3H), 6.91 (t, 1H), 4.33 (t, 2H), 2.94 (t, 2H).

Synthesis of (R,E)-N-[3-(4-chloro-2-fluoro-phenoxy)propylidene]-2-methyl-propane-2-sulfinamide [Step 3]: To a stirred solution of (R)-2-methylpropane-2-sulfinamide (24-4, 10.80 g, 8.9 mmol) in dichloromethane (60 mL) were added PPTS (1.37 g, 5.5 mmol), anhydrous magnesium sulphate (5.4 g, 44.6 mmol) and 3-(4-chloro-2-fluoro-phenoxy)propanal (24-3, 1.98 g, 9.8 mmol) under nitrogen atmosphere at ice cold condition. The reaction was allowed to warm to ambient temperature and stirred for 16 h. The reaction mixture was filtered through a pad of celite and washed with dichloromethane several times. The combined filtrate was evaporated under reduced pressure and the obtained residue was purified by flash column chromatography to afford (S,E)-N-[3-(4-chloro-2-fluoro-phenoxy)propylidene]-2-methyl-propane-2-sulfinamide (24-5, 1.2 g). ¹H NMR (400 MHz, DMSO-d₆) δ_{H}; 8.03 (t, 1H), 7.42 (t, 1H), 7.26-7.19 (m, 2H), 4.41-4.36 (m, 2H), 3.01-2.98 (m, 2H), 1.08 (s, 9H).

Synthesis of (R)—N—[(R)-3-(4-chloro-2-fuorophenoxy)-1-(4,4,5,5-tetramethyl-1,3,2-dioxaborolan-2-yl)propyl)-2-methylpropane-2-sulfinamide [Step 4]: To a suspension of tricyclohexylphosphine tetrafluoroborate (47 mg, 0.13 mmol) in toluene (2 mL) were added CuSO₄·5H₂O (32 mg, 0.13 mmol) dissolved in water (1.3 mL) and benzylamine (0.05 mL, 0.43 mmol) and stirred vigorously for 40 min. The solution was diluted with toluene (12.7 mL) and to this solution were added (R,E)-N-[3-(4-chloro-2-fluoro-phenoxy)propylidene]-2-methyl-propane-2-sulfinamide (24-5, 1.3 g, 4.25 mmol) and bispinacolatediborane (2.2 g, 8.5 mmol) and stirred at ambient temperature for 16 h. After diluting with ethyl acetate, the precipitate was filtered through a short pad of deactivated silica gel (SiO₂/H₂O 100:35, m/m) and washed with ethyl acetate. The filtrate was concentrated under reduced pressure and the crude product was purified by flash column chromatography using deactivated silica gel (SiO₂/H₂O 100:35, m/m) to afford (R)—N—[(R)-3-(4-chloro-2-fluorophenoxy)-1-(4,4,5,5-tetramethyl-1,3,2-dioxaborolan-2-yl)propyl)-2-methyl propane-2-sulfinamide (24-6, 1.0 g). ¹H NMR (400 MHz, DMSO-d₆) δ_{H}: 7.43-7.40 (m, 1H), 7.21-7.17 (m, 2H), 4.97 (d, 1H), 4.19-4.09 (m, 2H), 3.00 (d, 1H), 1.97 (d, 2H), 1.15 (s, 9H), 1.06 (s, 12H).

Synthesis of (R)-3-(4-chloro-2-fluorophenoxy)-1-(4,4,5,5-tetramethyl-1,3,2-dioxaborolan-2-yl)propan-1-amine hydrochloride [Step 5]: To a stirred solution of (R)—N—((R)-3-(4-chloro-2-fluorophenoxy)-1-(4,4,5,5-tetramethyl-1,3,2-dioxaborolan-2-yl)propyl)-2-methylpropane-2-sulfinamide (24-6, 850 mg, 1.96 mmol) in 1,4-dioxane (4 mL), was added methanol (0.8 mL, 19.6 mmol) followed by the dropwise addition of 4 M HCl in dioxane (0.50 mL, 1.96 mmol) at 0° C. The resulting reaction mixture was allowed to stir at ambient temperature for 2 h. The reaction mixture was evaporated under reduced pressure and lyophilized to give (R)-3-(4-chloro-2-fluorophenoxy)-1-(4,4,5,5-tetramethyl-1,3,2-dioxaborolan-2-yl)propan-1-amine hydrochloride (24-7, 300 mg). ¹H NMR (400 MHz, DMSO-d₆) δ_{H}: 7.87 (s, 2H), 7.44 (d, 1H), 7.27-7.15 (m, 2H), 4.15-4.11 (m, 2H), 2.92 (s, 1H) 2.06 (t, 2H), 1.06 (s, 12H). The crude product forward for next step without further purification.

Synthesis of tert-butyl(-1-(((-3-4-chloro-2-fluorophenoxy)-1-(4,4,5,5,-tetramethyl-1,3,2-dioxaborolan-2-yl)propyl)amino)-3-methoxy-1-oxopropan-2-yl)carbamate [Step 6]: To a stirred solution (R)-2-(tert-butoxycarbonylamino)-3-methoxy-propanoic acid (24-8, 150 mg, 0.68 mmol) in THF (1 mL) was added IBCF (0.098 mL, 0.75 mmol) followed by NMM (0.09 mL, 0.82 mmol) at −15° C. and stirred for 30 min. A solution of (R)-3-(4-chloro-2-fluoro-phenoxy)-1-(4,4,5,5-tetramethyl-1,3,2-dioxaborolan-2-yl)propan-1-amine hydrochloride (24-7, 250 mg, 0.68 mmol) in THF (0.5 mL) was added dropwise followed by NMM (0.09 mL, 0.82 mmol) to the reaction mixture at −15° C. and it was allowed to warm to 0° C. and stirred for 2 h. The reaction mixture was diluted with ethyl acetate and washed with 0.1 M HCl, 5% aqueous K₂CO₃ solution, water and brine, dried over anhydrous Na₂SO₄ and concentrated under reduced pressure to afford tert-butyl(-1-(((-3-4-chloro-2-fluorophenoxy)-1-(4,4,5,5,-tetramethyl-1,3,2-dioxaborolan-2-yl)propyl)amino)-3-methoxy-1-oxopropan-2-yl)carbamate (24-9, 350 mg). [M−H]⁻: 529.4. The crude product forward for next step without further purification.

Synthesis of (R)-2-amino-N—((R)-3-(4-chloro-2-fluorophenoxy)-1-(4,4,5,5-tetramethyl-1,3,2-dioxaborolan-2-yl)propyl)-3-methoxypropanamide hydrochloride [Step 7]: To a solution of tert-butyl(-1-(((-3-4-chloro-2-fluorophenoxy)-1-(4,4,5,5,-tetramethyl-1,3,2-dioxaborolan-2-yl) propyl)amino)-3-methoxy-1-oxopropan-2-yl)carbamate (24-9, 300 mg, 0.57 mmol) in 1,4-dioxane (3 mL) was added 4 M HCl in 1,4-dioxane (1.4 mL, 5.7 mmol) at 0° C. and allowed to stir to ambient temperature for 3 h. The reaction mixture was concentrated under reduced pressure and lyophilized to afford (R)-2-amino-N—((R)-3-(4-chloro-2-fluorophenoxy)-1-(4,4,5,5-tetramethyl-1,3,2-dioxaborolan-2-yl)propyl)-3-methoxypropanamide hydrochloride (24-10, 180 mg). The crude product was used in next step without further purification. [M−H]⁻: 429.3. [M-82]⁻: 347 (mass of corresponding boronic acid).

Synthesis of N—((R)-1-((R)-3-(4-chloro-2-fluorophenoxy)-1-(4,4,5,5-tetramethyl-1,3,2-dioxaborolan-2-yl)propyl)-3-methoxy-1-oxopropan-2-yl)pyrazine-2-carboxamide [Step 8]: To a stirred solution of (2R)-2-amino-N-[(1R)-3-(4-chloro-2-fluoro-phenoxy)-1-(4,4,5,5-tetramethyl-1,3,2-dioxaborolan-2-yl)propyl]-3-methoxy-propanamide hydrochloride (24-10, 180 mg, 0.39 mmol) in dichloromethane (3 mL) was added NMM (0.17 mL, 1.5 mmol), followed by the addition of pyrazine-2-carbonyl chloride (24-11, 55 mg, 0.39 mmol) and stirred for 15 min at 0° C. and allowed to stir to ambient temperature for 2 h. The reaction mixture was diluted with dichloromethane and washed with aq. solution of NaHCO₃ and finally with brine, dried over Na₂SO₄ and concentrated under reduced pressure to afford N—((R)-1-((R)-3-(4-chloro-2-fluorophenoxy)-1-(4,4,5,5-tetramethyl-1,3,2-dioxaborolan-2-yl)propyl)-3-methoxy-1-oxopropan-2-yl)pyrazine-2-carboxamide (24-12, 180 mg). [M−H]⁻: 535.5. Crude product forward for next step without further purification.

Synthesis of ((R)-3-(4-chloro-2-fluorophenoxy)-1-((R)-3-methoxy-2-(pyrazine-2-carboxamido)propanamido)propyl)boronic acid [Step 9]: To a solution of N—((R)-1-((R)-3-(4-chloro-2-fluorophenoxy)-1-(4,4,5,5-tetramethyl-1,3,2-dioxaborolan-2-yl)propyl)-3-methoxy-1-oxopropan-2-yl)pyrazine-2-carboxamide (24-12, 180 mg, 0.34 mmol) and methylboronic acid (201 mg, 3.4 mmol) in acetone (3 mL) was added 0.2 M HCl (1.7 mL, 0.34 mmol) at 0° C. The reaction mixture was allowed to stir at ambient temperature for 5 h. All volatiles were evaporated under reduced pressure and the crude product purified by prep HPLC purification and lyophilized to give ((R)-3-(4-chloro-2-fluorophenoxy)-1-((R)-3-methoxy-2-(pyrazine-2-carboxamido) propanamido)propyl)boronic acid (Compound 24, 20 mg). ¹H NMR (400 MHz, DMSO-d₆+2 drop D₂O at 80° C.): δ_{H} 9.16 (d, 1H), 8.86 (d, 1H), 871 (d, 1H), 7.13-7.08 (m, 3H), 4.67 (t, 1H), 4.03 (t, 2H), 3.71-3.68 (m, 1H), 3.66-3.63 (m, 1H), 3.25 (t, 1H), 3.23 (s, 3H), 2.01-1.91 (m, 2H). [M−H]⁻: 453.0.

### Example 25: ((R)-3-(4-chloro-3-fluorophenoxy)-1-((R)-3-methoxy-2-(pyrazine-2-carboxamido)propanamido)propyl)boronic acid

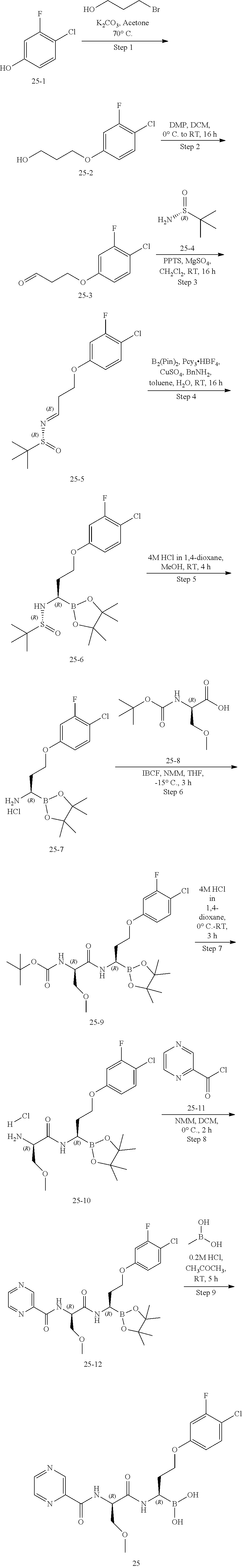

Synthesis of 3-(4-chloro-3-fluorophenoxy)propan-1-ol [Step 1]: To a stirred solution of 4-chloro-3-fluorophenol (25-1, 4 g, 27.3 mmol) in acetone (40 mL) was added 3-bromopropan-1-ol (4.17 g, 30 mmol) and K₂CO₃ (4.53 g, 32.8 mmol) at ambient temperature. The reaction mixture was allowed to warm to 70° C. and stirred for 27 h. The reaction mixture was filtered through celite bed and washed with acetone. The combined solvent was concentrated under reduced pressure and the residue was purified by flash column chromatography to afford 3-(4-chloro-3-fluorophenoxy)propan-1-ol (25-2, 4 g). ¹H NMR (400 MHz, DMSO-d₆) δ_{H}:7.44 (t, 1H), 7.06-7.02 (m, 1H), 6.83-6.80 (m, 1H), 4.56-4.54 (m, 1H), 4.06-4.02 (m, 2H), 3.55-3.51 (m, 2H), 1.87-1.80 (m, 2H).

Synthesis of 3-(4-chloro-3-fluorophenoxy)propanal [Step 2]: To a stirred solution of 3-(4-chloro-3-fluorophenoxy)propan-1-ol (25-2, 4 g, 19.50 mmol) in dichloromethane (80 mL) was added Dess-Martin periodinane (12.44 g, 29.30 mmol) at 0° C. The reaction mixture was allowed to warm to ambient temperature and stirred for 16 h. The reaction mixture was filtered through celite bed and thoroughly washed with dichloromethane. Combined filtrate was washed with aqueous NaHCO₃. The organic extract was collected and washed with water, brine, dried over anhydrous Na₂SO₄, filtered and concentrated under reduced pressure and the residue was purified by flash column chromatography to afford 3-(4-chloro-3-fluorophenoxy)propanal (25-3, 2 g). ¹H NMR (400 MHz CDCl₃) δ_{H}: 9.85 (s, 1H), 7.26-7.23 (m, 1H), 6.72-6.68 (m, 1H), 6.65-6.62 (m, 1H), 4.26 (t, 2H), 2.91 (t, 2H).

Synthesis of (R,E)-N-(3-(4-chloro-3-fluorophenoxy)propylidene)-2-methylpropane-2-sulfinamide [Step 3]: To a stirred solution of (R)-2-methylpropane-2-sulfinamide (25-4, 700 mg, 5.78 mmol) in dichloromethane (15 mL) were added pyridinium p-toluenesulfonate (290 mg, 1.16 mmol), anhydrous magnesium sulphate (3.48 g, 28.9 mmol) and 3-(4-chloro-3-fluorophenoxy)propanal (25-3, 1.29 g, 6.35 mmol) at 0° C. The reaction mixture was allowed to stir at ambient temperature for 16 h. The reaction mixture was filtered through a pad of celite and washed with dichloromethane several times. The combined filtrate was evaporated and the residue was purified by flash column chromatography to afford (R,E)-N-(3-(4-chloro-3-fluorophenoxy)propylidene)-2-methylpropane-2-sulfinamide (25-5, 1.2 g). ¹H NMR (400 MHz CDCl₃) δ_{H}: 8.14 (t, 1H), 7.25-7.23 (m, 1H), 6.69-6.56 (m, 2H), 4.29-4.21 (m, 2H), 3.00-2.97 (m, 2H), 1.18 (s, 9H).

Synthesis of (R)—N—((R)-3-(4-chloro-3-fluorophenoxy)-1-(4,4,5,5-tetramethyl-1,3,2-dioxaborolan-2-yl)propyl)-2-methylpropane-2-sulfinamide [Step 4]: To a suspension of tricyclohexylphosphine tetrafluoroborate (43 mg, 0.12 mmol) in toluene (3 mL), were added CuSO₄·5H₂O (29 mg, 0.12 mmol) in 0.9 mL of water) and benzylamine (0.04 mL, 0.39 mmol) and stirred vigorously for 40 min. It was diluted with toluene (8 mL). To that catalyst mixture were added (R,E)-N-(3-(4-chloro-3-fluorophenoxy)propylidene)-2-methylpropane-2-sulfinamide (25-5, 1.2 g, 3.92 mmol) and bispinacolatediborane (1.99 g, 7.85 mmol) and stirred at ambient temperature for 16 h. After diluting with ethyl acetate, the precipitate was filtered through a short pad of deactivated silica gel (SiO₂/H₂O 100/H₂O m/in) and washed with ethyl acetate. The combined filtrate was concentrated under reduced pressure and the residue was purified by flash column chromatography using deactivated silica gel (SiO₂/H₂O 100/H₂O m/m) to afford (R)—N—((R)-3-(4-chloro-3-fluorophenoxy)-1-(4,4,5,5-tetramethyl-1,3,2-dioxaborolan-2-yl)propyl)-2-methylpropane-2-sulfinamide (25-6, 1.5 g). [M−H]⁻: 432.3.

Synthesis of (R)-3-(4-chloro-3-fluorophenoxy)-1-(4,4,5,5-tetramethyl-1,3,2-dioxaborolan-2-yl)propan-1-amine hydrochloride [Step 5]: To a stirred solution of (R)—N—((R)-3-(4-chloro-3-fluorophenoxy)-1-(4,4,5,5-tetramethyl-1,3,2-dioxaborolan-2-yl)propyl)-2-methylpropane-2-sulfinamide (25-6, 1.5 g, 3.46 mmol) in 1,4-dioxane (15 mL) and methanol (1.4 mL, 34.6 mmol) was added 4 M HCl in 1,4-dioxane (0.88 mL, 3.46 mmol) dropwise at 0° C. The reaction mixture was allowed to warm to ambient temperature and stirred for 3 h. Volatiles were evaporated under reduced pressure and lyophilized to afford (R)-3-(4-chloro-3-fluorophenoxy)-1-(4,4,5,5-tetramethyl-1,3,2-dioxaborolan-2-yl)propan-1-amine hydrochloride (25-7, 550 mg). ¹H NMR (400 MHz, CD₃OD) δ_{H}:7.38-7.32 (m, 1H), 6.91-6.76 (m, 2H), 4.16-4.07 (m, 2H), 3.03 (t, 1H), 2.26-2.14 (m, 2H), 1.24 (s, 12H).

Synthesis of tert-butyl ((R)-1-(((R)-3-(4-chloro-3-fluorophenoxy)-1-(4,4,5,5-tetramethyl-1,3,2-dioxaborolan-2-yl)propyl)amino)-3-methoxy-1-oxopropan-2-yl)carbamate [Step 6]: To a stirred solution of N-(tert-butoxycarbonyl)-O-methyl-D-serine (25-8, 359 mg, 1.64 mmol) in THF (5 mL) was added IBCF (0.20 mL, 1.50 mmol) followed by NMM (0.22 mL, 1.64 mmol) at −15° C. and stirred for 30 min. A solution of (R)-3-(4-chloro-3-fluorophenoxy)-1-(4,4,5,5-tetramethyl-1,3,2-dioxaborolan-2-yl)propan-1-amine hydrochloride (25-7, 500 mg, 1.37 mmol) in THF (5 mL) was added dropwise followed by NMM (0.22 mL, 1.64 mmol) to the reaction mixture at −15° C. and then allowed to warm to 0° C. and stirred for 2 h. The reaction mixture was diluted with ethyl acetate and washed with 0.1 M HCl (twice), 5% aqueous K₂CO₃ solution (twice), water (twice) and brine, dried over anhydrous Na₂SO₄ and concentrated under reduced pressure at 30° C. to give tert-butyl ((R)-1-(((R)-3-(4-chloro-3-fluorophenoxy)-1-(4,4,5,5-tetramethyl-1,3,2-dioxaborolan-2-yl)propyl)amino)-3-methoxy-1-oxopropan-2-yl)carbamate (25-9, 550 mg). This product was forwarded to the next step without further purification. [M−H]⁻: 529.3.

Synthesis of (R)-2-amino-N—((R)-3-(4-chloro-3-fluorophenoxy)-1-(4,4,5,5-tetramethyl-1,3,2-dioxaborolan-2-yl)propyl)-3-methoxypropanamide hydrochloride [Step 7]: To a stirred solution of tert-butyl ((R)-1-(((R)-3-(4-chloro-3-fluorophenoxy)-1-(4,4,5,5-tetramethyl-1,3,2-dioxaborolan-2-yl)propyl)amino)-3-methoxy-1-oxopropan-2-yl)carbamate (25-9, 550 mg, 1.04 mmol) in 1,4-dioxane (5 mL) was added 4 M HCl in 1,4-dioxane (2.55 mL, 11.34 mmol) dropwise at 0° C. The reaction mixture was allowed to warm to ambient temperature and stirred for 3 h. Volatiles were evaporated under reduced pressure and lyophilized to afford (R)-2-amino-N—((R)-3-(4-chloro-3-fluorophenoxy)-1-(4,4,5,5-tetramethyl-1,3,2-dioxaborolan-2-yl)propyl)-3-methoxy propanamide hydrochloride (25-10, 370 mg). [M−H]⁻: 429.4.

Synthesis of N—((R)-1-(((R)-3-(4-chloro-3-fluorophenoxy)-1-(4,4,5,5-tetramethyl-1,3,2-dioxaborolan-2-yl)propyl)amino)-3-methoxy-1-oxopropan-2-yl)pyrazine-2-carboxamide [Step 8]: To a stirred solution of (R)-2-amino-N—((R)-3-(4-chloro-3-fluorophenoxy)-1-(4,4,5,5-tetramethyl-1,3,2-dioxaborolan-2-yl)propyl)-3-methoxypropanamide hydrochloride (25-10, 370 mg, 0.79 mmol) in dichloromethane (5 mL) was added NMM (0.43 mL, 3.96 mmol) at 0° C. followed by the addition of pyrazine-2-carbonyl chloride (25-11, 113 mg, 0.79 mmol) and stirred for 15 min at 0° C. The reaction mixture was allowed to warm to ambient temperature and stirred for 2 h. The reaction mixture was diluted with dichloromethane and washed with ice-cold water, brine, dried over anhydrous Na₂SO₄, filtered and concentrated under reduced pressure and the product purified by prep HPLC purification and lyophilized to afford N—((R)-1-(((R)-3-(4-chloro-3-fluorophenoxy)-1-(4,4,5,5-tetramethyl-1,3,2-dioxaborolan-2-yl)propyl)amino)-3-methoxy-1-oxopropan-2-yl)pyrazine-2-carboxamide (25-12, 60 mg). [M−H]⁻: 535.4.

Synthesis of ((R)-3-(4-chloro-3-fluorophenoxy)-1-((R)-3-methoxy-2-(pyrazine-2-carboxamido)propanamido)propyl)boronic acid [Step 9]: To a stirred solution of N—((R)-1-(((R)-3-(4-chloro-3-fluorophenoxy)-1-(4,4,5,5-tetramethyl-1,3,2-dioxaborolan-2-yl)propyl)amino)-3-methoxy-1-oxopropan-2-yl)pyrazine-2-carboxamide (25-12, 60 mg, 0.11 mmol) and methylboronic acid (13, 67 mg, 1.12 mmol) in acetone (1 mL) was added 0.2 M HCl (0.55 mL) and stirred at ambient temperature for 5 h. All volatiles were evaporated under reduced pressure and purified by prep HPLC and lyophilized to afford ((R)-3-(4-chloro-3-fluorophenoxy)-1-((R)-3-methoxy-2-(pyrazine-2-carboxamido)propanamido)propyl)boronic acid (Compound 25, 22 mg). [M−H]⁻: 453. ¹H NMR (400 MHz, DMSO-d₆+2 drop D₂O at 80° C.) δ_{H}: 9.16 (s, 1H), 8.86 (d, 1H), 8.71 (s, 1H), 7.38 (t, 1H), 6.90-6.87 (m, 1H), 6.76-6.73 (m, 1H), 4.67 (t, 1H), 3.98 (t, 2H), 3.72-3.68 (m, 1H), 3.65-3.61 (m, 1H), 3.30-3.25 (m, 1H), 3.23 (s, 3H), 2.03-1.89 (in, 2H).

### Example 26: ((S)-3-(4-chloro-3-fluorophenoxy)-1-((R)-3-methoxy-2-(pyrazine-2-carboxamido)propanamido)propyl)boronic acid

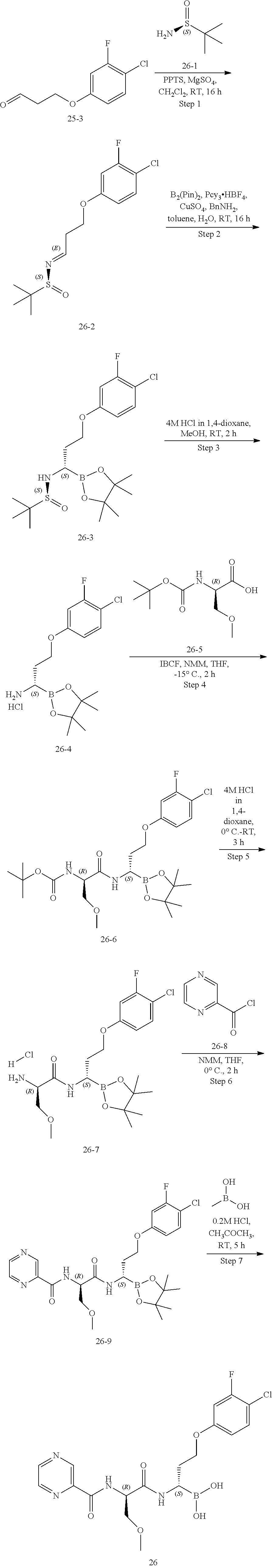

Synthesis of (S,E)-N-(3-(4-chloro-3-fluorophenoxy)propylidene)-2-methylpropane-2-sulfinamide [Step 1]: To a stirred solution of (S)-2-methylpropane-2-sulfinamide (26-2, 435 mg, 3.59 mmol) in dichloromethane (15 mL) were added pyridinium p-toluenesulfonate (180 mg, 0.72 mmol), anhydrous magnesium sulphate (2.16 g, 17.9 mmol) and 3-(4-chloro-3-fluorophenoxy)propanal (25-3, 800 mg, 3.95 mmol) at 0° C. The reaction mixture was allowed to stir at ambient temperature for 16 h. The reaction mixture was filtered through a pad of celite and washed with dichloromethane several times. The combined filtrate was evaporated and the residue was purified by flash column chromatography to afford (S,E)-N-(3-(4-chloro-3-fluorophenoxy)propylidene)-2-methylpropane-2-sulfinamide (26-2, 800 mg). ¹H NMR (400 MHz, CDCl₃) δ_{H}: 8.14 (t, 1H), 7.25-7.23 (m, 1H), 6.69-6.59 (m, 2H), 4.29-4.19 (m, 2H), 3.00-2.97 (m, 2H), 1.18 (s, 9H).

Synthesis of (S)—N—((S)-3-(4-chloro-3-fluorophenoxy)-1-(4,4,5,5-tetramethyl-1,3,2-dioxaborolan-2-yI)propyl)-2-methylpropane-2-sulfinamide [Step 2]: To a suspension of tricyclohexylphosphine tetrafluoroborate (25 mg, 0.07 mmol) in toluene (2 mL), were added CuSO₄·5H₂O (17 mg, 0.07 mmol, in 0.7 mL of water) and benzylamine (0.02 mL, 0.23 mmol) and stirred vigorously for 40 min. It was diluted with toluene (5 mL). (S,E)-N-(3-(4-chloro-3-fluorophenoxy)propylidene)-2-methylpropane-2-sulfinamide (26-2, 700 mg, 2.29 mmol) and bispinacolatediborane (1.16 g, 4.58 mmol) were added to the catalyst mixture and stirred at ambient temperature for 16 h. After diluting with ethyl acetate, the precipitate was filtered through a short pad of deactivated silica gel and washed with ethyl acetate. The combined filtrate was concentrated under reduced pressure and the residue was purified by flash column chromatography using deactivated silica gel to afford (S)—N—((S)-3-(4-chloro-3-fluorophenoxy)-1-(4,4,5,5-tetramethyl-1,3,2-dioxaborolan-2-yl)propyl)-2-methylpropane-2-sulfinamide (26-3, 1 g). [M−H]⁻: 432.3.

Synthesis of (S)-3-(4-chloro-3-fluorophenoxy)-1-(4,4,5,5-tetramethyl-1,3,2-dioxaborolan-2-yl)propan-1-amine hydrochloride [Step 3]: To a stirred solution of (S)—N—((S)-3-(4-chloro-3-fluorophenoxy)-1-(4,4,5,5-tetramethyl-1,3,2-dioxaborolan-2-yl)propyl)-2-methylpropane-2-sulfinamide (26-3, 1 g, 2.31 mmol) in 1,4-dioxane (10 mL) and methanol (0.92 mL, 23.1 mmol) was added 4 M HCl in 1,4-dioxane (0.58 mL, 2.31 mmol) dropwise at 0° C. The reaction mixture was allowed to warm to ambient temperature and stirred for 2 h. Volatiles were evaporated under reduced pressure and lyophilized to afford (S)-3-(4-chloro-3-fluorophenoxy)-1-(4,4,5,5-tetramethyl-1,3,2-dioxaborolan-2-yl)propan-1-amine hydrochloride (26-4, 450 mg). ¹H NMR (400 MHz, CD₃OD) δ_{H}: 7.38-7.33 (m, 1H), 6.91-6.76 (m, 2H), 4.16-4.09 (m, 2H), 3.09 (t, 1H), 2.20-2.16 (m, 2H), 1.24 (s, 12H).

Synthesis of tert-butyl ((R)-1-(((S)-3-(4-chloro-3-fluorophenoxy)-1-(4,4,5,5-tetramethyl-1,3,2-dioxaborolan-2-yl)propyl)amino)-3-methoxy-1-oxopropan-2-yl)carbamate [Step 4]: To a stirred solution of N-(tert-butoxycarbonyl)-O-methyl-D-serine (26-5, 323 mg, 1.48 mmol) in THF (4 mL) was added IBCF (0.18 mL, 1.35 mmol) followed by NMM (0.20 mL, 1.48 mmol) at −15° C. and stirred for 30 min. A solution of (S)-3-(4-chloro-3-fluorophenoxy)-1-(4,4,5,5-tetramethyl-1,3,2-dioxaborolan-2-yl)propan-1-amine hydrochloride (26-4, 450 mg, 1.23 mmol) in THF (4 mL) was added dropwise followed by NMM (0.20 mL, 1.48 mmol) to the reaction mixture at −15° C. and then allowed to warm to 0° C. and stirred for 2 h. The reaction mixture was diluted with ethyl acetate and washed with 0.1 M HCl (twice), 5% aqueous K₂CO₃ solution (twice), water (twice) and brine, dried over anhydrous Na₂SO₄ and concentrated under reduced pressure at 30° C. to give tert-butyl ((R)-1-(((S)-3-(4-chloro-3-fluorophenoxy)-1-(4,4,5,5-tetramethyl-1,3,2-dioxaborolan-2-yl)propyl)amino)-3-methoxy-1-oxopropan-2-yl)carbamate (26-6, 500 mg). This product was forwarded to the next step without further purification. [M−H]: 529.4.

Synthesis of (R)-2-amino-N—((S)-3-(4-chloro-3-fluorophenoxy)-1-(4,4,5,5-tetramethyl-1,3,2-dioxaborolan-2-yl)propyl)-3-methoxypropanamide hydrochloride [Step 5]: To a stirred solution of tert-butyl ((R)-1-(((S)-3-(4-chloro-3-fluorophenoxy)-1-(4,4,5,5-tetramethyl-1,3,2-dioxaborolan-2-yl)propyl)amino)-3-methoxy-1-oxopropan-2-yl)carbamate (26-6, 500 mg, 0.94 mmol) in 1,4-dioxane (5 mL), was added 4 M HCl in 1,4-dioxane (2.6 mL, 10.4 mmol) dropwise at 0° C. The reaction mixture was allowed to warm to ambient temperature and stirred for 3 h. Volatiles were evaporated under reduced pressure and lyophilized to afford (R)-2-amino-N—((S)-3-(4-chloro-3-fluorophenoxy)-1-(4,4,5,5-tetramethyl-1,3,2-dioxaborolan-2-yl)propyl)-3-methoxy propanamide hydrochloride (26-7, 330 mg). [M−H]⁻: 429.5.

Synthesis of N—((R)-1-(((S)-3-(4-chloro-3-fluorophenoxy)-1-(4,4,5,5-tetramethyl-1,3,2-dioxaborolan-2-yl)propyl)amino)-3-methoxy-1-oxopropan-2-yl)pyrazine-2-carboxamide [Step 6]: To a stirred solution of (R)-2-amino-N—((S)-3-(4-chloro-3-fluorophenoxy)-1-(4,4,5,5-tetramethyl-1,3,2-dioxaborolan-2-yl)propyl)-3-methoxypropanamide hydrochloride (26-7, 330 mg, 0.70 mmol) in dichloromethane (5 mL) was added NMM (0.39 mL, 3.53 mmol) at 0° C. followed by the addition of pyrazine-2-carbonyl chloride (26-8, 101 mg, 0.70 mmol) and stirred for 15 min at 0° C. The reaction mixture was allowed to warm to ambient temperature and stirred for 2 h. The reaction mixture was diluted with dichloromethane and washed with ice-cold water, brine, dried over anhydrous Na₂SO₄, filtered and concentrated under reduced pressure and the product purified by prep HPLC purification and lyophilized to afford N—((R)-1-(((S)-3-(4-chloro-3-fluorophenoxy)-1-(4,4,5,5-tetramethyl-1,3,2-dioxaborolan-2-yl)propyl)amino)-3-methoxy-1-oxopropan-2-yl)pyrazine-2-carboxamide (26-9, 53 mg). [M−H]⁻: 535.5.

Synthesis of ((S)-3-(4-chloro-3-fluorophenoxy)-1-((R)-3-methoxy-2-(pyrazine-2-carboxamido)propanamido)propyl)boronic acid [Step 7]: To a stirred solution of N—((R)-1-(((S)-3-(4-chloro-3-fluorophenoxy)-1-(4,4,5,5-tetramethyl-1,3,2-dioxaborolan-2-yl)propyl)amino)-3-methoxy-1-oxopropan-2-yl)pyrazine-2-carboxamide (26-9, 60 mg, 0.11 mmol) and methylboronic acid (11, 67 mg, 1.12 mmol) in acetone (1 mL) was added 0.2 M HCl (0.55 mL) and stirred at ambient temperature for 5 h. All volatiles were evaporated under reduced pressure and purified by prep HPLC and lyophilized to afford ((S)-3-(4-chloro-3-fluorophenoxy)-1-((R)-3-methoxy-2-(pyrazine-2-carboxamido)propanamido)propyl)boronic acid (Compound 26, 13 mg). [M−H]⁻: 452.9. ¹H NMR (400 MHz, DMSO-d₆+2 drop D₂O at 80° C.) δ_{H}: 9.17 (s, 1H), 8.86 (d, 1H), 8.71 (s, 1H), 7.36 (t, 1H), 6.91-6.87 (m, 1H), 6.76-6.73 (m, 1H), 4.67 (t, 1H), 3.98 (t, 2H), 3.74-3.69 (m, 1H), 3.67-3.63 (m, 1H), 3.27 (s, 3H), 3.26-3.18 (m, 1H), 2.01-1.90 (m, 2H).

### Example 27: ((R)-3-(4-chloro-2-methylphenoxy)-1-((R)-3-methoxy-2-(pyrazine-2-carboxamido)propanamido)propyl)boronic acid

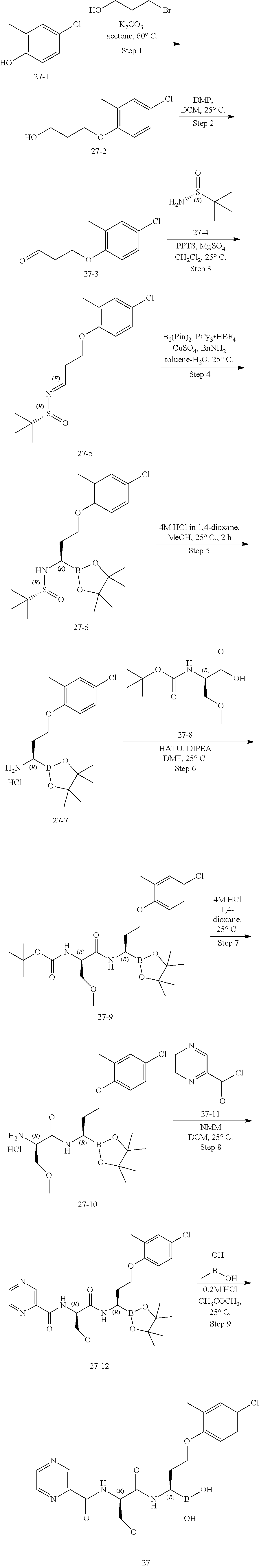

Synthesis of 3-(4-chloro-2-methylphenoxy)propan-1-ol [Step 1]: To a stirred solution of 4-chloro-2-methylphenol (27-1, 5.0 g, 35.1 mmol) in acetone (40 mL) were added K₂CO₃ (5.8 g, 42.1 mmol) and 3-bromopropan-1-ol (3.4 mL, 38.6 mmol) and the reaction mixture was heated at 60° C. After 16 h, the reaction mixture was concentrated under reduced pressure, diluted with water and extracted with ethyl acetate (twice). Combined organic extract was dried over anhydrous Na₂SO₄, filtered and concentrated under reduced pressure. The crude compound was purified by combiflash chromatography to afford 3-(4-chloro-2-methylphenoxy)propan-1-ol (27-2, 5.0 g). [M+H]⁺: 201.2.

Synthesis of 3-(4-chloro-2-methylphenoxy)propanal [Step 2]: To a stirred solution of 3-(4-chloro-2-methylphenoxy)propan-1-ol (27-2, 5.0 g, 24.9 mmol) in dichloromethane (100 mL) was added Dess-Martin periodinane (15.6 g, 37.4 mmol) at 0° C., and the reaction mixture was allowed to warm at ambient temperature. After 2 h, the reaction mixture was filtered through a pad of celite, and the celite pad was washed with dichloromethane several times. Combined filtrate was washed successively with aqueous NaHCO₃, water and brine. The organic extract was dried over anhydrous Na₂SO₄, filtered and concentrated under reduced pressure. The crude product was purified by flash column chromatography to afford 3-(4-chloro-2-methylphenoxy)propanal (27-3, 4.0 g). [M+H]⁺: 199.0.

Synthesis of (R,E)-N-(3-(4-chloro-2-methylphenoxy)propylidene)-2-methylpropane-2-sulfinamide [Step 3]: To a stirred solution of (R)-2-methylpropane-2-sulfinamide (27-4, 1.1 g, 9.1 mmol) in dichloromethane (50 mL), PPTS (114 mg, 0.5 mmol), 3-(4-chloro-2-methylphenoxy)propanal (27-3, 1.8 g, 9.9 mmol) and MgSO₄ (5.5 g, 45.4 mmol) were added under nitrogen atmosphere at ice cold condition. The reaction mixture was stirred at ambient temperature for 16 h. The reaction mixture was filtered through a pad of celite and it was washed with dichloromethane. Filtrate was concentrated under reduced pressure, and the crude product was purified through combiflash chromatography to afford (R,E)-N-(3-(4-chloro-2-methylphenoxy)propylidene)-2-methylpropane-2-sulfinamide (27-5, 2.0 g). [M+H]⁺: 302.0.

Synthesis of (R)—N—((R)-3-(4-chloro-2-methylphenoxy)-1-(4,4,5,5-tetramethyl-1,3,2-dioxaborolan-2-yl)propyl)-2-methylpropane-2-sulfinamide [Step 4]: To a solution of PCy₃·HBF₄ (73 mg, 0.2 mmol) in toluene (12 mL) was added 30 mM aqueous solution of CuSO₄·5H₂O (50 mg, 0.2 mmol) followed by benzylamine (0.1 mL, 0.7 mmol) and the reaction mixture was stirred vigorously for 20 min. The reaction mixture was placed in an ice bath, and added a solution of (R,E)-N-(3-(4-chloro-2-methylphenoxy)propylidene)-2-methylpropane-2-sulfinamide (27-5, 2.0 g, 6.6 mmol) in toluene (12 mL) followed by B₂(Pin)₂ (3.4 g, 13.3 mmol). The reaction mixture was allowed to warm to ambient temperature, and stirred for 16 h. Reaction mixture was filtered through a small bed of deactivated silica gel, washing the bed with ethyl acetate. Combine filtrate was concentrated at 25° C. under reduced pressure to give product which was purified by column chromatography using deactivated silica gel to afford (R)—N—((R)-3-(4-chloro-2-methylphenoxy)-1-(4,4,5,5-tetramethyl-1,3,2-dioxaborolan-2-yl)propyl)-2-methyl propane-2-sulfinamide (27-6, 2.0 g). [M−H]⁻: 428.4.

Synthesis of (R)-3-(4-chloro-2-methylphenoxy)-1-(4,4,5,5-tetramethyl-1,3,2-dioxaborolan-2-yl)propan-1-amine hydrochloride [Step 5]: To a stirred solution of (R)—N—((R)-3-(4-chloro-2-methylphenoxy)-1-(4,4,5,5-tetramethyl-1,3,2-dioxaborolan-2-yl)propyl)-2-methylpropane-2-sulfinamide (27-6, 1.7 g, 3.9 mmol) in 1,4-dioxane (10 mL) was added methanol (1.6 mL, 39.6 mmol) followed by HCl (0.9 mL, 4 M in 1,4-dioxane, 3.6 mmol) at 0° C. and stirred for 2 h at ambient temperature. The reaction mixture was concentrated under reduced pressure to afford (R)-3-(4-chloro-2-methylphenoxy)-1-(4,4,5,5-tetramethyl-1,3,2-dioxaborolan-2-yl)propan-1-amine hydrochloride (27-7, 1.0 g). [M−H]⁻: 242.3.

Synthesis of tert-butyl ((R)-1-(((R)-3-(4-chloro-2-methylphenoxy)-1-(4,4,5,5-tetramethyl-1,3,2-dioxaborolan-2-yl)propyl)amino)-3-methoxy-1-oxopropan-2-yl)carbamate [Step 6]: To a stirred solution of N-(tert-butoxycarbonyl)-O-methyl-D-serine (27-8, 300 mg, 1.4 mmol) in DMF (5 mL) were added HATU (780 mg, 2.1 mmol) and (R)-3-(4-chloro-2-methylphenoxy)-1-(4,4,5,5-tetramethyl-1,3,2-dioxaborolan-2-yl)propan-1-amine hydrochloride (27-7, 595 mg, 1.6 mmol) followed by N,N-diisopropylethylamine (0.6 mL, 3.4 mmol) at 0° C., and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford tert-butyl ((R)-1-(((R)-3-(4-chloro-2-methylphenoxy)-1-(4,4,5,5-tetramethyl-1,3,2-dioxaborolan-2-yl)propyl)amino)-3-methoxy-1-oxopropan-2-yl)carbamate (27-9, 500 mg) which was used in the next step without further purification. [M+H]⁺: 527.2.

Synthesis of (R)-2-amino-N—((R)-3-(4-chloro-2-methylphenoxy)-1-(4,4,5,5-tetramethyl-1,3,2-dioxaborolan-2-yl)propyl)-3-methoxypropanamide hydrochloride [Step 7]: To a stirred solution of tert-butyl ((R)-1-(((R)-3-(4-chloro-2-methylphenoxy)-1-(4,4,5,5-tetramethyl-1,3,2-dioxaborolan-2-yl)propyl)amino)-3-methoxy-1-oxopropan-2-yl)carbamate (27-9, 1.5 g, 2.8 mmol) in 1,4-dioxane (10 mL), was added HCl (7.1 mL, 4M in 1,4-dioxane, 28.4 mmol) dropwise at 0° C. and the reaction mixture was stirred at ambient temperature. After 16 h, the reaction mixture was concentrated under reduced pressure to afford (R)-2-amino-N—((R)-3-(4-chloro-2-methylphenoxy)-1-(4,4,5,5-tetramethyl-1,3,2-dioxaborolan-2-yl)propyl)-3-methoxypropanamide hydrochloride (27-10, 1.0 g), which was used in the next step without further purification. [M-83]⁻: 343.5, mass of the corresponding boronic acid.

Synthesis of N—((R)-1-(((R)-3-(4-chloro-2-methylphenoxy)-1-(4,4,5,5-tetramethyl-1,3,2-dioxaborolan-2-yl)propyl)amino)-3-methoxy-1-oxopropan-2-yl)pyrazine-2-carboxamide [Step 8]: To a stirred solution of (R)-2-amino-N—((R)-3-(4-chloro-2-methylphenoxy)-1-(4,4,5,5-tetramethyl-1,3,2-dioxaborolan-2-yl)propyl)-3-methoxypropanamide hydrochloride (27-10, 1.0 g, 2.2 mmol) in dichloromethane (15 mL) was added NMM (1.2 mL, 10.8 mmol) at 0° C. and stirred for 15 min. To this resulting solution was added pyrazine-2-carbonyl chloride (27-11, 308 mg, 2.16 mmol) in one portion, and the reaction mixture was stirred at ambient temperature for 2 h. Reaction mixture was diluted with dichloromethane and washed with aqueous solution of NaHCO₃ and finally with brine. Organic extract was dried over anhydrous Na₂SO₄, filtered and concentrated under reduced pressure to afford N—((R)-1-(((R)-3-(4-chloro-2-methylphenoxy)-1-(4,4,5,5-tetramethyl-1,3,2-dioxaborolan-2-yl)propyl)amino)-3-methoxy-1-oxopropan-2-yl)pyrazine-2-carboxamide (27-12, 600 mg). [M−H]⁻: 531.4, and [M-83]⁻: 449.3 for the corresponding boronic acid.

Synthesis of ((R)-3-(4-chloro-2-methylphenoxy)-1-((R)-3-methoxy-2-(pyrazine-2-carboxamido)propanamido)propyl)boronic acid [Step 9]: To a stirred solution of N-[(1R)-2-[[(1R)-3-(4-chloro-2-methyl-phenoxy)-1-(4,4,5,5-tetramethyl-1,3,2-dioxaborolan-2-yl)propyl]amino]-1-(methoxymethyl)-2-oxo-ethyl]pyrazine-2-carboxamide (27-12, 300 mg, 0.6 mmol) in acetone (12 mL) was added methylboronic acid (506 mg, 8.5 mmol) aqueous 0.2 M HCl (12 mL, 2.4 mmol) at 0° C., and the reaction mixture was allowed to warm to ambient temperature for 12 h. The reaction mixture was concentrated under reduced pressure, lyophilized, purified by PREP-HPLC and finally lyophilized again to afford ((R)-3-(4-chloro-2-methylphenoxy)-1-((R)-3-methoxy-2-(pyrazine-2-carboxamido)propanamido)propyl)boronic acid (Compound 27, 100 mg). [M−H]⁻: 449.4. ¹H NMR (400 MHz, CD₃OD): δ_{H} 9.23 (s, 1H), 8.81-8.80 (m, 1H), 8.70-8.69 (m, 1H), 7.06-7.04 (m, 2H), 6.83-6.81 (m, 1H), 5.00 (t, 1H), 4.02-4.01 (m, 2H), 3.91-3.88 (m, 1H), 3.80-3.77 (m, 1H), 3.39 (s, 3H), 2.95 (t, 1H), 2.14 (s, 3H), 2.01-2.00 (m, 1H), 1.93-1.92 (m, 1H).

### Example 28: ((S)-3-(4-chloro-2-methylphenoxy)-1-((R)-3-methoxy-2-(pyrazine-2-carboxamido)propanamido)propyl)boronic acid

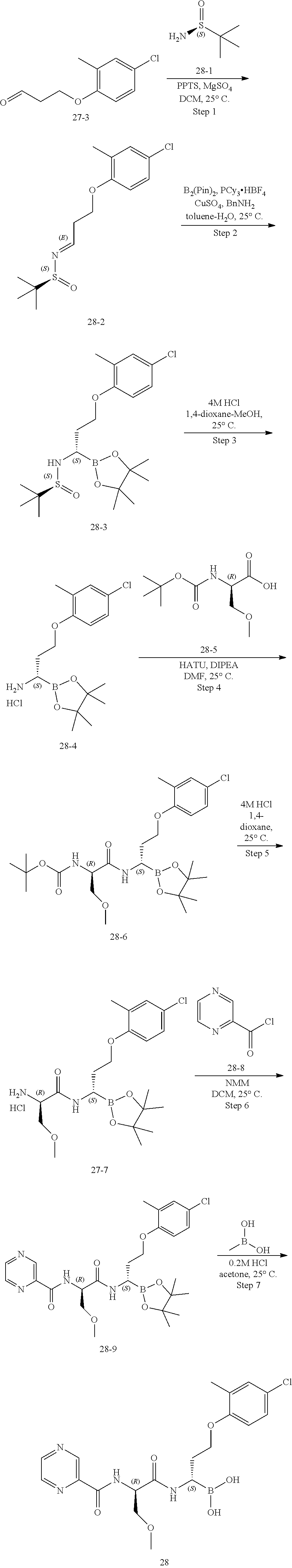

Synthesis of (S,E)-N-(3-(4-chloro-2-methylphenoxy)propylidene)-2-methylpropane-2-sulfinamide [Step 1]: To an ice-cold solution of (S)-2-methylpropane-2-sulfinamide (28-1, 1.1 g, 9.1 mmol) in DCM (50 mL) was added PPTS (114 mg, 0.4 mmol), 3-(4-chloro-2-methylphenoxy)propanal (27-3, 2.0 g, 10.0 mmol) and anhydrous MgSO₄ (5.5 g, 45.4 mmol) and the reaction mixture was stirred at ambient temperature. After 16 h, the reaction mixture was filtered through a pad of celite washing the pad with DCM. The filtrate was concentrated under reduced pressure, and purified by column chromatography to afford (S,E)-N-(3-(4-chloro-2-methylphenoxy)propylidene)-2-methylpropane-2-sulfinamide (28-2, 2.0 g). [M+H]⁺: 302.0.

Synthesis of (S)—N—((S)-3-(4-chloro-2-methylphenoxy)-1-(4,4,5,5-tetramethyl-1,3,2-dioxaborolan-2-yI)propyl)-2-methylpropane-2-sulfinamide [Step 2]: To an ice-cold solution of PCy₃·HBF₄ (66 mg, 0.2 mmol) in toluene (2 mL) was added CuSO₄·5H₂O (45 mg, 0.2 mmol), water (1.5 mL) and benzylamine (0.1 mL, 0.6 mmol). After 20 min, a solution of (S,E)-N-(3-(4-chloro-2-methylphenoxy)propylidene)-2-methylpropane-2-sulfinamide (28-2, 1.80 g, 6.0 mmol) in toluene (12 mL) was added slowly, followed by the addition of bis(pinacolato)diboron (3.0 g, 11.9 mmol). The reaction mixture was gradually warmed to ambient temperature, and stirred for 16 h. The reaction mixture was filtered through a pad of deactivated silica gel, and concentrated under reduced pressure. Purification of the compound on deactivated silica gel afforded (S)—N—((S)-3-(4-chloro-2-methylphenoxy)-1-(4,4,5,5-tetramethyl-1,3,2-dioxaborolan-2-yl)propyl)-2-methyl propane-2-sulfinamide (28-3, 1.80 g). [M−H]⁻: 428.1.

Synthesis of (S)-3-(4-chloro-2-methylphenoxy)-1-(4,4,5,5-tetramethyl-1,3,2-dioxaborolan-2-yl)propan-1-amine hydrochloride [Step 3]: To an ice-cold solution of (S)—N—((S)-3-(4-chloro-2-methylphenoxy)-1-(4,4,5,5-tetramethyl-1,3,2-dioxaborolan-2-yl)propyl)-2-methylpropane-2-sulfinamide (28-3, 1.8 g, 4.2 mmol) in 1,4-dioxane (10 mL) was added methanol (1.7 mL, 41.9 mmol) followed by HCl (1.1 mL, 4 M in 1,4-dioxane, 4.4 mmol). Reaction mixture was stirred at ambient temperature for 2 h. The reaction mixture was concentrated under reduced pressure and lyophilized to afford (S)-3-(4-chloro-2-methylphenoxy)-1-(4,4,5,5-tetramethyl-1,3,2-dioxaborolan-2-yl)propan-1-amine hydrochloride (28-4, 1.40 g), which was used in the next step without further purification.

Synthesis of tert-butyl ((R)-1-(((S)-3-(4-chloro-2-methylphenoxy)-1-(4,4,5,5-tetramethyl-1,3,2-dioxaborolan-2-yl)propyl)amino)-3-methoxy-1-oxopropan-2-yl)carbamate [Step 4]: To a solution of N-(tert-butoxycarbonyl)-O-methyl-D-serine (28-5, 400 mg, 1.8 mmol) and (S)-3-(4-chloro-2-methylphenoxy)-1-(4,4,5,5-tetramethyl-1,3,2-dioxaborolan-2-yl)propan-1-amine hydrochloride (28-4, 1.0 g, 2.7 mmol) was added HATU (1.0 mg, 2.7 mmol) followed by DIPEA (0.8 mL, 4.6 mmol) and the reaction mixture was stirred at ambient temperature. After 1 h, the reaction mixture was quenched with cold brine, and extracted with ethyl acetate (twice).

Combined organic extract was dried over anhydrous Na₂SO₄, filtered and concentrated under reduced pressure to afford tert-butyl ((R)-1-(((S)-3-(4-chloro-2-methylphenoxy)-1-(4,4,5,5-tetramethyl-1,3,2-dioxaborolan-2-yl)propyl)amino)-3-methoxy-1-oxopropan-2-yl)carbamate (28-6, 900 mg), which was used in the next step without further purification. [M−H]⁻: 525.3, and 443.5 for the corresponding boronic acid.

Synthesis of (R)-2-amino-N—((S)-3-(4-chloro-2-methylphenoxy)-1-(4,4,5,5-tetramethyl-1,3,2-dioxaborolan-2-yl)propyl)-3-methoxypropanamide hydrochloride [Step 5]: To an ice-cold solution of afford tert-butyl ((R)-1-(((S)-3-(4-chloro-2-methylphenoxy)-1-(4,4,5,5-tetramethyl-1,3,2-dioxaborolan-2-yl)propyl)amino)-3-methoxy-1-oxopropan-2-yl)carbamate (28-6, 900 mg, 1.7 mmol) in 1,4-dioxane (12 mL) was added HCl (12 mL, 4M in 1,4-dioxane, 48.0 mmol) and the reaction mixture was stirred at ambient temperature. After 16 h, LCMS indicated formation of the desired product. The reaction mixture was concentrated under reduced pressure to afford (R)-2-amino-N—((S)-3-(4-chloro-2-methylphenoxy)-1-(4,4,5,5-tetramethyl-1,3,2-dioxaborolan-2-yl) propyl)-3-methoxypropanamide hydrochloride (28-7, 700 mg) which was used in the next step without further purification. [M−H]⁻: 425.5, and 343.2 for the corresponding boronic acid.

Synthesis of N—((R)-1-(((S)-3-(4-chloro-2-methylphenoxy)-1-(4,4,5,5-tetramethyl-1,3,2-dioxaborolan-2-yl)propyl)amino)-3-methoxy-1-oxopropan-2-yl)pyrazine-2-carboxamide [Step 6]: To an ice-cold solution of (R)-2-amino-N—((S)-3-(4-chloro-2-methylphenoxy)-1-(4,4,5,5-tetramethyl-1,3,2-dioxaborolan-2-yl)propyl)-3-methoxypropanamide hydrochloride (28-7, 650 mg, 1.4 mmol) in DCM (20 mL) was added NMM (0.8 mL, 7.0 mmol) followed by pyrazine-2-carbonyl chloride (28-8, 200 mg, 1.4 mmol). After stirring for 2 h at ambient temperature, the reaction mixture was quenched with cold water and extracted with DCM (twice). Combined organic phase was washed with brine, dried over anhydrous Na₂SO₄, filtered and concentrated under reduced pressure to afford N—((R)-1-(((S)-3-(4-chloro-2-methylphenoxy)-1-(4,4,5,5-tetramethyl-1,3,2-dioxaborolan-2-yl)propyl)amino)-3-methoxy-1-oxopropan-2-yl)pyrazine-2-carboxamide (28-9, 600 mg) which was used in the next step without further purification. [M−H]⁻: 531.4, and 449.2 for the corresponding boronic acid.

Synthesis of ((S)-3-(4-chloro-2-methylphenoxy)-1-((R)-3-methoxy-2-(pyrazine-2-carboxamido)propanamido)propyl)boronic acid [Step 7]: To a stirred solution of N—((R)-1-(((S)-3-(4-chloro-2-methylphenoxy)-1-(4,4,5,5-tetramethyl-1,3,2-dioxaborolan-2-yl)propyl)amino)-3-methoxy-1-oxopropan-2-yl)pyrazine-2-carboxamide (28-9, 300 mg, 0.6 mmol) in acetone (16 mL) was added methylboronic acid (506 mg, 8.4 mmol) followed by aqueous 0.2 M aqueous HCl (16 mL, 3.2 mmol) at 0° C. The reaction mixture was stirred at ambient temperature for 16 h. The reaction mixture was concentrated under reduced pressure, lyophilized, purified by reverse phase preparative HPLC, and finally lyophilized again to afford ((S)-3-(4-chloro-2-methylphenoxy)-1-((R)-3-methoxy-2-(pyrazine-2-carboxamido)propanamido)propyl) boronic acid (Compound 28, 125 mg). [M−H]⁻: 449.4. ¹H NMR (400 MHz, CD₃OD): δ_{H} 9.23 (s, 1H), 8.81-8.80 (m, 1H), 8.70-8.69 (m, 1H), 7.06-7.04 (m, 2H), 6.83-6.81 (m, 1H), 5.00 (t, 1H), 4.02-4.01 (m, 2H), 3.91-3.88 (m, 1H), 3.80-3.77 (m, 1H), 3.39 (s, 3H), 2.95 (t, 1H), 2.14 (s, 3H), 2.01-2.00 (m, 1H), 1.93-1.92 (m, 1H).

### Example 29: ((S)-3-(4-chloro-2-fluorophenoxy)-1-((R)-3-methoxy-2-(pyrazine-2-carboxamido)propanamido)propyl)boronic acid

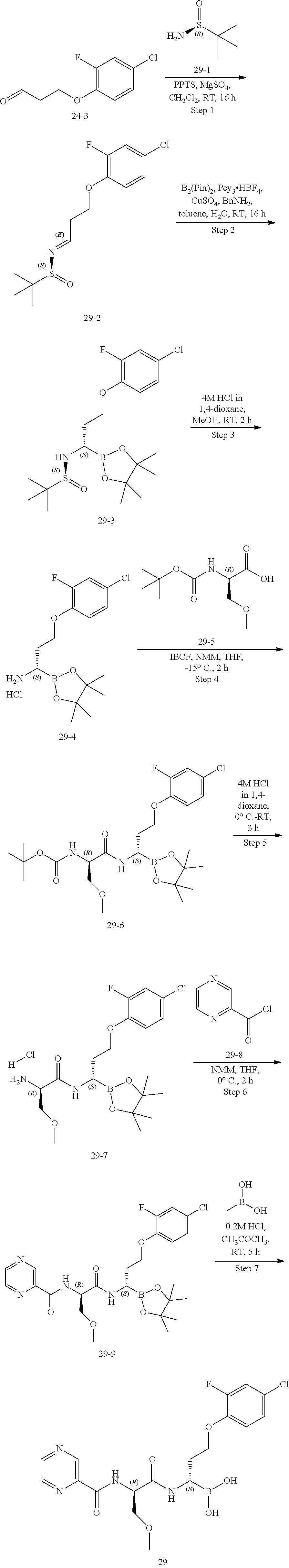

Synthesis of (S,E)-N-(3-(4-chloro-2-fluorophenoxy)propylidene)-2-methylpropane-2-sulfinamide [Step 1]: To a stirred solution of (S)-2-methylpropane-2-sulfinamide (29-1, 1.08 g, 8.91 mmol) in dichloromethane (40 mL) were added pyridinium p-toluenesulfonate (1.37 g, 5.45 mmol), anhydrous magnesium sulphate (5.36 g, 44.6 mmol) and 3-(4-chloro-2-fluorophenoxy)propanal (24-3, 1.99 g, 9.81 mmol) at 0° C. The reaction mixture was allowed to stir at ambient temperature for 16 h. The reaction mixture was filtered through a pad of celite and washed with dichloromethane several times. The combined filtrate was evaporated and the residue was purified by flash column chromatography to afford (S,E)-N-(3-(4-chloro-2-fluorophenoxy)propylidene)-2-methylpropane-2-sulfinamide (29-2, 1.2 g). ¹H NMR (400 MHz, DMSO-d₆) δ_{H}: 8.02 (t, 1H), 7.42-7.39 (m, 1H), 7.26-7.19 (m, 2H), 4.41-4.36 (m, 2H), 3.02-2.96 (m, 2H), 1.08 (s, 9H).

Synthesis of (S)—N—((S)-3-(4-chloro-2-fluorophenoxy)-1-(4,4,5,5-tetramethyl-1,3,2-dioxaborolan-2-yl)propyl)-2-methylpropane-2-sulfinamide [Step 2]: To a suspension of tricyclohexylphosphine tetrafluoroborate (43 mg, 0.12 mmol) in toluene (3 mL), were added CuSO₄·5H₂O (29 mg, 0.12 mmol, in 1 mL of water) and benzylamine (0.04 mL, 0.39 mmol) and stirred vigorously for 40 min. It was diluted with toluene (7 mL). To this catalyst mixture were added (S,E)-N-(3-(4-chloro-2-fluorophenoxy)propylidene)-2-methylpropane-2-sulfinamide (29-2, 1.2 g, 3.92 mmol) and bispinacolatediborane (1.99 g, 7.85 mmol) and stirred at ambient temperature for 16 h. After diluting with ethyl acetate, the precipitate was filtered through a short pad of deactivated silica gel and washed with ethyl acetate. The combined filtrate was concentrated under reduced pressure and the residue was purified by flash column chromatography using deactivated silica gel to afford (S)—N—((S)-3-(4-chloro-2-fluorophenoxy)-1-(4,4,5,5-tetramethyl-1,3,2-dioxaborolan-2-yl)propyl)-2-methylpropane-2-sulfinamide (29-3, 1.2 g). ¹H NMR (400 MHz, CD₃OD) δ_{H}: 7.17-7.09 (m, 3H), 4.21-4.15 (m, 2H), 3.20 (t, 1H), 2.17-2.08 (m, 2H), 1.29 (s, 9H), 1.24 (s, 12H).

Synthesis of (S)-3-(4-chloro-2-fluorophenoxy)-1-(4,4,5,5-tetramethyl-1,3,2-dioxaborolan-2-yl)propan-1-amine hydrochloride [Step 3]: To a stirred solution of (S)—N—((S)-3-(4-chloro-2-fluorophenoxy)-1-(4,4,5,5-tetramethyl-1,3,2-dioxaborolan-2-yl)propyl)-2-methylpropane-2-sulfinamide (29-3, 1.2 g, 2.77 mmol) in 1,4-Dioxane (12 mL) and methanol (1.1 mL, 27.7 mmol), was added 4 M HCl in 1,4-dioxane (0.70 mL, 2.77 mmol) dropwise at 0° C. The reaction mixture was allowed to warm to ambient temperature and stirred for 2 h. Volatiles were evaporated under reduced pressure and lyophilized to afford (S)-3-(4-chloro-2-fluorophenoxy)-1-(4,4,5,5-tetramethyl-1,3,2-dioxaborolan-2-yl)propan-1-amine hydrochloride (29-4, 550 mg). ¹H NMR (400 MHz, CD₃OD) δ_{H}: 7.21-7.05 (m, 3H), 4.23-4.12 (m, 2H), 3.09 (t, 1H), 2.24-2.18 (m, 2H), 1.25 (s, 12H).

Synthesis of tert-butyl ((R)-1-(((S)-3-(4-chloro-2-fluorophenoxy)-1-(4,4,5,5-tetramethyl-1,3,2-dioxaborolan-2-yl)propyl)amino)-3-methoxy-1-oxopropan-2-yl)carbamate [Step 4]: To a stirred solution of N-(tert-butoxycarbonyl)-O-methyl-D-serine (29-5, 359 mg, 1.64 mmol) in THF (5 mL) was added IBCF (0.20 mL, 1.50 mmol) followed by NMM (0.22 mL, 1.64 mmol) at −15° C. and stirred for 30 min. A solution of (S)-3-(4-chloro-2-fluorophenoxy)-1-(4,4,5,5-tetramethyl-1,3,2-dioxaborolan-2-yl)propan-1-amine hydrochloride (29-4, 550 mg, 1.37 mmol) in THF (5 mL) was added dropwise followed by NMM (0.22 mL, 1.64 mmol) to the reaction mixture at −15° C. and then allowed to warm to 0° C. and stirred for 2 h. The reaction mixture was diluted with ethyl acetate and washed with 0.1 M HCl (twice), 5% aqueous K₂CO₃ solution (twice), water (twice) and brine, dried over anhydrous Na₂SO₄ and concentrated under reduced pressure at 30° C. to give tert-butyl ((R)-1-(((S)-3-(4-chloro-2-fluorophenoxy)-1-(4,4,5,5-tetramethyl-1,3,2-dioxaborolan-2-yl)propyl)amino)-3-methoxy-1-oxopropan-2-yl)carbamate (29-6, 620 mg). This product was forwarded to the next step without further purification. [M−H]: 529.3.

Synthesis of (R)-2-amino-N—((S)-3-(4-chloro-2-fluorophenoxy)-1-(4,4,5,5-tetramethyl-1,3,2-dioxaborolan-2-yl)propyl)-3-methoxypropanamide hydrochloride [Step 5]: To a stirred solution of tert-butyl ((R)-1-(((S)-3-(4-chloro-2-fluorophenoxy)-1-(4,4,5,5-tetramethyl-1,3,2-dioxaborolan-2-yl)propyl)amino)-3-methoxy-1-oxopropan-2-yl)carbamate (29-6, 620 mg, 1.17 mmol) in 1,4-dioxane (6 mL), was added 4 M HCl in 1,4-dioxane (3.2 mL, 12.8 mmol) dropwise at 0° C. The reaction mixture was allowed to warm to ambient temperature and stirred for 3 h. Volatiles were evaporated under reduced pressure and lyophilized to afford (R)-2-amino-N—((S)-3-(4-chloro-2-fluorophenoxy)-1-(4,4,5,5-tetramethyl-1,3,2-dioxaborolan-2-yl)propyl)-3-methoxy propanamide hydrochloride (29-7, 400 mg). [M−H]⁻: 429.4.

Synthesis of N—((R)-1-(((S)-3-(4-chloro-2-fluorophenoxy)-1-(4,4,5,5-tetramethyl-1,3,2-dioxaborolan-2-yl)propyl)amino)-3-methoxy-1-oxopropan-2-yl)pyrazine-2-carboxamide [Step 6]: To a stirred solution of (R)-2-amino-N—((S)-3-(4-chloro-2-fluorophenoxy)-1-(4,4,5,5-tetramethyl-1,3,2-dioxaborolan-2-yl)propyl)-3-methoxypropanamide hydrochloride (29-7, 400 mg, 0.86 mmol) in dichloromethane (5 mL) was added NMM (0.38 mL, 3.42 mmol) at 0° C. followed by the addition of pyrazine-2-carbonyl chloride (29-8, 122 mg, 0.86 mmol) and stirred for 15 min at 0° C. The reaction mixture was allowed to warm to ambient temperature and stirred for 2 h. The reaction mixture was diluted with dichloromethane and washed with ice-cold water, brine, dried over anhydrous Na₂SO₄, filtered and concentrated under reduced pressure to afford N—((R)-1-(((S)-3-(4-chloro-2-fluorophenoxy)-1-(4,4,5,5-tetramethyl-1,3,2-dioxaborolan-2-yl)propyl) amino)-3-methoxy-1-oxopropan-2-yl)pyrazine-2-carboxamide (29-9, 400 mg). [M−H]⁻: 535.4.

Synthesis of ((S)-3-(4-chloro-2-fluorophenoxy)-1-((R)-3-methoxy-2-(pyrazine-2-carboxamido)propanamido)propyl)boronic acid [Step 7]: To a stirred solution of N—((R)-1-(((S)-3-(4-chloro-2-fluorophenoxy)-1-(4,4,5,5-tetramethyl-1,3,2-dioxaborolan-2-yl)propyl)amino)-3-methoxy-1-oxopropan-2-yl)pyrazine-2-carboxamide (29-9, 400 mg, 0.74 mmol) and methylboronic acid (11, 446 mg, 7.45 mmol) in acetone (5 mL) was added 0.2 M HCl (5 mL) and stirred at ambient temperature for 5 h. All volatiles were evaporated under reduced pressure and purified by prep HPLC and lyophilized to afford ((S)-3-(4-chloro-2-fluorophenoxy)-1-((R)-3-methoxy-2-(pyrazine-2-carboxamido)propanamido)propyl)boronic acid (Compound 29, 74 mg). [M−H]⁻: 453.3; ¹H NMR (400 MHz, CD₃OD) δ_{H}: 9.23 (s, 1H), 8.81 (d, 1H), 8.70 (s, 1H), 7.05 (t, 3H), 5.00 (d, 1H), 4.09 (t, 2H), 3.94-3.90 (m, 1H), 3.80-3.76 (m, 1H), 3.40 (s, 3H), 2.92 (t, 1H), 2.02-1.90 (m, 2H).

### Example 30: ((R)-1-((2R,3S)-3-methoxy-2-(pyrazine-2-carboxamido)butanamido)-3-phenoxypropyl)boronic acid

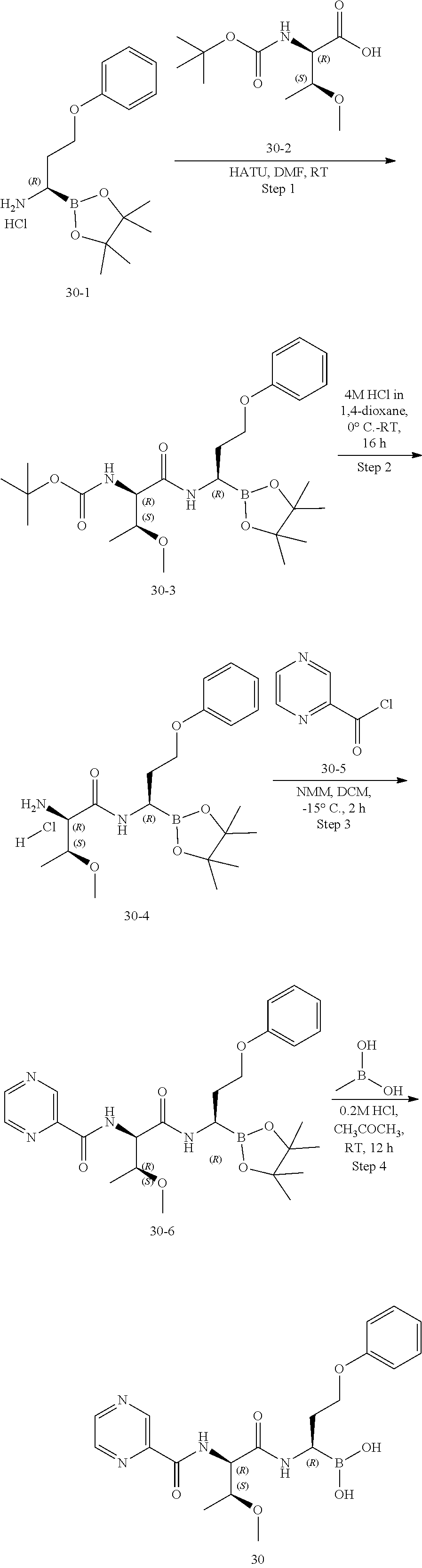

Synthesis of tert-butyl ((2R,3S)-3-methoxy-1-oxo-1-(((R)-3-phenoxy-1-(4,4,5,5-tetramethyl-1,3,2-dioxaborolan-2-yl)propyl)amino)butan-2-yl)carbamate [Step 1]: N-(tert-butoxy carbonyl)-O-methyl-D-threonine (30-2, 300 mg, 1.3 mmol), (R)-3-phenoxy-1-(4,4,5,5-tetramethyl-1,3,2-dioxaborolan-2-yl)propan-1-amine hydrochloride (30-1, 444 mg, 1.4 mmol) and O-(7-azabenzotriazol-1-yl)-N,N,N′,N′-tetramethyluronium hexafluorophosphate, HATU (586 mg, 1.5 mmol) were suspended in dimethylformamide (5 mL) and cooled to 0° C. N,N-Diisopropylethylamine (0.7 mL, 3.9 mmol) was added and stirred at ambient temperature for 2h. The reaction mixture was diluted with ethyl acetate and washed with cold water (thrice) and brine, dried over anhydrous Na₂SO₄ and concentrated under reduced pressure to afford tert-butyl ((2R,3S)-3-methoxy-1-oxo-1-(((R)-3-phenoxy-1-(4,4,5,5-tetramethyl-1,3,2-dioxaborolan-2-yl) propyl)amino)butan-2-yl)carbamate (30-3, 600 mg). [M−H]⁻: 491.4.

Synthesis of (2R,3S)-2-amino-3-methoxy-N—((R)-3-phenoxy-1-(4,4,5,5-tetramethyl-1,3,2-dioxaborolan-2-yl)propyl)butanamide hydrochloride [Step 2]: To a stirred solution of tert-butyl ((2R,3S)-3-methoxy-1-oxo-1-(((R)-3-phenoxy-1-(4,4,5,5-tetramethyl-1,3,2-dioxaborolan-2-yl)propyl)amino)butan-2-yl)carbamate (30-3, 720 mg, 1.5 mmol) in 1,4-dioxane (7 mL) was added 4 M HCl in 1,4-dioxane (3.7 mL, 14.6 mmol) at 0° C. under nitrogen atmosphere and stirred at ambient temperature for 2 h. The reaction mixture was concentrated under reduced pressure and triturated with diethyl ether to afford (2R,3S)-2-amino-3-methoxy-N—((R)-3-phenoxy-1-(4,4,5,5-tetramethyl-1,3,2-dioxaborolan-2-yl)propyl)butanamide hydrochloride (30-4, 600 mg). [M−H]⁻: 391.3, [M-83]⁻: 309.4. LCMS showed corresponding boronic acid mass peak also.

Synthesis of N-((2R,3S)-3-methoxy-1-oxo-1-(((R)-3-phenoxy-1-(4,4,5,5-tetramethyl-1,3,2-dioxaborolan-2-yl)propyl)amino)butan-2-yl)pyrazine-2-carboxamide [Step 3]: To a stirred solution of (2R,3S)-2-amino-3-methoxy-N—((R)-3-phenoxy-1-(4,4,5,5-tetramethyl-1,3,2-dioxaborolan-2-yl)propyl)butanamide hydrochloride (30-4, 680 mg, 1.6 mmol) in dichloromethane (6 mL) was added N-methylmorpholine (0.3 mL, 4.8 mmol) at 0° C. Pyrazine-2-carbonyl chloride (30-5, 339 mg, 2.4 mmol) was added into the reaction mixture and stirred at ambient temperature for 2 h. The reaction mixture was diluted with dichloromethane and washed with saturated aqueous NaHCO₃ and brine, dried over anhydrous Na₂SO₄ and concentrated under reduced pressure. The crude product was purified by prep HPLC and lyophilized to afford N-((2R,3S)-3-methoxy-1-oxo-1-(((R)-3-phenoxy-1-(4,4,5,5-tetramethyl-1,3,2-dioxaborolan-2-yI)propyl)amino) butan-2-yl)pyrazine-2-carboxamide (30-6, 28 mg). [M−H]⁻: 497.4, [M-83]⁻: 415.4. LCMS showed corresponding boronic acid mass peak also.

Synthesis of ((R)-1-((2R,3S)-3-methoxy-2-(pyrazine-2-carboxamido)butanamido)-3-phenoxypropyl)boronic acid [Step 4]: To a stirred solution N-((2R,3S)-3-methoxy-1-oxo-1-(((R)-3-phenoxy-1-(4,4,5,5-tetramethyl-1,3,2-dioxaborolan-2-yl)propyl)amino)butan-2-yl) pyrazine-2-carboxamide (30-6, 28 mg, 0.06 mmol) and methylboronic acid (7, 34 mg, 0.6 mmol) in acetone (3 mL) was added 0.2 M HCl (3.0 mL) at 0° C. and stirred at ambient temperature for 16 h. The volatiles were evaporated under reduced pressure and purified by prep HPLC and lyophilized to afford ((R)-1-((2R,3S)-3-methoxy-2-(pyrazine-2-carboxamido)butanamido)-3-phenoxypropyl)boronic acid (Compound 30, 15 mg). [M−H]⁻: 415.1. ¹H NMR (400 MHz, DMSO-d₆+2 drop D₂O at 80° C.) δ_{H}: 9.18 (s, 1H), 8.87-8.86 (m, 1H), 8.73 (s, 1H), 7.26-7.22 (m, 2H), 6.91-6.85 (m, 3H), 4.54-4.53 (m, 1H), 3.99-3.96 (m, 2H), 3.86-3.84 (m, 1H), 3.32-3.27 (m, 4H), 2.03-1.92 (m, 2H), 1.09-1.08 (m, 3H).

### Example 31: ((R)-1-((R)-3-methoxy-2-(morpholine-4-carboxamido)propanamido)-4-phenyl butyl)boronic acid

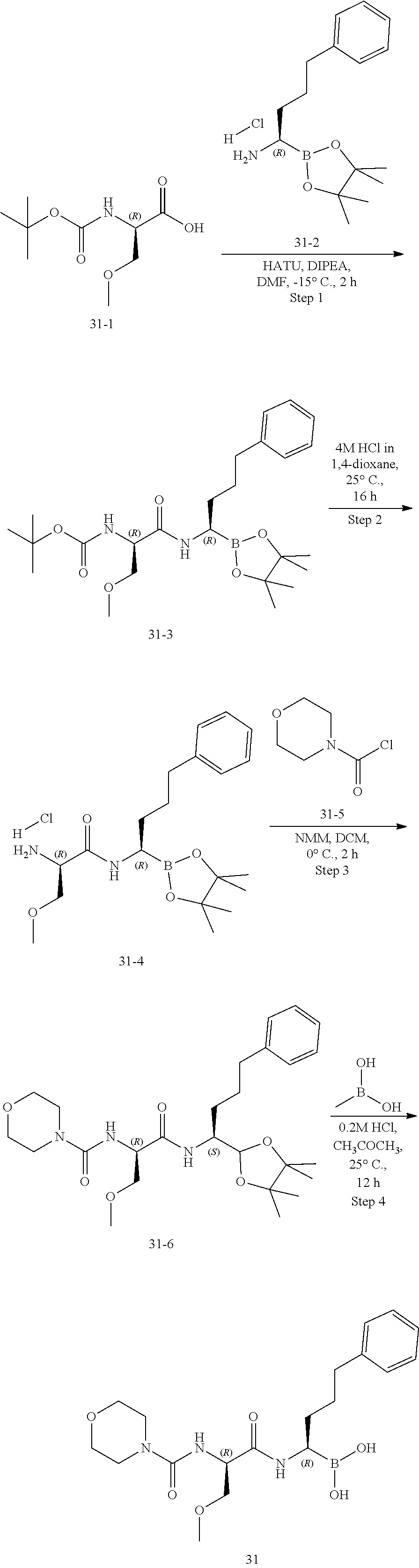

Synthesis of tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate [Step 1]: To a stirred solution of (2R)-2-(tert-butoxycarbonylamino)-3-methoxy-propanoic acid (31-1, 400 mg, 1.8 mmol) in DMF (8 mL) was added HATU (1.39 g, 3.65 mmol) followed by DIPEA (1.0 mL, 5.5 mmol) at 0° C. and stirred for 30 min. (1R)-4-phenyl-1-(4,4,5,5-tetramethyl-1,3,2-dioxaborolan-2-yl)butan-1-amine hydrochloride (31-2, 653 mg, 2.4 mmol) was added to it and the reaction mixture was stirred at 0° C. for 2 h. It was quenched with 5% aqueous K₂CO₃ solution and extracted with EtOAc (thrice). Combined organic layer was washed with water and brine, dried over anhydrous Na₂SO₄ and evaporated under reduced pressure to afford tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate (31-3, 550 mg). [M+H]⁺: 476.9.

Synthesis of (R)-2-amino-3-methoxy-N—((R)-4-phenyl-1-(4,4,5,5-tetramethyl-1,3,2-dioxaborolan-2-yl)butyl)propenamide hydrochloride [Step 2]: To a stirred solution of tert-butyl ((R)-3-methoxy-1-oxo-1-(((R)-4-phenyl-1-(4,4,5,5-tetramethyl-1,3,2-dioxaborolan-2-yl) butyl)amino)propan-2-yl)carbamate (31-3, 250 mg, 0.4 mmol) in 1,4-dioxane (3 mL) was added 4 M HCl in 1,4-dioxane (4 mL) at 0° C. It was gradually warmed to ambient temperature and stirred for 16 h. Volatiles were removed under reduced pressure to afford (R)-2-amino-3-methoxy-N—((R)-4-phenyl-1-(4,4,5,5-tetramethyl-1,3,2-dioxaborolan-2-yl)butyl)propenamide hydrochloride (31-4, 200 mg). [M−H]⁻: 375.4.

Synthesis of N—((R)-3-methoxy-1-oxo-1-(((R)-4-phenyl-1-(4,4,5,5-tetramethyl-1,3,2-dioxaborolan-2-yl)butyl)amino)propan-2-yl)morpholine-4-carboxamide [Step 3]: To a stirred solution of (R)-2-amino-3-methoxy-N—((R)-4-phenyl-1-(4,4,5,5-tetramethyl-1,3,2-dioxaborolan-2-yl)butyl)propenamide hydrochloride (31-4, 400 mg, 0.9 mmol) in dichloromethane (10 mL) was added N-methyl morpholine (0.5 mL, 4.6 mmol) at 0° C. Morpholine-4-carbonyl chloride (31-5, 140 mg, 0.3 mmol) was added to the reaction mixture and stirred at ambient temperature for 1.5 h. The reaction mixture was diluted with water and extracted with DCM. Combined organic layer was washed with water and brine, dried over anhydrous Na₂SO₄ and evaporated under reduced pressure. The crude product was purified by prep HPLC purification and lyophilized to afford N—((R)-3-methoxy-1-oxo-1-(((R)-4-phenyl-1-(4,4,5,5-tetramethyl-1,3,2-dioxaborolan-2-yl)butyl)amino)propan-2-yl)morpholine-4-carboxamide (31-6, 56 mg). [M−H]⁻: 488.3.

Synthesis of ((R)-1-((R)-3-methoxy-2-(morpholine-4-carboxamido)propanamido)-4-phenylbutyl)boronic acid [Step 4]: To a stirred solution of N—((R)-3-methoxy-1-oxo-1-(((S)-4-phenyl-1-(4,4,5,5-tetramethyl-1,3-dioxolan-2-yl)butyl)amino)propan-2-yl)morpholine-4-carboxamide (31-6, 72 mg, 0.14 mmol) and methylboronic acid (88 mg, 1.5 mmol) in acetone (2 mL) was added 0.2 M HCl (2 mL) and stirred at ambient temperature for 16 h. Volatiles were evaporated under reduced pressure and the crude product was purified by prep HPLC purification and lyophilized to afford ((R)-1-((R)-3-methoxy-2-(morpholine-4-carboxamido)propanamido)-4-phenylbutyl)boronic acid (Compound 31, 40 mg). [M−H]⁻: 406.4. ¹H NMR (400 MHz, DMSO-d₆+2 drops of D₂O, 80° C.): δ_{H} 7.26-7.22 (m, 2H), 7.16-7.14 (m, 3H), 4.30-4.27 (m, 1H), 3.55-3.49 (m, δ_{H}), 3.30-3.27 (m, 4H), 3.24-3.23 (m, 3H), 2.57-2.50 (m, 2H), 1.58-1.49 (m, 4H).

### Example 32: ((R)-4-(4-fluorophenyl)-1-((R)-3-methoxy-2-(pyrazine-2-carboxamido) propanamido)butyl)boronic acid

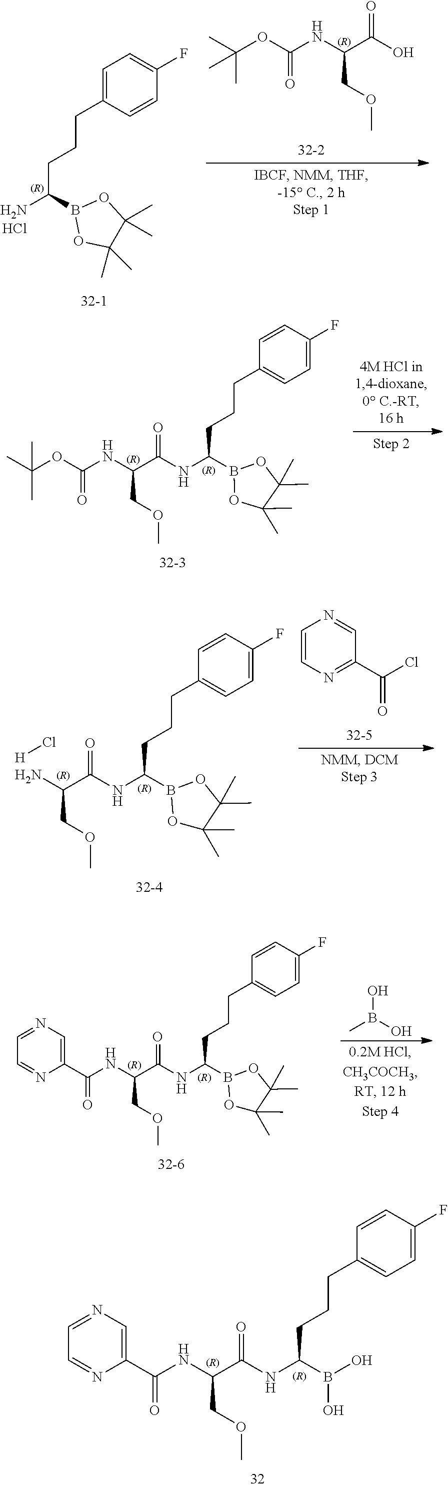

Synthesis of tert-butyl ((R)-1-(((R)-4-(4-fluorophenyl)-1-(4,4,5,5-tetramethyl-1,3,2-dioxaborolan-2-yl)butyl)amino)-3-methoxy-1-oxopropan-2-yl)carbamate [Step 1]: To a stirred solution of N-(tert-butoxycarbonyl)-O-methyl-D-serine (32-2, 495 mg, 2.3 mmol) in tetrahydrofuran (5 mL) was added isobutyl chloroformate (0.2 mL, 1.7 mmol) and N-Methylmorpholine (0.23 mL, 2.1 mmol) at −15° C. and stirred at same temperature for 30 min. A solution of (R)-4-(4-fluorophenyl)-1-(4,4,5,5-tetramethyl-1,3,2-dioxaborolan-2-yl)butan-1-amine hydrochloride (32-1, 620 mg, 1.9 mmol) in tetrahydrofuran (5 mL) followed by N-methylmorpholine (NMM, 0.21 mL, 1.9 mmol) were added to the reaction mixture at −15° C. The reaction mixture was gradually warmed to 0° C. and stirred for 2 h. The reaction mixture was neutralized with 0.1 M HCl and extracted with ethyl acetate (twice). The combined organic extract was washed with aqueous 5% K₂CO₃ solution, water and brine, dried over anhydrous Na₂SO₄ and concentrated under reduced pressure to afford tert-butyl ((R)-1-(((R)-4-(4-fluorophenyl)-1-(4,4,5,5-tetramethyl-1,3,2-dioxaborolan-2-yl)butyl)amino)-3-methoxy-1-oxopropan-2-yl)carbamate (32-3, 1.1 g). [M−H]⁻: 493.2.

Synthesis of (R)-2-amino-N—((R)-4-(4-fluorophenyl)-1-(4,4,5,5-tetramethyl-1,3,2-dioxaborolan-2-yl)butyl)-3-methoxypropanamide hydrochloride [Step 2]: To a stirred solution of tert-butyl ((R)-1-(((R)-4-(4-fluorophenyl)-1-(4,4,5,5-tetramethyl-1,3,2-dioxaborolan-2-yl)butyl)amino)-3-methoxy-1-oxopropan-2-yl)carbamate (32-3, 1.1 g, 2.2 mmol) in 1,4-dioxane (10 mL) was added 4 M HCl in 1,4-dioxane (6 mL, 22.2 mmol) at ice cold condition and stirred at 0° C. for 2 h. The reaction mixture was evaporated under reduced pressure and triturated with n-pentane to afford (R)-2-amino-N—((R)-4-(4-fluorophenyl)-1-(4,4,5,5-tetramethyl-1,3,2-dioxaborolan-2-yl)butyl)-3-methoxypropanamide hydrochloride (32-4, 1 g). [M−H]: 393.3.

Synthesis of N—((R)-1-(((R)-4-(4-fluorophenyl)-1-(4,4,5,5-tetramethyl-1,3,2-dioxaborolan-2-yl)butyl)amino)-3-methoxy-1-oxopropan-2-yl)pyrazine-2-carboxamide [Step 3]: To a stirred solution of (R)-2-amino-N—((R)-4-(4-fluorophenyl)-1-(4,4,5,5-tetramethyl-1,3,2-dioxaborolan-2-yl)butyl)-3-methoxypropanamide hydrochloride (32-4, 600 mg, 1.4 mmol) in dichloromethane (6 mL) was added N-methylmorpholine (0.3 mL, 4.2 mmol) at −15° C. followed by the addition of pyrazine-2-carbonyl chloride (32-5, 298 mg, 2.1 mmol) and stirred for 2 h. The reaction mixture was diluted with dichloromethane and washed with water and brine, dried over anhydrous sodium sulfate and concentrated under reduced pressure. The product was purified by prep HPLC and lyophilized to afford N—((R)-1-(((R)-4-(4-fluorophenyl)-1-(4,4,5,5-tetramethyl-1,3,2-dioxaborolan-2-yl)butyl)amino)-3-methoxy-1-oxopropan-2-yl)pyrazine-2-carboxamide (32-6, 80 mg). [M−H]⁻: 499.4, [M-83]⁻: 417.3. LCMS showed corresponding boronic acid mass peak also.

Synthesis of ((R)-4-(4-fluorophenyl)-1-((R)-3-methoxy-2-(pyrazine-2-carboxamido) propanamido)butyl)boronic acid [Step 4]: To a stirred solution of N—((R)-1-(((R)-4-(4-fluorophenyl)-1-(4,4,5,5-tetramethyl-1,3,2-dioxaborolan-2-yl)butyl)amino)-3-methoxy-1-oxopropan-2-yl)pyrazine-2-carboxamide (32-6, 80 mg, 0.2 mmol) in acetone (10 mL) was added 0.2 M HCl (10 mL) and methylboronic acid (96 mg, 1.6 mmol)) at ice cold condition and stirred at ambient temperature for 16 h. The volatiles were evaporated under reduced pressure and the crude product was purified by prep HPLC and lyophilized to afford ((R)-4-(4-fluorophenyl)-1-((R)-3-methoxy-2-(pyrazine-2-carboxamido)propanamido)butyl)boronic acid (Compound 32, 42 mg). [M−H]⁻: 4173. ¹H NMR (400 MHz, CD₃OD) d_{H}: 9.23 (s, 1H), 8.81-8.80 (m, 1H), 8.71-8.70 (m, 1H), 7.17-7.14 (m, 2H), 6.95-6.91 (m, 2H), 4.98 (t, 1H), 3.89-3.86 (m, 1H), 3.79-3.75 (m, 1H), 3.38 (s, 3H), 2.65-2.63 (m, 1H), 2.61-2.55 (m, 2H), 1.67-1.45 (m, 4H).

### Example 33: ((R)-1-((R)-4-methoxy-2-(pyrazine-2-carboxamido)butanamido)-4-phenylbutyl) boronic acid

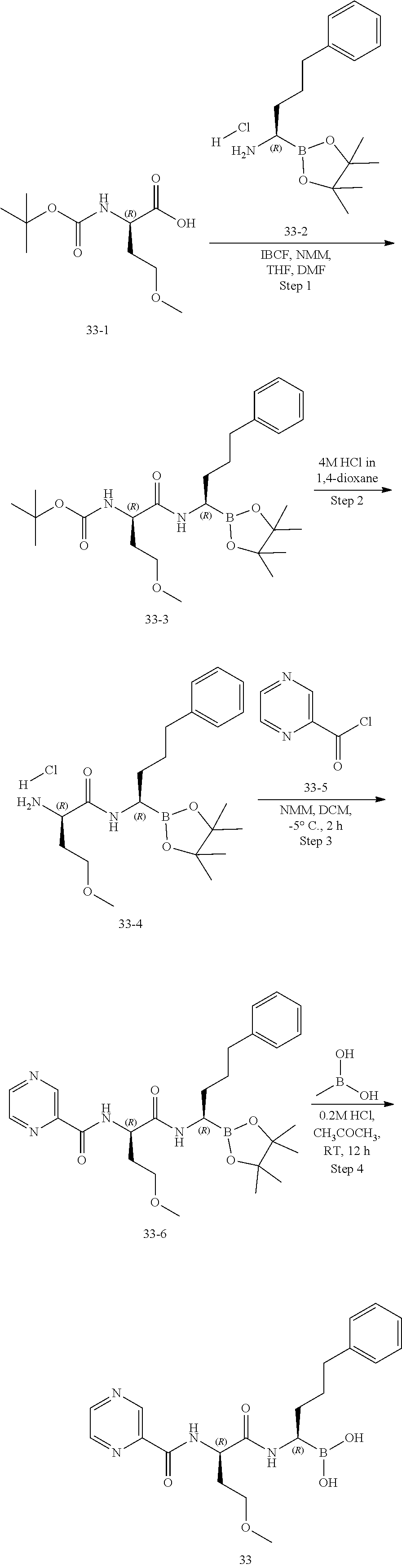

Synthesis of tert-butyl ((R)-4-methoxy-1-oxo-1-(((R)-4-phenyl-1-(4,4,5,5-tetramethyl-1,3,2-dioxaborolan-2-yl)butyl)amino)butan-2-yl)carbamate [Step 1]: To a stirred solution of N-(tert-butoxycarbonyl)-O-methyl-D-homoserine (33-1, 330 mg, 1.4 mmol) in tetrahydrofuran (8 mL) was added IBCF (0.2 mL, 1.4 mmol) and NMM (0.2 mL, 1.4 mmol) at −15° C. and stirred for 30 min. A solution of (R)-4-phenyl-1-(4,4,5,5-tetramethyl-1,3,2-dioxaborolan-2-yl)butan-1-amine, hydrochloride (33-2, 400 mg, 1.3 mmol) in dimethylformamide (1 mL) was added to it followed by NMM (0.17 mL, 1.3 mmol) at −15° C. The reaction mixture was gradually warmed to 0° C. and stirred for 2 h. The reaction mass was diluted with ethyl acetate and washed with 0.1 M HCl (twice), 5% aqueous K₂CO₃ solution (twice), water (twice) and brine, dried over anhydrous Na₂SO₄ and evaporated under reduced pressure to afford tert-butyl ((R)-4-methoxy-1-oxo-1-(((R)-4-phenyl-1-(4,4,5,5-tetramethyl-1,3,2-dioxaborolan-2-yl)butyl)amino)butan-2-yl)carbamate (33-3, 500 mg). [M+H]⁺=490.8.

Synthesis of (R)-2-amino-4-methoxy-N—((R)-4-phenyl-1-(4,4,5,5-tetramethyl-1,3,2-dioxaborolan-2-yl)butyl)butanamide hydrochloride [Step 2]: To a solution of tert-butyl ((R)-4-methoxy-1-oxo-1-(((R)-4-phenyl-1-(4,4,5,5-tetramethyl-1,3,2-dioxaborolan-2-yl)butyl)amino) butan-2-yl)carbamate (33-3, 500 mg, 1.0 mmol) in 1, 4 dioxane (6 mL) was added 4 M HCl in 1,4-dioxane (5.0 mL, 20.0 mmol) at 0° C. and stirred at 25° C. for 16 h. Volatiles were removed under reduced pressure to give (R)-2-amino-4-methoxy-N—((R)-4-phenyl-1-(4,4,5,5-tetramethyl-1,3,2-dioxaborolan-2-yl)butyl)butanamide hydrochloride (33-4, 400 mg). [M+H]⁺=389.4.

Synthesis of N—((R)-4-methoxy-1-oxo-1-(((R)-4-phenyl-1-(4,4,5,5-tetramethyl-1,3,2-dioxaborolan-2-yl)butyl)amino)butan-2-yl)pyrazine-2-carboxamide [Step 3]: To a stirred solution of (R)-2-amino-4-methoxy-N—((R)-4-phenyl-1-(4,4,5,5-tetramethyl-1,3,2-dioxaborolan-2-yl)butyl)butanamide hydrochloride (33-4, 400 mg, 1.0 mmol) in dichloromethane (5 mL) was added NMM (0.3 mL, 2.0 mmol) at −5° C. and stirred for 15 min. Pyrazine-2-carbonyl chloride (33-5, 160 mg, 1.1 mmol) was added to it and stirred at 0° C. for 2 h. The reaction mass was diluted with dichloromethane and washed with water and brine, dried over anhydrous Na₂SO₄ and evaporated under reduced pressure. The compound was purified by PREP-HPLC purification and lyophilized to afford N—((R)-4-methoxy-1-oxo-1-(((R)-4-phenyl-1-(4,4,5,5-tetramethyl-1,3,2-dioxaborolan-2-yl)butyl)amino)butan-2-yl)pyrazine-2-carboxamide (33-6, 60 mg). [M−H]⁻=495.3, 413.3. LCMS showed mixture of boronate ester and corresponding boronic acid mass peak.

Synthesis of ((R)-1-((R)-4-methoxy-2-(pyrazine-2-carboxamido)butanamido)-4-phenyl butyl)boronic acid [Step 4]: To a stirred solution of N—((R)-4-methoxy-1-oxo-1-(((R)-4-phenyl-1-(4,4,5,5-tetramethyl-1,3,2-dioxaborolan-2-yl)butyl)amino)butan-2-yl)pyrazine-2-carboxamide (33-6, 50 mg, 0.10 mmol) and methylboronic acid (60 mg, 1 mmol) in acetone (2 mL) was added 0.2 M HCl (2 mL) and stirred at ambient temperature for overnight. Volatiles were removed under reduced pressure and purified by prep HPLC purification to afford ((R)-1-((R)-4-methoxy-2-(pyrazine-2-carboxamido)butanamido)-4-phenylbutyl)boronic acid (Compound 33, 24 mg). [M−H]⁻=413.3. ¹H NMR (400 MHz, CD₃OD): δ_{H} 9.21 (s, 1H), 8.80 (d, 1H), 8.73-8.68 (m, 1H), 7.25-7.06 (m, 5H), 4.94 (t, 1H), 3.57-3.48 (m, 2H), 3.46-3.30 (m, 2H), 2.60 (q, 3H), 2.35-2.09 (m, 2H), 1.80-1.38 (m, 4H).

### Example 34: [(1R)-4-phenyl-1-[(2S)-3-(phenylsulfanyl)-2-(pyrazin-2-ylformamido) propanamido]butyl]boronic acid

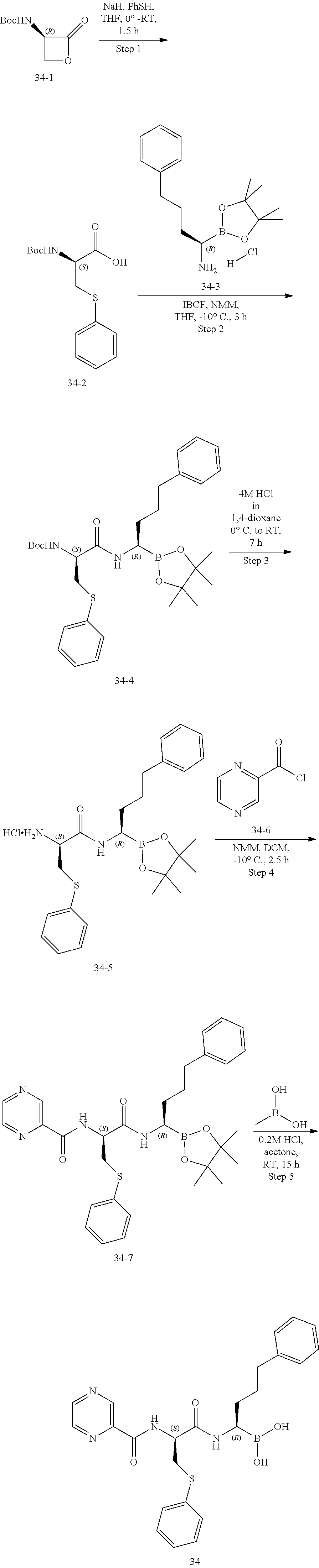

Synthesis of (2S)-2-{[(tert-butoxy)carbonyl]amino}-3-(phenylsulfanyl)propanoic acid [Step 1]: Under nitrogen, to the stirred solution of thiophenol (0.6 ml, 5.9 mmol) in THF (10 mL) was added NaH (235 mg, 5.9 mmol) at 0° C. and stirred at same temperature for 15 min (white suspension was observed). To this was then added solution of tert-butyl N-[(3R)-2-oxooxetan-3-yl]carbamate (34-1, 1.0 g, 5.3 mmol) in THF (10 mL) and then stirred at room temperature for 1.5 h. Reaction mixture was diluted with cold water and washed with EtOAc (10 mL). Aqueous part was acidified with NaHSO₄ under ice cold condition and extracted with EtOAc (100 mL×2). Combined organic part was washed with brine, dried over Na₂SO₄, filtered and concentrated under reduced pressure at 30° C. Resulting crude solid was triturated with 10% Et₂O in hexane (10 mL×2) and dried to afford (2S)-2-{[(tert-butoxy)carbonyl]amino}-3-(phenylsulfanyl)propanoic acid (34-2, 1.2 g). [M+H]⁺: 298.2

Synthesis of tert-butyl N-[(1S)-1-{[(1R)-4-phenyl-1-(tetramethyl-1,3,2-dioxaborolan-2-yl) butyl]carbamoyl}-2-(phenylsulfanyl)ethyl]carbamate [Step 2]: To a solution of 2S)-2-{[(tert-butoxy)carbonyl]amino}-3-(phenylsulfanyl)propanoic acid (34-2, 670 mg, 2.25 mmol) in THF (5 mL), were added NMM (0.26 mL, 1.95 mmol) and IBCF (0.24 mL, 1.78 mmol) dropwise at −10° C. and stirred for 30 mins at −10° C. To this reaction mixture solution of (1R)-4-phenyl-1-(tetramethyl-1,3,2-dioxaborolan-2-yl)butan-1-amine hydrochloride (34-3, 500 mg, 1.6 mmol) in THF (10 mL) and NMM (0.26 mL, 1.94 mmol) were added drop wise and stirred at −10° C. for 2 h. The reaction mixture was diluted with ethyl acetate and the organic layer was washed with cold 0.1 M HCl solution (10 mL×2), followed by 10% aq K₂CO₃ solution (10 mL) and finally by brine. Organic part was dried over Na₂SO₄ and evaporated under reduced pressure at 30° C. to afford tert-butyl N—[(1S)-1-{[(1R)-4-phenyl-1-(tetramethyl-1,3,2-dioxaborolan-2-yl)butyl]carbamoyl}-2-(phenyl sulfanyl)ethyl]carbamate (34-4, 1 g crude). [M−H]⁻: 553.5.

Synthesis of (2S)-2-amino-N-[(1R)-4-phenyl-1-(tetramethyl-1,3,2-dioxaborolan-2-yl)butyl]-3-(phenylsulfanyl)propanamide hydrochloride [Step 3]: To the solution of tert-butyl N-[(1S)-1-{[(1R)-4-phenyl-1-(tetramethyl-1,3,2-dioxaborolan-2-yl)butyl]carbamoyl}-2-(phenylsulfanyl)ethyl]carbamate (34-4, 1.0 g crude material) in 1,4-dioxane (5 mL) was added 4 M HCl in 1,4-dioxane (4.5 mL, 14.43 mmol) dropwise at 0° C. and then stirred at room temperature for 7 h. Reaction mixture was concentrated under reduced pressure at 30° C. Residue was co-distilled with hexane (two times) and dried to afford (2S)-2-amino-N-[(1R)-4-phenyl-1-(tetramethyl-1,3,2-dioxaborolan-2-yl)butyl]-3-(phenylsulfanyl)propanamide hydrochloride (34-5, 1 g crude). [M−H]⁻: 453.4 (Mass of boronic acid derivative of (2S)-2-amino-N-[(1R)-4-phenyl-1-(tetramethyl-1,3,2-dioxaborolan-2-yl)butyl]-3-(phenylsulfanyl) propanamide was observed as major peak).

Synthesis of (2S)—N-[(1R)-4-phenyl-1-(tetramethyl-1,3,2-dioxaborolan-2-yl)butyl]-3-(phenyl sulfanyl)-2-(pyrazin-2-ylformamido)propanamide [Step 4]: To the solution of ((2S)-2-amino-N-[(1R)-4-phenyl-1-(tetramethyl-1,3,2-dioxaborolan-2-yl)butyl]-3-(phenylsulfanyl)propanamide hydrochloride (34-5, 250 mg, 0.509 mmol) in DCM (10 mL) was added NMM (0.18 mL, 1.32 mmol) at 0° C. To this was added pyrazine-2-carbonyl chloride (34-6, 87 mg, 0.6 mmol) and slowly warmed to room temperature and stirred for 2.5 h. Reaction mixture was then diluted with DCM and washed with water and brine. DCM layer was dried over Na₂SO₄, filtered and concentrated under reduced pressure at 30° C. to afford crude which was purified by RP-prep HPLC purification. Pure fraction was lyophilized to afford (2S)—N-[(1R)-4-phenyl-1-(tetramethyl-1,3,2-dioxaborolan-2-yl)butyl]-3-(phenylsulfanyl)-2-(pyrazin-2-ylformamido)propanamide (34-7, 40 mg). [M−H]⁻: 559.3 (Mass of boronic acid derivative of (2S)—N-[(1R)-4-phenyl-1-(tetramethyl-1,3,2-dioxaborolan-2-yl)butyl]-3-(phenyl sulfanyl)-2-(pyrazin-2-ylformamido)propanamide was observed as major peak).

Synthesis of [(1R)-4-phenyl-1-[(2S)-3-(phenylsulfanyl)-2-(pyrazin-2-ylformamido) propanamido]butyl]boronic acid [Step 5]: To the solution of (2S)—N-[(1R)-4-phenyl-1-(tetramethyl-1,3,2-dioxaborolan-2-yl)butyl]-3-(phenylsulfanyl)-2-(pyrazin-2-ylformamido) propanamide (34-7, 40 mg, 0.7 mmol) in acetone was added methylboronic acid (43 mg, 0.72 mmol) and HCl (0.2 M in water, 1 mL) dropwise at 0° C. and then stirred at room temperature for 16 h. Reaction mass was concentrated under reduced pressure at 30° C. to near dryness and then lyophilized to get solid crude which was purified by RP PREP-HPLC. Pure fraction was lyophilized to afford [(1R)-4-phenyl-1-[(2S)-3-(phenylsulfanyl)-2-(pyrazin-2-ylformamido) propanamido]butyl]boronic acid (Compound 34, 20 mg). [M−H]⁻: 477.3. ¹H NMR (400 MHz, CD₃OD): δ 9.17 (d, 1H), 8.78-8.77 (m, 1H), 8.65-8.64 (m, 1H), 7.42-7.40 (d, 2H), 7.26-7.21 (m, 2H), 7.19-7.11 (m, δ_{H}), 4.94 (m, 1H), 5.58-3.53 (m, 1H), 3.47-3.41 (m, 1H), 2.65 (m, 1H), 2.61-2.55 (m, 2H), 1.65-1.49 (m, 4H).

### Example 35: ((R)-1-((S)-3-(N,N-dimethylsulfamoyl)-2-(pyrazine-2-carboxamido) propanamido)-4-phenylbutyl)boronic acid

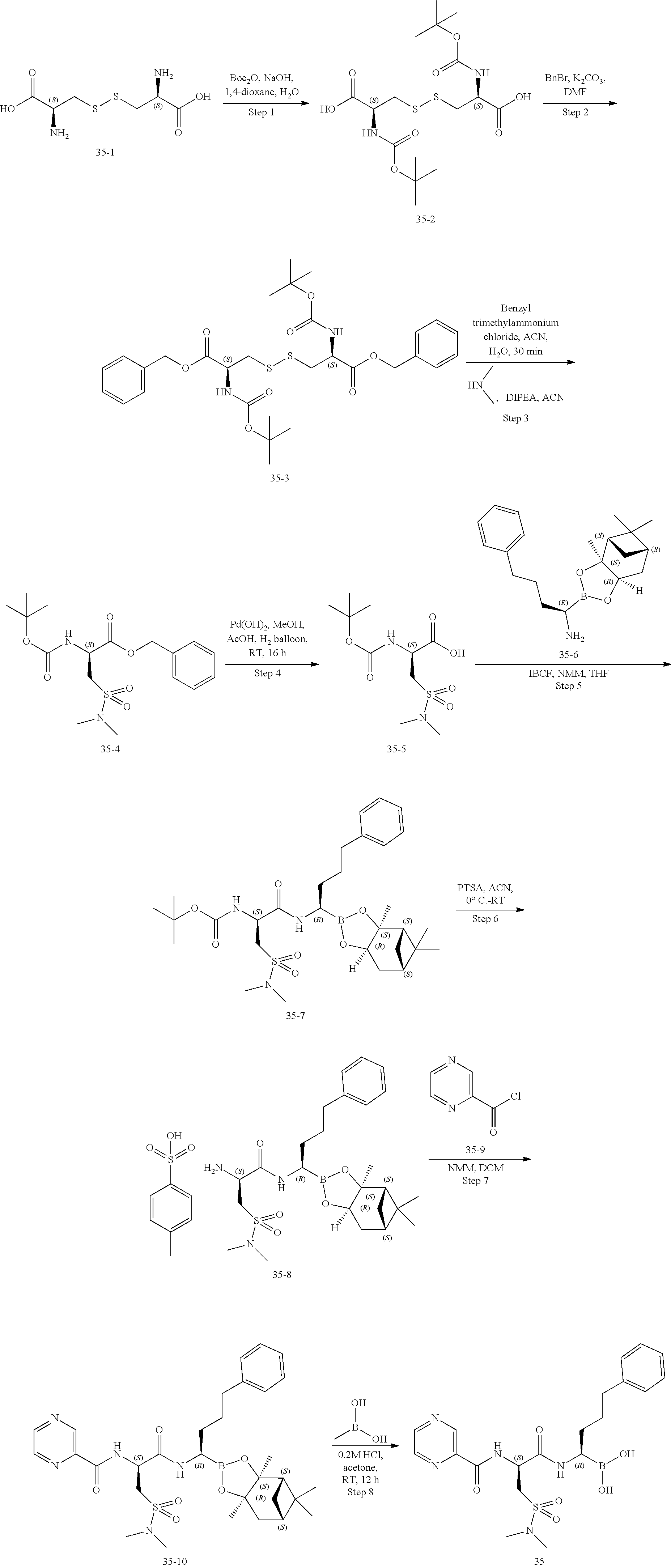

Synthesis of (2S,2′Sa)-3,3′-disulfanediylbis(2-((tert-butoxycarbonyl)amino)propanoic acid) [Step 1]: To a stirred suspension of (2S,2'S)-3,3′-disulfanediylbis(2-aminopropanoic acid) (35-1, 6.50 g, 27.0 mmol) in 1,4-dioxane (35 m) was added NaOH (1 M in water, 135 mL, 135 mmol) followed by Boc-anhydride (19 mL, 81.1 mmol) drop wise at 0° C. Resulting solution was stirred at ambient temperature for 16 h. The reaction mixture was washed with diethyl ether and the aqueous part was acidified with saturated aqueous solution of NaHSO₄ and extracted with EtOAc (twice). Combined organic phase was dried over anhydrous Na2SO4, filtered and concentrated under reduced pressure to afford (2S,2'S)-3,3′-disulfanediylbis(2-((tert-butoxycarbonyl) amino)propanoic acid) (35-2, 10.0 g). ¹H NMR (400 MHz, DMSO-da) n 12.78 (m, 2H), 7.19-7.17 (d, 2H), 4.16 (m, 2H), 3.56 (s, 1H), 3.11-3.07 (m, 2H), 2.91-2.85 (m, 2H), 1.37 (s, 18H). Note: extra proton present in NMR.

Synthesis of dibenzyl 3,3′-disulfanediyl(2S,2'S)-bis(2-((tert-butoxycarbonyl)amino) propanoate) [Step 2]: To a stirred solution of (2S,2'S)-3,3′-disulfanediylbis(2-((tert-butoxycarbonyl)amino)propanoic acid) (35-2, 1.5 g, 3.40 mmol) in DMF (10 mL) was added K₂CO₃ (2.3 g, 17 mmol) followed by benzyl bromide (1.6 mL, 13.6 mmol) at 0° C. After stirring for 16 h at ambient temperature the reaction mixture poured over crushed ice and extracted with EtOAc (twice). Combined organic phase was washed with water followed by brine, dried over anhydrous Na₂SO₄, filtered and concentrated under reduced pressure. The product was purified by column chromatography to afford dibenzyl 3,3′-disulfanediyl(2S,2'S)-bis(2-((tert-butoxy carbonyl)amino)propanoate) (35-3, 1.4 g). ¹H NMR (400 MHz, DMSO-d₆) δ 7.46-7.44 (d, 1H), 7.35-7.28 (m, 10H), 5.15-5.12 (m, 4H), 4.49-4.47 (d, 1H), 4.30 (m, 2H), 3.12-3.10 (m, 2H), 2.96-2.91 (m, 2H), 1.36 (s, 18H).

Synthesis of benzyl (tert-butoxycarbonyl)(N,N-dimethylsulfamoyl)-L-alaninate [Step 3]: To the stirred solution of dibenzyl 3,3′-disulfanediyl(2S,2'S)-bis(2-((tert-butoxycarbonyl)amino) propanoate) (35-3, 1.40 g, 2.26 mmol) in acetonitrile (40 mL) was added Water (0.8 mL) followed by N-benzyltriethyl ammonium chloride (1.54 g, 6.77 mmol) at 0° C. Resulting reaction mixture was stirred at 0° C. for 15 min and added 1,3-dichloro-5,5-dimethylhydantoin (889 mg, 4.51 mmol) portion wise at 0° C. and allowed to stir for 30 min at same temperature. To this resulting solution were added dimethyl amine (203 mg, 4.5 mmol) followed by DIPEA (0.61 mL, 4.51 mmol) at 0° C. and stirred for 1 h at ambient temperature. The reaction mixture was quenched with cold water and extracted with ethyl acetate (twice). The combined organic phase was washed with brine, dried over anhydrous Na₂SO₄, filtered and concentrated under reduced pressure to get crude. The crude material was purified by column chromatography to afford benzyl (tert-butoxycarbonyl)(N,N-dimethylsulfamoyl)-L-alaninate (35-4, 550 mg). ¹H NMR (400 MHz, DMSO-d₆) δ 7.50-7.48 (d, 1H), 7.37 (s, 5H), 5.18-5.11 (m, 2H), 4.45 (m, 1H), 3.46-3.45 (m, 2H), 2.75 (s, 6H), 1.37 (s, 9H).

Synthesis of (tert-butoxycarbonyl)(N,N-dimethylsulfamoyl)-L-alanine [Step 4]: To the solution of benzyl (tert-butoxycarbonyl)(chlorosulfonyl)-L-alaninate (35-4, 550 mg, 1.42 mmol) in methanol (50 mL) and AcOH (1 mL) was added 20% Pd(OH)₂ (Dry) (220 mg, 1.54 mmol) and then stirred under hydrogen balloon pressure for 16 h at ambient temperature. The reaction mixture was filtered through a small bed of celite and the filtrate was concentrated under reduced pressure. The product was washed with 10% Et₂O in Hexane (two times) and dried afford (tert-butoxycarbonyl)(N,N-dimethylsulfamoyl)-L-alanine (35-5, 350 mg). [M−H]⁻=295.07.

Synthesis of tert-butyl ((S)-3-(N,N-dimethylsulfamoyl)-1-oxo-1-(((R)-4-phenyl-1-((3aS,4S,6S,7aR)-3a,5,5-trimethylhexahydro-4,6-methanobenzo[d][1,3,2]dioxaborol-2-yl) butyl)amino)propan-2-yl)carbamate [Step 5]: To a stirred solution of (tert-butoxy carbonyl)(N,N-dimethylsulfamoyl)-L-alanine (35-5, 428 mg, 1.44 mmol) in THF (5 mL), were added NMM (0.22 mL, 1.65 mmol) and IBCF (0.21 mL, 1.53 mmol) dropwise at −10° C. and stirred for 30 mins at −10° C. To this reaction mixture solution of (R)-4-phenyl-1-((3aS,4S,6S,7aR)-3a,5,5-trimethylhexahydro-4,6-methanobenzo[d][1,3,2]dioxaborol-2-yl)butan-1-amine (35-6, 500 mg, 1.37 mmol) in THF (10 mL) and NMM (0.22 mL, 1.65 mmol) were added at −10° C., then stirred for 1 h at same temperature and finally at ambient temperature for 1.5 h. The reaction mixture was diluted with ethyl acetate and was washed with cold aqueous 0.1 N HCl solution (two times), followed by 10% aqueous K₂CO₃ solution (two times) and finally with brine. Organic phase was dried over Na₂SO₄ and evaporated under reduced pressure at 30° C. to afford tert-butyl ((S)-3-(N,N-dimethylsulfamoyl)-1-oxo-1-(((R)-4-phenyl-1-((3aS,4S,6S,7aR)-3a,5,5-trimethyl hexahydro-4,6-methanobenzo[d][1,3,2]dioxaborol-2-yl)butyl)amino)propan-2-yl)carbamate (35-7, 800 mg). [M−H]⁻=604.5.

Synthesis of (S)-2-amino-3-(N,N-dimethylsulfamoyl)-N—((R)-4-phenyl-1-((3aS,4S,6S,7aR)-3a,5,5-trimethylhexahydro-4,6-methanobenzo[d][1,3,2]dioxaborol-2-yl)butyl)propenamide [Step 6]: To the solution of tert-butyl ((S)-3-(N,N-dimethylsulfamoyl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl) amino)propan-2-yl)carbamate (35-7, 800 mg, 1.32 mmol) in acetonitrile (10 mL) was added PTSA (502 mg, 2.64 mmol) at 0° C. and then stirred at ambient temperature for 2 h. Reaction was monitored by LCMS. Reaction mass was concentrated under reduced pressure to afford (S)-2-amino-3-(N,N-dimethylsulfamoyl)-N—((R)-4-phenyl-1-((3aS,4S,6S,7aR)-3a,5,5-trimethyl hexahydro-4,6-methanobenzo[d][1,3,2]dioxaborol-2-yl)butyl)propanamide (35-8, 1.1 g). This product was forwarded to the next step without further purification. [M−H]⁻=504.6.

Synthesis of N—((S)-3-(N,N-dimethylsulfamoyl)-1-oxo-1-(((R)-4-phenyl-1-((3aS,4S,6S,7aR)-3a,5,5,7a-tetramethylhexahydro-4,6-methanobenzo[d][1,3,2]dioxaborol-2-yl)butyl)amino) propan-2-yl)pyrazine-2-carboxamide [Step 7]: To a stirred solution of (S)-2-amino-3-(N,N-dimethylsulfamoyl)-N—((R)-4-phenyl-1-((3aS,4S,6S,7aR)-3a,5,5-trimethylhexahydro-4,6-methano benzo[d][1,3,2]dioxaborol-2-yl)butyl)propanamide (35-8, 1.25 g) in DCM (25 mL) was added NMM (0.50 mL, 3.69 mmol) at 0-5° C. After 10 min pyrazine-2-carbonyl chloride (35-9, 342 mg, 2.40 mmol) was added into that reaction mixture. The reaction was stirred at ambient temperature for 2 h. The reaction mixture was diluted DCM (100 ml) and washed with saturated aqueous solution of NaHCO₃, followed by brine solution. The organic phase was dried over anhydrous Na₂SO₄, filtered and concentrated under reduced pressure to afford crude material which was purified by reverse phase PREP-HPLC to afford N—((S)-3-(N,N-dimethylsulfamoyl)-1-oxo-1-(((R)-4-phenyl-1-((3aS,4S,6S,7aR)-3a,5,5,7a-tetramethylhexahydro-4,6-methanobenzo [d][1,3,2]dioxaborol-2-yl)butyl)amino)propan-2-yl)pyrazine-2-carboxamide (35-10, 75 mg). [M−H]⁻=610.4.

Synthesis of ((R)-1-((S)-3-(N,N-dimethylsulfamoyl)-2-(pyrazine-2-carboxamido) propanamido)-4-phenylbutyl)boronic acid [Step 8]: To a solution of N—((S)-3-(N,N-dimethylsulfamoyl)-1-oxo-1-(((R)-4-phenyl-1-((3aS,4S,6S,7aR)-3a,5,5,7a-tetramethylhexahydro-4,6-methanobenzo[d][1,3,2]dioxaborol-2-yl)butyl)amino)propan-2-yl)pyrazine-2-carboxamide (35-10, 55 mg, 0.09 mmol) in acetone (5 mL) was added methylboronic acid (54 mg, 0.899 mmol), followed by drop wise addition of 0.2 M aqueous HCl (1 mL). The reaction mixture was allowed to stir at ambient temperature for 16 h. Reaction was monitored by LCMS. Reaction mass was concentrated under reduced pressure, lyophilized and finally purified by reverse phase prep HPLC. Pure fraction was concentrated under reduced pressure at 30° C. and lyophilized to afford ((R)-1-((S)-3-(N,N-dimethylsulfamoyl)-2-(pyrazine-2-carboxamido)propanamido)-4-phenyl butyl)boronic acid (Compound 35.19 mg). ¹H NMR (400 MHz, CD₃OD) δ 9.24 (s, 11H), 8.82-8.80 (d, 1H), 8.71 (d, 1H), 7.19-7.09 (m, 5H), 5.27-5.25 (m, 1H), 3.69-3.62 (m, 2H), 2.85 (s, 4H), 2.79-2.71 (m, 1H), 2.67 (s, 2H), 2.63-2.56 (m, 2H), 1.69-1.55 (m, 4H). Note: Extra protons present in NMR. [M−H]⁻=476.4.

### Example 36: ((R)-1-((R)-3-(N,N-dimethylsulfamoyl)-2-(pyrazine-2-carboxamido) propanamido)-4-phenylbutyl)boronic acid

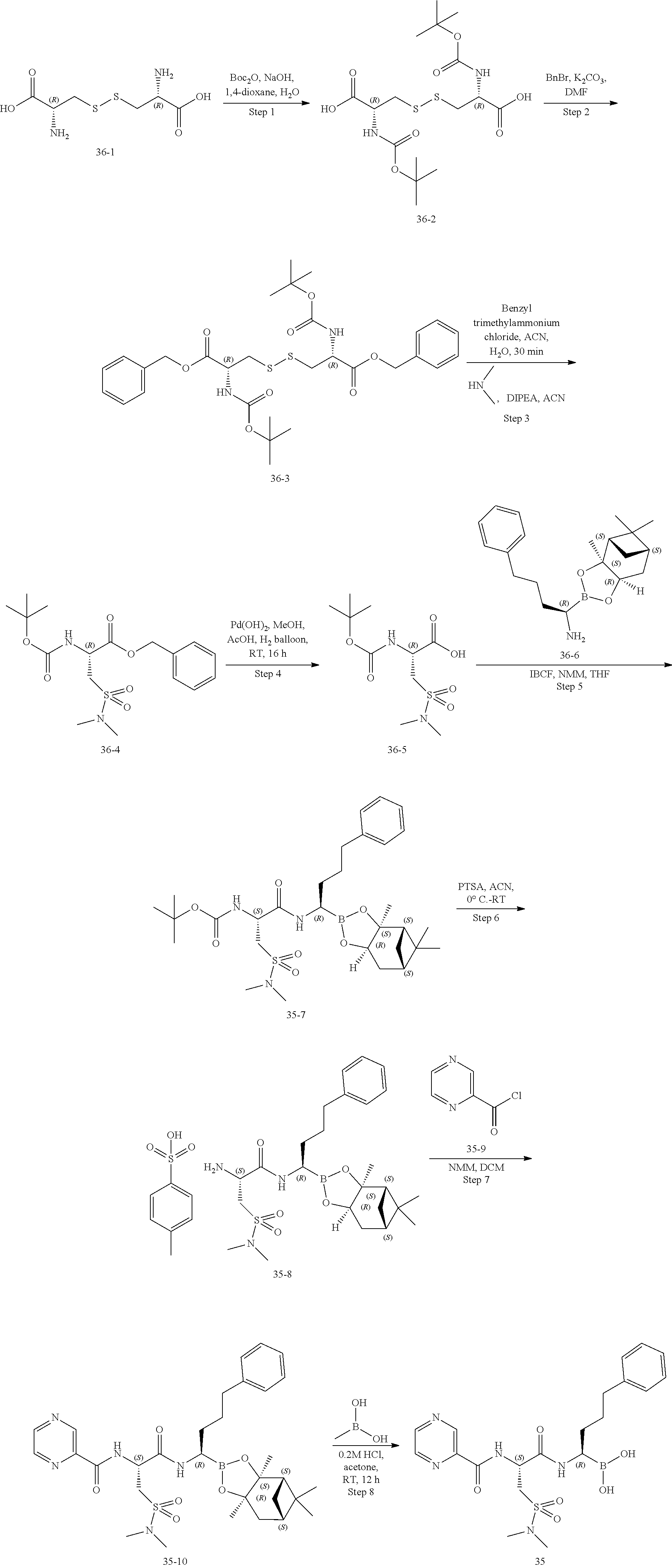

Synthesis of (2R,2′R)-3,3′-disulfanediylbis(2-((tert-butoxycarbonyl)amino)propanoic acid) [Step 1]: To a stirred suspension of (2R,2′R)-3,3′-disulfanediylbis(2-aminopropanoic acid) (36-1, 5.00 g, 20.8 mmol) in 1,4-Dioxane (25 mL) was added NaOH (1 M in water, 104 mL, 104 mmol) followed by Boc-anhydride (14 mL, 62.4 mmol) drop wise at 0° C. Resulting solution was stirred at ambient temperature for 16 h. The reaction mixture was washed with diethyl ether and the aqueous part was acidified with saturated aqueous solution of NaHSO₄ and extracted with EtOAc (twice). Combined organic phase was dried over anhydrous Na₂SO₄, filtered and concentrated under reduced pressure to afford (2R,2′R)-3,3′-disulfanediylbis(2-((tert-butoxycarbonyl) amino)propanoic acid) (36-2, 7.0 g). ¹H NMR (400 MHz, DMSO-d₆) δ 12.79 (m, 2H), 7.19-7.17 (d, 2H), 4.17-4.14 (m, 2H), 3.56 (s, 1H), 3.11-3.07 (m, 2H), 2.91-2.85 (m, 2H), 1.37 (s, 18H).

Synthesis of dibenzyl 3,3′-disulfanediyl(2R,2′R)-bis(2-((tert-butoxycarbonyl)amino) propanoate) [Step 2]: To a stirred solution of (2R,2′R)-3,3′-disulfanediylbis(2-((tert-butoxycarbonyl)amino)propanoic acid) (36-2, 5.00 g, 11.3 mmol) in DMF (25 mL) was added K₂CO₃ (18.5 g, 56.7 mmol) followed by benzyl bromide (5.4 mL, 45.4 mmol) at 0° C. After stirring for 16 h at ambient temperature the reaction mixture poured over crushed ice and extracted with EtOAc (twice). Combined organic phase was washed with water followed by brine, dried over anhydrous Na₂SO₄ and concentrated to afford crude product which was purified by column chromatography to afford dibenzyl 3,3′-disulfanediyl(2R,2′R)-bis(2-((tert-butoxycarbonyl) amino)propanoate) (36-3, 6.00 g). ¹H NMR (400 MHz, DMSO-d₆) δ 7.45-7.43 (d, 2H), 7.35-7.30 (m, 10H), 5.15-5.13 (m, 4H), 4.33-4.28 (m, 2H), 3.14-3.09 (m, 2H), 2.97-2.88 (m, 2H), 1.36 (s, 18H).

Synthesis of benzyl (tert-butoxycarbonyl)(N,N-dimethylsulfamoyl)-D-alaninate [Step 3]: To the stirred solution of dibenzyl 3,3′-disulfanediyl(2R,2′R)-bis(2-((tert-butoxycarbonyl)amino) propanoate) (36-3, 2.50 g, 4.03 mmol) in acetonitrile (60 mL)) was added Water (0.8 mL) followed by N-benzyltriethyl ammonium chloride (2.75 g, 12.1 mmol) at 0° C. Resulting reaction mixture was stirred at 0° C. for 15 min and added 1,3-dichloro-5,5-dimethylhydantoin (1.59 g, 8.05 mmol) portion wise at 0° C. and allowed to stir for 30 min at same temperature. To this solution next were added dimethyl amine (145 mg, 3.22 mmol) followed by DIPEA (0.44 mL, 3.22 mmol) at 0° C. and stirred for 1 h at ambient temperature. The reaction mixture was quenched with cold water and extracted with ethyl acetate (twice). The combined organic phase was washed with brine, dried over anhydrous Na₂SO₄ and concentrated and the product was purified by column chromatography to afford benzyl (tert-butoxycarbonyl)(chlorosulfonyl)-D-alaninate (36-4, 400 mg). ¹H NMR (400 MHz, DMSO-d₆) δ 7.52-7.50 (d, 1H), 7.36 (s, 5H), 5.15-5.13 (m, 2H), 4.44 (m, 1H), 4.03 (m, 1H), 3.46-3.44 (m, 2H), 2.75 (s, δ_{H}), 1.36 (s, 9H). Note: Some extra protons are present in aliphatic region.

Synthesis of (tert-butoxycarbonyl)(N,N-dimethylsulfamoyl)-D-alanine [Step 4]: To the solution of benzyl (tert-butoxycarbonyl)(chlorosulfonyl)-D-alaninate (1.20 g, 3.11 mmol) in methanol (100 mL) and AcOH (0.05 mL) was added 20% Pd(OH)₂ (Dry) (480 mg, 3.37 mmol) and then stirred under H₂ balloon pressure for 16 h at ambient temperature. The reaction mixture was filtered through a small bed of celite and the filtrate was concentrated under reduced pressure. The product was washed with 10% Et₂O in Hexane (two times) and dried afford (tert-butoxycarbonyl)(N,N-dimethylsulfamoyl)-D-alanine (36-5, 700 mg). [M−H]⁻=295.13.

Synthesis of tert-butyl ((R)-3-(N,N-dimethylsulfamoyl)-1-oxo-1-(((R)-4-phenyl-1-((3aS,4S,6S,7aR)-3a,5,5-trimethylhexahydro-4,6-methanobenzo[d][1,3,2]dioxaborol-2-yl) butyl)amino)propan-2-yl)carbamate [Step 5]: To a stirred solution of (tert-butoxycarbonyl)(N,N-dimethylsulfamoyl)-D-alanine (36-5, 642 mg) in THF (5 mL), were added NMM (0.34 mL, 2.47 mmol) and IBCF (0.31 mL, 2.29 mmol) dropwise at −10° C. and stirred for 30 mins at −10° C. To this reaction mixture solution of (R)-4-phenyl-1-((3aS,4S,6S,7aR)-3a,5,5-trimethylhexahydro-4,6-methanobenzo[d][1,3,2]dioxaborol-2-yl)butan-1-amine (36-6, 75 mg, 2.06 mmol) in THF (10 mL) and NMM (0.34 mL, 2.47 mmol) were added at −10° C., then stirred for 1 h at same temperature and finally at ambient temperature for 1.5 h. The reaction mixture was diluted with ethyl acetate and was washed with cold aqueous 0.1 N HCl solution, followed by 10% aqueous K₂CO₃ solution and finally by brine. Organic phase was dried over Na₂SO₄ and evaporated under reduced pressure at 30° C. to afford tert-butyl ((R)-3-(N,N-dimethylsulfamoyl)-1-oxo-1-(((R)-4-phenyl-1-((3aS,4S,6S,7aR)-3a,5,5-trimethylhexahydro-4,6-methanobenzo[d][1,3,2]dioxaborol-2-yl)butyl) amino)propan-2-yl)carbamate (36-7, 1.2 g). [M−H]⁻=6043.

(R)-2-amino-3-(N,N-dimethylsulfamoyl)-N—((R)-4-phenyl-1-((3aS,4S,6S,7aR)-3a,5,5 trimethyl hexahydro-4,6-methanobenzo[d][1,3,2]dioxaborol-2-yl)butyl)propanamide-4-methyl benzenesulfonate [Step 6]: To the solution tert-butyl ((R)-3-(N,N-dimethylsulfamoyl)-1-oxo-1-(((R)-4-phenyl-1-((3aS,4S,6S,7aR)-3a,5,5-trimethylhexahydro-4,6-methanobenzo [d][1,3,2]dioxaborol-2-yl)butyl)amino)propan-2-yl)carbamate (36-7, 1.20 g, 1.98 mmol) in acetonitrile (10 mL) was added PTSA (753 mg, 3.96 mmol) at 0° C. and then stirred at ambient temperature for 2 h. Reaction was monitored by LCMS. Reaction mass was concentrated under reduced pressure to afford (R)-2-amino-3-(N,N-dimethylsulfamoyl)-N—((R)-4-phenyl-1-((3aS,4S,6S,7aR)-3a,5,5 trimethylhexahydro-4,6-methanobenzo[d][1,3,2]dioxaborol-2-yl)butyl) propanamide-4-methylbenzenesulfonate (36-8, 1.7 g). This product was forwarded to the next step without further purification. [M−H]⁻=504.4.

Synthesis of N—((R)-3-(N,N-dimethylsulfamoyl)-1-oxo-1-(((R)-4-phenyl-1-((3aS,4S,6S,7aR)-3a,5,5,7a-tetramethylhexahydro-4,6-methanobenzo[d][1,3,2]dioxaborol-2-yl)butyl)amino) propan-2-yl)pyrazine-2-carboxamide [Step 7]: To a stirred solution of afford (R)-2-amino-3-(N,N-dimethylsulfamoyl)-N—((R)-4-phenyl-1-((3aS,4S,6S,7aR)-3a,5,5 trimethylhexahydro-4,6-methanobenzo[d][1,3,2]dioxaborol-2-yl)butyl)propanamide 4-methylbenzenesulfonate (36-8, 1.25 g) in DCM (20 mL) was added NMM (0.50 mL, 3.69 mmol) at 0-5° C. After 10 min pyrazine-2-carbonyl chloride (36-9, 342 mg, 2.40 mmol) was added into that reaction mixture. The reaction was stirred at ambient temperature for 2 h. The reaction mixture was diluted DCM (100 ml) and washed with saturated aqueous solution of NaHCO₃, followed by brine solution. The organic phase was dried over anhydrous Na₂SO₄, filtered and concentrated under reduced pressure to afford N—((R)-3-(N,N-dimethylsulfamoyl)-1-oxo-1-(((R)-4-phenyl-1-((3aS,4S,6S,7aR)-3a,5,5,7a-tetramethylhexahydro-4,6-methanobenzo[d][1,3,2]dioxaborol-2-yl)butyl)amino)propan-2-yl) pyrazine-2-carboxamide (36-10, 600 mg). The product was directly used in next step next step without purification. [M−H]⁻=610.4.

Synthesis of ((R)-1-((R)-3-(N,N-dimethylsulfamoyl)-2-(pyrazine-2-carboxamido) propanamido)-4-phenylbutyl)boronic acid [Step 8]: To a solution of N—((R)-3-(N,N-dimethylsulfamoyl)-1-oxo-1-(((R)-4-phenyl-1-((3aS,4S,6S,7aR)-3a,5,5,7a-tetramethylhexahydro-4,6-methanobenzo[d][1,3,2]dioxaborol-2-yl)butyl)amino)propan-2-yl)pyrazine-2-carboxamide (36-10, 313 mg, 0.51 mmol) in acetone (3 mL) was added methylboronic acid (11, 306 mg, 5.12 mmol), followed by drop wise addition of 0.2 M aqueous HCl (2.5 mL). The reaction mixture was allowed to stir at ambient temperature for 16 h. Reaction was monitored by LCMS. Reaction mass was concentrated under reduced pressure and lyophilized and finally purified by reverse phase PREP-HPLC. Pure fraction was concentrated under reduced pressure at 30° C. and lyophilized to afford ((R)-1-((R)-3-(N,N-dimethylsulfamoyl)-2-(pyrazine-2-carboxamido)propanamido)-4-phenylbutyl)boronic acid (Compound 36, 80 mg). ¹H NMR (400 MHz, CD₃OD) δ 9.24 (s, 1H), 8.81-8.80 (d, 1H), 8.70 (s, 1H), 7.21-7.18 (m, 2H), 7.14-7.07 (m, 3H), 5.26-5.23 (m, 1H), 3.74-3.61 (m, 2H), 2.85 (m, δ_{H}), 2.77-2.74 (m, 1H), 2.61-2.56 (m, 2H), 1.64-1.47 (in, 4H). [M−H]⁻=476.3.

### Example 37: ((R)-1-((R)-4-(methylsulfonyl)-2-(pyrazine-2-carboxamido)butanamido)-4-phenylbutyl)boronic acid

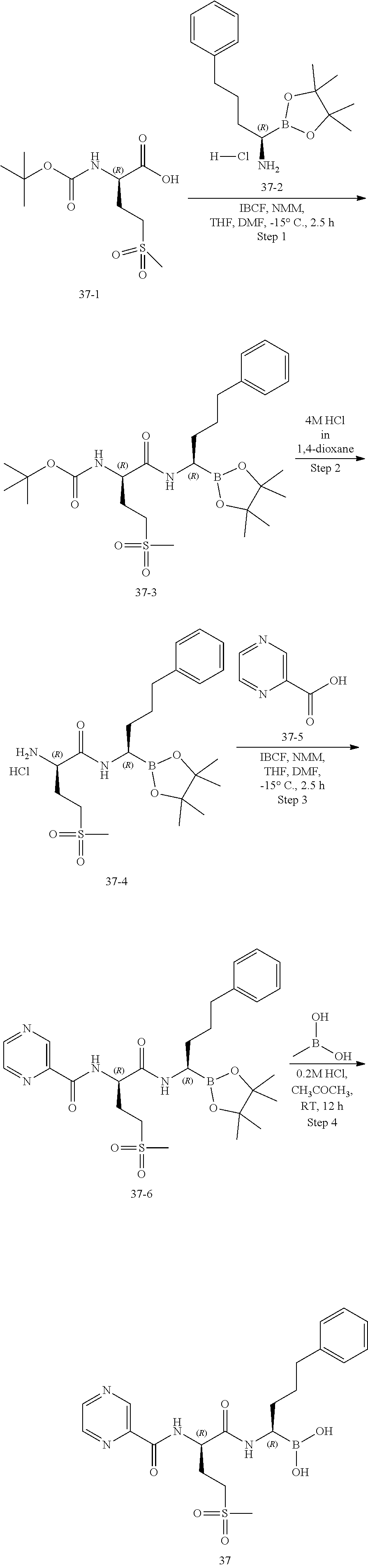

Synthesis of tert-butyl ((R)-4-(methylsulfonyl)-1-oxo-1-(((R)-4-phenyl-1-(4,4,5,5-tetramethyl-1,3,2-dioxaborolan-2-yl)butyl)amino)butan-2-yl)carbamate [Step 1]: To a stirred solution of (R)-2-((tert-butoxycarbonyl)amino)-4-(methylsulfonyl)butanoic acid (37-1, 495 mg, 1.7 mmol) in THF (5 mL) was added IBCF (0.24 mL, 1.7 mmol) followed by NMM (0.19 mL, 1.7 mmol) at −15° C. and stirred for 30 min. A solution of (1R)-4-phenyl-1-(4,4,5,5-tetramethyl-1,3,2-dioxaborolan-2-yl)butan-1-amine;hydrochloride (37-2, 500 mg, 1.6 mmol) in dimethylformamide (1 mL) was added dropwise followed by NMM (0.18 mL, 1.6 mmol) to the reaction mixture at −15° C. and then allowed to warm to 0° C. and stirred for 2 h. The reaction mixture was diluted with ethyl acetate and washed with 0.1 M HCl (twice), 5% aqueous K₂CO₃ solution (twice), water (twice) and brine, dried over anhydrous Na₂SO₄ and concentrated under reduced pressure to afford tert-butyl ((R)-4-(methylsulfonyl)-1-oxo-1-(((R)-4-phenyl-1-(4,4,5,5-tetramethyl-1,3,2-dioxaborolan-2-yl)butyl)amino)butan-2-yl)carbamate (37-3, 650 mg). [M−H]⁻=537.2.

Synthesis of (R)-2-amino-4-(methylsulfonyl)-N—((R)-4-phenyl-1-(4,4,5,5-tetramethyl-1,3,2-dioxaborolan-2-yl)butyl)butanamide [Step 2]: To a stirred solution of tert-butyl ((R)-4-(methylsulfonyl)-1-oxo-1-(((R)-4-phenyl-1-(4,4,5,5-tetramethyl-1,3,2-dioxaborolan-2-yl)butyl) amino)butan-2-yl)carbamate (37-3, 800 mg, 1.5 mmol) in 1,4-dioxane (8 mL) was added 4 M HCl in 1,4-dioxane (3.7 mL) dropwise at ice cold condition. The reaction mixture was stirred at ambient temperature for 12 h. Volatiles were evaporated under reduced pressure to afford (R)-2-amino-4-(methylsulfonyl)-N—((R)-4-phenyl-1-(4,4,5,5-tetramethyl-1,3,2-dioxaborolan-2-yl)butyl) butanamide (37-4, 700 mg). [M−H]⁻=437.4.

Synthesis of N—((R)-4-(methylsulfonyl)-1-oxo-1-(((R)-4-phenyl-1-(4,4,5,5-tetramethyl-1,3,2-dioxaborolan-2-yl)butyl)amino)butan-2-yl)pyrazine-2-carboxamide [Step 3]: To a stirred solution of pyrazine-2-carboxylic acid (37-5, 220 mg, 1.7 mmol) in THF (8 mL) was added IBCF (0.23 mL, 1.7 mmol) followed by NMM (0.19 mL, 1.7 mmol) at −15° C. and stirred for 30 min. A solution of (2R)-2-amino-4-methylsulfonyl-N-[(1R)-4-phenyl-1-(4,4,5,5-tetramethyl-1,3,2-dioxaborolan-2-yl)butyl]butanamide;hydrochloride (37-4, 760 mg, 1.60 mmol) in dimethylformamide (1 mL) was added dropwise followed by NMM (0.18 mL, 1.6 mmol) to the reaction mixture at −15° C. and then allowed to warm to 0° C. and stirred for 2 h. The reaction mixture was diluted with ethyl acetate and washed with 0.1N HCl (twice), 5% aqueous K₂CO₃ solution (twice), water (twice) and brine, dried over anhydrous Na₂SO₄ and concentrated under reduced pressure. The product was purified by PREP-HPLC purification to afford N—((R)-4-(methylsulfonyl)-1-oxo-1-(((R)-4-phenyl-1-(4,4,5,5-tetramethyl-1,3,2-dioxaborolan-2-yl)butyl) amino)butan-2-yl)pyrazine-2-carboxamide (37-6, 60 mg). [M−H]⁻=543.4 and 461.2. LCMS showed mixture of boronate ester and corresponding boronic acid.

Synthesis of ((R)-1-((R)-4-(methylsulfonyl)-2-(pyrazine-2-carboxamido)butanamido)-4-phenylbutyl)boronic acid [Step 4]: To a solution of N—((R)-4-(methylsulfonyl)-1-oxo-1-(((R)-4-phenyl-1-(4,4,5,5-tetramethyl-1,3,2-dioxaborolan-2-yl)butyl)amino)butan-2-yl)pyrazine-2-carboxamide (37-6, 55 mg, 0.1 mmol) and methylboronic acid (60 mg, 1.0 mmol) in acetone (2 mL) was added 0.2 M HCl (2 mL) and stirred at ambient temperature for 16 h. All volatiles were evaporated under reduced pressure and purified by PREP-HPLC purification and lyophilized to give ((R)-1-((R)-4-(methylsulfonyl)-2-(pyrazine-2-carboxamido)butanamido)-4-phenylbutyl) boronic acid (Compound 37, 32 mg). [M−H]⁻=461.2. ¹H NMR (400 MHz, CD₃OD) δ_{H} 9.22 (s, 1H), 8.79 (d, 1H), 8.70 (s, 1H), 7.22-7.07 (m, 5H), 4.99 (t, 1H), 3.29-3.22 (m, 2H), 2.97 (s, 3H), 2.69 (t, 1H), 2.64-2.34 (m, 4H), 1.66-1.57 (m, 4H).

### Example 38: ((R)-1-((R)-3-methoxy-2-(pyrazine-2-carboxamido)propanamido)-3-methyl butyl)boronic acid

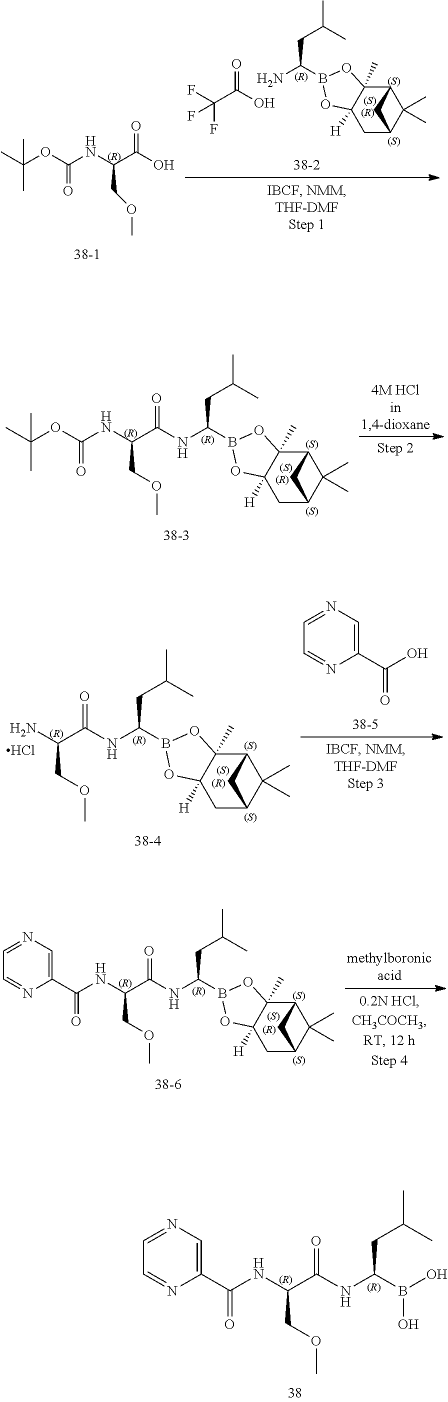

Synthesis of tert-butyl ((R)-3-methoxy-1-(((R)-3-methyl-1-((3aS,4S,6S,7aR)-3a,5,5-trimethyl hexahydro-4,6-methanobenzo[d][1,3,2]dioxaborol-2-yl)butyl)amino)-1-oxopropan-2-yl) carbamate [Step 1]: To a solution of N-(tert-butoxycarbonyl)-O-methyl-D-serine (38-1, 318 mg, 1.4 mmol) in THF (8 mL) was added IBCF (0.20 mL, 1.4 mmol) followed by NMM (0.20 mL, 1.4 mmol) at −15° C. After 45 min, a solution of (R)-3-methyl-1-((3aS,4S,6S,7aR)-3a,5,5-trimethylhexahydro-4,6-methanobenzo[d][1,3,2]dioxaborol-2-yl)butan-1-amine 2,2,2-trifluoro acetate (38-2, 500 mg, 1.3 mmol) in DMF (2 mL) was added dropwise followed by NMM (0.2 mL, 1.3 mmol). After stirring for 1 h at the same temperature, the reaction was diluted with EtOAc and washed successively with 0.1 N aq. HCl (×2), 5% aq. K2CO3 (×2), water (×2) and brine (×2). The organic phase was dried over anhydrous Na2SO4, filtered and concentrated under reduced pressure. The crude tert-butyl ((R)-3-methoxy-1-(((R)-3-methyl-1-((3aS,4S,6S,7aR)-3a,5,5-trimethylhexahydro-4,6-methanobenzo[d][1,3,2]dioxaborol-2-yl)butyl)amino)-1-oxopropan-2-yl)carbamate (38-3, 450 mg, 73%) was used in the next step without further purification. [M−H]⁻=465.5.

Synthesis of (R)-2-amino-3-methoxy-N—((R)-3-methyl-1-((3aS,4S,6S,7aR)-3a,5,5-trimethyl hexahydro-4,6-methanobenzo[d][1,3,2]dioxaborol-2-yl)butyl)propanamide hydrochloride [Step 2]: To an ice-cold solution of crude tert-butyl ((R)-3-methoxy-1-(((R)-3-methyl-1-((3aS,4S,6S,7aR)-3a,5,5-trimethylhexahydro-4,6-methanobenzo[d][1,3,2]dioxaborol-2-yl)butyl)amino)-1-oxopropan-2-yl)carbamate (38-3, 450 mg, 1.0 mmol) in 1,4-dioxane (10 mL) was added 4M HCl in 1,4-dioxane (10 mL, 40.0 mmol), and the mixture was stirred at 25° C. for 16 h. The reaction mixture was dried under vacuo to afford crude (R)-2-amino-3-methoxy-N—((R)-3-methyl-1-((3aS,4S,6S,7aR)-3a,5,5-trimethylhexahydro-4,6-methanobenzo[d][1,3,2]dioxaborol-2-yl)butyl)propanamide hydrochloride (38-4, 400 mg), which was used in the next step without further purification. [M−H]⁻=365.5.

Synthesis of N—((R)-3-methoxy-1-(((R)-3-methyl-1-((3aS,4S,6S,7aR)-3a,5,5-trimethyl hexa hydro-4,6-methanobenzo[d][1,3,2]dioxaborol-2-yl)butyl)amino)-1-oxopropan-2-yl) pyrazine-2-carboxamide [Step 3]: To a solution of pyrazine-2-carboxylic acid (38-5, 136 mg, 1.1 mmol) in THF (4 mL) was added IBCF (0.15 mL, 1.1 mmol) followed by NMM (0.15 mL, 1.1 mmol) at −15° C. After 45 min, a solution of (R)-2-amino-3-methoxy-N—((R)-3-methyl-1-((3aS,4S,6S,7aR)-3a,5,5-trimethylhexahydro-4,6-methanobenzo[d][1,3,2]dioxaborol-2-yl)butyl) propanamide hydrochloride (38-4, 400 mg, 1.0 mmol) in DMF (1 mL) was added dropwise followed by NMM (0.14 mL, 1.0 mmol). After stirring for 1 h at the same temperature, the reaction was diluted with EtOAc and washed successively with 0.1 N aq. HCl (×2), 5% aq. K2CO3 (×2), water (×2) and brine (×2). The organic phase was dried over anhydrous Na2SO4, filtered and concentrated under reduced pressure. The crude was purified by prep HPLC (RP) to afford N—((R)-3-methoxy-1-(((R)-3-methyl-1-((3aS,4S,6S,7aR)-3a,5,5-trimethylhexahydro-4,6-methano benzo[d][1,3,2]dioxaborol-2-yl)butyl)amino)-1-oxopropan-2-yl)pyrazine-2-carboxamide (38-6, 220 mg). [M+H]⁺=472.8.

Synthesis of ((R)-1-((R)-3-methoxy-2-(pyrazine-2-carboxamido)propanamido)-3-methyl butyl)boronic acid [Step 4]: To a solution of N—((R)-3-methoxy-1-(((R)-3-methyl-1-((3aS,4S,6S,7aR)-3a,5,5-trimethylhexahydro-4,6-methanobenzo[d][1,3,2]dioxaborol-2-yl)butyl)amino)-1-oxopropan-2-yl)pyrazine-2-carboxamide (38-6, 200 mg, 0.4 mmol) in acetone (5 mL) was added methylboronic acid (380 mg, 6.4 mmol) followed by 0.2N HCl (5.0 mL, 0.4 mmol) at 0° C. After stirring for 16 h at 25° C., the reaction mixture was concentrated under reduced pressure, and then lyophilized. Purified of the material by prep HPLC (RP) followed by lyophilization afforded ((R)-1-((R)-3-methoxy-2-(pyrazine-2-carboxamido)propanamido)-3-methylbutyl)boronic acid (Compound 38, 90 mg). [M−H]⁻=337.08. ¹H NMR (400 MHz, CD₃OD): δ 9.25-9.24 (m, 1H), 8.82-8.81 (m, 1H), 8.71-8.70 (m, 1H), 5.00 (t, 1H), 3.89 (dd, 1H), 3.78 (dd, 1H), 3.40 (s, 3H), 2.75 (t, 1H), 1.65 (m, 1H), 1.37-1.33 (m, 2H), 0.90-0.88 (m, δ_{H}).

### Examples 39 and 40: ((R)-1-((R)-3-hydroxy-2-(pyrazine-2-carboxamido)propanamido)-4-(4-hydroxyphenyl)butyl)boronic acid and ((R)-4-(4-hydroxyphenyl)-1-((R)-3-methoxy-2-(pyrazine-2-carboxamido)propanamido)butyl)boronic acid

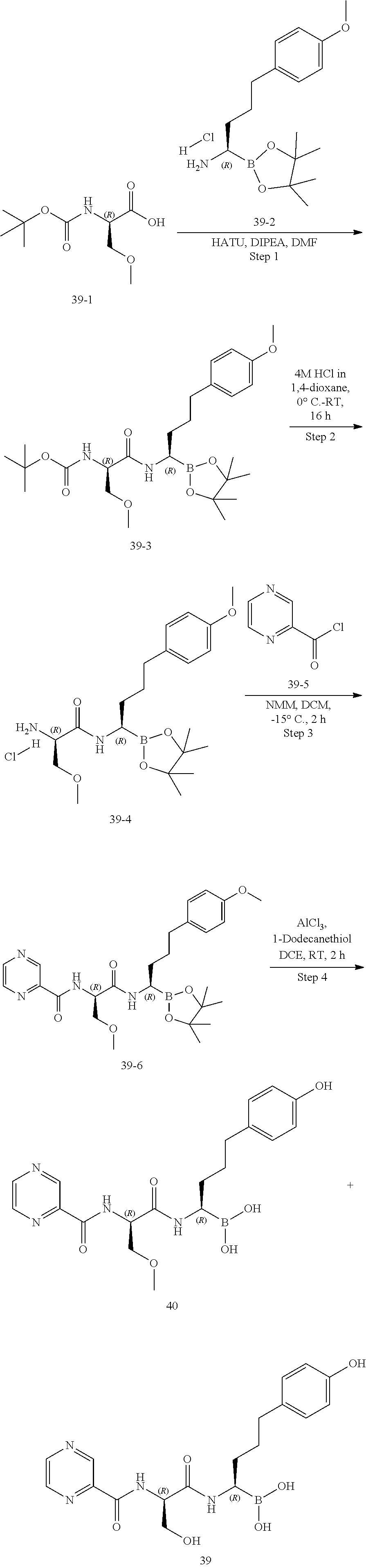

Synthesis of tert-butyl ((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)carbamate [Step 1]: To a stirred solution of N-(tert-butoxycarbonyl)-O-methyl-D-serine (39-1, 353 mg, 1.6 mmol) in THF (2 mL) was added IBCF (0.12 mL, 1.3 mmol) followed by NMM (0.2 mL, 1.6 mmol) at −15° C. and stirred for 30 min. A solution of (R)-4-(4-methoxyphenyl)-1-(4,4,5,5-tetramethyl-1,3,2-dioxaborolan-2-yl)butan-1-amine hydrochloride (39-2, 500 mg, 1.5 mmol) in THF (1 mL) was added dropwise followed by NMM (0.2 mL, 1.5 mmol) to the reaction mixture at −15° C. and the reaction mixture was allowed to warm to 0° C. and stirred for 2 h. The reaction mixture was diluted with ethyl acetate and washed with 0.1N HCl (twice), 5% aqueous K₂CO₃ solution (twice), water (twice) and brine. The organic extract was dried over anhydrous Na₂SO₄ and concentrated under reduced pressure to afford tert-butyl ((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)carbamate (39-3, 520 mg). [M−H]⁻=505.4.

Synthesis of (R)-2-amino-3-methoxy-N—((R)-4-(4-methoxyphenyl)-1-(4,4,5,5-tetramethyl-1,3,2-dioxaborolan-2-yl)butyl)propanamide hydrochloride [Step 2]: To a stirred solution of tert-butyl ((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)carbamate (39-3, 530 mg, 1.1 mmol) in 1,4-dioxane (5 mL) was added 4N HCl in 1,4-dioxane (2.6 mL, 10.5 mmol) at 0° C. under nitrogen atmosphere. The reaction mixture was stirred at ambient temperature for 2 h. The reaction mixture was concentrated under reduced pressure and triturated with diethyl ether and dried to afford (R)-2-amino-3-methoxy-N—((R)-4-(4-methoxyphenyl)-1-(4,4,5,5-tetramethyl-1,3,2-dioxaborolan-2-yl)butyl)propanamide hydrochloride (39-4, 412 mg). [M−H]⁻=405.5.

Synthesis of N—((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)pyrazine-2-carboxamide [Step 3]: To a stirred solution of (R)-2-amino-3-methoxy-N—((R)-4-(4-methoxyphenyl)-1-(4,4,5,5-tetramethyl-1,3,2-dioxaborolan-2-yl)butyl)propanamide hydrochloride (39-4, 530 mg, 1.2 mmol) in 0CM (5 mL) was added NMM (0.3 mL, 3.6 mmol) at 0° C. Pyrazine-2-carbonyl chloride (39-5, 256 mg, 1.8 mmol) was added to the reaction mixture and stirred at ambient temperature for 2 h. The reaction mixture was diluted with DCM and washed with saturated aqueous NaHCO₃ solution and brine, dried over anhydrous Na₂SO₄ and concentrated under reduced pressure to afford N—((R)-3-methoxy-1-(((R)-4-(4-methoxyphenyl)-1-(4,4,5,5-tetramethyl-1,3,2-dioxaborolan-2-yl)butyl) amino)-1-oxopropan-2-yl)pyrazine-2-carboxamide (39-6, 445 mg). [M−H]⁻=511.3.

Synthesis of ((R)-4-(4-hydroxyphenyl)-1-((R)-3-methoxy-2-(pyrazine-2-carboxamido) propanamido)butyl)boronic acid and ((R)-1-((R)-3-hydroxy-2-(pyrazine-2-carboxamido) propanamido)-4-(4-hydroxyphenyl)butyl)boronic acid [Step 4]: To a stirred solution of AlCl₃ (1.7 g, 12.6 mmol) in DCE (20 mL) was added 1-dodecanethiol (1.5 mL, 6.3 mmol) at 0° C. under argon atmosphere and stirred at same temperature for 30 min. A solution of N—((R)-3-methoxy-1-(((R)-4-(4-methoxyphenyl)-1-(4,4,5,5-tetramethyl-1,3,2-dioxaborolan-2-yl)butyl)amino)-1-oxopropan-2-yl)pyrazine-2-carboxamide (39-6, 270 mg, 0.5 mmol) in DCE (5 mL) was added drop wise and stirred at ambient temperature for 1 h. The reaction mixture was filtered and concentrated under reduced pressure. The product was purified by prep HPLC purification and lyophilized to afford:

((R)-1-((R)-3-hydroxy-2-(pyrazine-2-carboxamido)propanamido)-4-(4-hydroxyphenyl) butyl)boronic acid (Compound 39, 7 mg). [M−H]⁻: 401.5. ¹H NMR (400 MHz, DMSO-d₆+2 drop D₂O at 80° C.) δ_{H}: 9.18 (s, 1H), 8.86 (s, 1H), 8.73 (s, 1H), 6.93-6.91 (m, 2H), 6.65-6.63 (m, 2H), 4.50 (t, 1H), 3.75-3.74 (m, 1H), 3.71-3.70 (m, 1H), 2.50-2.49 (m, 3H), 1.52-1.49 (m, 4H); and

((R)-4-(4-hydroxyphenyl)-1-((R)-3-methoxy-2-(pyrazine-2-carboxamido)propanamido) butyl)boronic acid (Compound 40, 5 mg). [M−H]⁻: 415.3. ¹H NMR (400 MHz, DMSO-d₆+2 drop D₂O at 80° C.) δ_{H}: 9.17 (s, 1H), 8.85 (s, 1H), 8.71 (s, 1H), 6.92-6.91 (m, 2H), 6.65-6.63 (m, 2H), 4.67-4.65 (m, 1H), 3.70-3.67 (m, 1H), 3.64-3.60 (m, 1H), 3.25 (s, 3H), 2.54-2.41 (m, 3H), 1.51-1.48 (m, 4H).

### Example 41: ((R)-1-((R)-3-methoxy-2-((R)-tetrahydro-2H-pyran-2-carboxamido) propanamido)-4-phenylbutyl)boronic acid

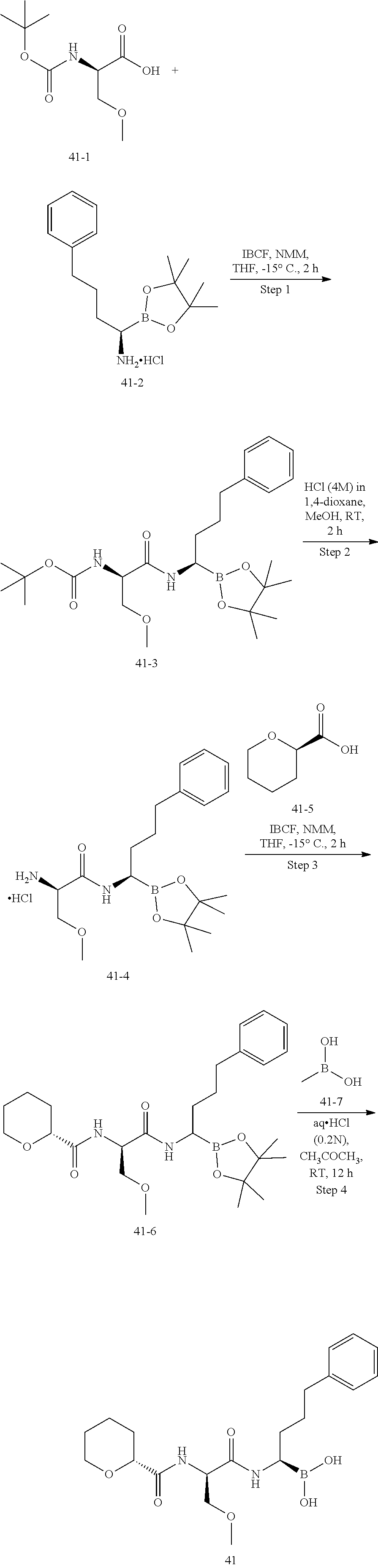

Synthesis of tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate [Step 1]: To a stirred solution of N-(tert-butoxycarbonyl)-O-methyl-D-serine (41-1, 500 mg, 2.28 mmol) in tetrahydrofuran (10 mL) at −15° C. were added isobutyl chloroformate (0.3 mL, 2.28 mmol) and N-methyl morpholine (0.28 mL, 2.50 mmol). The reaction mixture was stirred for 30 min and a solution of (R)-4-phenyl-1-(4,4,5,5-tetramethyl-1,3,2-dioxaborolan-2-yl) butan-1-amine hydrochloride (41-2, 710 mg, 2.28 mmol) and N-methyl morpholine (0.3 mL, 2.50 mmol) in tetrahydrofuran (1 mL) was added at the same temperature. The resulting reaction mixture warmed to 0° C. and allowed to stir for additional 2 h. The reaction mixture was neutralized with aqueous solution of hydrochloric acid (0.1 N) and extracted with ethyl acetate. The combined extracts were washed with 5% aqueous solution of potassium carbonate, water, brine, dried over anhydrous sodium sulphate, filtered and evaporated under reduced pressure to afford tert-butyl ((R)-3-methoxy-1-oxo-1-(((R)-4-phenyl-1-(4,4,5,5-tetramethyl-1,3,2-dioxaborolan-2-yl) butyl) amino) propan-2-yl) carbamate (41-3, 520 mg). The product was carried to next step without further purification. [M−H]⁻: 475.4.

Synthesis of (R)-2-amino-3-methoxy-N—((R)-4-phenyl-1-(4,4,5,5-tetramethyl-1,3,2-dioxaborolan-2-yl)butyl)propanamide hydrochloride [Step 2]: To a solution of tert-butyl ((R)-3-methoxy-1-oxo-1-(((R)-4-phenyl-1-(4,4,5,5-tetramethyl-1,3,2-dioxaborolan-2-yl)butyl)amino) propan-2-yl)carbamate (41-3, 520 mg, 1.09 mmol) in 1,4-dioxane (5 mL) at 0° C. was added hydrochloric acid (4 M in 1,4-dioxane, 2.5 mL, 10.91 mmol). The reaction mixture was allowed to stir at 25° C. for 6 h. The volatiles were evaporated under reduced pressure to give (R)-2-amino-3-methoxy-N—((R)-4-phenyl-1-(4,4,5,5-tetramethyl-1,3,2-dioxaborolan-2-yl)butyl) propanamide hydrochloride (41-4, 350 mg). The product was carried to next step without further purification. [M−H]⁻: 374.4.

Synthesis of (R)—N—((R)-3-methoxy-1-oxo-1-(((R)-4-phenyl-1-(4,4,5,5-tetramethyl-1,3,2-dioxaborolan-2-yl)butyl)amino)propan-2-yl)tetrahydro-2H-pyran-2-carboxamide [Step 3]: To a stirred solution of (R)-tetrahydro-2H-pyran-2-carboxylic acid (41-5, 130 mg, 0.99 mmol) in tetrahydrofuran (8 mL) at −15° C. were added isobutyl chloroformate (0.15 mL, 0.99 mmol) and N-methyl morpholine (0.12 mL, 1.09 mmol). The reaction mixture was stirred at the same temperature for 30 min and a solution of (R)-2-amino-3-methoxy-N—((R)-4-phenyl-1-(4,4,5,5-tetramethyl-1,3,2-dioxaborolan-2-yl)butyl)propenamide hydrochloride (41-4, 412 mg, 0.99 mmol) and N-methyl morpholine (0.12 mL, 1.09 mmol) in tetrahydrofuran (1 mL) was added. The resulting reaction mixture warmed to 0° C. and allowed to stir for additional 2 h. The reaction mixture was neutralized with aqueous solution of hydrochloric acid (0.1 N) and extracted with ethyl acetate. The combined extracts were washed with 5% aqueous solution of potassium carbonate, water, brine, dried over anhydrous sodium sulphate, filtered and evaporated under reduced pressure to afford (R)—N—((R)-3-methoxy-1-oxo-1-(((R)-4-phenyl-1-(4,4,5,5-tetramethyl-1,3,2-dioxaborolan-2-yl)butyl)amino)propan-2-yl)tetrahydro-2H-pyran-2-carboxamide (41-6, 210 mg). The product was carried to next step without further purification. [M−H]⁻: 487.4.

Synthesis of ((R)-1-((R)-3-methoxy-2-((R)-tetrahydro-2H-pyran-2-carboxamido) propanamido)-4-phenylbutyl)boronic acid [Step-4]: To a stirred solution of (R)—N—((R)-3-methoxy-1-oxo-1-(((R)-4-phenyl-1-(4,4,5,5-tetramethyl-1,3,2-dioxaborolan-2-yl)butyl)amino) propan-2-yl)tetrahydro-2H-pyran-2-carboxamide (41-6, 210 mg, 0.43 mmol) and methyl boronic acid (41-7, 257 mg, 4.3 mmol) in acetone (3 mL), was added aqueous solution of hydrochloric acid (0.2 N, 3 mL) at 0° C. The reaction mixture was stirred at ambient temperature for 16 h. The volatiles were evaporated under reduced pressure and the product was purified by reverse phase prep-HPLC purification to give ((R)-1-((R)-3-methoxy-2-(pyrazine-2-carboxamido)propanamido)-4-phenylbutyl)boronic acid (Compound 41, 12 mg). [M−H]⁻=405.3. ¹H NMR (400 MHz, DMSO-d6 and two drops of D₂O at 80° C.): δ_{H} 7.26-7.23 (m, 2H), 7.19-7.14 (m, 3H), 4.41 (t, 1H), 3.99-3.96 (m, 1H), 3.78-3.53 (m, 1H), 3.49-3.45 (m, 2H), 3.25-3.19 (m, 4H), 2.60-2.55 (m, 2H), 1.87-1.78 (m, 2H), 1.56-1.50 (m, 8H), 1.35-1.30 (m, 1H).

### Example 42: ((R)-1-((R)-3-methoxy-2-(6-methylpicolinamido)propanamido)-3-phenoxy propyl)boronic acid

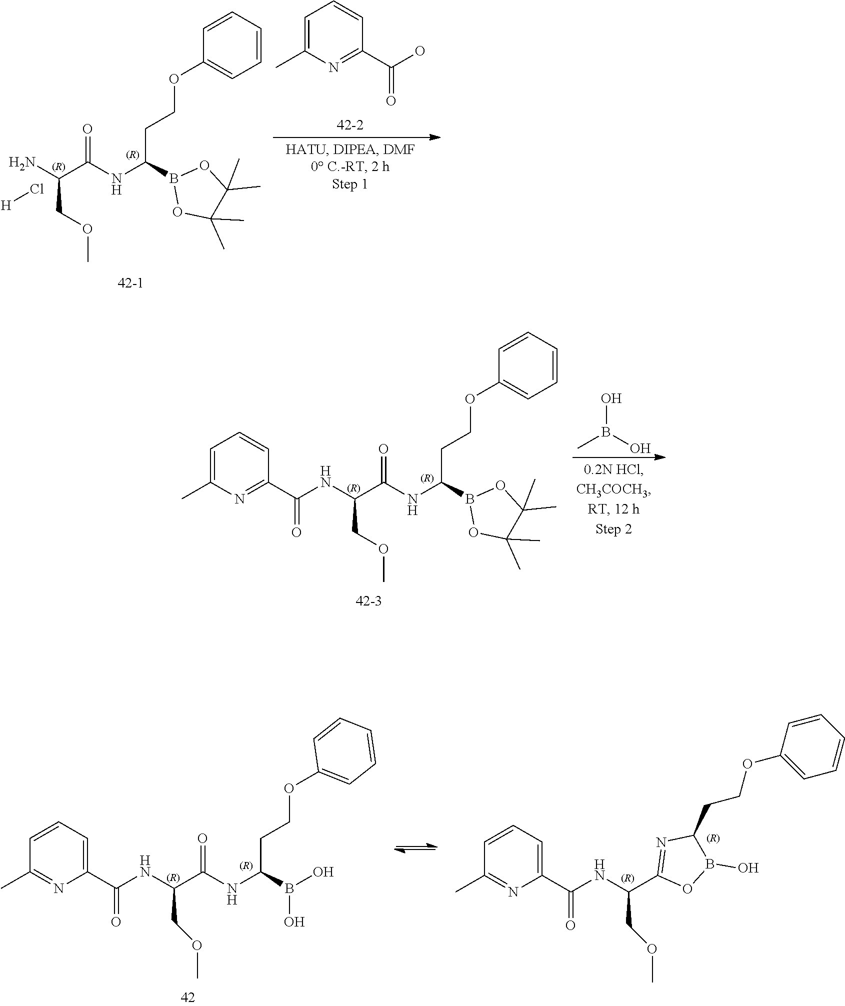

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)-6-methylpicolinamide [Step 1]: To a stirred solution of 6-methylpicolinic acid (42-2, 160 mg, 1.67 mmol) in DMF (5 mL) were added HATU (887 mg, 2.33 mmol) and (R)-2-amino-3-methoxy-N—((R)-3-phenoxy-1-(4,4,5,5-tetramethyl-1,3,2-dioxaborolan-2-yl)propyl)propanamide hydrochloride (42-1, 580 mg, 1.40 mmol) followed by N,N-diisopropylethylamine (0.63 mL, 3.50 mmol) at 0° C. and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)-6-methyl picolinamide (42-3, 700 mg) which was used in the next step without further purification. [M−H]⁻=496.2.

Synthesis of ((R)-1-((R)-3-methoxy-2-(6-methylpicolinamido)propanamido)-3-phenoxy propyl)boronic acid [Step 2]: To a solution of N—((R)-3-methoxy-1-oxo-1-(((R)-3-phenoxy-1-(4,4,5,5-tetramethyl-1,3,2-dioxaborolan-2-yl)propyl)amino)propan-2-yl)-6-methylpicolinamide (42-3, 500 mg, 1.01 mmol) in acetone (5 mL) was added methylboronic acid (602 mg, 10.10 mmol) followed by addition of 0.2 N HCl (5 mL) at 0° C. The reaction mixture was stirred at ambient temperature for 5 h. Volatiles were evaporated under reduced pressure and purified by PREP HPLC purification and lyophilized to give ((R)-1-((R)-3-methoxy-2-(6-methylpicolinamido) propanamido)-3-phenoxypropyl)boronic acid (42, 30 mg). [M−H]⁻=414.1. ¹H NMR (400 MHz, MeOD) δ_{H}: 7.91-7.89 (m, 1H), 7.84 (t, 1H), 7.45 (d, 1H), 7.19 (t, 2H), 6.86-6.79 (m, 3H), 4.98 (t, 1H), 4.03 (t, 2H), 3.92-3.84 (m, 1H), 3.79-3.76 (m, 1H), 3.39 (s, 3H), 2.91 (t, 1H), 2.60 (s, 3H), 2.02-1.89 (m, 2H). The analytical data suggests that the final compound is in equilibrium with the oxaborolane derivative.

### Example 43: ((R)-1-((R)-3-methoxy-2-(picolinamido)propanamido)-3-phenoxypropyl) boronic acid

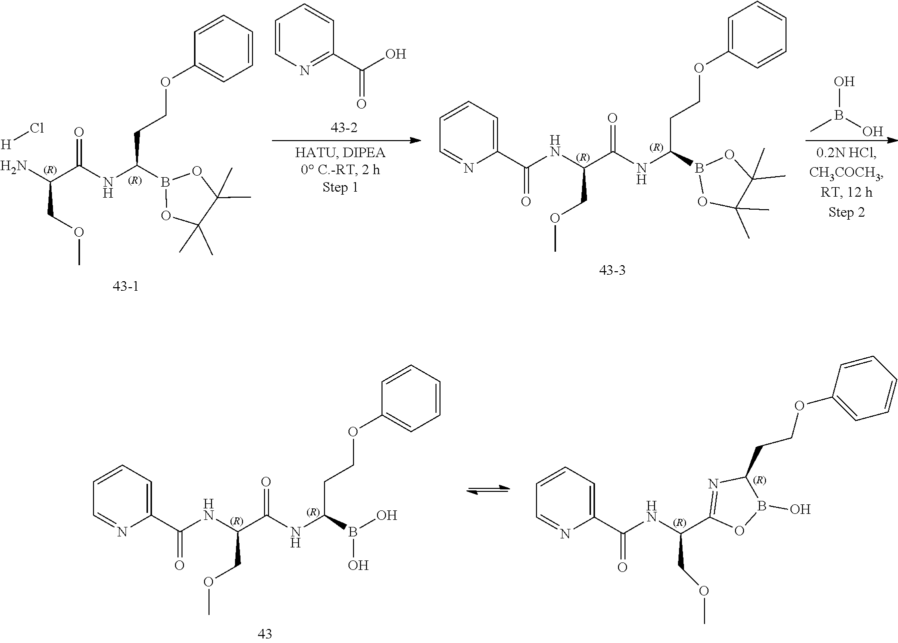

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)picolinamide [Step 1]: To a stirred solution of (R)-2-amino-3-methoxy-N—((R)-3-phenoxy-1-(4,4,5,5-tetramethyl-1,3,2-dioxaborolan-2-yl)propyl) propanamide hydrochloride (43-1, 2.83 g, 6.82 mmol) and pyridine-2-carboxylic acid (43-2, 700 mg, 5.69 mmol) in DMF (15 mL) were added HATU (4.32 g, 11.4 mmol) and DIPEA (3.1 mL, 17.1 mmol) at 0° C. and stirred for 2 h. The reaction mixture was quenched with water and extracted with ethyl acetate. Organic part was further washed with water (thrice) and brine, dried over anhydrous sodium sulfate and evaporated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl) picolinamide (43-3, 800 mg). [M−H]⁻: 481.9. Corresponding boronic acid mass peak was also observed.

Synthesis of ((R)-1-((R)-3-methoxy-2-(picolinamido)propanamido)-3-phenoxypropyl) boronic acid [Step 2]: To a solution of N—((R)-3-methoxy-1-oxo-1-(((R)-3-phenoxy-1-(4,4,5,5-tetramethyl-1,3,2-dioxaborolan-2-yl)propyl)amino)propan-2-yl)picolinamide (43-3, 400 mg, 0.828 mmol) in acetone (20 mL) was added methyl boronic acid (495 mg, 8.28 mmol) followed by drop wise addition of 0.2N HCl (6 mL) and stirred at ambient temperature for overnight. Volatiles were evaporated under reduced pressure and the crude product was purified by prep HPLC purification and lyophilized to afford ((R)-1-((R)-3-methoxy-2-(picolinamido)propanamido)-3-phenoxy propyl)boronic acid (Compound 43, 40 mg). [M−H]⁻: 400.3. 1H NMR (400 MHz, MeOD): δ_{H} 8.66 (d, 1H), 8.11 (m, 1H), 7.99-7.95 (m, 1H), 7.60-7.57 (m, 1H), 7.23 (t, 2H), 6.90-6.76 (m, 3H), 4.98 (s, 1H), 4.04-3.77 (m, 4H), 3.47 (s, 3H), 2.89 (m, 1H), 2.09 (m, 2H). The analytical data suggests that the final compound is in equilibrium with the oxaborolane derivative.

### Example 44: ((R)-1-((R)-3-methoxy-2-(5-methylnicotinamido)propanamido)-3-phenoxy propyl)boronic acid

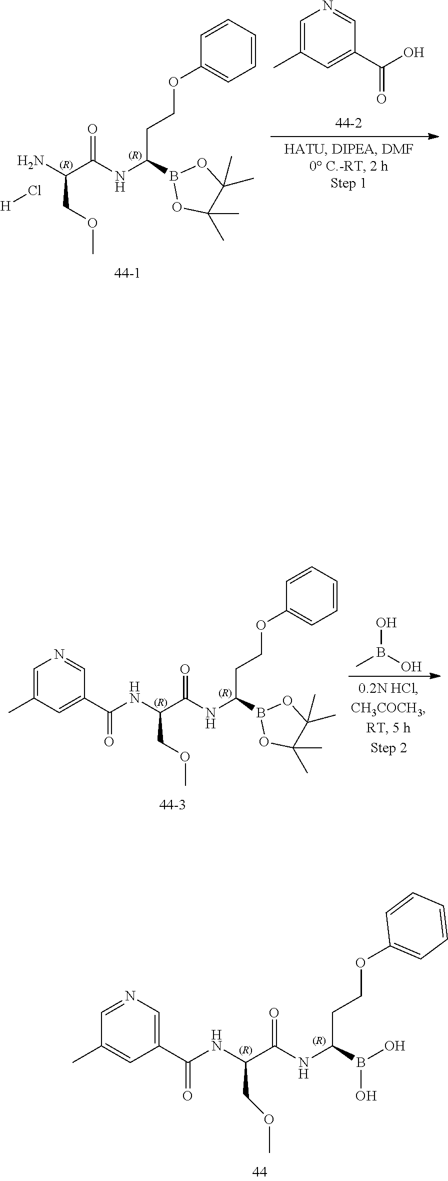

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)-5-methylnicotinamide [Step 1]: To a stirred solution of 5-methylnicotinic acid (44-2, 83 mg, 0.60 mmol) in DMF (5 mL) were added HATU (460 mg, 1.21 mmol) and (R)-2-amino-3-methoxy-N—((R)-3-phenoxy-1-(4,4,5,5-tetramethyl-1,3,2-dioxaborolan-2-yl)propyl)propanamide hydrochloride (44-1, 301 mg, 0.73 mmol) followed by N,N-diisopropylethylamine (0.33 mL, 1.81 mmol) at 0° C. and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)-5-methyl nicotinamide (44-3, 250 mg) which was used in the next step without further purification. [M−H]⁻=496.4.

Synthesis of ((R)-1-((R)-3-methoxy-2-(5-methylnicotinamido)propanamido)-3-phenoxy propyl)boronic acid [Step 2]: To a solution of N—((R)-3-methoxy-1-oxo-1-(((R)-3-phenoxy-1-(4,4,5,5-tetramethyl-1,3,2-dioxaborolan-2-yl)propyl)amino)propan-2-yl)-5-methylnicotinamide (3, 250 mg, 0.51 mmol) in acetone (3 mL) was added methylboronic acid (301 mg, 5.02 mmol) followed by addition of 0.2 N HCl (2.5 mL) at 0° C. The reaction mixture was stirred at ambient temperature for 5 h. Volatiles were evaporated under reduced pressure and purified by PREP HPLC purification and lyophilized to give ((R)-1-((R)-3-methoxy-2-(5-methyl nicotinamido)propanamido)-3-phenoxypropyl)boronic acid (Compound 44, 35 mg). [M−H]⁻=414.1. 1H NMR (400 MHz, MeOD) δ_{H}: 8.84 (s, 1H), 8.58 (s, 1H), 8.19 (s, 1H), 7.21 (t, 2H), 6.89-6.85 (m, 3H), 4.99 (t, 1H), 4.04 (t, 2H), 3.87-3.77 (m, 2H), 3.39 (s, 3H), 2.92 (t, 1H), 2.44 (s, 3H), 2.02-1.90 (m, 2H).

### Example 45: ((R)-1-((R)-3-methoxy-2-(pyrazine-2-carboxamido)propanamido)-3-phenyl propyl)boronic acid

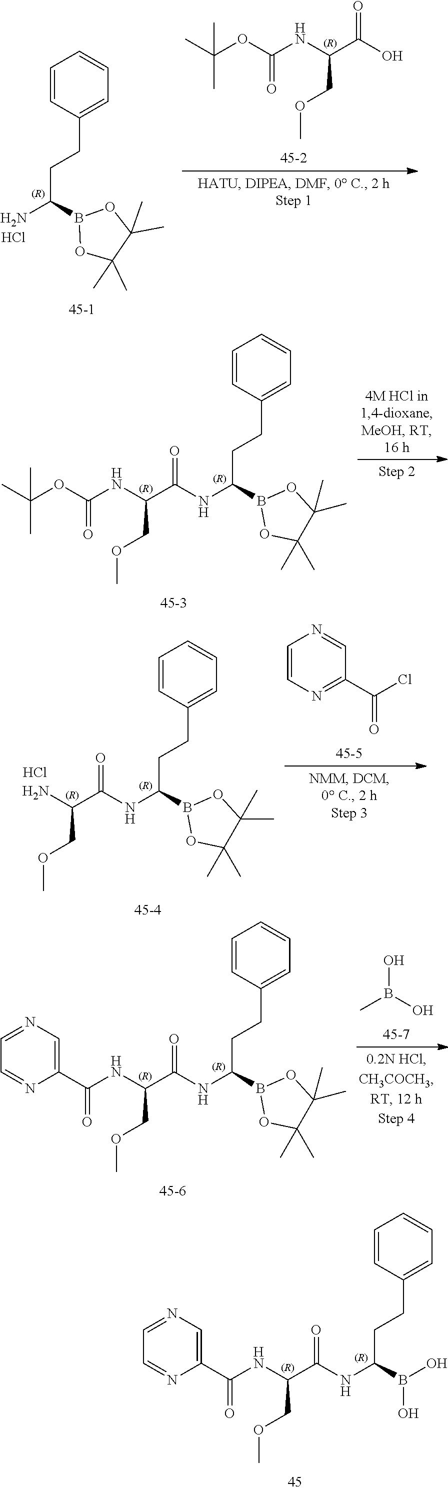

Synthesis of tert-butyl ((R)-3-methoxy-1-oxo-1-(((R)-3-phenyl-1-(4,4,5,5-tetramethyl-1,3,2-dioxaborolan-2-yl)propyl)amino)propan-2-yl)carbamate [Step 1]: To a stirred solution of N-(tert-butoxycarbonyl)-O-methyl-D-serine (45-2, 300 mg, 1.4 mmol) and (R)-3-phenyl-1-(4,4,5,5-tetramethyl-1,3,2-dioxaborolan-2-yl)propan-1-amine, hydrochloride (45-1, 490 mg, 1.6 mmol) in DMF (3 mL) were added HATU (780 mg, 2.0 mmol) and DIPEA (0.7 mL, 4.1 mmol) at 0° C. and stirred for 2 h. The reaction mixture was diluted with ethyl acetate and washed with water (five times) and brine, dried over anhydrous Na₂SO₄ and concentrated under reduced pressure to give tert-butyl ((R)-3-methoxy-1-oxo-1-(((R)-3-phenyl-1-(4,4,5,5-tetramethyl-1,3,2-dioxaborolan-2-yl)propyl)amino)propan-2-yl)carbamate (45-3, 600 mg, 1.3 mmol). [M−H]⁻: 461.4.

Synthesis of (R)-2-amino-3-methoxy-N—((R)-3-phenyl-1-(4,4,5,5-tetramethyl-1,3,2-dioxaborolan-2-yl)propyl)propanamide, hydrochloride [Step 2]: To a stirred solution of tert-butyl ((R)-3-methoxy-1-oxo-1-(((R)-3-phenyl-1-(4,4,5,5-tetramethyl-1,3,2-dioxaborolan-2-yl) propyl)amino)propan-2-yl)carbamate (45-3, 600 mg, 1.3 mmol) in 1,4-dioxane (2 mL) was added 4N HCl in 1,4-dioxine (6.5 mL, 26.0 mmol) at 0° C. and stirred at ambient temperature for 16 h. The reaction mixture was concentrated under reduced pressure and triturated with n-pentane and dried to give (R)-2-amino-3-methoxy-N—((R)-3-phenyl-1-(4,4,5,5-tetramethyl-1,3,2-dioxaborolan-2-yl)propyl)propanamide, hydrochloride (45-4, 500 mg). [M−H]⁻: 279.4. Corresponding boronic acid mass was found.

Synthesis of N—((R)-3-methoxy-1-oxo-1-(((R)-3-phenyl-1-(4,4,5,5-tetramethyl-1,3,2-dioxaborolan-2-yl)propyl)amino)propan-2-yl)pyrazine-2-carboxamide [Step 3]: To a stirred solution of (R)-2-amino-3-methoxy-N—((R)-3-phenyl-1-(4,4,5,5-tetramethyl-1,3,2-dioxaborolan-2-yl)propyl)propanamide, hydrochloride (45-4, 500 mg, 1.2 mmol) in dichloromethane (5 mL) was added NMM (0.3 mL, 2.5 mmol) at 0-5° C. and stirred for 10 min. Pyrazine-2-carbonyl chloride (45-5, 270 mg, 1.9 mmol) was added to the reaction mixture at 0° C. and stirred for 2 h. The reaction mixture was diluted 0CM and washed with 5% aqueous NaHCO₃ solution and brine, dried over anhydrous Na₂SO₄ and concentrated under reduced pressure to afford N—((R)-3-methoxy-1-oxo-1-(((R)-3-phenyl-1-(4,4,5,5-tetramethyl-1,3,2-dioxaborolan-2-yl)propyl)amino) propan-2-yl)pyrazine-2-carboxamide (45-6, 500 mg). [M−H]⁻=467.4. Corresponding boronic acid mass peak was also observed.

Synthesis of ((R)-1-((R)-3-methoxy-2-(pyrazine-2-carboxamido)propanamido)-3-phenyl propyl)boronic acid [Step 4]: To a stirred solution of N—((R)-3-methoxy-1-oxo-1-(((R)-3-phenyl-1-(4,4,5,5-tetramethyl-1,3,2-dioxaborolan-2-yl)propyl)amino)propan-2-yl)pyrazine-2-carboxamide (45-6, 300 mg, 0.6 mmol) in acetone (3 mL) was added methylboronic acid (45-7, 380 mg, 6.4 mmol) followed by 0.2N HCl (2.1 mL, 0.64 mmol) at 0° C. and stirred at ambient temperature for 16 h. The volatiles were evaporated under reduced pressure and the crude product was purified by prep HPLC purification and lyophilized to afford ((R)-1-((R)-3-methoxy-2-(pyrazine-2-carboxamido)propanamido)-3-phenylpropyl)boronic acid (Compound 45, 28 mg). [M−H]⁻: 399.1. ¹H NMR (400 MHz, MeOD) δ_{H}: 9.24 (d, 1H), 8.81 (d, 1H), 8.71-8.70 (m, 1H), 7.23-7.10 (m, 5H), 5.01 (t, 1H), 3.92-3.89 (m, 1H), 3.80-3.77 (m, 1H), 3.40 (s, 3H), 2.68-2.61 (m, 3H), 1.98-1.80 (m, 2H).

### Example 46: ((R)-1-((R)-3-methoxy-2-(2-(trifluoromethyl)pyrimidine-4-carboxamido) propanamido)-3-phenoxypropyl)boronic acid

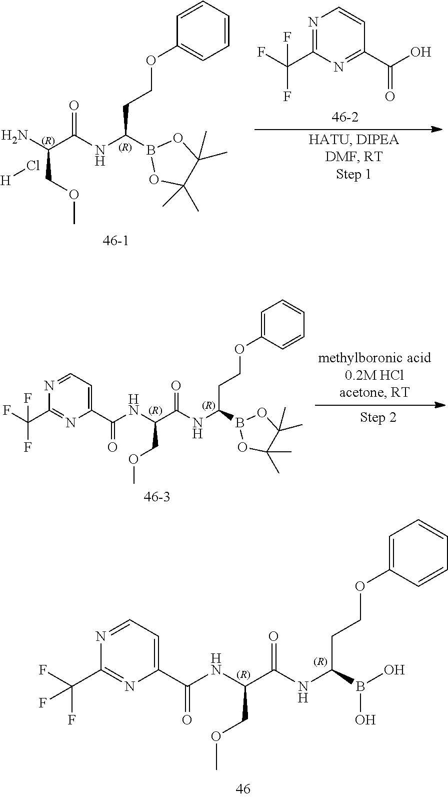

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)-2-(trifluoromethyl)pyrimidine-4-carboxamide [Step 1]: 2-(trifluoromethyl)pyrimidine-4-carboxylic acid (46-1, 80 mg, 0.4 mmol), (R)-2-amino-3-methoxy-N—((R)-3-phenoxy-1-(4,4,5,5-tetramethyl-1,3,2-dioxaborolan-2-yl)propyl)propanamide hydrochloride (46-2, 208 mg, 0.5 mmol) and O-(7-azabenzotriazol-1-yl)-N,N,N′,N′-tetramethyluronium hexafluorophosphate, HATU (238 mg, 0.6 mmol) were suspended in N,N-dimethyl formamide (2 mL) and the mixture was cooled to 0° C. N,N-Diisopropylethylamine (0.2 mL, 1.3 mmol) was added to the reaction mixture and stirred for 2 h at ambient temperature. The reaction mixture was diluted with ethyl acetate (25 mL), washed with cold water (5×10 mL) and brine (2×5 mL). The organic extract was dried over anhydrous Na₂SO₄ and concentrated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)-2-(trifluoromethyl)pyrimidine-4-carboxamide (46-3, 200 mg). [M−H]⁻: 551.3. and [M-83]-=469.0.

Synthesis of ((R)-1-((R)-3-methoxy-2-(2-(trifluoromethyl)pyrimidine-4-carboxamido) propanamido)-3-phenoxypropyl)boronic acid [Step 2]: To a stirred solution of N—((R)-3-methoxy-1-oxo-1-(((R)-3-phenoxy-1-(4,4,5,5-tetramethyl-1,3,2-dioxaborolan-2-yl)propyl)amino) propan-2-yl)-2-(trifluoromethyl)pyrimidine-4-carboxamide (46-3, 200 mg, 0.4 mmol) and methylboronic acid (217 mg, 4 mmol) in acetone (10 mL) was added 0.2N HCl (10 mL) at ice cold condition and stirred at ambient temperature for 16 h. Volatiles were evaporated under reduced pressure and the crude product was purified by prep HPLC purification and lyophilized to afford ((R)-1-((R)-3-methoxy-2-(2-(trifluoromethyl)pyrimidine-4-carboxamido)propanamido)-3-phenoxypropyl)boronic acid (Compound 46, 25 mg). [M−H]⁻: 469.1; ¹H NMR (400 MHz, DMSO-d6) δ_{H}: 9.31 (d, 1H), 8.28 (d, 1H), 7.27-7.23 (m, 2H), 6.91-6.85 (m, 3H), 4.73-4.69 (m, 1H), 3.93-3.89 (m, 2H), 3.71-3.61 (m, 2H), 3.31-3.27 (m, 1H), 3.20 (s, 3H), 2.01-1.85 (in, 2H).

### Example 47: ((R)-1-((R)-3-methoxy-2-(6-methylnicotinamido)propanamido)-3-phenoxy propyl)boronic acid

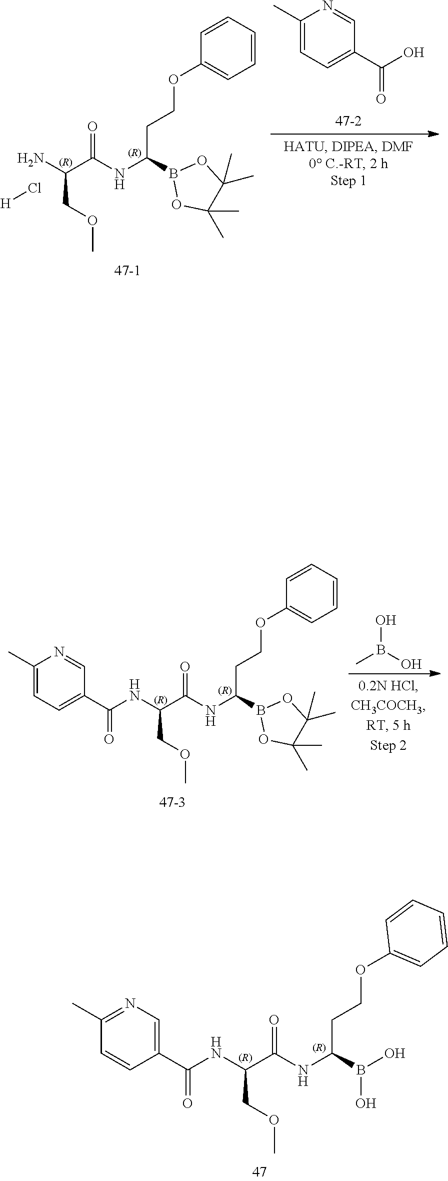

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)-6-methylnicotinamide [Step 1]: To a stirred solution of 6-methylnicotinic acid (47-2, 80 mg, 0.58 mmol) in DMF (5 mL) were added HATU (444 mg, 1.67 mmol) and (R)-2-amino-3-methoxy-N—((R)-3-phenoxy-1-(4,4,5,5-tetramethyl-1,3,2-dioxaborolan-2-yl)propyl)propanamide hydrochloride (47-1, 290 mg, 0.70 mmol) followed by N,N-diisopropylethylamine (0.31 mL, 1.75 mmol) at 0° C. and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)-6-methyl nicotinamide (47-3, 260 mg) which was used in the next step without further purification. [M−H]⁻=496.2.

Synthesis of ((R)-1-((R)-3-methoxy-2-(6-methylnicotinamido)propanamido)-3-phenoxy propyl)boronic acid [Step 2]: To a solution of N—((R)-3-methoxy-1-oxo-1-(((R)-3-phenoxy-1-(4,4,5,5-tetramethyl-1,3,2-dioxaborolan-2-yl)propyl)amino)propan-2-yl)-6-methylnicotinamide (47-3, 260 mg, 0.53 mmol) in acetone (3 mL) was added methylboronic acid (313 mg, 5.22 mmol) followed by addition of 0.2 N HCl (2.7 mL) at 0° C. The reaction mixture was stirred at ambient temperature for 5 h. Volatiles were evaporated under reduced pressure and purified by PREP HPLC purification and lyophilized to give ((R)-1-((R)-3-methoxy-2-(6-methyl nicotinamido)propanamido)-3-phenoxypropyl)boronic acid (Compound 47, 29 mg). [M−H]⁻=414.3. 1H NMR (400 MHz, MeOD) δ_{H}: 8.89 (d, 1H), 8.18-8.15 (m, 1H), 7.40 (d, 1H), 7.23-7.19 (m, 2H), 6.89-6.85 (m, 3H), 4.98 (t. 1H), 4.04 (t, 2H), 3.83-3.79 (m, 2H), 3.38 (s, 3H), 2.91 (t, 1H), 2.59 (s, 3H), 1.99-1.91 (m, 2H).

### Example 48: of ((R)-1-((R)-3-methoxy-2-(2-methylnicotinamido)propanamido)-3-phenoxy propyl)boronic acid

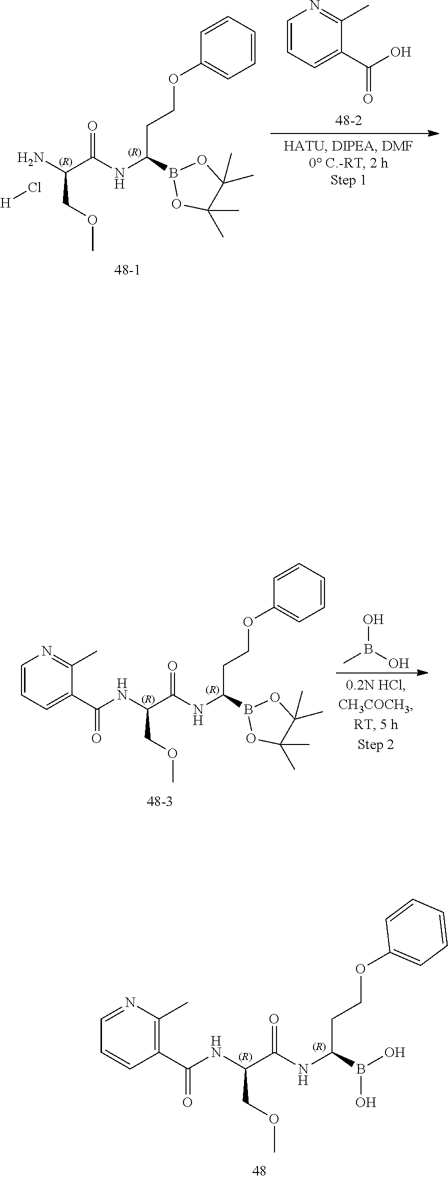

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)-2-methylnicotinamide [Step 1]: To a stirred solution of 2-methylnicotinic acid (48-2, 82 mg, 0.59 mmol) in DMF (5 mL) were added HATU (455 mg, 1.20 mmol) and (R)-2-amino-3-methoxy-N—((R)-3-phenoxy-1-(4,4,5,5-tetramethyl-1,3,2-dioxaborolan-2-yl)propyl)propanamide hydrochloride (48-1, 298 mg, 0.78 mmol) followed by the addition of N,N-diisopropylethylamine (0.32 mL, 1.80 mmol) at 0° C., and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)-2-methylnicotinamide (48-3, 230 mg) which was used in the next step without further purification. [M−H]⁻=496.2.

Synthesis of ((R)-1-((R)-3-methoxy-2-(2-methylnicotinamido)propanamido)-3-phenoxy propyl)boronic acid [Step 2]: To a solution of N—((R)-3-methoxy-1-oxo-1-(((R)-3-phenoxy-1-(4,4,5,5-tetramethyl-1,3,2-dioxaborolan-2-yl)propyl)amino)propan-2-yl)-2-methylnicotinamide (48-3, 230 mg, 0.46 mmol) in acetone (3 mL) was added methylboronic acid (277 mg, 4.62 mmol) followed by addition of 0.2 N HCl (2.35 mL) at 0° C. The reaction mixture was stirred at ambient temperature for 5 h. Volatiles were evaporated under reduced pressure and purified by PREP HPLC purification and lyophilized to give ((R)-1-((R)-3-methoxy-2-(2-methyl nicotinamido)propanamido)-3-phenoxypropyl)boronic acid (Compound 48, 31 mg). [M−H]⁻=414.3. 1H NMR (400 MHz, MeOD) δH: 8.49-8.48 (m, 1H), 7.85 (d, 1H), 7.33-7.30 (m, 1H), 7.24-7.20 (m, 2H), 6.92-6.86 (m, 3H), 4.97 (t, 1H), 4.06 (t, 2H), 3.79-3.74 (m, 2H), 3.38 (s, 3H), 2.97 (t, 1H), 2.59 (s, 3H), 2.05-2.01 (m, 1H), 1.96-1.92 (m, 1H).

### Example 49: ((R)-1-((R)-3-methoxy-2-(nicotinamido)propanamido)-3-phenoxypropyl) boronic acid

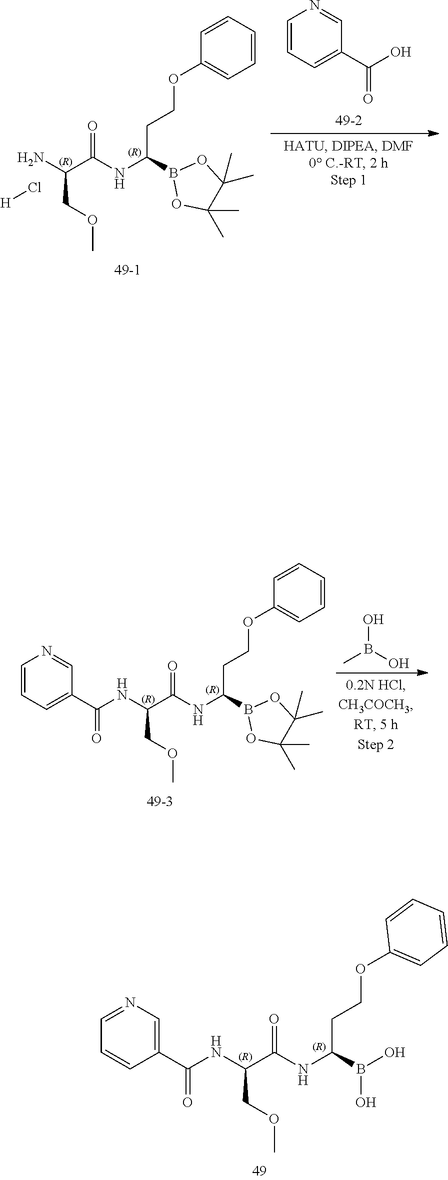

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)nicotinamide [Step 1]: To a stirred solution of nicotinic acid (49-2, 76 mg, 0.62 mmol) in DMF (5 mL) were added HATU (469 mg, 1.23 mmol) and (R)-2-amino-3-methoxy-N—((R)-3-phenoxy-1-(4,4,5,5-tetramethyl-1,3,2-dioxaborolan-2-yl) propyl)propanamide hydrochloride (49-1, 307 mg, 0.74 mmol) followed by addition of N,N-diisopropylethylamine (0.33 mL, 1.85 mmol) at 0° C., and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)nicotinamide (49-3, 310 mg) which was used in the next step without further purification. [M−H]⁻=482.3.

Synthesis of ((R)-1-((R)-3-methoxy-2-(nicotinamido)propanamido)-3-phenoxypropyl) boronic acid [Step 2]: To a solution of N—((R)-3-methoxy-1-oxo-1-(((R)-3-phenoxy-1-(4,4,5,5-tetramethyl-1,3,2-dioxaborolan-2-yl)propyl)amino)propan-2-yl)nicotinamide (49-3, 310 mg, 0.64 mmol) in acetone (3 mL) was added methylboronic acid (384 mg, 6.41 mmol) followed by addition of 0.2 N HCl (3.25 mL) at 0° C. The reaction mixture was stirred at ambient temperature for 5 h. Volatiles were evaporated under reduced pressure and purified by PREP HPLC purification and lyophilized to give ((R)-1-((R)-3-methoxy-2-(nicotinamido)propanamido)-3-phenoxypropyl) boronic acid (Compound 49, 30 mg). [M−H]⁻=400.2. 1H NMR (400 MHz, MeOD) δH: 9.05 (s, 2H), 8.74-8.73 (m, 1H), 8.38-8.36 (m, 1H), 7.62 (bs, 1H), 7.21 (t, 2H), 6.89-6.85 (m, 3H), 5.01-4.99 (m, 1H), 4.04 (t, 2H), 3.85-3.78 (m, 2H), 3.40 (s, 3H), 2.92 (t, 1H), 2.02-1.99 (m, 1H), 1.94-1.90 (m, 1H).

### Example 50: ((R)-1-((R)-2-(2,4-dimethyloxazole-5-carboxamido)-3-methoxypropanamido)-4-phenylbutyl)boronic acid

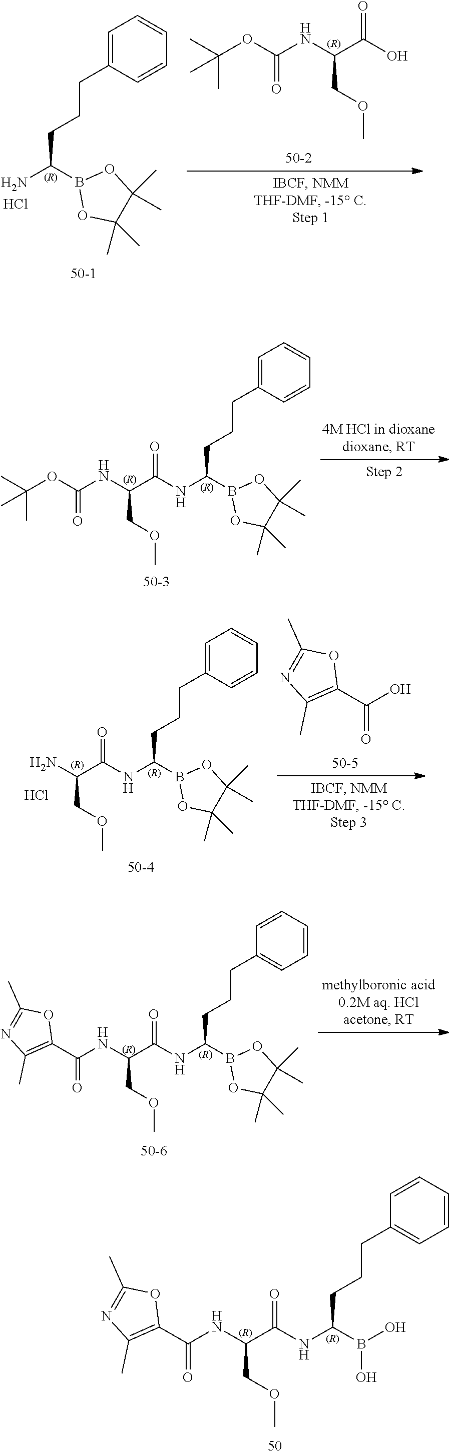

Synthesis of tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate [Step 1]: To a solution of N-(tert-butoxycarbonyl)-O-methyl-D-serine (50-2, 193 mg, 0.9 mmol) in THF (6 mL) was added IBCF (0.12 mL, 0.9 mmol) followed by NMM (0.12 mL, 0.9 mmol) at −15° C. After 45 min, a solution of (R)-4-phenyl-1-(4,4,5,5-tetramethyl-1,3,2-dioxaborolan-2-yl)butan-1-amine hydrochloride (50-1, 250 mg, 0.8 mmol) in DMF (2 mL), and NMM (0.11 mL, 0.8 mmol) were added sequentially.

After 1 h, the reaction mixture was diluted with ethyl acetate, and washed successively with 0.1 N aqueous HCl (twice), 5% aqueous K₂CO₃, water and brine. The organic extract was dried over anhydrous Na₂SO₄, filtered and concentrated under reduced pressure to afford tert-butyl ((R)-3-methoxy-1-oxo-1-(((R)-4-phenyl-1-(4,4,5,5-tetramethyl-1,3,2-dioxaborolan-2-yl)butyl)amino) propan-2-yl) carbamate (50-3, 350 mg), which was used in the next step without further purification. [M−H]⁻=475.4.

Synthesis of (R)-2-amino-3-methoxy-N—((R)-4-phenyl-1-(4,4,5,5-tetramethyl-1,3,2-dioxaborolan-2-yl)butyl)propanamide hydrochloride [Step 2]: To an ice-cold solution of tert-butyl ((R)-3-methoxy-1-oxo-1-(((R)-4-phenyl-1-(4,4,5,5-tetramethyl-1,3,2-dioxaborolan-2-yl) butyl)amino)propan-2-yl)carbamate (50-3, 350 mg, 0.7 mmol) in 1,4-dioxane (4 mL) was added 4M HCl in dioxane (8.0 mL, 32.0 mmol) and the reaction mixture was stirred at ambient temperature. After 5 h, the reaction mixture was concentrated under reduced pressure to afford (R)-2-amino-3-methoxy-N—((R)-4-phenyl-1-(4,4,5,5-tetramethyl-1,3,2-dioxaborolan-2-yl)butyl) propanamide hydrochloride (50-4, 280 mg), which was used in the next step without further purification. [M−H]⁻=375.6, and 293.4 for the corresponding boronic acid.

Synthesis of N—((R)-3-methoxy-1-oxo-1-(((R)-4-phenyl-1-(4,4,5,5-tetramethyl-1,3,2-dioxaborolan-2-yl)butyl)amino)propan-2-yl)-2,4-dimethyloxazole-5-carboxamide [Step 3]: To a solution of 2,4-dimethyloxazole-5-carboxylic acid (50-5, 169 mg, 1.2 mmol) in THF (8 mL) was added IBCF (0.2 mL, 1.2 mmol) followed by NMM (0.2 mL, 1.2 mmol) at −15° C. After 45 min, a solution of (R)-2-amino-3-methoxy-N—((R)-4-phenyl-1-(4,4,5,5-tetramethyl-1,3,2-dioxaborolan-2-yl)butyl)propanamide hydrochloride (50-4, 450 mg, 1.1 mmol) in DMF (3 mL), and NMM (0.2 mL, 1.1 mmol) were added successively. After stirring for 1 h at the same temperature, the reaction mixture was diluted with ethyl acetate and washed successively with 0.1N aqueous HCl, 5% aqueous K₂CO₃, water and brine. The organic phase was dried over anhydrous Na₂SO₄, filtered and concentrated under reduced pressure to afford N—((R)-3-methoxy-1-oxo-1-(((R)-4-phenyl-1-(4,4,5,5-tetramethyl-1,3,2-dioxaborolan-2-yl)butyl)amino)propan-2-yl)-2,4-dimethyl oxazole-5-carboxamide (50-6, 300 mg), which was used in the next step without further purification. [M−H]⁻=498.3, and 416.4 for the corresponding boronic acid.

Synthesis of ((R)-1-((R)-2-(2,4-dimethyloxazole-5-carboxamido)-3-methoxypropanamido)-4-phenylbutyl)boronic acid [Step 4]: To an ice-cold solution of N—((R)-3-methoxy-1-oxo-1-(((R)-4-phenyl-1-(4,4,5,5-tetramethyl-1,3,2-dioxaborolan-2-yl)butyl)amino)propan-2-yl)-2,4-dimethyl oxazole-5-carboxamide (50-6, 300 mg, 0.6 mmol) and methylboronic acid (539 mg, 9.0 mmol) in acetone (10 mL) was added 0.2N aqueous HCl (15.0 mL, 3.0 mmol), and the reaction mixture was stirred at ambient temperature. After 2 h, the reaction mixture was concentrated under reduced pressure and then lyophilized. The compound was purified by reverse phase preparative HPLC to afford ((R)-1-((R)-2-(2,4-dimethyloxazole-5-carboxamido)-3-methoxypropanamido)-4-phenylbutyl)boronic acid (Compound 50, 23 mg). [M−H]⁻=416.1. ¹H NMR (400 MHz, CD₃OD): δ_{H} 7.23-7.09 (m, 5H), 4.93 (t, 1H), 3.80-3.71 (m, 2H), 3.35 (s, 3H), 2.64-2.58 (m, 3H), 2.48 (s, 3H), 2.37 (s, 3H), 1.70-1.62 (m, 2H), 1.56-1.48 (m, 2H).

### Example 51: ((R)-1-((R)-2-(4-chlorobenzamido)-3-methoxy propanamido)-4-phenylbutyl) boronic acid

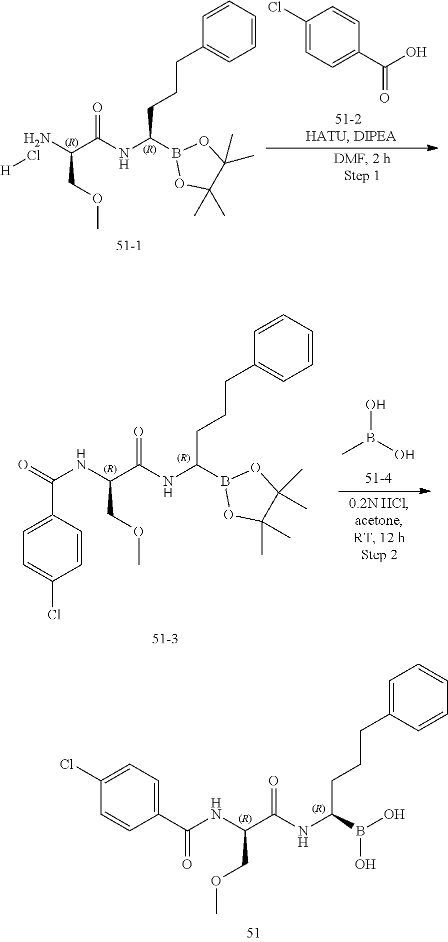

Synthesis of 4-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide [Step 1]: To a stirred solution of 4-chlorobenzoic acid (51-2, 100 mg, 0.64 mmol) in DMF (5 mL) were added HATU (486 mg, 1.28 mmol) and (R)-2-amino-3-methoxy-N—((R)-4-phenyl-1-(4,4,5,5-tetramethyl-1,3,2-dioxaborolan-2-yl)butyl)propanamide hydrochloride (51-1, 316 mg, 0.77 mmol) followed by N,N-diisopropylethylamine (0.34 mL, 1.92 mmol) at 0° C. and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to give product which was purified by PREP HPLC to afford 4-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (51-3, 60 mg) which was used in the next step without further purification. [M−H]⁻=513.4, mass for corresponding boronic acid was also observed.

Synthesis of ((R)-1-((R)-2-(4-chlorobenzamido)-3-methoxypropanamido)-4-phenylbutyl) boronic acid [Step 2]: To a solution of 4-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (51-3, 60 mg, 0.12 mmol) in acetone (2 mL) was added methylboronic acid (51-4, 70 mg, 1.17 mmol) followed by addition of 0.2 N HCl (0.60 mL) at 0° C. The reaction mixture was stirred at ambient temperature for 5 h. Volatiles were evaporated under reduced pressure and purified by PREP HPLC purification and lyophilized to give ((R)-1-((R)-2-(4-chlorobenzamido)-3-methoxy propanamido)-4-phenylbutyl)boronic acid (Compound 51, 38 mg). [M−H]⁻=431.3. ¹H NMR (400 MHz, MeOD) δ_{H}: 7.86 (d, 2H), 7.50 (d, 2H), 7.25-7.21 (m, 2H), 7.19-7.17 (m, 2H), 7.14-7.11 (m, 1H), 4.98 (t, 1H), 3.83-3.75 (m, 2H), 3.38 (s, 3H), 2.65-2.60 (m, 3H), 1.70-1.64 (m, 2H), 1.59-1.48 (m, 2H).

### Example 52: ((R)-1-((R)-3-methoxy-2-(pyrimidine-5-carboxamido) propanamido)-4-phenyl butyl)boronic acid

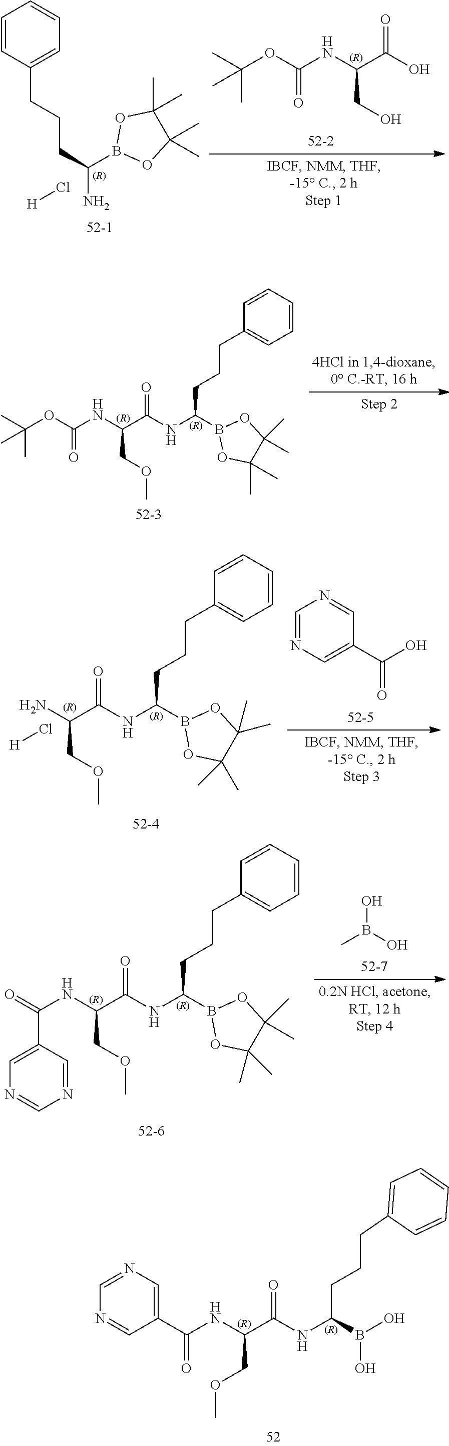

Synthesis of tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate [Step 1]: To a stirred solution of (tert-butoxycarbonyl)-D-serine (52-2, 600 mg, 2.74 mmol) in DMF (10 mL), HATU (2.08 g, 5.47 mmol) and (R)-4-phenyl-1-(4,4,5,5-tetramethyl-1,3,2-dioxaborolan-2-yl)butan-1-amine hydrochloride (52-1, 1.02 g, 3.28 mmol) were added followed by the addition of DIPEA (1.5 mL, 8.21 mmol) at 0° C. The reaction mixture was stirred at ambient temperature for 2 h. The reaction mixture was diluted with ice cold water and extracted with ethyl acetate (two times). The combined organic extract was washed with brine and dried over anhydrous Na₂SO₄, filtered and concentrated under reduced pressure to afford tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate (52-3, 1.2 g). This product was forwarded to the next step without further purification. [M+H]⁺: 475.1.

Synthesis of (R)-2-amino-3-methoxy-N—((R)-4-phenyl-1-(4,4,5,5-tetramethyl-1,3,2-dioxaborolan-2-yl)butyl)propanamide hydrochloride [Step 2]: To the solution of tert-butyl ((R)-3-methoxy-1-oxo-1-(((R)-4-phenyl-1-(4,4,5,5-tetramethyl-1,3,2-dioxaborolan-2-yl)butyl) amino)propan-2-yl)carbamate (52-3, 1.2 g, 2.52 mmol) in 1,4-dioxane (10 mL) was added HCl (4 M in dioxane, 6.6 mL, 25.2 mmol) at 0° C. and stirred at ambient temperature for 6 h. Reaction mixture was concentrated well under reduced pressure and lyophilized to afford (R)-2-amino-3-methoxy-N—((R)-4-phenyl-1-(4,4,5,5-tetramethyl-1,3,2-dioxaborolan-2-yl)butyl)propanamide hydrochloride (52-4, 900 mg). This product was used in next step without further purification. [M−H]⁻: 375.5.

Synthesis of N—((R)-3-methoxy-1-oxo-1-(((R)-4-phenyl-1-(4,4,5,5-tetramethyl-1,3,2-dioxaborolan-2-yl)butyl)amino)propan-2-yl)pyrimidine-5-carboxamide [Step 3]: To a stirred solution of pyrimidine-5-carboxylic acid (52-5, 25 mg, 0.2 mmol) in DMF (1 mL), HATU (153 mg, 0.40 mmol) and solution of (R)-2-amino-3-methoxy-N—((R)-4-phenyl-1-(4,4,5,5-tetramethyl-1,3,2-dioxaborolan-2-yl)butyl)propanamide hydrochloride (91 mg, 0.22 mmol) in DMF (0.5 mL) were added followed by DIPEA (0.11 mL, 0.60 mmol) at 0° C. The reaction mixture was stirred at ambient temperature for 2.5 h. The reaction mixture was diluted with ice cold water and extracted with ethyl acetate (two times). The combined organic extract was washed with brine and dried over anhydrous Na2SO4, filtered and concentrated under reduced pressure to afford N—((R)-3-methoxy-1-oxo-1-(((R)-4-phenyl-1-(4,4,5,5-tetramethyl-1,3,2-dioxaborolan-2-yl)butyl)amino) propan-2-yl)pyrimidine-5-carboxamide (52-6, 150 mg). This product was not purified and used in subsequent step. [M−H]⁻: 481.5.

Synthesis ((R)-1-((R)-3-methoxy-2-(pyrimidine-5-carboxamido)propanamido)-4-phenyl butyl)boronic acid [Step 4]: N—((R)-3-methoxy-1-oxo-1-(((R)-4-phenyl-1-(4,4,5,5-tetramethyl-1,3,2-dioxaborolan-2-yl)butyl)amino)propan-2-yl)pyrimidine-5-carboxamide (52-6, 0.15 mg, 0.17 mmol) and methylboronic acid (52-7, 102 mg, 1.71 mmol) in acetone (1 mL) was added HCl (0.2 M solution in water, 1.0 mL) at 0° C. The reaction mixture was allowed to stir at ambient temperature for 5 h. Reaction mixture was concentrated under reduced pressure and finally lyophilized. Resulting product was purified by reverse phase PREP HPLC purification and lyophilized to afford (Compound 52.12 mg). [M−H]⁻: 399.3. 1H NMR (400 MHz, CD₃OD): 9.28 (s, 1H), 9.18 (s, 2H), 7.23-7.10 (m, 5H), 4.97 (m, 1H), 3.82-3.75 (m, 2H), 3.36 (s, 3H), 2.63-2.59 (m, 3H), 1.8-1.4 (m, 5H).

### Example 53: ((R)-1-((R)-3-methoxy-2-(pyrazine-2-carboxamido) propanamido)-4-oxo-4-phenylbutyl)boronic acid

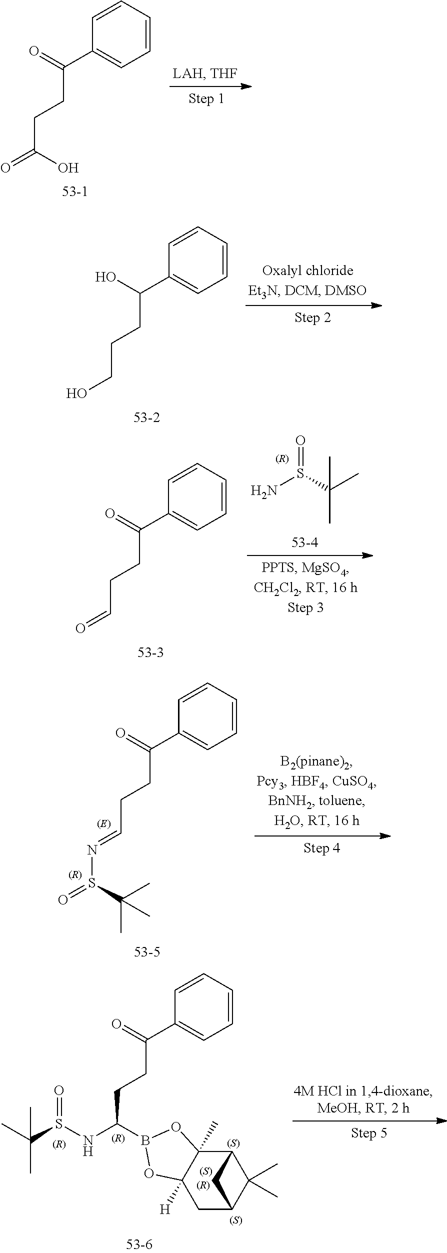

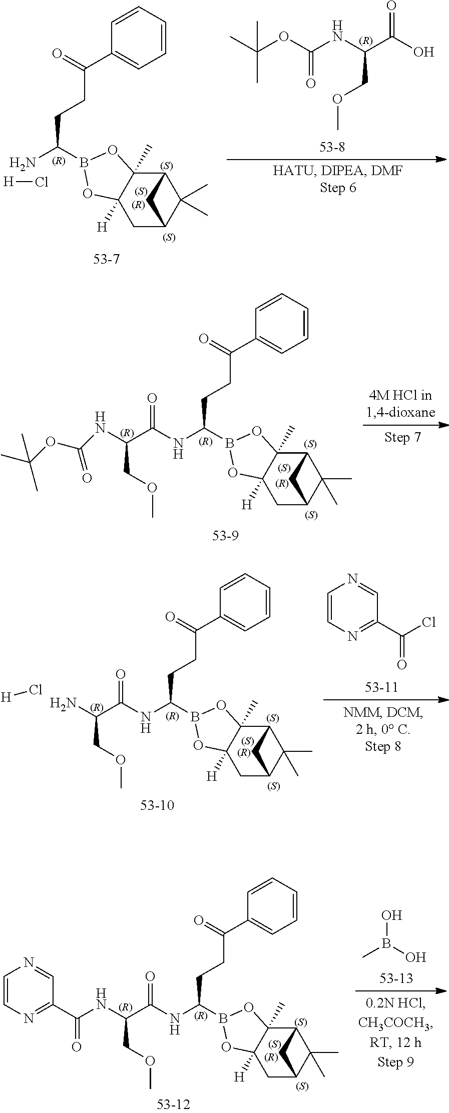

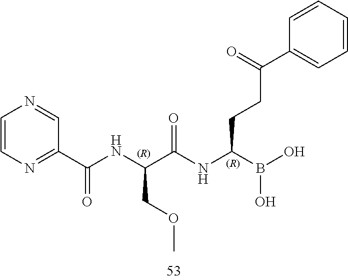

Synthesis of 1-phenylbutane-1,4-diol [Step 1]: To a stirred solution of 4-oxo-4-phenyl-butanoic acid (53-1, 500 mg, 2.81 mmol) in THF (30 mL) was added lithium aluminum hydride, 1M in THF (11 mL, 11.2 mmol) at 0° C. and stirred at ambient temperature for 16 h. The reaction mixture was quenched with sodium sulfate decahydrate and filtered over celite pad. Filtrate was evaporated under reduce pressure to get 1-phenylbutane-1,4-diol (53-2, 450 mg). 1H NMR (400 MHz, DMSO-d6): δ_{H} 7.29 (s, 2H), 5.11 (m, 11H), 4.50-4.49 (m, 1H), 4.34 (t, 1H), 3.38-3.29 (m, 2H), 1.60-1.35 (m, 4H).

Synthesis of 4-oxo-4-phenylbutanal [Step 2]: To a stirred solution of oxalyl chloride (1.1 mL, 13.2 mmol) in dichloromethane (30 mL) was added DMSO (1.4 mL, 19.9 mmol) over 15 min at −78° C. stirred for 30 min. 1-phenylbutane-1,4-diol (53-2, 550 mg, 3.31 mmol) was added and stirred for further 30 min. Et3N (4.6 mL, 33.1 mmol) was added to it. The reaction mixture was cooled to 0° C. and stirred for 1 h. The reaction mixture was diluted with 70 mL of water and extracted with DCM. Combined organic layer was dried over Na₂SO₄ and concentrated under reduced pressure to afford 4-oxo-4-phenylbutanal (53-3, 420 mg). ¹H NMR (400 MHz, DMSO) δ_{H}: 9.74 (s, 1H), 7.98 (d, 2H), 7.66-7.63 (m, 1H), 7.53 (t, 2H), 3.44-3.12 (m, 2H), 2.88-2.68 (m, 2H).

Synthesis of (R,E)-2-methyl-N-(4-oxo-4-phenylbutylidene)propane-2-sulfinamide [Step 3]: To a solution of (R)-2-methylpropane-2-sulfinamide (53-4, 300 mg, 2.48 mmol) in dichloromethane (10 mL) under nitrogen atmosphere were added PPTS (12 mg, 0.05 mmol) and MgSO4 (1.49 g, 12.4 mmol) followed by a solution of 4-oxo-4-phenylbutanal (53-3, 520 mg, 3.18 mmol) in DCM (5 mL) at ice cold condition and stirred at ambient temperature for 16 h. The reaction mixture was filtered through celite bed and the bed was washed with EtOAc. Combined filtrate liquid was concentrated under reduced pressure. The crude was purified by flash chromatography to afford (R,E)-2-methyl-N-(4-oxo-4-phenylbutylidene)propane-2-sulfinamide (53-5, 270 mg). [M+H]⁺: 266.1.

Synthesis of (R)-2-methyl-N—((R)-4-oxo-4-phenyl-1-((3aS,4S,6S,7aR)-3a,5,5-trimethylhexa hydro-4,6-methanobenzo[d][1,3,2]dioxaborol-2-yl)butyl)propane-2-sulfinamide [Step 4]: To a solution of PCy₃·HBF₄ (9 mg, 0.02 mmol) in toluene (1 mL), a solution of CuSO₄·5H₂O (9 mg, 0.04 mmol) in water (0.5 mL) and benzyl amine (0.008 mL, 0.08 mmol) were added and stirred for 30 min. A solution of (R,E)-2-methyl-N-(4-oxo-4-phenylbutylidene)propane-2-sulfinamide (53-5, 200 mg, 0.8 mmol) and bis pinanediborane (270 mg, 0.83 mmol) in toluene (3 mL) was to it and stirred at 25° C. for 16 h. The reaction mixture was diluted with ethyl acetate and filtered through a short pad of deactivated silicagel (SiO2:H₂O, 100:35 w/w). The combined filtrate was concentrated under vacuum and purified by prep-HPLC purification to afford (R)-2-methyl-N—((R)-4-oxo-4-phenyl-1-((3aS,4S,6S,7aR)-3a,5,5-trimethylhexahydro-4,6-methanobenzo[d][1,3,2]dioxaborol-2-yl)butyl)propane-2-sulfinamide (53-6, 80 mg). [M−H]⁻: 444.4.

Synthesis of (R)-4-amino-1-phenyl-4-((3aS,4S,6S,7aR)-3a,5,5-trimethylhexahydro-4,6-methanobenzo[d][1,3,2]dioxaborol-2-yl)butan-1-one hydrochloride [Step 5]: To a stirred solution of R)-2-methyl-N—((R)-4-oxo-4-phenyl-1-((3aS,4S,6S,7aR)-3a,5,5-trimethylhexahydro-4,6-methanobenzo[d][1,3,2]dioxaborol-2-yl)butyl)propane-2-sulfinamide (53-6, 70 mg, 0.15 mmol) in 1, 4-dioxane (2 mL) and methanol (0.06 mL), 4M HCl in 1,4-dioxane (3 mL, 12 mmol) was added in an ice cold condition and stirred at 25° C. for 2 h. Volatiles were removed under reduced pressure to afford (R)-4-amino-1-phenyl-4-((3aS,4S,6S,7aR)-3a,5,5-trimethylhexa hydro-4,6-methanobenzo[d][1,3,2]dioxaborol-2-yl)butan-1-one hydrochloride (53-7, 60 mg). The crude product was used for next step without further purification.

Synthesis of tert-butyl ((R)-3-methoxy-1-oxo-1-(((R)-4-oxo-4-phenyl-1-((3aS,4S,6S,7aR)-3a,5,5-trimethylhexahydro-4,6-methanobenzo[d][1,3,2]dioxaborol-2-yl)butyl)amino) propan-2-yl)carbamate [Step 6]: To a stirred solution of N-(tert-butoxycarbonyl)-O-methyl-D-serine (53-8, 50 mg, 0.22 mmol) in DMF (3 mL) was added HATU (130 mg, 0.34 mmol) followed by DIPEA (0.08 mL, 0.45 mmol) at 0° C. and stirred for 30 min, (R)-4-amino-1-phenyl-4-((3aS,4S,6S,7aR)-3a,5,5-trimethylhexahydro-4,6-methanobenzo[d][1,3,2]dioxaborol-2-yl)butan-1-one hydrochloride (53-7, 90 mg, 0.3 mmol) was added to it and stirred at 0° C. for 2 h. It was diluted with EtOAc and washed with 5% aqueous K₂CO₃ water and brine dried over anhydrous Na₂SO₄ and evaporated under reduced pressure to afford tert-butyl ((R)-3-methoxy-1-oxo-1-(((R)-4-oxo-4-phenyl-1-((3aS,4S,6S,7aR)-3a,5,5-trimethylhexahydro-4,6-methanobenzo[d][1,3,2]dioxaborol-2-yl)butyl)amino)propan-2-yl)carbamate (53-9, 120 mg). [M−H]⁻=541.4

Synthesis of (R)-2-amino-3-methoxy-N—((R)-4-oxo-4-phenyl-1-((3aS,4S,6S,7aR)-3a,5,5-trimethylhexahydro-4,6-methanobenzo[d][1,3,2]dioxaborol-2-yl)butyl)propenamide hydrochloride [Step 7]: To a stirred solution of tert-butyl ((R)-3-methoxy-1-oxo-1-(((R)-4-oxo-4-phenyl-1-((3aS,4S,6S,7aR)-3a,5,5-trimethylhexahydro-4,6-methanobenzo[d][1,3,2]dioxaborol-2-yl)butyl)amino)propan-2-yl)carbamate (53-9, 100 mg, 0.2 mmol) in 1,4 dioxane (3 mL) was added 4M HCl in 1,4-dioxane (1 mL) at 0° C. and stirred for 16 h at ambient temperature. Volatiles were removed under reduced pressure to afford (R)-2-amino-3-methoxy-N—((R)-4-oxo-4-phenyl-1-((3aS,4S,6S,7aR)-3a,5,5-trimethylhexahydro-4,6-methanobenzo[d][1,3,2]dioxaborol-2-yl)butyl) propenamide hydrochloride (53-10, 80 mg). [M−H]⁻: 441.5.

Synthesis of N—((R)-3-methoxy-1-oxo-1-(((R)-4-oxo-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide [Step 8]: To a stirred solution of (R)-2-amino-3-methoxy-N—((R)-4-oxo-4-phenyl-1-((3aS,4S,6S,7aR)-3a,5,5-trimethylhexahydro-4,6-methanobenzo[d][1,3,2]dioxaborol-2-yl)butyl)propenamide hydrochloride (53-10, 100 mg, 0.21 mmol) in dichloromethane (6 mL) was added NMM (0.05 mL, 0.4 mmol) at 0° C. Pyrazine-2-carbonyl chloride (53-11, 44 mg, 0.3 mmol) was added to the reaction mixture and stirred at ambient temperature for 1.5 h. Reaction mixture was diluted DCM and washed with water and brine, dried over anhydrous Na₂SO₄ and evaporated under reduced pressure. The product was purified by prep HPLC purification and lyophilized to afford N—((R)-3-methoxy-1-oxo-1-(((R)-4-oxo-4-phenyl-1-((3aS,4S,6S,7aR)-3a,5,5-trimethylhexahydro-4,6-methanobenzo[d][1,3,2]dioxaborol-2-yl)butyl) amino)propan-2-yl)pyrazine-2-carboxamide (53-12, 12 mg). [M+H]⁴: 549.5

Synthesis of ((R)-1-((R)-3-methoxy-2-(pyrazine-2-carboxamido)propanamido)-4-oxo-4-phenylbutyl)boronic acid [Step 9]: To a stirred solution of N—((R)-3-methoxy-1-oxo-1-(((R)-4-oxo-4-phenyl-1-((3aS,4S,6S,7aR)-3a,5,5-trimethylhexahydro-4,6-methanobenzo[d][1,3,2]dioxaborol-2-yl)butyl)amino)propan-2-yl)pyrazine-2-carboxamide (53-12, 50 mg, 0.09 mmol) and methylboronic acid (53-13, 54 mg, 0.9 mmol) in acetone (2.0 mL) was added 0.2N HCl (2.0 mL) and stirred at ambient temperature for 16 h. Volatiles were evaporated under reduced pressure and purified by prep HPLC purification and lyophilized to afford ((R)-1-((R)-3-methoxy-2-(pyrazine-2-carboxamido)propanamido)-4-oxo-4-phenylbutyl)boronic acid (Compound 53, 17 mg). [M−H]⁻: 413.4. ¹H NMR (400 MHz, CD₃OD) δ_{H}: 9.24 (s, 1H), 8.81 (s, 1H), 8.71 (s, 1H), 7.98 (d, 2H), 7.57-7.44 (m, 3H), 4.99 (s, 1H), 3.91-3.80 (m, 2H), 3.39 (s, 1H), 3.11-3.04 (m, 3H), 2.76 (s, 2H), 1.88 (t, 3H), 1.28 (s, 2H).

### Example 54: ((R)-1-((R)-2-(3-chlorobenzamido)-3-methoxy propanamido)-4-phenylbutyl) boronic acid

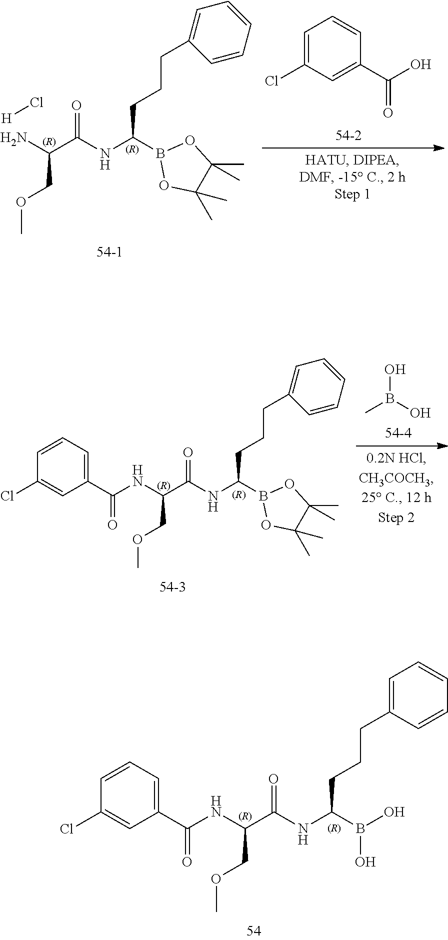

Synthesis of 3-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide [Step 1]: To a stirred solution of 3-chlorobenzoic acid (54-2, 50 mg, 0.6 mmol) in DMF (4 mL) was added HATU (182 mg, 0.5 mmol) followed by DIPEA (0.14 mL, 0.8 mmol) at 0° C. and stirred for 30 min. (R)-2-amino-3-methoxy-N—((R)-4-phenyl-1-(4,4,5,5-tetramethyl-1,3,2-dioxaborolan-2-yl)butyl)propanamide hydrochloride (54-1, 158 mg, 0.4 mmol) was added to it and stirred at 0° C. for 2 h. It was quenched with 5% aqueous K₂CO₃ solution and extracted with EtOAc (thrice). Combined organic layer was washed with water and brine, dried over anhydrous Na₂SO₄ and evaporated under reduced pressure to afford 3-chloro-N—((R)-3-methoxy-1-oxo-1-(((R)-4-phenyl-1-(4,4,5,5-tetramethyl-1,3,2-dioxa borolan-2-yl)butyl)amino)propan-2-yl)benzamide (54-3, 30 mg). [M−H]⁻: 513.4. Corresponding boronic acid mass peak was also observed.

Synthesis of ((R)-1-((R)-2-(3-chlorobenzamido)-3-methoxypropanamido)-4-phenylbutyl) boronic acid [Step 2]: To a stirred solution of 3-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (54-3, 30 mg, 0.0583 mmol) and methylboronic acid (54-4, 35 mg, 0.583 mmol) in acetone (1.0 mL) was added 0.2N HCl (1.0 mL) and stirred at ambient temperature for 16 h. Volatiles were evaporated under reduced pressure and the crude was purified by prep HPLC purification and lyophilized to afford (R)-1-((R)-2-(3-chlorobenzamido)-3-methoxypropanamido)-4-phenylbutyl)boronic acid (Compound 54.12 mg). [M−H]⁻: 431.3. 1H NMR (400 MHz, DMSO-d6 with 2 Drops of D₂O, 80° C.): δ_{H}: 7.90 (s, 1H), 7.82-7.80 (m, 1H), 7.59-7.57 (m, 1H), 7.51-7.47 (m, 1H), 7.24-7.21 (m, 2H), 7.14-7.12 (m, 3H), 4.65-4.62 (m, 1H), 3.67-3.62 (m, 2H), 3.27 (s, 3H), 2.53-2.49 (m, 1H), 1.57-1.46 (m, δ_{H}).

### Example 55: ((R)-1-((R)-3-(methoxy-d3)-2-(pyrazine-2-carboxamido) propanamido)-4-phenylbutyl)boronic acid

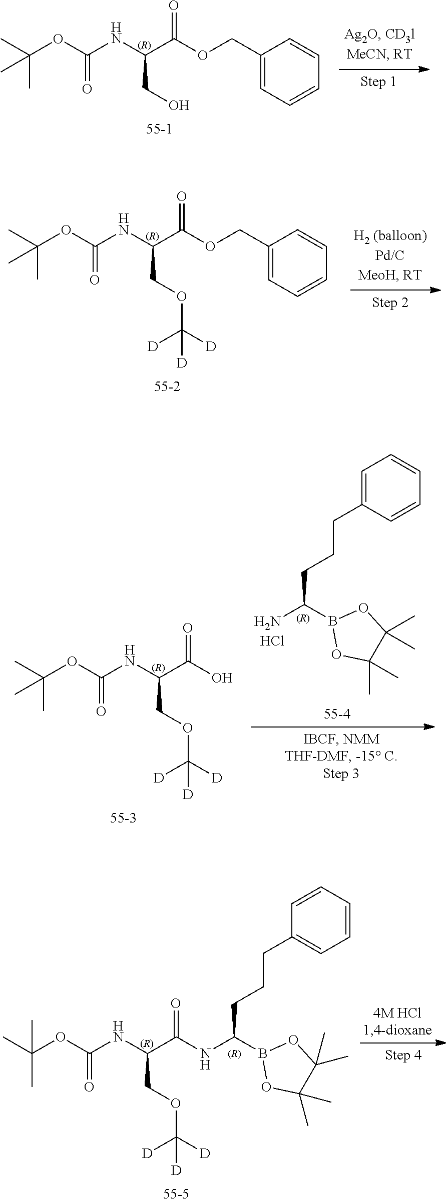

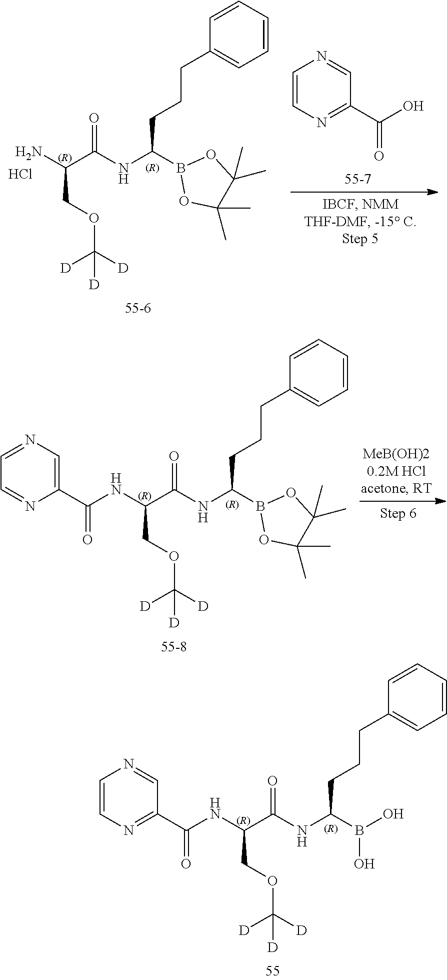

Synthesis of benzyl N-(tert-butoxycarbonyl)-O-(methyl-d3)-D-serinate [Step 1]: To a solution of benzyl (tert-butoxycarbonyl)-D-serinate (55-1, 500 mg, 1.7 mmol) in MeCN (8 mL) was added Ag₂O (589 mg, 2.5 mmol) followed by CD₃₁ (0.3 mL, 5.0 mmol), and the reaction mixture was stirred at ambient temperature in the dark. After 16 h, the reaction mixture was filtered through a pad of celite washing with ethyl acetate, and the filtrate was concentrated under reduced pressure. The compound was purified by combi-flash to afford benzyl N-(tert-butoxycarbonyl)-O-(methyl-d3)-D-serinate (55-2, 200 mg). [M+H]⁺=313.2.

Synthesis of N-(tert-butoxycarbonyl)-O-(methyl-d3)-D-serine [Step 2]: To a solution of benzyl N-(tert-butoxycarbonyl)-O-(methyl-d3)-D-serinate (55-2, 200 mg, 0.6 mmol) in methanol (5 mL) was added Pd/C (20 mg, 10 wt %) under N₂. The reaction vessel was evacuated and refilled with H₂ (twice), and kept under a positive pressure of H₂. After stirring for 2 h at ambient temperature, reaction mixture was filtered through a pad of celite, and concentrated under reduced pressure to afford N-(tert-butoxycarbonyl)-O-(methyl-d3)-D-serine (55-3, 120 mg), which was used in the next step without further purification. ¹H NMR (400 MHz, DMSO-d₆): δ_{H} 12.62 (brs, 1H), 6.90-6.88 (m, 1H), 4.11-4.09 (m, 1H), 3.57-3.49 (m, 2H), 3.16 (s, 2H), 1.38 (s, 9H).

Synthesis of tert-butyl ((R)-3-(methoxy-d3)-1-oxo-1-(((R)-4-phenyl-1-(4,4,5,5-tetramethyl-1,3,2-dioxaborolan-2-yl)butyl)amino)propan-2-yl)carbamate [Step 3]: To a solution of N-(tert-butoxycarbonyl)-O-(methyl-d3)-D-serine (55-3, 120 mg, 0.5 mmol) in THF (4 mL) was added IBCF (0.1 mL, 0.6 mmol) followed by NMM (0.1 mL, 0.6 mmol) at −15° C. After 45 min, a solution of (R)-4-phenyl-1-(4,4,5,5-tetramethyl-1,3,2-dioxaborolan-2-yl)butan-1-amine hydrochloride (55-4, 168 mg, 0.5 mmol) in DMF (1 mL) and NMM (75 μL, 0.5 mmol) were added successively. After stirring for 1 h at the same temperature, the reaction mixture was diluted with ethyl acetate and washed successively with 0.1M aqueous HCl, 5% aqueous K₂CO₃, water, and brine. The organic extract was dried over anhydrous Na₂SO₄, filtered and concentrated under reduced pressure to afford tert-butyl ((R)-3-(methoxy-d3)-1-oxo-1-(((R)-4-phenyl-1-(4,4,5,5-tetramethyl-1,3,2-dioxaborolan-2-yl)butyl)amino)propan-2-yl)carbamate (55-5, 230 mg), which was used in the next step without further purification. [M+H]⁺=480.0.

Synthesis of (R)-2-amino-3-(methoxy-d3)-N—((R)-4-phenyl-1-(4,4,5,5-tetramethyl-1,3,2-dioxaborolan-2-yl)butyl)propanamide hydrochloride [Step 4]: tert-butyl ((R)-3-(methoxy-d3)-1-oxo-1-(((R)-4-phenyl-1-(4,4,5,5-tetramethyl-1,3,2-dioxaborolan-2-yl)butyl)amino)propan-2-yl) carbamate (55-5, 230 mg, 0.5 mmol) in 1,4-dioxane (4 mL) was added 4M HCl in dioxane (4.0 mL, 16.0 mmol) and the reaction mixture was stirred at ambient temperature. After 16 h, the reaction mixture was concentrated under reduced pressure to afford (R)-2-amino-3-(methoxy-d3)-N—((R)-4-phenyl-1-(4,4,5,5-tetramethyl-1,3,2-dioxaborolan-2-yl)butyl)propanamide hydrochloride (55-6, 190 mg) which was used in the next step without further purification. [M−H]⁻=378.4.

Synthesis of N—((R)-3-(methoxy-d3)-1-oxo-1-(((R)-4-phenyl-1-(4,4,5,5-tetramethyl-1,3,2-dioxaborolan-2-yl)butyl)amino)propan-2-yl)pyrazine-2-carboxamide [Step 5]: To a solution of pyrazine-2-carboxylic acid (55-7, 56 mg, 0.4 mmol) in THF (6 mL) was added IBCF (60 μL mL, 0.4 mmol) followed by NMM (60 μL, 0.4 mmol) at −15° C. After stirring for 45 min at the same temperature, a solution of (R)-2-amino-3-(methoxy-d3)-N—((R)-4-phenyl-1-(4,4,5,5-tetramethyl-1,3,2-dioxaborolan-2-yl)butyl)propanamide hydrochloride (55-6, 170 mg, 0.4 mmol) in DMF (2 mL) and NMM (55 μL, 0.4 mmol) were added successively. After stirring for 40 min at the same temperature, the reaction mixture was diluted with ethyl acetate and washed successively with 0.1N aqueous HCl, 5% aqueous K₂CO₃, water and brine. The organic phase was dried over anhydrous Na₂SO₄, filtered and concentrated under reduced pressure to afford N—((R)-3-(methoxy-d3)-1-oxo-1-(((R)-4-phenyl-1-(4,4,5,5-tetramethyl-1,3,2-dioxaborolan-2-yl)butyl) amino)propan-2-yl)pyrazine-2-carboxamide (55-8, 190 mg), which was used in the next step without further purification. [M+H]⁺=486.5.

Synthesis of ((R)-1-((R)-3-(methoxy-d3)-2-(pyrazine-2-carboxamido)propanamido)-4-phenylbutyl)boronic acid [Step 6]: To an ice-cold solution of N—((R)-3-(methoxy-d3)-1-oxo-1-(((R)-4-phenyl-1-(4,4,5,5-tetramethyl-1,3,2-dioxaborolan-2-yl)butyl)amino)propan-2-yl)pyrazine-2-carboxamide (55-8, 190 mg, 0.4 mmol) and methylboronic acid (350 mg, 5.9 mmol) in acetone (10 mL) was added 0.2N aqueous HCl (10 mL, 2.0 mmol), and the reaction mixture was stirred at ambient temperature. After 16 h, the reaction mixture was concentrated under reduced pressure, and lyophilized. The compound was purified by reverse phase preparative HPLC to afford ((R)-1-((R)-3-(methoxy-d3)-2-(pyrazine-2-carboxamido)propanamido)-4-phenylbutyl)boronic acid (Compound 55, 52 mg). [M−H]⁻=402.4. ¹H NMR (400 MHz, CD₃OD): δ_{H} 9.24 (s, 1H), 8.81 (s, 1H), 8.70 (s, 1H), 7.22-7.08 (m, 5H), 5.00-4.96 (m, 1H), 3.89-3.85 (m, 1H), 3.78-3.75 (m, 1H), 2.66-2.57 (m, 3H), 1.67-1.48 (m, 4H).

### Example 56: ((R)-1-((R)-2-(2,5-dichlorobenzamido)-3-methoxy propanamido)-4-phenyl butyl)boronic acid

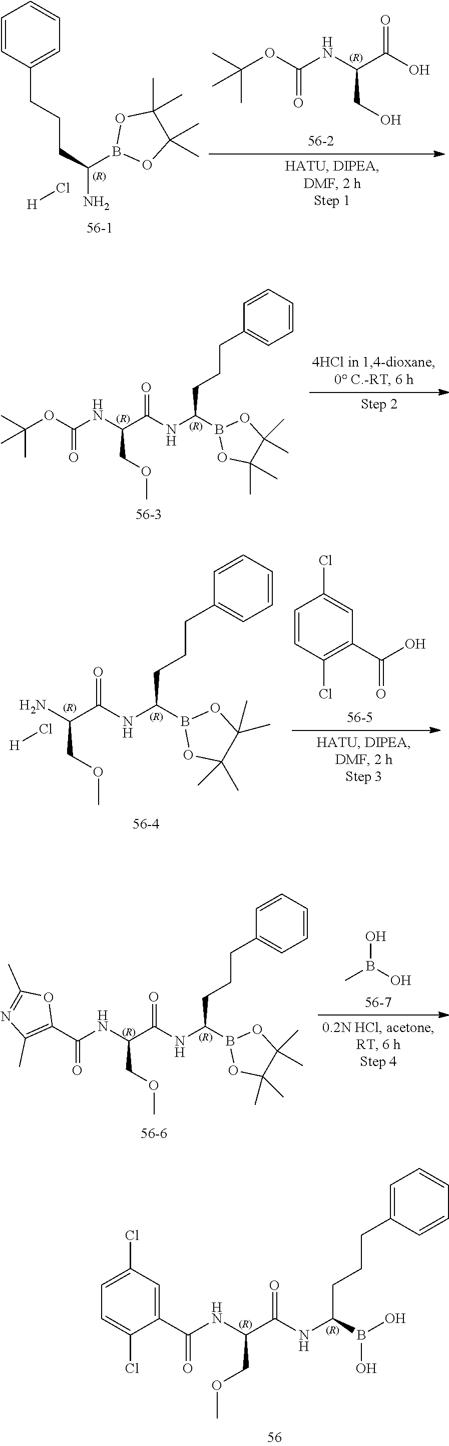

Synthesis of tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate [Step 1]: To a stirred solution of (tert-butoxycarbonyl)-D-serine (56-2, 600 mg, 2.74 mmol) in DMF (10 mL) were added HATU (2.08 g, 5.74 mmol) and (R)-4-phenyl-1-(4,4,5,5-tetramethyl-1,3,2-dioxaborolan-2-yl)butan-1-amine hydrochloride (56-1, 1.02 g, 3.28 mmol) followed by N,N-diisopropylethylamine (1.5 mL, 8.21 mmol) at 0° C., and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford tert-butyl ((R)-3-methoxy-1-oxo-1-(((R)-4-phenyl-1-(4,4,5,5-tetramethyl-1,3,2-dioxaborolan-2-yl)butyl)amino)propan-2-yl)carbamate (56-3, 1.2 g) which was used in the next step without further purification. [M−H]⁻=475.1.

Synthesis of (R)-2-amino-3-methoxy-N—((R)-4-phenyl-1-(4,4,5,5-tetramethyl-1,3,2-dioxaborolan-2-yl)butyl)propanamide hydrochloride [Step 2]: To a stirred solution of tert-butyl ((R)-3-methoxy-1-oxo-1-(((R)-4-phenyl-1-(4,4,5,5-tetramethyl-1,3,2-dioxaborolan-2-yl) butyl)amino)propan-2-yl)carbamate (56-3, 1.2 g, 2.52 mmol) in 1,4-dioxane (12 mL), was added HCl (6.6 mL, 4M in 1,4-dioxane, 25.2 mmol) dropwise at 0° C. and the reaction mixture was stirred at ambient temperature. After 6 h, the reaction mixture was concentrated under reduced pressure to afford (R)-2-amino-3-methoxy-N—((R)-4-phenyl-1-(4,4,5,5-tetramethyl-1,3,2-dioxaborolan-2-yl) butyl)propanamide hydrochloride (56-4, 900 mg), which was used in the next step without further purification. [M−H]⁻=375.4, found [M-83]⁻=293.3, mass of the corresponding boronic acid.

Synthesis of 2,5-dichloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide [Step 3]: To a stirred solution of 2,5-dichlorobenzoic acid (56-5, 120 mg, 0.63 mmol) in DMF (5 mL) were added HATU (478 mg, 1.26 mmol) and (R)-2-amino-3-methoxy-N—((R)-4-phenyl-1-(4,4,5,5-tetramethyl-1,3,2-dioxaborolan-2-yl)butyl)propanamide hydrochloride (56-4, 311 mg, 0.75 mmol) followed by N,N-diisopropylethylamine (0.34 mL, 1.88 mmol) at 0° C., and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure which was purified by PREP HPLC to afford 2,5-dichloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (56-6, 55 mg). [M−H]⁻=547.4. and [M-83]⁻=465.4 for the corresponding boronic acid.

Synthesis of ((R)-1-((R)-2-(2,5-dichlorobenzamido)-3-methoxypropanamido)-4-phenyl butyl)boronic acid [Step 4]: To a solution of 2,5-dichloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (56-6, 55 mg, 0.10 mmol) in acetone (1 mL) was added methylboronic acid (56-7, 60 mg, 1.00 mmol) followed by addition of 0.2 N HCl (0.5 mL) at 0° C. The reaction mixture was stirred at ambient temperature for 5 h. Volatiles were evaporated under reduced pressure and purified by PREP HPLC purification and lyophilized to give ((R)-1-((R)-2-(2,5-dichlorobenzamido)-3-methoxypropanamido)-4-phenylbutyl)boronic acid (Compound 56, 27 mg). [M−H]⁻=465.3. ¹H NMR (400 MHz, MeOD) (H: 7.57 (s, 1H), 7.46 (d, 2H), 7.24-7.11 (m, 5H), 4.92 (t, 1H), 3.77-3.74 (m, 1H), 3.72-3.68 (m, 1H), 3.35 (s, 3H), 2.69-2.67 (m, 1H), 2.64-2.59 (m, 2H), 1.69-1.57 (m, 4H).

### Example 57: ((R)-1-((R)-2-(2-chlorobenzamido)-3-methoxy propanamido)-4-phenylbutyl) boronic acid

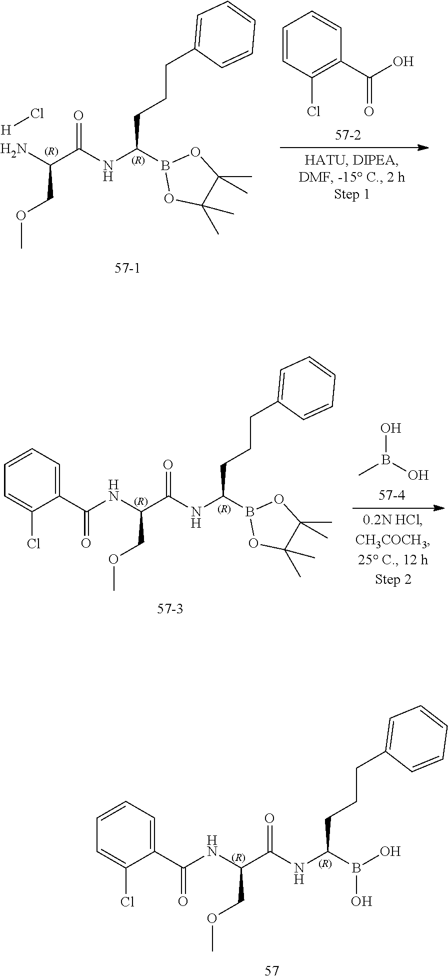

Synthesis of 2-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide [Step 1]: To a stirred solution of 2-chlorobenzoic acid (57-2, 50 mg, 0.6 mmol) in DMF (4 mL) was added HATU (182 mg, 0.5 mmol) followed by DIPEA (0.14 mL, 0.8 mmol) at 0° C. and stirred for 30 min. (R)-2-amino-3-methoxy-N—((R)-4-phenyl-1-(4,4,5,5-tetramethyl-1,3,2-dioxaborolan-2-yl)butyl)propanamide hydrochloride (57-1, 158 mg, 0.4 mmol) was added to it and stirred at 0° C. for 2 h. It was quenched with 5% aqueous K2CO₃ solution and extracted with EtOAc (thrice). Combined organic layer was washed with water and brine, dried over anhydrous Na₂SO₄ and evaporated under reduced pressure to afford 2-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (57-3, 30 mg). [M−H]⁻: 513.4. Corresponding boronic acid mass peak was also observed.

Synthesis of ((R)-1-((R)-2-(2-chlorobenzamido)-3-methoxypropanamido)-4-phenylbutyl) boronic acid [Step 2]: To a stirred solution of 2-chloro-N—((R)-3-methoxy-1-oxo-1-(((R)-4-phenyl-1-(4,4,5,5-tetramethyl-1,3,2-dioxaborolan-2-yl)butyl)amino)propan-2-yl)benzamide (57-3, 30 mg, 0.06 mmol) and methylboronic acid (57-4, 35 mg, 0.6 mmol) in acetone (1.0 mL) was added 0.2N HCl (1.0 mL) and stirred at ambient temperature for 16 h. Volatiles were evaporated under reduced pressure and the crude was purified by prep HPLC purification and lyophilized to afford ((R)-1-((R)-2-(2-chlorobenzamido)-3-methoxypropanamido)-4-phenylbutyl)boronic acid (Compound 57, 17 mg). [M−H]⁻: 431.3. 1H NMR (400 MHz, CD3OD) δ_{H}: 7.50-7.35 (m, 4H), 7.23-7.09 (m, 5H), 4.95 (m, 1H), 3.77-3.69 (m, 2H), 3.35 (s, 3H), 2.69-2.59 (m, 3H), 1.71-1.50 (m, 5H).

### Example 58: ((1R)-1-((2R)-3-methoxy-2-(tetrahydro-2H-pyran-2-carboxamido) propanamido)-4-phenylbutyl)boronic acid

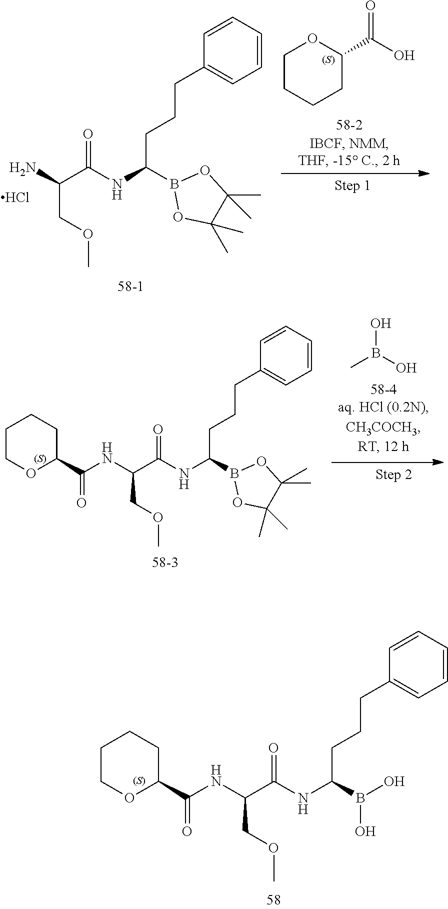

Synthesis of (S)—N—((R)-3-methoxy-1-oxo-1-(((R)-4-phenyl-1-(4,4,5,5-tetramethyl-1,3,2-dioxaborolan-2-yl)butyl)amino)propan-2-yl)tetrahydro-2H-pyran-2-carboxamide, [Step 1]: To a stirred solution of (S)-tetrahydro-2H-pyran-2-carboxylic acid (58-2, 113 mg, 0.87 mmol) in tetrahydrofuran (5 mL) at −15° C., isobutyl chloroformate (0.08 mL, 0.65 mmol) and N-methyl morpholine (0.08 mL, 1.09 mmol) were added. The reaction mixture was stirred at the same temperature for 30 min and a solution of (R)-2-amino-3-methoxy-N—((R)-4-phenyl-1-(4,4,5,5-tetramethyl-1,3,2-dioxaborolan-2-yl)butyl)propenamide hydrochloride (58-1, 300 mg, 0.72 mmol) and N-methyl morpholine (0.08 mL, 0.79 mmol) in tetrahydrofuran (1 mL) was added. The resulting reaction mixture warmed to 0° C. and allowed to stir for additional 2 h. The reaction mixture was neutralized with aqueous solution of hydrochloric acid (0.1 N) and extracted with ethyl acetate. The combined extracts were washed with 5% aqueous solution of potassium carbonate, water, brine, dried over anhydrous sodium sulphate, filtered and evaporated under reduced pressure to afford (S)—N—((R)-3-methoxy-1-oxo-1-(((R)-4-phenyl-1-(4,4,5,5-tetramethyl-1,3,2-dioxaborolan-2-yl)butyl)amino)propan-2-yl)tetrahydro-2H-pyran-2-carboxamide (58-3, 250 mg). The product was carried to next step without further purification. [M−H]⁻: 487.4.

Synthesis of ((R)-1-((R)-3-methoxy-2-((S)-tetrahydro-2H-pyran-2-carboxamido) propanamido)-4-phenylbutyl)boronic acid, [Step 2]: To a stirred solution of (S)—N—((R)-3-methoxy-1-oxo-1-(((R)-4-phenyl-1-(4,4,5,5-tetramethyl-1,3,2-dioxaborolan-2-yl)butyl)amino) propan-2-yl)tetrahydro-2H-pyran-2-carboxamide (58-3, 250 mg, 0.51 mmol) and methyl boronic acid (58-4, 306 mg, 5.11 mmol) in acetone (3 mL), aqueous solution of hydrochloric acid (0.2 N, 3 mL) was added at 0° C. The reaction mixture was stirred at ambient temperature for 16 h and volatiles were evaporated under reduced pressure. The product was purified by reverse phase prep-HPLC purification to give ((R)-1-((R)-3-methoxy-2-((S)-tetrahydro-2H-pyran-2-carboxamido) propanamido)-4-phenylbutyl)boronic acid (Compound 58, 21 mg). [M−H]⁻=405.3, ¹H NMR (400 MHz, DMSO-d6 and two drops of D₂O at 80° C.): δ_{H} 7.26-7.23 (m, 2H), 7.16-7.12 (m, 3H), 4.41 (t, 1H), 3.99-3.96 (m, 1H), 3.78-3.75 (m, 1H), 3.57-3.53 (m, 1H), 3.49-3.45 (m, 2H) 3.22 (s, 3H), 3.18-3.17 (m, 1H), 2.57-2.54 (m, 1H), 1.87-1.78 (m, 2H), 1.58-1.46 (m, 8H), 1.36-1.30 (m, 1H).

### Example 59: ((R)-1-((R)-3-methoxy-2-(2-methylpyrimidine-4-carboxamido)propanamido)-3-phenoxypropyl)boronic acid

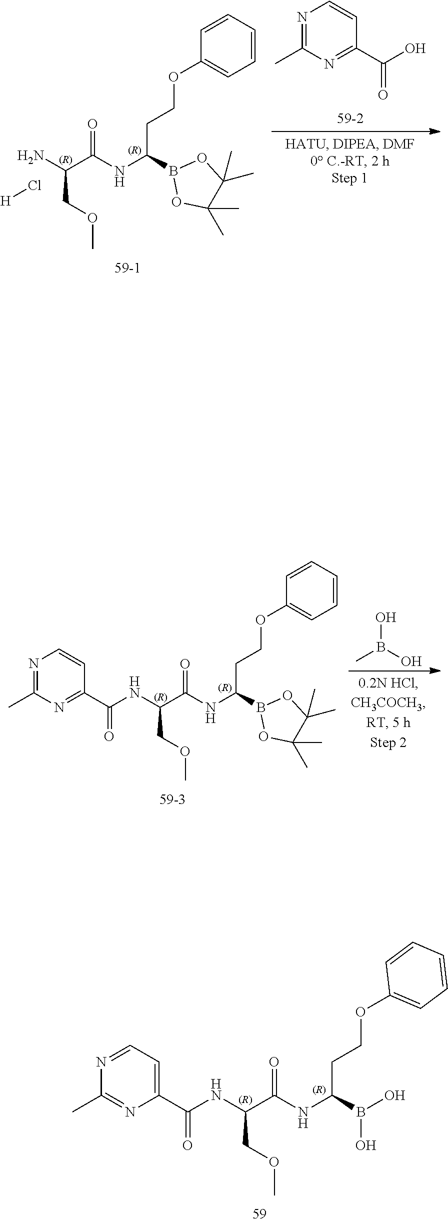

Synthesis of N—((R)-3-methoxy-1-oxo-1-(((R)-3-phenoxy-1-(4,4,5,5-tetramethyl-1,3,2-dioxaborolan-2-yl)propyl)amino)propan-2-yl)-2-methylpyrimidine-4-carboxamide, [Step 1]: To a stirred solution of 2-methylpyrimidine-4-carboxylic acid (59-2, 84 mg, 0.60 mmol) in DMF (5 mL) were added HATU (462 mg, 1.22 mmol) and (R)-2-amino-3-methoxy-N—((R)-3-phenoxy-1-(4,4,5,5-tetramethyl-1,3,2-dioxaborolan-2-yl)propyl)propanamide hydrochloride (59-1, 303 mg, 0.73 mmol) followed by N,N-diisopropylethylamine (0.31 mL, 1.82 mmol) at 0° C. and the reaction mixture was stirred at ambient temperature for 2 h. The reaction mixture was diluted with ice cold water and extracted with ethyl acetate. The organic extract was washed with brine, dried over anhydrous Na₂SO₄, filtered and concentrated under reduced pressure to afford N—((R)-3-methoxy-1-oxo-1-(((R)-3-phenoxy-1-(4,4,5,5-tetramethyl-1,3,2-dioxaborolan-2-yl)propyl)amino)propan-2-yl)-2-methylpyrimidine-4-carboxamide (59-3, 230 mg) which was used in the next step without further purification. [M−H]⁻=497.2.

Synthesis of ((R)-1-((R)-3-methoxy-2-(2-methylpyrimidine-4-carboxamido)propanamido)-3-phenoxypropyl)boronic acid, [Step 2]: To a solution of N—((R)-3-methoxy-1-oxo-1-(((R)-3-phenoxy-1-(4,4,5,5-tetramethyl-1,3,2-dioxaborolan-2-yl)propyl)amino)propan-2-yl)-2-methylpyrimidine-4-carboxamide (59-3, 330 mg, 0.66 mmol) in acetone (3 mL) was added methylboronic acid (397 mg, 6.62 mmol) followed by addition of 0.2 N HCl (3.3 mL) at 0° C. The reaction mixture was stirred at ambient temperature for 5 h. Volatiles were evaporated under reduced pressure and purified by PREP HPLC purification and lyophilized to give ((R)-1-((R)-3-methoxy-2-(2-methylpyrimidine-4-carboxamido)propanamido)-3-phenoxypropyl)boronic acid (Compound 59, 38 mg). [M−H]⁻=415. 1H NMR (400 MHz, MeOD) δ_{H}: 8.90 (d, 1H), 7.88 (d, 1H), 7.20 (t, 2H), 6.88-6.84 (m, 3H), 4.97 (bs, 1H), 4.03 (t, 2H), 3.92-3.89 (m, 1H), 3.80-3.77 (m, 1H), 3.39 (s, 3H), 2.92 (t, 1H), 2.78 (s, 3H), 2.02-1.87 (m, 2H).

### Example 60: ((R)-1-((R)-3-methoxy-2-(pyrazine-2-carboxamido)propanamido)-2-((1R,2S)-2-phenylcyclopropyl)ethyl)boronic acid

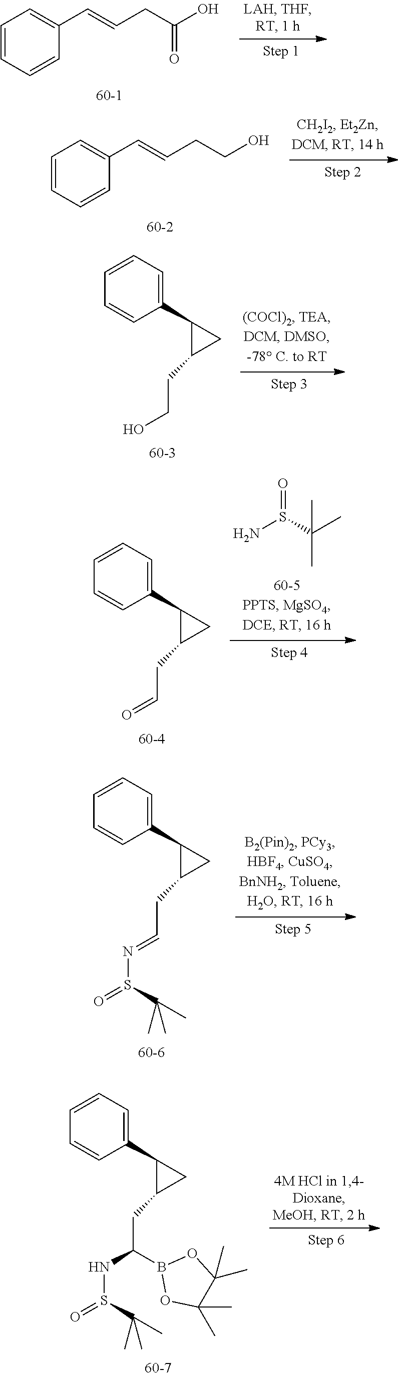

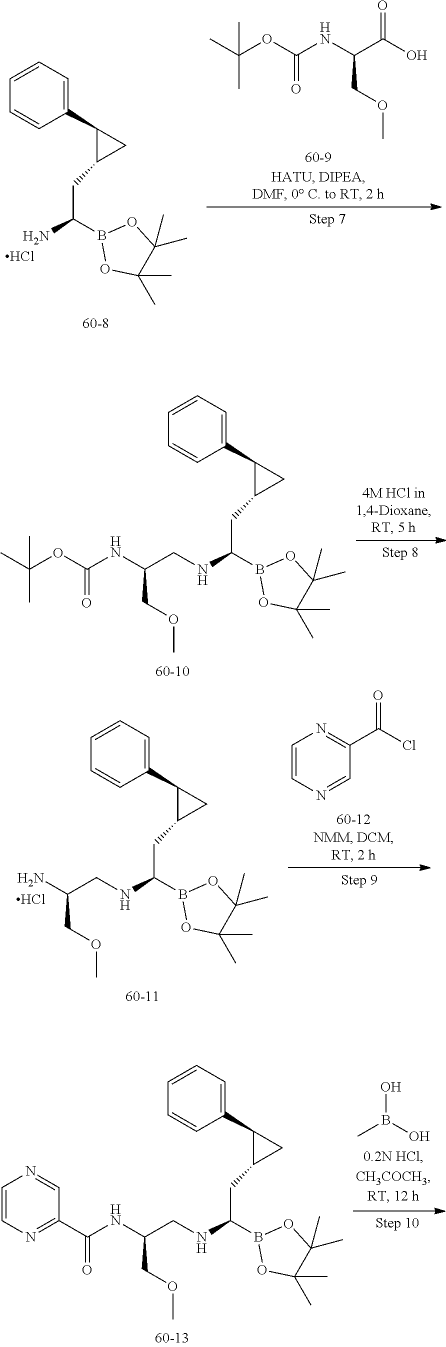

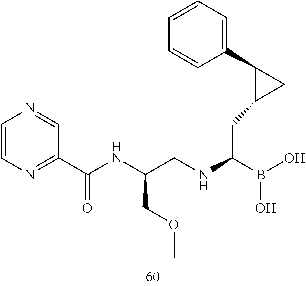 [SMILES: COC[C@H](CN[C@@H](C[C@H]1C[C@@H]1c1ccccc1)B(O)O)NC(=O)c1cnccn1]

Synthesis of (E)-4-phenylbut-3-en-1-ol, [Step 1]: To a stirred solution of (E)-4-phenylbut-3-enoic acid (60-1, 12.0 g, 74.0 mmol) in tetrahydrofuran (150 mL) at 0° C., a solution of lithium aluminium hydride (2 M) in tetrahydrofuran (37 mL, 74.0 mmol) was added dropwise. The reaction mixture was stirred at ambient temperature for 1 h and quenched with ice-cold water (12 mL) and 15% aqueous solution of sodium hydroxide (12 mL). The reaction mixture was filtered and washed with ethyl acetate. The filtrate was washed with water followed by brine. The combined organic extracts were dried over anhydrous sodium sulphate, filtered and evaporated under reduced. The product was purified by column chromatography to give (E)-4-phenylbut-3-en-1-ol (60-2, 6.0 g, 36.4 mmol). ¹H NMR (400 MHz, DMSO-d₆): 5H 7.38-7.37 (m, 2H), 7.32-7.28 (m, 2H), 7.21-7.18 (m, 1H), 6.45-6.41 (m, 1H), 6.33-6.25 (m, 1H), 4.59-4.56 (m, 1H), 3.54-3.49 (m, 2H), 2.36-2.33 (in, 2H).

Synthesis of rac-2-((1R,2S)-2-phenylcyclopropyl)ethan-1-ol, [Step 2]: In a two neck round bottom flask, dichloromethane (50 mL) was cooled to 0° C. and diethylzinc (ca. 15% in hexane) (45 mL, 45.5 mmol) was added and diiodomethane (3.7 mL, 45.5 mmol) was added. The reaction mixture was stirred for 30 min at 0° C. and (E)-4-phenylbut-3-en-1-ol (60-2, 1.5 g, 10.1 mmol) in dichloromethane (1 mL) was added dropwise. The reaction mixture was stirred at ambient temperature for 14 h, quenched with 1 N aqueous hydrochloric acid and extracted with ethyl acetate. The combined organic extracts were washed with water followed by brine, dried over anhydrous sodium sulphate and evaporated. The product was filtered through a bed of silica gel and filtered was evaporated to give rac-2-((1R,2S)-2-phenylcyclopropyl)ethan-1-ol (60-3, 900 mg). [M]=162.2.

Synthesis of rac-2-((1R,2S)-2-phenylcyclopropyl)acetaldehyde, [Step-3]: To a solution of dimethyl sulfoxide (0.66 mL, 9.25 mmol) in dichloromethane (20 mL) at −78° C., oxalyl chloride (0.52 mL, 6.16 mmol) was added dropwise. The reaction mixture was stirred at −78° C. for 30 min and a solution of rac-2-((1R,2S)-2-phenylcyclopropyl)ethan-1-ol (60-3, 500 mg, 3.08 mmol) in dichloromethane (5 ml) was added dropwise. The reaction mixture was stirred at same temperature for additional 30 min and triethylamine (2.1 mL, 15.4 mmol) was added. The reaction mixture was slowly warmed to room temperature, stirred for 30 min at ambient temperature, quenched with ice-cold water and extracted with dichloromethane. Combined organic extract were washed with brine, dried over anhydrous sodium sulphate and evaporated, filtered and concentrated under reduced pressure to give rac-2-((1R,2S)-2-phenylcyclopropyl)acetaldehyde (60-4, 400 mg, 2.00 mmol). The product was carried to next step without further purification. [M]=160.1.

Synthesis of rac-(R)-2-methyl-N—((E)-2-((1R,2S)-2-phenylcyclopropyl)ethylidene)propane-2-sulfinamide, [Step-4]: To a solution of (R)-2-methylpropane-2-sulfinamide (60-5, 1.51 g, 12.5 mmol) in dichloromethane (60 mL) at 0° C., PPTS (627 mg, 2.50 mmol), magnesium sulfate (7.5 g, 62.4 mmol) and rac-2-((1R,2S)-2-phenylcyclopropyl)acetaldehyde (60-4, 2.0 g, 12.5 mmol) were added under nitrogen atmosphere. The reaction mixture was allowed to stir at ambient temperature for 16 h and filtered through a pad of celite. The combined filtrate was dried over anhydrous sodium sulphate and evaporated, filtered and concentrated under reduced pressure. The product was purified by column chromatography to give rac-(R)-2-methyl-N—((E)-2-((1R,2S)-2-phenylcyclopropyl)ethylidene)propane-2-sulfinamide (60-6, 2.0 g). [M+H]⁺=264.15.

Synthesis of rac-(R)-2-methyl-N—((R)-2-((1R,2S)-2-phenylcyclopropyl)-1-(4,4,5,5-tetramethyl-1,3,2-dioxaborolan-2-yl)ethyl)propane-2-sulfinamide, [Step-5]: To a suspension of tricyclohexylphosphine tetra fluoro borate (84 mg, 0.23 mmol) in toluene (8 mL) were added copper sulphate pentahydrate (57 mg, 0.23 mmol) in water (2 mL), benzylamine (0.083 mL, 0.76 mmol) was added. The reaction mixture was stirred for 40 min at ambient temperature and a solution of rac-(R)-2-methyl-N—((E)-2-((1R,2S)-2-phenylcyclopropyl)ethylidene)propane-2-sulfinamide (60-6, 2.0 g, 7.59 mmol) in toluene (12 mL) and bis (pinacolato) diboron (3.9 g, 15.2 mmol) were added. The reaction mixture stirred at ambient temperature for 16 h, diluted with ethyl acetate, the precipitate was filtered through a short pad of deactivated silica gel. The filtrate was concentrated in vacuo and the residue was purified by column chromatography using deactivated silica gel to give rac-(R)-2-methyl-N-[rac-(1R)-2-[rac-(1R,2S)-2-phenylcyclopropyl]-1-(4,4,5,5-tetramethyl-1,3,2-dioxaborolan-2-yl)ethyl]propane-2-sulfinamide (60-7, 1.9 g). [M+H]⁺=392.20.

Synthesis of rac-(R)-2-((1R,2S)-2-phenylcyclopropyl)-1-(4,4,5,5-tetramethyl-1,3,2-dioxaborolan-2-yl)ethan-1-amine hydrochloride, [Step-6]: To the solution of rac-(R)-2-methyl-N-[rac-(1R)-2-[rac-(1R,2S)-2-phenylcyclopropyl]-1-(4,4,5,5-tetramethyl-1,3,2-dioxaborolan-2-yl)ethyl]propane-2-sulfinamide (60-7, 1.9 g, 4.85 mmol) in 1,4-Dioxane (25 mL) were added methanol (2.0 mL, 48.5 mmol) and 4 M HCl in 1,4-Dioxane (1.2 mL, 4.85 mmol) at 0° C. The resulting reaction mixture was allowed to stir at ambient temperature for 2 h and evaporated under reduced pressure and lyophilized to give rac-(R)-2-((1R,2S)-2-phenylcyclopropyl)-1-(4,4,5,5-tetramethyl-1,3,2-dioxaborolan-2-yl)ethan-1-amine hydrochloride (60-8, 1.7 g). The product was carried to next step without further purification.

Synthesis of rac-tert-butyl ((R)-3-methoxy-1-oxo-1-(((R)-2-((1R,2S)-2-phenylcyclopropyl)-1-(4,4,5,5-tetramethyl-1,3,2-dioxaborolan-2-yl)ethyl)amino)propan-2-yl)carbamate, [Step-7]: To a stirred solution of N-(tert-butoxycarbonyl)-O-methyl-D-serine (60-9, 1.1 g, 4.99 mmol) in dimethylformamide (20 mL), HATU (2.5 g, 6.57 mmol), N,N-Diisopropylethylamine (2.7 mL, 15.8 mmol) and rac-(R)-2-((1R,2S)-2-phenylcyclopropyl)-1-(4,4,5,5-tetramethyl-1,3,2-dioxaborolan-2-yl)ethan-1-amine hydrochloride (60-8, 1.7 g, 5.25 mmol) were added at 0° C. The reaction mixture was allowed to stir at ambient temperature for 2 h, diluted with ice-cold water and extracted with ethyl acetate. Combined organic extract were washed with brine, dried over anhydrous sodium sulphate, filtered and concentrated under reduced pressure to give rac-tert-butyl ((R)-3-methoxy-1-oxo-1-(((R)-2-((1R,2S)-2-phenylcyclopropyl)-1-(4,4,5,5-tetramethyl-1,3,2-dioxaborolan-2-yl)ethyl)amino)propan-2-yl)carbamate (60-10, 2.4 g). The product was carried to next step without further purification. [M+H]⁺=489.4.

Synthesis of rac-(R)-2-amino-3-methoxy-N—((R)-2-((1R,2S)-2-phenylcyclopropyl)-1-(4,4,5,5-tetramethyl-1,3,2-dioxaborolan-2-yl)ethyl)propanamide hydrochloride, [Step-8]: To a stirred solution of rac-tert-butyl ((R)-3-methoxy-1-oxo-1-(((R)-2-((1R,2S)-2-phenylcyclopropyl)-1-(4,4,5,5-tetramethyl-1,3,2-dioxaborolan-2-yl)ethyl)amino)propan-2-yl)carbamate (60-10, 2.4 g, 4.91 mmol) in 1, 4-Dioxane (25 mL) was added hydrochloric acid 4 M in 1, 4-Dioxane (13 mL, 49.1 mmol) at 0° C. The reaction mixture was stirred at ambient temperature for 5 h and volatiles were evaporated under reduced pressure and lyophilized to give rac-(R)-2-amino-3-methoxy-N—((R)-2-((1R,2S)-2-phenylcyclopropyl)-1-(4,4,5,5-tetramethyl-1,3,2-dioxaborolan-2-yl)ethyl)propanamide hydrochloride (60-11, 1.6 g). The product was carried to next step without further purification. [M−H]⁻=387.5 and 305.4 [corresponding to boronic acid as major].

Synthesis of N—((R)-3-methoxy-1-oxo-1-(((R)-2-((1R,2S)-2-phenylcyclopropyl)-1-(4,4,5,5-tetramethyl-1,3,2-dioxaborolan-2-yl)ethyl)amino)propan-2-yl)pyrazine-2-carboxamide, [Step 9]: To the stirred solution of rac-(R)-2-amino-3-methoxy-N—((R)-2-((1R,2S)-2-phenylcyclopropyl)-1-(4,4,5,5-tetramethyl-1,3,2-dioxaborolan-2-yl)ethyl)propanamide hydrochloride (60-11, 1.6 g, 3.77 mmol) in dichloromethane (30 mL) was added N-methyl morpholine (1.7 mL, 15.1 mmol) at 0° C. The reaction mixture was stirred at same temperature for 15 min and pyrazine-2-carbonyl chloride (60-12, 0.59 g, 4.14 mmol) was added in one portion.

The reaction mixture was stirred at ambient temperature for 2 h, diluted with dichloromethane and washed with cold water and followed by brine wash. Combined organic extracts were dried over anhydrous sodium sulphate, filtered and concentrated under reduced pressure. The product was purified by prep-HPLC to give N—((R)-3-methoxy-1-oxo-1-(((R)-2-((1R,2S)-2-phenylcyclopropyl)-1-(4,4,5,5-tetramethyl-1,3,2-dioxaborolan-2-yl)ethyl)amino)propan-2-yl)pyrazine-2-carboxamide (60-13, 120 mg). [M−H]⁻=493.4 and 411.3 [corresponding to boronic acid]. 1H NMR and LCMS indicated the product is present as mixture of boronate ester and corresponding boronic acid.

Synthesis of rac-((R)-1-((R)-3-methoxy-2-(pyrazine-2-carboxamido)propanamido)-2-((1R,2S)-2-phenylcyclopropyl)ethyl)boronic acid, [Step 10]: To a stirred solution of N—((R)-3-methoxy-1-oxo-1-(((R)-2-((1R,2S)-2-phenylcyclopropyl)-1-(4,4,5,5-tetramethyl-1,3,2-dioxaborolan-2-yl)ethyl)amino)propan-2-yl)pyrazine-2-carboxamide (60-13, 100 mg, 0.202 mmol) and methylboronic acid (121 mg, 2.02 mmol) in acetone (3 mL) was added 0.2 N aqueous hydrochloric acid (6 mL) at ice-cold condition and stirred at ambient temperature for 12 h. Volatiles were evaporated under reduced pressure and the product was purified by prep-HPLC and lyophilized to give rac-((R)-1-((R)-3-methoxy-2-(pyrazine-2-carboxamido)propanamido)-2-((1R,2S)-2-phenylcyclopropyl)ethyl)boronic acid (Compound 60, 30 mg). [M−H]⁻: 411.2, 1 H NMR (400 MHz, DMSO-d6 and 2 drops of D₂O): δ_{H} 9.18 (d, 1H), 8.89 (d, 1H), 8.76 (s, 1H), 7.20-7.16 (m, 2H), 7.09-7.05 (m, 1H), 7.00-6.98 (m, 2H), 4.70-4.68 (m, 1H), 3.67-3.54 (m, 2H), 3.25-3.16 (m, 4H), 1.67-1.60 (m, 2H), 1.53-1.48 (m, 1H), 1.04 (m, 1H), 0.79-0.71 (m, 2H).

### Example 61

### Biological/Biochemical Evaluation:

The inhibitory activity of the compounds of the present invention against LONP1, 20S proteasome and other proteases are determined by assays known to persons of ordinary skill in the art (see, e.g., Fishovitz, J. et al. “Active-Site-Directed Chemical Tools for Profiling Mitochondrial Lon Protease” ACS Chem. Biol. 6, 781-788 (2011)).

In this Example, LONP1 (NM_004793.4) activity was measured by a FRET-based assay for protease activity using a fluorogenic peptide DabcylYRGIT(2Abu)SORQK(5-FAM) (Cambridge Research Biochemicals) as substrate. LONP1 activity is followed by an increase in fluorescence signal due to the degradation of the peptide. Inhibition of LONP1 protease activity by an inhibitor compound of the disclosure elicits a decrease in the fluorescent signal.

The assay is performed in a 384-well plate (Greiner, cat. #781076) using the following reagents and conditions: substrate (3 μM) was incubated for 1 hour at 37° C. in the presence of LONP1 (15 nM as monomer), 25 mM Tris pH 8.0, 10 mM MgCl₂, 0.03 mg/mL ESA, 0.5 mM DTT, 0.0003% Tween-20, 10 mM NaCl, 0.06 mM ATP and 0.5 mM EGTA in a 15 μL final volume. The LONP1-containing mix (10 μL) was incubated with the test compound for 15 min at 37° C. before adding the peptide-containing mix (5 μL). Solutions were dispensed using a small cassette-Multidrop Combi (Thermo Scientific). Fluorescence was measured using a PheraStar plate reader (BMG Labtech) FI-FRET EX 485 nm Em 520 nm.

The IC₅₀ values for binding to LONP1 are summarized in Table 2 below. Each value is based on an average of a minimum of two repeats.

TABLE 2
IC50 assay data for compounds of the disclosure
binding to LONP1; A: <0.05 μM; B: 0.05-
0.5 μM; C: 0.5-5 μM; D >5 μM

| Compound | Activity IC₅₀ |
|---|---|
| No. | (μM) |
| 1 | A |
| 2 | B |
| 3 | A |
| 4 | A |
| 5 | A |
| 6 | A |
| 7 | A |
| 8 | B |
| 9 | A |
| 10 | A |
| 11 | A |
| 12 | A |
| 13 | A |
| 14 | A |
| 15 | B |
| 16 | B |
| 17 | B |
| 18 | B |
| 19 | C |
| 20 | A |
| 21 | A |
| 22 | D |
| 23 | A |
| 24 | A |
| 25 | A |
| 26 | C |
| 27 | B |
| 28 | D |
| 29 | C |
| 30 | B |
| 31 | A |
| 32 | A |
| 33 | A |
| 34 | A |
| 35 | A |
| 36 | A |
| 37 | A |
| 38 | B |
| 39 | C |
| 40 | C |
| 41 | B |
| 42 | A |
| 43 | A |
| 44 | A |
| 45 | B |
| 46 | — |
| 47 | — |
| 48 | — |
| 49 | — |
| 50 | — |
| 51 | A |
| 52 | A |
| 53 | C |
| 54 | A |
| 55 | A |
| 56 | A |
| 57 | A |
| 58 | — |
| 59 | — |
| 60 | — |

In one embodiment, beneficial compounds of this disclosure have IC₅₀ values of less than 5 μM. In another embodiment, beneficial compounds of this disclosure have IC₅₀ less than 2.5 μM. In another embodiment, beneficial compounds of this disclosure have IC₅₀ less than 1 μM. In another embodiment, beneficial compounds of this disclosure have IC₅₀ less than 0.5 μM. In another embodiment, beneficial compounds of this disclosure have IC₅₀ less than 0.1 μM. In another embodiment, beneficial compounds of this disclosure have IC₅₀ less than 0.05 μM. In another embodiment, beneficial compounds of this disclosure have IC₅₀ less than 0.01 μM.

### Cell Viability Assay:

### Materials and Kits:

Cell Proliferation Kit I (MTT), Merck, Cat #11465007001
DMEM GlutaMax, Thermo Fisher Scientific, Cat #31966021—for expansion and assay
DMEM GlutaMax, low glucose, Thermo Fisher Scientific, Cat #21885025—for expansion for cell viability assay
FBS, Gibco, Cat #A3840402

### Assay Procedure:

One day before treatment, 3,000-5,000/mL of 143b cells are placed in aliquots of 100 μL per well in flat bottom ThermoFisher 96 well plate. The starting seeding number is optimised in relation to the batch of cells and medium. The assay lasts for 8 days from seeding to MTT assay, and so the seeding number must be selected to avoid over-confluency at the last day of the assay.

On day 0, 100 μl of medium (Cat #21885025) is transferred to compound/DMSO plate, then the compound/DMSO-containing medium is transferred to the plates with pre-seeded cells.

Incubate for 7 days at 37° C., 5% C02 incubator.

On day 7, the medium is discarded. 100 μl of MTT labelling reagent mixed 1:10 in culture medium (Cat #21885025) is added and incubated for 4 hours at 37° C., 5% C02 incubator. 100 μl of MTT solubilization solution is added, mixed well and incubated overnight at 37° C.

Absorbance is measured at 570 nm on a plate reader.

### Compound Plate Setup:

The compounds are dispensed in a 96-well Greiner plate (cat no. 651201).
Volume of each compound solution: 200 nL
Final conc of DMSO: 0.1% in all wells
Starting concentration: 10 mM (final concentration on assay plate: 10 μM). In total, 8 doses and three replicates per dose per compound.
Dilution factor: 3.162

The compounds are dissolved in DMSO and dispensed according to the concentration titrations and experimental design (indicated above).

Two plates of the same compounds are dispensed and the remaining plates are retained as a backup.

Compounds can be dispensed in an Echo dispenser and sealed immediately so that they are not exposed to air and contamination. The protocol is performed under the LAF bench.

On the first day of treatment (Day 0), compound plates are opened under the LAF bench. 100 μl of assay medium (Cat #21885025) is added to each well and 100.2 μl of medium+compound/DMSO is transferred to the assay plates containing pre-seeded cells.

1. A compound of structural Formula I 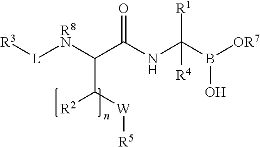
or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹¹, CONR¹¹R¹², NR¹¹R¹², SR¹¹, SO2NR¹¹R¹², C1-C4 alkyl, C1-C4 haloalkyl, C1-C4 alkoxy, phenyl, or a 5- or 6-membered heteroaryl;
n is 1 or 2;
each occurrence of R² is independently selected from H or C1-C4 alkyl;
L is C(O), C(O)O, C(O)NR⁶, S(O)₂, or a bond;
R³ is C1-C4 alkyl optionally substituted with one or more substituents each independently selected from the group consisting of deuterium, halogen, cyano, hydroxyl, C1-C4 alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹¹, CONR¹¹R¹², NR¹¹R¹², SR¹¹, SO2NR¹¹R¹², C₁-C₄ alkoxyl, or C₁-C₄ alkyl that is optionally substituted with one to three substituents selected from deuterium, halogen, cyano, hydroxyl, or C₁-C₄ alkoxyl;
W is selected from the group consisting of O, S, S(O), SO₂ and S(O)(NH);
R⁴ is selected from hydrogen, deuterium or C1-C2 alkyl;
R⁵ is hydrogen, deuterium, C₁-C₄ alkyl or C₁-C₄ alkoxyl wherein each alkyl or alkoxyl is optionally substituted with one or more substituents each independently selected from the group consisting of deuterium, halogen, cyano, hydroxyl, C₁-C₄ alkoxyl, C₁-C₅ alkyl-alkoxyl or phenyl; or
R⁵ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is NR⁹R¹⁰;
R⁶ is hydrogen, deuterium or C₁-C₂ alkyl optionally substituted with one or more of deuterium, halogen, hydroxyl and phenyl, wherein phenyl is optionally substituted with one or more substituent selected from halogen, hydroxyl and C₁-C₂ alkyl;
R⁷ is hydrogen, or R⁷ and R¹, together with the boron atom to which OR⁷ is attached form a 5-membered heteroalkyl ring;
R⁸ is selected from hydrogen, deuterium, or C₁-C₂ alkyl optionally substituted with one or more substituents each independently selected from the group consisting of halogen, hydroxyl, cyano, methoxyl and phenyl; or
R⁸, L and R³ together with the N to which R⁸ and L are attached form a saturated or unsaturated heterocycloalkyl group optionally having one or more additional heteroatoms selected from N, O and S, wherein the heterocycloalkyl is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl, and is optionally fused to an aryl or heteroaryl group which is optionally substituted with one or more substituents selected from halogen, cyano, hydroxyl, C₁-C₄ alkoxyl, or C₁-C₄ alkyl, wherein C₁-C₄ alkyl is optionally substituted with one to three substituents selected from halogen, cyano and C₁-C₄ alkoxyl, or is optionally fused to a cycloalkyl or heterocycloalkyl group which is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl;
R⁹ and R¹⁰ are each independently selected from hydrogen or C₁-C₆ alkyl that is optionally substituted with one to three substituents selected from halogen, cyano, or C₁-C₄ alkoxyl; and
R¹¹ and R¹² are each independently selected from hydrogen, deuterium, C1-C4 alkyl; C1-C4 haloalkyl, C1-C5 alkyl-alkoxyl, C3-C7 cycloalkyl, or R¹¹ and R¹² together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

2. The compound according to claim 1, wherein R¹ is selected from methyl, ethyl, n-propyl, i-propyl, n-butyl or tert-butyl, each optionally substituted with a phenyl ring.

3. The compound according to claim 1, wherein R¹ is selected from methyl, n-propyl, n-butyl or tert-butyl.

4. The compound according to claim 1, wherein R¹ is selected from phenyl-(CH₂)₂—, phenyl-(CH₂)₃—, phenyl-(CO)(CH₂)—, or phenyl-(CO)(CH₂)₂—; wherein phenyl is optionally substituted with a substituent selected from halogen, cyano, hydroxyl, C₁-C₂ alkyl, C₁-C₂ haloalkyl or C₁-C₂ alkoxyl.

5. The compound according to claim 1, wherein each occurrence of R² is independently selected from hydrogen or methyl.

6. The compound according to claim 1, wherein R⁵ is selected from the group consisting of optionally substituted methyl, phenyl, cyclopropyl, pyridinyl, benzyl, or NMe₂.

7. The compound according to claim 1, wherein R⁵ is methyl, or methyl substituted with one, two or three deuterium atoms.

8. The compound according to claim 1, wherein R⁵ is phenyl.

9. The compound according to claim 1, wherein R⁵ is hydrogen.

10. The compound according to claim 1, wherein R⁵ is cyclopropyl.

11. The compound according to claim 1, wherein R⁵ is pyridinyl.

12. The compound according to claim 1, wherein R⁵ is benzyl.

13. The compound according to claim 1, wherein R⁵ is NMe₂.

14. The compound according to claim 1, wherein n is 1.

15. The compound according to claim 1, wherein n is 2.

16. The compound according to claim 1, wherein L is C(O).

17. The compound according to claim 1, wherein R³ is C₁-C₄ alkyl, a 5- or 6-membered heteroaryl, C₆ aryl, a 5- or 6-membered heterocycloalkyl or C₆ cycloalkyl, and wherein R³ is optionally substituted.

18. The compound according to claim 1, wherein R³ is methyl, ethyl, n-propyl, i-propyl, n-butyl, or tert-butyl, each optionally substituted with a phenyl ring.

19. The compound according to claim 1, wherein R³ is selected from methyl, i-propyl and tert-butyl.

20. The compound according to claim 1, wherein R³ is selected from phenyl, phenyl-(CH₂)— and phenyl-(CH₂)₂—, wherein the phenyl group is optionally substituted.

21. The compound according to claim 1, wherein R³ is selected from aryl, heteroaryl, cycloalkyl or heterocycloalkyl selected from pyrazinyl, tetrahydropyrrolyl, tetrahydrofuranyl, tetrahydropyranyl, cyclohexanyl, oxazolyl and morpholinyl, wherein said aryl, heteroaryl, cycloalkyl or heterocycloalkyl is optionally substituted.

22. The compound according to claim 21, wherein R³ is selected from phenyl, pyridinyl, pyrazinyl, tetrahydropyranyl or morpholinyl, each of which is optionally substituted.

23. The compound according to claim 22, wherein R³ is phenyl, which is optionally substituted.

24. The compound according to claim 22, wherein R³ is pyridinyl, which is optionally substituted.

25. The compound according to claim 22, wherein R³ is pyrazinyl, which is optionally substituted.

26. The compound according to claim 22, wherein R³ is morpholinyl, which is optionally substituted.

27. The compound according to claim 1, wherein said substituent is selected from one to three of halogen, hydroxyl, C1-C4 alkyl and C1-C4 alkoxyl.

28. The compound according to claim 1, wherein said substituent is selected from one or two of halogen, methyl, tert-butyl and methoxyl.

29. The compound according to claim 1, wherein R³ is selected from phenyl, 2-chlorophenyl, 3-chlorophenyl, 4-chlorophenyl, 2, 5-dichlorophenyl, pyridinyl, 2-methylpyridinyl, 2-methoxylpyridinyl, morpholinyl, and pyrazinyl.

30. The compound according to claim 1, wherein R⁷ is hydrogen.

31. The compound according to claim 1, wherein R⁶ and/or R⁸ is hydrogen.

32. The compound according to claim 1, wherein R⁸, L and R³ together with the N to which R⁸ and L are attached form a heterocycloalkyl group, wherein the heterocycloalkyl is substituted with oxo and is optionally fused to an aryl.

33. The compound according to claim 1, wherein R⁸, L and R³ together with the N to which R⁸ and L are attached form a heterocycloalkyl group, wherein the heterocycloalkyl is substituted with oxo and is fused to an aryl, wherein said aryl is optionally substituted.

34. The compound according to claim 1, wherein R¹¹ and R¹² are each independently selected from hydrogen, deuterium, C1-C2 alkyl; C1-C2 haloalkyl, C1-C2 alkyl-alkoxyl or C3-C7 cycloalkyl, wherein C3-C7 cycloalkyl is optionally substituted with one or more substituent selected from deuterium, F, Cl, hydroxyl, oxo, CN, C1-C2 alkyl, C1-C2 haloalkyl or C1-C2 alkoxyl.

35. The compound according to claim 1, wherein R¹¹ and R¹² together with the N to which they are attached form 3 to 7 membered heterocyclic ring optionally having one or two additional heteroatoms selected from N, O and S, which is optionally substituted with one or more substituent selected from deuterium, F, Cl, hydroxyl, oxo, CN, C1-C2 alkyl, C1-C2 haloalkyl or C1-C2 alkoxyl.

36. The compound according to claim 1, wherein halogen is selected from fluoro or chloro.

37. The compound according to claim 1, wherein halogen is chloro.

38. The compound according to claim 1, wherein halogen is fluoro.

39. The compound according to claim 1, which is selected from any one of:
((R)-1-((R)-3-methoxy-2-(pyrazine-2-carboxamido)propanamido)-4-phenylbutyl) boronic acid;
((R)-2-(benzyloxy)-1-((R)-3-methoxy-2-(pyrazine-2-carboxamido)propanamido)ethyl) boronic acid;
((R)-1-((R)-2-benzamido-3-methoxypropanamido)-4-phenylbutyl) boronic acid;
((R)-1-((R)-3-methoxy-2-(6-methoxypicolinamido)propanamido)-4-phenylbutyl) boronic acid;
((R)-1-((R)-3-methoxy-2-(pyrazine-2-carboxamido)propanamido)-4-(4-methoxyphenyl) butyl) boronic acid;
((R)-1-((2R,3S)-3-methoxy-2-(pyrazine-2-carboxamido)butanamido)-4-phenylbutyl) boronic acid;
((R)-1-((R)-3-methoxy-2-((S)-5-oxopyrrolidine-2-carboxamido)propanamido)-4-phenyl butyl) boronic acid;
((R)-1-((R)-3-methoxy-2-((R)-5-oxopyrrolidine-2-carboxamido)propanamido)-4-phenyl butyl) boronic acid;
((R)-1-((R)-3-methoxy-2-(picolinamido)propanamido)-4-phenylbutyl) boronic acid;
((R)-1-((R)-3-methoxy-2-(6-methylpicolinamido)propanamido)-4-phenylbutyl) boronic acid;
((R)-1-((R)-3-(benzyloxy)-2-(pyrazine-2-carboxamido)propanamido)-4-phenylbutyl) boronic acid;
((R)-4-(4-chlorophenyl)-1-((R)-3-methoxy-2-(pyrazine-2-carboxamido)propanamido) butyl) boronic acid;
((R)-4-phenyl-1-((R)-2-(pyrazine-2-carboxamido)-3-(pyridin-2-yloxy)propanamido)butyl) boronic acid;
((R)-1-((R)-3-methoxy-2-(pyrazine-2-carboxamido)propanamido)-3-phenoxypropyl) boronic acid;
((R)-4-(3-chlorophenyl)-1-((R)-3-methoxy-2-(pyrazine-2-carboxamido)propanamido) butyl) boronic acid;
((R)-1-((R)-2-(1,3-dioxoisoindolin-2-yl)-3-methoxypropanamido)-4-phenylbutyl) boronic acid;
((R)-1-((R)-3-methoxy-2-(pyrazine-2-carboxamido)propanamido)-4-(3-methoxyphenyl) butyl) boronic acid;
((R)-2-cyclopropyl-1-((R)-3-methoxy-2-(pyrazine-2-carboxamido)propanamido)ethyl) boronic acid;
((R)-1-((R)-2-(2,5-dioxopyrrolidin-1-yl)-3-methoxypropanamido)-4-phenylbutyl) boronic acid;
((R)-1-((R)-3-cyclopropoxy-2-(pyrazine-2-carboxamido)propanamido)-4-phenylbutyl) boronic acid;
((R)-3-(4-chlorophenoxy)-1-((R)-3-methoxy-2-(pyrazine-2-carboxamido)propanamido) propyl) boronic acid;
((S)-2-((R)-3-methoxy-2-(pyrazine-2-carboxamido)propanamido)-5-phenylpentan-2-yl) boronic acid;
((R)-3-(4-fluorophenoxy)-1-((R)-3-methoxy-2-(pyrazine-2-carboxamido)propanamido) propyl) boronic acid;
((R)-3-(4-chloro-2-fluorophenoxy)-1-((R)-3-methoxy-2-(pyrazine-2-carboxamido) propanamido)propyl) boronic acid;
((R)-3-(4-chloro-3-fluorophenoxy)-1-((R)-3-methoxy-2-(pyrazine-2-carboxamido) propanamido)propyl) boronic acid;
((S)-3-(4-chloro-3-fluorophenoxy)-1-((R)-3-methoxy-2-(pyrazine-2-carboxamido) propanamido)propyl) boronic acid;
((R)-3-(4-chloro-2-methylphenoxy)-1-((R)-3-methoxy-2-(pyrazine-2-carboxamido) propanamido)propyl) boronic acid;
((S)-3-(4-chloro-2-methylphenoxy)-1-((R)-3-methoxy-2-(pyrazine-2-carboxamido) propanamido)propyl) boronic acid;
((S)-3-(4-chloro-2-fluorophenoxy)-1-((R)-3-methoxy-2-(pyrazine-2-carboxamido) propanamido)propyl) boronic acid;
((R)-1-((2R,3S)-3-methoxy-2-(pyrazine-2-carboxamido)butanamido)-3-phenoxypropyl) boronic acid;
((R)-1-((R)-3-methoxy-2-(morpholine-4-carboxamido)propanamido)-4-phenylbutyl) boronic acid;
((R)-4-(4-fluorophenyl)-1-((R)-3-methoxy-2-(pyrazine-2-carboxamido)propanamido) butyl) boronic acid;
((R)-1-((R)-4-methoxy-2-(pyrazine-2-carboxamido)butanamido)-4-phenylbutyl) boronic acid;
[(1R)-4-phenyl-1-[(2S)-3-(phenylsulfanyl)-2-(pyrazin-2-ylformamido)propanamido]butyl]boronic acid;
((R)-1-((S)-3-(N,N-dimethylsulfamoyl)-2-(pyrazine-2-carboxamido)propanamido)-4-phenylbutyl) boronic acid;
((R)-1-((R)-3-(N,N-dimethylsulfamoyl)-2-(pyrazine-2-carboxamido)propanamido)-4-phenylbutyl) boronic acid;
((R)-1-((R)-4-(methylsulfonyl)-2-(pyrazine-2-carboxamido)butanamido)-4-phenylbutyl) boronic acid;
((R)-1-((R)-3-methoxy-2-(pyrazine-2-carboxamido)propanamido)-3-methylbutyl) boronic acid;
((R)-1-((R)-3-hydroxy-2-(pyrazine-2-carboxamido)propanamido)-4-(4-hydroxyphenyl) butyl) boronic acid;
((R)-4-(4-hydroxyphenyl)-1-((R)-3-methoxy-2-(pyrazine-2-carboxamido)propanamido) butyl) boronic acid;
((R)-1-((R)-3-methoxy-2-((R)-tetrahydro-2H-pyran-2-carboxamido)propanamido)-4-phenylbutyl) boronic acid;
((R)-1-((R)-3-methoxy-2-(6-methylpicolinamido)propanamido)-3-phenoxypropyl) boronic acid;
((R)-1-((R)-3-methoxy-2-(picolinamido)propanamido)-3-phenoxypropyl) boronic acid;
((R)-1-((R)-3-methoxy-2-(5-methylnicotinamido)propanamido)-3-phenoxypropyl) boronic acid;
((R)-1-((R)-3-methoxy-2-(pyrazine-2-carboxamido)propanamido)-3-phenylpropyl) boronic acid;
((R)-1-((R)-3-methoxy-2-(2-(trifluoromethyl)pyrimidine-4-carboxamido)propanamido)-3-phenoxypropyl) boronic acid;
((R)-1-((R)-3-methoxy-2-(6-methylnicotinamido)propanamido)-3-phenoxypropyl) boronic acid;
((R)-1-((R)-3-methoxy-2-(2-methylnicotinamido)propanamido)-3-phenoxypropyl) boronic acid;
((R)-1-((R)-3-methoxy-2-(nicotinamido)propanamido)-3-phenoxypropyl) boronic acid;
((R)-1-((R)-2-(2,4-dimethyloxazole-5-carboxamido)-3-methoxypropanamido)-4-phenyl butyl) boronic acid;
((R)-1-((R)-2-(4-chlorobenzamido)-3-methoxy propanamido)-4-phenylbutyl)boronic acid;
((R)-1-((R)-3-methoxy-2-(pyrimidine-5-carboxamido) propanamido)-4-phenylbutyl) boronic acid;
((R)-1-((R)-3-methoxy-2-(pyrazine-2-carboxamido) propanamido)-4-oxo-4-phenylbutyl) boronic acid;
((R)-1-((R)-2-(3-chlorobenzamido)-3-methoxy propanamido)-4-phenylbutyl)boronic acid;
((R)-1-((R)-3-(methoxy-d3)-2-(pyrazine-2-carboxamido) propanamido)-4-phenylbutyl) boronic acid;
((R)-1-((R)-2-(2,5-dichlorobenzamido)-3-methoxy propanamido)-4-phenylbutyl)boronic acid;
((R)-1-((R)-2-(2-chlorobenzamido)-3-methoxy propanamido)-4-phenylbutyl)boronic acid;
((1R)-1-((2R)-3-methoxy-2-(tetrahydro-2H-pyran-2-carboxamido)propanamido)-4-phenyl butyl)boronic acid;
((R)-1-((R)-3-methoxy-2-(2-methylpyrimidine-4-carboxamido)propanamido)-3-phenoxy propyl)boronic acid; and
((R)-1-((R)-3-methoxy-2-(pyrazine-2-carboxamido)propanamido)-2-((1R,2S)-2-phenyl cyclopropyl)ethyl)boronic acid.

40. The compound according to claim 1, which is selected from any one of structures 1 to 60 or an oxaborolane isomer thereof.

41. The compound according to claim 1, which is selected from a compound of the group consisting of:
(i) compound 1, 3, 4, 5, 6, 7, 9, 10, 11, 12, 13, 14, 20, 21, 23, 24, 25, 31, 32, 33, 34, 35, 36, 37, 42, 43, 44, 51, 52, 54, 55, 56 and 57;
(ii) compound 2, 8, 15, 16, 17, 18, 27, 30, 38, 41 and 45;
(iii) compound 19, 22, 26, 28, 29, 39, 40 and 53; or
(iv) compound 1, 3, 4, 6, 9, 10, 11, 12, 13, 14, 20, 21, 25, 32, 33, 34, 36, 37, 44, 51, 55, 56 and 57.

42. The compound according to claim 1, wherein the compound is an inhibitor of LONP1.

43. A pharmaceutical composition comprising one or more compounds according to claim 1 or pharmaceutically acceptable salt, solvate, stereoisomer or mixture of stereoisomers, tautomer, isotopic form, or pharmaceutically active metabolite thereof, or combinations thereof, and one or more pharmaceutically acceptable carrier.

44. A pharmaceutical composition comprising a compound according to Formula I, 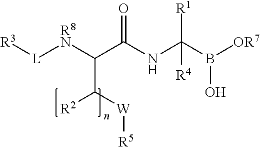
or a pharmaceutically acceptable salt, solvate, stereoisomer or mixture of stereoisomers, tautomer, isotopic form, pharmaceutically active metabolite thereof, or combinations thereof, wherein:
R¹ is selected from the group consisting of: deuterium, C1-C4 alkyl, C1-C4 alkoxyl, C₁-C₄ oxoalkyl, C1-C5 alkyl-alkoxyl, wherein each alkyl, oxoalkyl or alkoxyl is optionally substituted with C3-C6 cycloalkyl, phenyl, phenoxy, or a 5- or 6-membered heteroaryl, wherein said phenyl, phenoxy, or heteroaryl are each optionally substituted with one or more substituent selected from deuterium, halogen, hydroxyl, CN, CO2H, CO2R¹¹, CONR¹¹R¹², NR¹¹R¹², SR¹¹, SO2NR¹¹R¹², C1-C4 alkyl, C1-C4 haloalkyl, C1-C4 alkoxy, phenyl, or a 5- or 6-membered heteroaryl;
n is 1 or 2;
each occurrence of R² is independently selected from H or C1-C4 alkyl;
L is C(O), C(O)O, C(O)NR⁶, S(O)₂, or a bond;
R³ is C₁-C₄ alkyl optionally substituted with one or more substituents each independently selected from the group consisting of deuterium, halogen, cyano, hydroxyl, C₁-C₄ alkoxyl, 5 or 6 membered aryl (e.g. phenyl) or 5 or 6 membered heteroaryl; or
R³ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, oxo, C₁-C₄ alkoxyl, or C₁-C₄ alkyl that is optionally substituted with one to three substituents selected from deuterium, halogen, cyano, hydroxyl, or C₁-C₄ alkoxyl; or
R³ is aryl or heteroaryl having one or more heteroatoms selected from N, O and S, wherein aryl or heteroaryl is optionally substituted with one or more substituents selected from deuterium, halogen, cyano, hydroxyl, OR, CO2H, CO2R¹¹, CONR¹¹R¹², NR¹¹R¹², SR¹¹, SO2NR¹¹R¹², C₁-C₄ alkoxyl, or C₁-C₄ alkyl that is optionally substituted with one to three substituents selected from deuterium, halogen, cyano, hydroxyl, or C₁-C₄ alkoxyl;
W is selected from the group consisting of O, S, S(O), SO₂ and S(O)(NH);
R⁴ is selected from hydrogen, deuterium or C₁-C₂ alkyl;
R⁵ is hydrogen, deuterium, C₁-C₄ alkyl or C₁-C₄ alkoxyl wherein each alkyl or alkoxyl is optionally substituted with one or more substituents each independently selected from the group consisting of deuterium, halogen, cyano, hydroxyl, C₁-C₄ alkoxyl, C₁-C₅ alkyl-alkoxyl or phenyl; or
R⁵ is saturated or unsaturated cycloalkyl or saturated or unsaturated heterocycloalkyl having one or more heteroatoms selected from N, O and S, wherein the cycloalkyl or heterocycloalkyl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is aryl or heteroaryl having one or more heteroatoms selected from N, O and S, wherein aryl, or heteroaryl is optionally substituted with one or more substituents selected from deuterium, halogen, cyano, hydroxyl, C₁-C₄ alkoxyl, or C₁-C₄ alkyl that is optionally substituted with one to three substituents selected from deuterium, halogen, cyano, hydroxyl, or C₁-C₄ alkoxyl and C₁-C₅ alkyl-alkoxyl; or
R⁵ is NR⁹R¹⁰;
R⁶ is hydrogen, deuterium or C₁-C₂ alkyl optionally substituted with one or more of deuterium, halogen, hydroxyl and phenyl, wherein phenyl is optionally substituted with one or more substituent selected from halogen, hydroxyl and C₁-C₂ alkyl;
R⁷ is hydrogen, or R⁷ and R¹, together with the boron atom to which OR⁷ is attached form a 5-membered heteroalkyl ring;
R⁸ is selected from hydrogen, deuterium, or C₁-C₂ alkyl optionally substituted with one or more substituents each independently selected from the group consisting of halogen, hydroxyl, cyano, methoxyl and phenyl; or
R⁸, L and R³ together with the N to which R⁸ and L are attached form a saturated or unsaturated heterocycloalkyl group optionally having one or more additional heteroatoms selected from N, O and S, wherein the heterocycloalkyl is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl, and is optionally fused to an aryl or heteroaryl group which is optionally substituted with one or more substituents selected from halogen, cyano, hydroxyl, C₁-C₄ alkoxyl, or C₁-C₄ alkyl, wherein C₁-C₄ alkyl is optionally substituted with one to three substituents selected from halogen, cyano and C₁-C₄ alkoxyl, or is optionally fused to a cycloalkyl or heterocycloalkyl group which is optionally substituted with one or more substituents selected from halogen, cyano, hydroxyl, C₁-C₄ alkoxyl, oxo, or C₁-C₄ alkyl, wherein C₁-C₄ alkyl is optionally substituted with one to three substituents selected from halogen, cyano and C₁-C₄ alkoxyl;
R⁹ and R¹⁰ are each independently selected from hydrogen or C₁-C₆ alkyl that is optionally substituted with one to three substituents selected from halogen, cyano, or C₁-C₄ alkoxyl; and
R¹¹ and R¹² are each independently selected from hydrogen, deuterium, C1-C4 alkyl; C1-C4 haloalkyl, C1-C5 alkyl-alkoxyl, C3-C7 cycloalkyl, or R¹¹ and R¹² together with the N to which they are attached form 3 to 7 membered heterocyclic ring optionally having one or more additional heteroatoms selected from N, O and S, wherein the C3-C7 cycloalkyl or 3 to 7 membered heterocyclic ring is optionally substituted with one or more substituent selected from deuterium, halogen, hydroxyl, oxo, CN, C1-C4 alkyl, C1-C4 haloalkyl or C1-C4 alkoxyl.

45. The pharmaceutical composition of claim 44, wherein the compound of Formula I is defined according to claim 2.

46. The compound according to claim 1 for use in the treatment of a disease or disorder.

47. The compound for use according to claim 46, wherein the disease or disorder is characterised by mitochondrial dysfunction, such as mitochondrial disorders, including a neurodegenerative disorder, a metabolic disorder and a disease associated with the aging process.

48. The compound for use according to claim 46, wherein the disease or disorder is an oncologic disease or disorder, such as a cancer and/or a proliferative disease or disorder.

49. The compound for use according to claim 48, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

50. The compound for use according to claim 46, wherein the use comprises administering the compound orally; topically; by inhalation; by intranasal administration; by intracerebroventricular; or systemically by intravenous, intraperitoneal, subcutaneous, or intramuscular injection.

51. The compound for use according to claim 46, wherein the use comprises administering to a subject one or more compounds according to claim 1, optionally in combination with one or more additional therapeutic agent.

52. The compound for use according to claim 51, wherein the administering comprises administering the one or more compounds according to claim 1 simultaneously, sequentially or separately from the one or more additional therapeutic agent.

53. A method for treating or preventing a disease or disorder in a subject where inhibition of LONP1 may be beneficial, wherein said method comprises administering to the subject one or more compounds according to claim 1.

54. The method according to claim 53, wherein the disease or disorder is characterized by mitochondrial dysfunction, such as mitochondrial disorders, including a neurodegenerative disorder, a metabolic disorder and a disease associated with the aging process.

55. The method according to claim 53, wherein the disease or disorder is an oncologic disease or disorder, such as a cancer and/or a proliferative disease or disorder.

56. The method according to claim 55, wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma colon cancer, colorectal cancer, cancer of connective tissue, esophageal cancer, embryonal carcinoma, fibrosarcoma, glioblastomas, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), liposarcoma, liver cancer, lung cancer, lymphoid cancers (e.g., Hodgkin's and non-Hodgkin's lymphomas, mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

57. The method according to claim 53, wherein one or more compounds according to claim 1 is administered in combination with one or more additional therapeutic agent.

58. The method according to claim 57, wherein the administering comprises administering the one or more compounds according to claim 1 simultaneously, sequentially or separately from the one or more additional therapeutic agent.

59. The method according to claim 53, wherein the method comprises administering the compound orally; topically; by inhalation; by intranasal administration; by intracerebroventricular; or systemically by intravenous, intraperitoneal, subcutaneous, or intramuscular injection.
